SEQUENCE LISTING (8 sequences)

SEQ ID NO: 1 - DNA, 30 bp, Artificial sequence
  synthetic oligonucleotide
ctaaagcttatgcagctcaagccgatggag

SEQ ID NO: 2 - DNA, 33 bp, Artificial sequence
  synthetic oligonucleotide
ctaagactcgagttaggctgccttgctgagagc

SEQ ID NO: 3 - DNA, 58 bp, Artificial sequence
  synthetic oligonucleotide
ctaaagcttatggactacaaggatgacgacgacaaagatgcagctcaagccgatggag

SEQ ID NO: 4 - DNA, 30 bp, Artificial sequence
  synthetic oligonucleotide
atcctcgagttaggctgccttgacgagagc

SEQ ID NO: 5 - DNA, 58 bp, Artificial sequence
  synthetic oligonucleotide
ctaaagcttatggactacaaggatgacgacgacaaagatggagggtcaacgctggctg

SEQ ID NO: 6 - DNA, 34 bp, Artificial sequence
  synthetic oligonucleotide
atcctcgagctatgctgcagaaagagcaatcgca

SEQ ID NO: 7 - DNA, 44 bp, Artificial sequence
  synthetic oligonucleotide
atcctcgagctatgctgccttagaaagagcaatcgcattaaatc

SEQ ID NO: 8 - protein, 6 aa, Homo sapiens
KTKEGV

(19) United States
(12) Patent Application Publication
(10) Pub. No.: US 2007/0293539 A1
(43) Pub. Date: Dec. 20, 2007

(54) Methods for the treatment of synucleinopathies

(76) Inventors: Peter T. Lansbury, Brookline, MA (US); Zhihua Liu, Salem, MA (US)

Correspondence Address: WOLF GREENFIELD & SACKS, P.C., 600 ATLANTIC AVENUE, BOSTON, MA, 02210-2206, US

(21) Appl. No.: 11/698,983

(22) Filed: Jan. 26, 2007

Related U.S. Application Data
(63) Continuation-in-part of application No. 11/084,715, filed on Mar. 18, 2005.
(63) Continuation-in-part of application No. 11/084,739, filed on Mar. 18, 2005.
(63) Continuation-in-part of application No. 11/084,740, filed on Mar. 18, 2005.
(63) Continuation-in-part of application No. 11/084,695, filed on Mar. 18, 2005.
(60) Provisional application No. 60/555,092, filed on Mar. 18, 2004.
(60) Provisional application No. 60/555,071, filed on Mar. 18, 2004.
(60) Provisional application No. 60/555,019, filed on Mar. 19, 2004.
(60) Provisional application No. 60/555,020, filed on Mar. 18, 2004.
(60) Provisional application No. 60/555,070, filed on Mar. 18, 2004.

(51) Int. Cl. A61K 31/13; A61K 31/445; A61P 25/16

(52) U.S. Cl. 514/319; 514/662

(57) ABSTRACT

Methods are provided of treating synucleinopathies, such as Parkinson's Disease, Diffuse Lewy Body Disease and Multiple System Atrophy, comprising administering to a synucleinopathic subject a farnesyl transferase inhibitor compound.

### RELATED APPLICATIONS

This applications claims priority under 35 U.S.C. § 120 to and is a continuation-in-part of U.S. patent application Ser. No. 11/084,715, filed Mar. 18, 2005, which claims priority under 35 U.S.C. § 119(e) to U.S. provisional patent application Ser. No. 60/555,092, filed Mar. 18, 2004; U.S. Ser. No. 11/084,739, filed Mar. 18, 2005, which claims priority under 35 U.S.C. § 119(e) to U.S. provisional patent application Ser. No. 60/555,071, filed Mar. 18, 2004; U.S. Ser. No. 11/084,740, filed Mar. 18, 2005, which claims priority under 35 U.S.C. § 119(e) to U.S. provisional patent application Ser. No. 60/555,019, filed Mar. 19, 2004, and U.S. Ser. No. 60/555,020, filed Mar. 18, 2004; and U.S. Ser. No. 11/084,695, filed Mar. 18, 2005, which claims priority under 35 U.S.C. § 119(e) to U.S. provisional patent application Ser. No. 60/555,070, filed Mar. 18, 2004; each of which is incorporated herein by reference.

### FEDERALLY SPONSORED RESEARCH

This invention was made with Government support under NIH (National Institute of Health) Grant No. NS38375. The Government may have certain rights to this invention.

### FIELD OF THE INVENTION

The present invention relates to therapeutic approaches to the treatment of synucleinopathies, such as Parkinson's Disease (PD), Diffuse Lewy Body Disease (DLBD) and Multiple System Atrophy (MSA).

### BACKGROUND OF THE INVENTION

Synucleinopathies are a diverse group of neurodegenerative disorders that share a common pathologic lesion containing aggregates of insoluble α-synuclein protein in selectively vulnerable populations of neurons and glia. Certain evidence links the formation of abnormal filamentous aggregates to the onset and progression of clinical symptoms and the degeneration of affected brain regions in neurodegenerative disorders including Parkinson's disease, diffuse Lewy body disease, and multiple system atrophy. The clinical treatments of these diseases include carbidopa-levodopa, anticholinergics and symptomatic medication, although for some synucleinopathies such as diffuse Lewy body disease a specific therapy does not exist. Most Parkinson's subjects that initially respond well to levodopa develop motor fluctuations and a “wearing-off” phenomenon, within five years. Given the severe debilitating nature of these disorders and their prevalence there is a clear need in the art for novel approaches towards treating and managing these diseases.

### SUMMARY OF THE INVENTION

The present invention relates to therapeutic approaches to the treatment of synucleinopathies, such as Parkinson's Disease (PD), Diffuse Lewy Body Disease (DLBD) and Multiple System Atrophy (MSA) by treatment with farnesyl transferase inhibitor compounds.

In one aspect, the invention provides methods for treating a synucleinopathic subject by administering a composition comprising a farnesyl transferase inhibitor compound in a therapeutically effective amount. In some embodiments, the composition includes one or more farnesyl transferase inhibitor compounds and their analogs disclosed herein and incorporated by reference, or one or more stereoisomeric forms or pharmaceutically acceptable acid or base addition salt forms thereof. In one embodiment, the composition includes one or more of farnesyl transferase inhibitor compounds of FIGS. 5-22, or a stereoisomeric form or a pharmaceutically acceptable acid or base addition salt form thereof.

In another aspect, the invention provides methods for treating a synucleinopathic subject by administering both a farnesyl transferase inhibitor compound and a second therapeutic compound in therapeutically effective amounts. The two compounds can be administered as a combination composition comprising both compounds. Alternatively, the two compounds can be administered separately (e.g. as two different compositions) either simultaneously or sequentially as described herein. In some embodiments, the farnesyl transferase inhibitor composition includes one or more farnesyl transferase inhibitor compounds disclosed herein, or one or more stereoisomeric forms or pharmaceutically acceptable acid or base addition salt forms thereof. In one embodiment, a farnesyl transferase inhibitor composition includes one or more farnesyl transferase inhibitor compounds shown in FIGS. 5-22, or a stereoisomeric form or a pharmaceutically acceptable acid or base addition salt form thereof. In some embodiments, the second therapeutic compound includes, but is not limited to dopamine agonists such as Pramipexole, and Memantine, Aricept, and other acetycholinesterase inhibitors.

According to the invention, FTI-277 lowers synuclein level in COS-7 cells and inhibits synuclein toxicity in SH-SY5Y cells. These cells are dopaminergic neuroblastoma cells and can be useful for analyzing Parkinson's Disease pathogenesis.

It should be appreciated that aspects and embodiments of the invention described herein in connection with one farnesyl transferase inhibitor also may be practiced using two or more farnesyl transferase inhibitors (e.g., between 2 and 50, between 2 and 25, between 2 and 10, 2, 3, 4, 5, 6, 7, 8, or 9). Similarly, aspects and embodiments of the invention described herein in connection with one other compound also may be practiced using two or more other compounds (e.g., between 2 and 50, between 2 and 25, between 2 and 10, 2, 3, 4, 5, 6, 7, 8, or 9).

### BRIEF DESCRIPTION OF THE DRAWINGS

FIG. 1 shows that UCH-L1 membrane association is regulated by its farnesylation.

FIG. 2 shows that C220S mutation abolished the inhibitory effect of UCH-L1 WT on α-synuclein degradation.

FIG. 3 shows that farnesyl transferase inhibitor can rescue the α-synuclein toxicity in infected SH-SY5Y cells.

FIG. 4 shows that FTI-277 rescued α-synuclein toxicity in SH-SY5Y cells by reducing the amount of α-synuclein accumulation.

FIG. 5 shows the formula of compounds: R115777, BMS 214662, SCH 66336, SCH 44342, and L778,123.

FIG. 6 shows structures of farnesyl transferase inhibitor compounds.

FIG. 7 shows structures of farnesyl transferase inhibitor compounds.

FIG. 8 shows structures of farnesyl transferase inhibitor compounds.

FIG. 9 shows structures of farnesyl transferase inhibitor compounds.

FIG. 10 shows structures of farnesyl transferase inhibitor compounds.

FIG. 11 shows structures of farnesyl transferase inhibitor compounds.

FIG. 12 shows structures of farnesyl transferase inhibitor compounds.

FIG. 13 shows structures of farnesyl transferase inhibitor compounds.

FIG. 14 shows structures of farnesyl transferase inhibitor compounds.

FIG. 15 shows structures of farnesyl transferase inhibitor compounds.

FIG. 16 shows structures of farnesyl transferase inhibitor compounds.

FIG. 17 shows structures of farnesyl transferase inhibitor compounds.

FIG. 18 shows structures of farnesyl transferase inhibitor compounds.

FIG. 19 shows structures of farnesyl transferase inhibitor compounds.

FIG. 20 shows structures of farnesyl transferase inhibitor compounds.

FIG. 21 shows structures of farnesyl transferase inhibitor compounds.

FIG. 22 shows structures of farnesyl transferase inhibitor compounds.

### DETAILED DESCRIPTION

The invention provides methods, compositions and articles of manufacture for treating synucleinopathic subjects. Methods of the invention are useful to accelerate the degradation of α-synuclein, the accumulation of which is pathogenic in synucleinopathies. The invention provides methods for treating a synucleinopathic subject, including the step of administering to the synucleinopathic subject a therapeutically effective amount of a farnesyl transferase inhibitor compound or a therapeutical preparation, composition, or formulation of the compound such as those described herein, including those in the Claims, Figures, and patents and publications listed herein. In preferred embodiments, the synucleinopathic subject is a human.

In one embodiment, the invention is a method for treating a synucleinopathic subject comprising administering to the synucleinopathic subject a farnesyl transferase inhibitor of the formula:

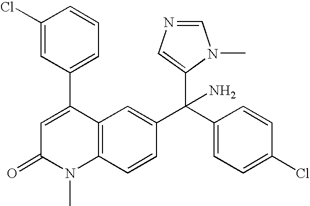

or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor of the formula:

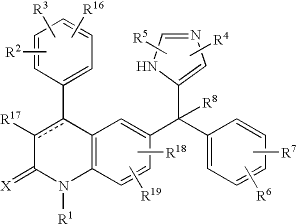

wherein the dotted line represents an optional bond;

X is oxygen or sulfur;

R¹ is hydrogen, C₁₋₁₂ alkyl, Ar¹, Ar²C₁₋₆ alkyl, quinolinylC₁₋₆ alkyl, pyridylC₁₋₆ alkyl, hydroxyC₁₋₆ alkyl, C₁₋₆ alkyloxyC₁₋₆ alkyl, mono- or di(C₁₋₆ alkyl)aminoC₁₋₆ alkyl, aminoC₁₋₆ alkyl, or a radical of formula -Alk¹-C(═O)—R⁹, -Alk¹-S(O)—R⁹ or -Alk¹-S(O)₂—R⁹, wherein Alk¹ is C₁₋₆ alkanediyl,

R⁹ is hydroxy, C₁₋₆ alkyl, C₁₋₆ alkyloxy, amino, C₁₋₈ alkylamino or C₁₋₈ alkylamino substituted with C₁₋₆ alkyloxycarbonyl;

R², R³ and R¹⁶ each independently are hydrogen, hydroxy, halo, cyano, C₁₋₆ alkyl, C₁₋₆ alkyloxy, hydroxyC₁₋₆ alkyloxy, C₁₋₆ alkyloxyC₁₋₆ alkyloxy, aminoC₁₋₆ alkyloxy, mono- or di(C₁₋₆ alkyl)aminoC₁₋₆ alkyloxy, Ar¹, Ar²C₁₋₆ alkyl, Ar² oxy, Ar²C₁₋₆ alkyloxy, hydroxycarbonyl, C₁₋₆ alkyloxycarbonyl, trihalomethyl, trihalomethoxy, C₂₋₆ alkenyl, 4,4-dimethyloxazolyl;

or when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);

R⁴ and R⁵ each independently are hydrogen, halo, Ar¹, C₁₋₆ alkyl, hydroxyC₁₋₆ alkyl, C₁₋₆ alkyloxyC₁₋₆ alkyl, C₁₋₆ alkyloxy, C₁₋₆ alkylthio, amino, hydroxycarbonyl, C₁₋₆ alkyloxycarbonyl, C₁₋₆ alkylS(O)C₁₋₆ alkyl or C₁₋₆ alkylS(O)₂ C₁₋₆ alkyl;

R⁶ and R⁷ each independently are hydrogen, halo, cyano, C₁₋₆ alkyl, C₁₋₆ alkyloxy, Ar² oxy, trihalomethyl, C₁₋₆ alkylthio, di(C₁₋₆ alkyl)amino, or

when on adjacent positions R⁶ and R⁷ taken together may form a bivalent radical of formula
—O—CH₂—O— (c-1), or
—CH═CH—CH═CH— (c-2);

R⁸ is hydrogen, C₁₋₆ alkyl, cyano, hydroxycarbonyl, C₁₋₆ alkyloxycarbonyl, C₁₋₆ alkylcarbonylC₁₋₆ alkyl, cyanoC₁₋₆ alkyl, C₁₋₆ alkyloxycarbonylC₁₋₆ alkyl, carboxyC₁₋₆ alkyl, hydroxyC₁₋₆ alkyl, aminoC₁₋₆ alkyl, mono- or di(C₁₋₆ alkyl)aminoC₁₋₆ alkyl, imidazolyl, haloC₁₋₆ alkyl, C₁₋₆ alkyloxyC₁₋₆ alkyl, aminocarbonylC₁₋₆ alkyl, or a radical of formula
—O—R¹⁰ (b-1),
—S—R¹⁰ (b-2),
—N—R¹¹R¹² (b-3),
wherein
R¹⁰ is hydrogen, C₁₋₆ alkyl, C₁₋₆ alkylcarbonyl, Ar¹, Ar²C₁₋₆ alkyl, C₁₋₆ alkyloxycarbonylC₁₋₆ alkyl, a radical or formula -Alk²-OR¹³ or -Alk²-NR¹⁴R¹⁵;
R¹¹ is hydrogen, C₁₋₁₂ alkyl, Ar¹ or Ar²C₁₋₆ alkyl;
R¹² is hydrogen, C₁₋₆ alkyl, C₁₋₁₆ alkylcarbonyl, C₁₋₆alkyloxycarbonyl, C₁₋₆ alkylaminocarbonyl, Ar¹, Ar²C₁₋₆ alkyl, C₁₋₆ alkylcarbonylC₁₋₆ alkyl, a natural amino acid, Ar¹ carbonyl, Ar²C₁₋₆ alkylcarbonyl, aminocarbonylcarbonyl, C₁₋₆ alkyloxyC₁₋₆ alkylcarbonyl, hydroxy, C₁₋₆ alkyloxy, aminocarbonyl, di(C₁₋₆ alkyl)aminoC₁₋₆ alkylcarbonyl, amino, C₁₋₆ alkylamino, C₁₋₆ alkylcarbonylamino, or a radical of formula -Alk²-OR¹³ or -Alk²-NR¹⁴R¹⁵;
wherein
Alk² is C₁₋₆ alkanediyl;
R¹³ is hydrogen, C₁₋₆ alkyl, C₁₋₆ alkylcarbonyl, hydroxyC₁₋₆ alkyl, Ar¹ or Ar²C₁₋₆ alkyl;
R¹⁴ is hydrogen, C₁₋₆ alkyl, Ar¹ or Ar²C₁₋₆ alkyl;
R¹⁵ is hydrogen, C₁₋₆ alkyl, C₁₋₆ alkylcarbonyl, Ar¹ or Ar²C₁₋₆ alkyl;

R¹⁷ is hydrogen, halo, cyano, C₁₋₆ alkyl, C₁₋₆ alkyloxycarbonyl, Ar¹;

R¹⁸ is hydrogen, C₁₋₆ alkyl, C₁₋₆ alkyloxy or halo;

R¹⁹ is hydrogen or C₁₋₆ alkyl;

Ar¹ is phenyl or phenyl substituted with C₁₋₆ alkyl, hydroxy, amino, C₁₋₆ alkyloxy or halo; and

Ar² is phenyl or phenyl substituted with C₁₋₆ alkyl, hydroxy, amino, C₁₋₆ alkyloxy or halo;

or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor of the formula:

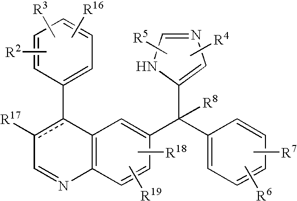

wherein R², R³ and R¹⁶ each independently are hydrogen, hydroxy, halo, cyano, C₁₋₆ alkyl, C₁₋₆ alkyloxy, hydroxyC₁₋₆ alkyloxy, C₁₋₆ alkyloxyC₁₋₆ alkyloxy, aminoC₁₋₆ alkyloxy, mono- or di(C₁₋₆ alkyl)aminoC₁₋₆ alkyloxy, Ar¹, Ar²C₁₋₆ alkyl, Ar² oxy, Ar²C₁₋₆ alkyloxy, hydroxycarbonyl, C₁₋₆ alkyloxycarbonyl, trihalomethyl, trihalomethoxy, C₂₋₆ alkenyl, 4,4-dimethyloxazolyl; or

when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);

R⁴ and R⁵ each independently are hydrogen, halo, Ar¹, C₁₋₆ alkyl, hydroxyC₁₋₆ alkyl, C₁₋₆ alkyloxyC₁₋₆ alkyl, C₁₋₆ alkyloxy, C₁₋₆ alkylthio, amino, hydroxycarbonyl, C₁₋₆ alkyloxycarbonyl, C₁₋₆ alkylS(O)C₁₋₆ alkyl or C₁₋₆ alkylS(O)₂ C₁₋₆ alkyl;

R⁶ and R⁷ each independently are hydrogen, halo, cyano, C₁₋₆ alkyl, C₁₋₆ alkyloxy, Ar² oxy, trihalomethyl, C₁₋₆ alkylthio, di(C₁₋₆ alkyl) amino, or

when on adjacent positions R⁶ and R⁷ taken together may form a bivalent radical of formula
—O—CH₂—O— (c-1), or
—CH═CH—CH═CH— (c-2);

R⁸ is hydrogen, C₁₋₆ alkyl, cyano, hydroxycarbonyl, C₁₋₆ alkyloxycarbonyl, C₁₋₆ alkylcarbonylC₁₋₆ alkyl, cyanoC₁₋₆ alkyl, C₁₋₆ alkyloxycarbonylC₁₋₆ alkyl, carboxyC₁₋₆ alkyl, hydroxyC₁₋₆ alkyl, aminoC₁₋₆ alkyl, mono- or di(C₁₋₆ alkyl)aminoC₁₋₆ alkyl, imidazolyl, haloC₁₋₆ alkyl, C₁₋₆ alkyloxyC₁₋₆ alkyl, aminocarbonylC₁₋₆ alkyl, or a radical of formula
—O—R¹⁰ (b-1),
—S—R¹⁰ (b-2),
—N—R¹¹R¹² (b-3),
wherein
R¹⁰ is hydrogen, C₁₋₆ alkyl, C₁₋₆ alkylcarbonyl, Ar¹, Ar²C₁₋₆ alkyl, C₁₋₆ alkyloxycarbonylC₁₋₆ alkyl, a radical or formula -Alk²-OR¹³ or -Alk²-NR¹⁴R¹⁵;
R¹¹ is hydrogen, C₁₋₁₂ alkyl, Ar¹ or Ar²C₁₋₆ alkyl;
R¹² is hydrogen, C₁₋₆ alkyl, C₁₋₆ alkylcarbonyl, C₁₋₆ alkyloxycarbonyl, C₁₋₆ alkylaminocarbonyl, Ar¹, Ar²C₁₋₆ alkyl, C₁₋₆ alkylcarbonylC₁₋₆ alkyl, a natural amino acid, Ar¹ carbonyl, Ar²C₁₋₆ alkylcarbonyl, aminocarbonylcarbonyl, C₁₋₆ alkyloxyC₁₋₆ alkylcarbonyl, hydroxy, C₁₋₆ alkyloxy, aminocarbonyl, di(C₁₋₆ alkyl) aminoC₁₋₆ alkylcarbonyl, amino, C₁₋₆ alkylamino, C₁₋₆ alkylcarbonylamino, or a radical of formula -Alk²-OR¹³ or -Alk²-NR¹⁴R¹⁵;
wherein Alk² is C₁₋₆ alkanediyl;
R¹³ is hydrogen, C₁₋₆ alkyl, C₁₋₆ alkylcarbonyl, hydroxyC₁₋₆ alkyl, Ar¹ or Ar²C₁₋₆ alkyl;
R¹⁴ is hydrogen, C₁₋₆ alkyl, Ar¹ or Ar²C₁₋₆ alkyl;
R¹⁵ is hydrogen, C₁₋₆ alkyl, C₁₋₆ alkylcarbonyl, Ar¹ or Ar²C₁₋₆ alkyl;
R¹⁷ is hydrogen, halo, cyano, C₁₋₆ alkyl, C₁₋₆ alkyloxycarbonyl, Ar¹;
R¹⁸ is hydrogen, C₁₋₆ alkyl, C₁₋₆ alkyloxy or halo;
R¹⁹ is hydrogen or C₁₋₆ alkyl;

a stereoisomeric form or a pharmaceutically acceptable acid or base addition salt form thereof, at a therapeutically effective dose and frequency.

In another embodiment the invention is a method for treating a synucleinopathic subject comprising administering to the synucleinopathic subject a farnesyl transferase inhibitor with the formula:

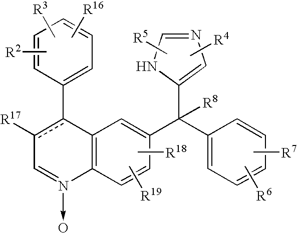

wherein R², R³ and R¹⁶ each independently are hydrogen, hydroxy, halo, cyano, C₁₋₆ alkyl, C₁₋₆ alkyloxy, hydroxyC₁₋₆ alkyloxy, C₁₋₆ alkyloxyC₁₋₆ alkyloxy, aminoC₁₋₆ alkyloxy, mono- or di(C₁₋₆ alkyl)aminoC₁₋₆ alkyloxy, Ar¹, Ar²C₁₋₆ alkyl, Ar² oxy, Ar²C₁₋₆ alkyloxy, hydroxycarbonyl, C₁₋₆ alkyloxycarbonyl, trihalomethyl, trihalomethoxy, C₂₋₆ alkenyl, 4,4-dimethyloxazolyl; or

when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);

R⁴ and R⁵ each independently are hydrogen, halo, Ar¹, C₁₋₆ alkyl, hydroxyC₁₋₆ alkyl, C₁₋₆ alkyloxyC₁₋₆ alkyl, C₁₋₆ alkyloxy, C₁₋₆ alkylthio, amino, hydroxycarbonyl, C₁₋₆ alkyloxycarbonyl, C₁₋₆ alkylS(O)C₁₋₆ alkyl or C₁₋₆ alkylS(O)₂ C₁₋₆ alkyl;

R⁶ and R⁷ each independently are hydrogen, halo, cyano, C₁₋₆ alkyl, C₁₋₆ alkyloxy, Ar² oxy, trihalomethyl, C₁₋₆ alkylthio, di(C₁₋₆ alkyl) amino, or

when on adjacent positions R⁶ and R⁷ taken together may form a bivalent radical of formula
—O—CH₂—O— (c-1), or
—CH═CH—CH═CH— (c-2);

R⁸ is hydrogen, C₁₋₆ alkyl, cyano, hydroxycarbonyl, C₁₋₆ alkyloxycarbonyl, C₁₋₆ alkylcarbonylC₁₋₆ alkyl, cyanoC₁₋₆ alkyl, C₁₋₆ alkyloxycarbonylC₁₋₆ alkyl, carboxyC₁₋₆ alkyl, hydroxyC₁₋₆ alkyl, aminoC₁₋₆ alkyl, mono- or di(C₁₋₆ alkyl)aminoC₁₋₆ alkyl, imidazolyl, haloC₁₋₆ alkyl, C₁₋₆ alkyloxyC₁₋₆ alkyl, aminocarbonylC₁₋₆ alkyl, or a radical of formula
—O—R¹⁰ (b-1),
—S—R¹⁰ (b-2),
—N—R¹¹R¹² (b-3),
wherein
R¹⁰ is hydrogen, C₁₋₆ alkyl, C₁₋₆ alkylcarbonyl, Ar¹, Ar²C₁₋₆ alkyl, C₁₋₆ alkyloxycarbonylC₁₋₆ alkyl, a radical or formula -Alk²-OR¹³ or -Alk²-NR¹⁴R¹⁵;
R¹¹ is hydrogen, C₁₋₁₂ alkyl, Ar¹ or Ar²C₁₋₆ alkyl;
R¹² is hydrogen, C₁₋₆ alkyl, C₁₋₁₆ alkylcarbonyl, C₁₋₆ alkyloxycarbonyl, C₁₋₆ alkylaminocarbonyl, Ar¹, Ar²C₁₋₆ alkyl, C₁₋₆ alkylcarbonylC₁₋₆ alkyl, a natural amino acid, Ar¹ carbonyl, Ar²C₁₋₆ alkylcarbonyl, aminocarbonylcarbonyl, C₁₋₆ alkyloxyC₁₋₆ alkylcarbonyl, hydroxy, C₁₋₆ alkyloxy, aminocarbonyl, di(C₁₋₆ alkyl)aminoC₁₋₆ alkylcarbonyl, amino, C₁₋₆ alkylamino, C₁₋₆ alkylcarbonylamino, or a radical of formula -Alk²-OR¹³ or -Alk²-NR¹⁴R¹⁵;
wherein
Alk² is C₁₋₆ alkanediyl;
R¹³ is hydrogen, C₁₋₆ alkyl, C₁₋₆ alkylcarbonyl, hydroxyC₁₋₆ alkyl, Ar¹ or Ar²C₁₋₆ alkyl;
R¹⁴ is hydrogen, C₁₋₆ alkyl, Ar¹ or Ar²C₁₋₆ alkyl;
R¹⁵ is hydrogen, C₁₋₆ alkyl, C₁₋₆ alkylcarbonyl, Ar¹ or Ar²C₁₋₆ alkyl;

R¹⁷ is hydrogen, halo, cyano, C₁₋₆ alkyl, C₁₋₆ alkyloxycarbonyl, Ar¹;

R¹⁸ is hydrogen, C₁₋₆ alkyl, C₁₋₆ alkyloxy or halo;

R¹⁹ is hydrogen or C₁₋₆ alkyl;

or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor of the formula:

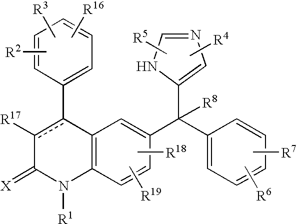

a stereoisomeric form thereof, a pharmaceutically acceptable acid or base addition salt thereof,

wherein the dotted line represents an optional bond;

X is oxygen or sulfur;

R¹ is hydrogen, C₁₋₁₂ alkyl, Ar¹, Ar²C₁₋₆ alkyl, quinolinylC₁₋₆-alkyl, pyridylC₁₋₆ alkyl, hydroxyC₁₋₆ alkyl, C₁₋₆ alkyloxyC₁₋₆ alkyl, mono- or di(C₁₋₆ alkyl)aminoC₁₋₆ alkyl, aminoC₁₋₆ alkyl, or a radical of formula -Alk¹-C(═O)—R⁹, -Alk¹-S(O)—R⁹ or -Alk¹-S(O)₂—R⁹, wherein Alk¹ is C₁₋₆ alkanediyl,

R⁹ is hydroxy, C₁₋₆ alkyl, C₁₋₆ alkyloxy, amino, C₁₋₈ alkylamino or C₁₋₈ alkylamino substituted with C₁₋₆ alkyloxycarbonyl;

R², R³ and R¹⁶ each independently are hydrogen, hydroxy, halo, cyano, C₁₋₆ alkyl, C₁₋₆ alkyloxy, hydroxyC₁₋₆ alkyloxy, C₁₋₆ alkyloxyC₁₋₆ alkyloxy, aminoC₁₋₆ alkyloxy, mono- or di(C₁₋₆ alkyl)aminoC₁₋₆ alkyloxy, Ar¹, Ar²C₁₋₆ alkyl, Ar² oxy, Ar²C₁₋₆ alkyloxy, hydroxycarbonyl, C₁₋₆ alkyloxycarbonyl, trihalomethyl, trihalomethoxy, C₂₋₆ alkenyl, 4,4-dimethyloxazolyl; or

when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);

R⁴ is hydrogen or C₁₋₆ alkyl;

R⁵ is hydrogen;

R⁶ and R⁷ each independently are hydrogen, halo, cyano, C₁₋₆ alkyl, C₁₋₆ alkyloxy, Ar² oxy, trihalomethyl, C₁₋₆ alkylthio, di(C₁₋₆ alkyl)amino, or

when on adjacent positions R⁶ and R⁷ taken together may form a bivalent radical of formula:
—O—CH₂—O— (c-1), or
—CH═CH—CH═CH— (c-2);

R⁸ is hydrogen, C₁₋₆ alkyl, cyano, hydroxycarbonyl, C₁₋₆ alkyloxycarbonyl, C₁₋₆ alkylcarbonylC₁₋₆ alkyl, cyanoC₁₋₆ alkyl, C₁₋₆ alkyloxycarbonylC₁₋₆ alkyl, carboxyC₁₋₆ alkyl, hydroxyC₁₋₆ alkyl, aminoC₁₋₆ alkyl, mono- or di(C₁₋₆ alkyl)aminoC₁₋₆ alkyl, imidazolyl, haloC₁₋₆ alkyl, C₁₋₆ alkyloxyC₁₋₆ alkyl, aminocarbonylC₁₋₆ alkyl, or a radical of formula:
—O—R¹⁰ (b-1),
—S—R¹⁰ (b-2),
—N—R¹¹R¹² (b-3),

wherein R¹⁰ is hydrogen, C₁₋₆ alkyl, C₁₋₆ alkylcarbonyl, Ar¹, Ar²C₁₋₆ alkyl, C₁₋₆ alkyloxycarbonylC₁₋₆ alkyl, a radical or formula -Alk²-OR¹³ or -Alk²-NR¹⁴R¹⁵;

R¹¹ is hydrogen, C₁₋₁₂ alkyl, Ar¹ or Ar²C₁₋₆ alkyl;

R¹² is hydrogen, C₁₋₆ alkyl, C₁₋₆ alkylcarbonyl, C₁₋₆ alkyloxycarbonyl, C₁₋₆ alkylaminocarbonyl, Ar¹, Ar²C₁₋₆ alkyl, C₁₋₆ alkylcarbonylC₁₋₆ alkyl, a natural amino acid, Ar¹ carbonyl, Ar²C₁₋₆ alkylcarbonyl, aminocarbonylcarbonyl, C₁₋₆ alkyloxyC₁₋₆ alkylcarbonyl, hydroxy, C₁₋₆ alkyloxy, aminocarbonyl, di(C₁₋₆ alkyl) aminoC₁₋₆ alkylcarbonyl, amino, C₁₋₆ alkylamino, C₁₋₆ alkylcarbonylamino, or a radical of formula -Alk²-OR¹³ or -Alk²-NR¹⁴R¹⁵;

wherein Alk² is C₁₋₆ alkanediyl;

R¹³ is hydrogen, C₁₋₆ alkyl, C₁₋₆ alkylcarbonyl, hydroxyC₁₋₆ alkyl, Ar¹ or Ar²C₁₋₆ alkyl;

R¹⁴ is hydrogen, C₁₋₆ alkyl, Ar¹ or Ar²C₁₋₆ alkyl;

R¹⁵ is hydrogen, C₁₋₆ alkyl, C₁₋₆ alkylcarbonyl, Ar¹ or Ar²C₁₋₆ alkyl;

R¹⁷ is hydrogen, halo, cyano, C₁₋₆ alkyl, C₁₋₆ alkyloxycarbonyl, Ar¹;

R¹⁸ is hydrogen, C₁₋₆ alkyl, C₁₋₆ alkyloxy or halo;

R¹⁹ is hydrogen or C₁₋₆ alkyl;

Ar¹ is phenyl or phenyl substituted with C₁₋₆ alkyl, hydroxy, amino, C₁₋₆ alkyloxy or halo; and

Ar² is phenyl or phenyl substituted with C₁₋₆ alkyl, hydroxy, amino, C₁₋₆ alkyloxy or halo;

or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor compound that is an enantiomer of 6-(amino(4-chlorophenyl)(1-methyl-1H-imidazol-5-yl)methyl)-4-(3-chlorophenyl)-1-methyl-2(1H)-quinolinone having an α_{D}²⁰ value of +22.86° (c=49.22 mg/5 ml, methanol) or a pharmaceutically acceptable acid addition salt thereof, at a therapeutically acceptable dose and frequency.

In another embodiment the invention is a method for treating a synucleinopathic subject comprising administering to the synucleinopathic subject a farnesyl transferase inhibitor of the formula:

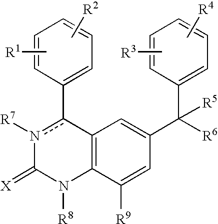

wherein

the dotted line represents an optional bond;

X is oxygen or sulfur;

R¹ and R² each independently are hydrogen, hydroxy, halo, cyano, C₁₋₆ alkyl, trihalomethyl, trihalomethoxy, C₂₋₆ alkenyl, C₁₋₆ alkyloxy, hydroxyC₁₋₆ alkyloxy, C₁₋₆ alkyloxyC₁₋₆ alkyloxy, C₁₋₆ alkyloxycarbonyl, aminoC₁₋₆ alkyloxy, mono- or di(C₁₋₆ alkyl)aminoC₁₋₆ alkyloxy, Ar¹, Ar¹ C₁₋₆ alkyl, Ar¹ oxy, Ar¹ C₁₋₆ alkyloxy;

R³ and R⁴ each independently are hydrogen, halo, cyano, C₁₋₆ alkyl, C₁₋₆ alkyloxy, Ar¹ oxy, C₁₋₆ alkylthio, di(C₁₋₆ alkyl)amino, trihalomethyl or trihalomethoxy;

R⁵ is hydrogen, halo, C₁₋₆ alkyl, cyano, haloC₁₋₆ alkyl, hydroxyC₁₋₆ alkyl, cyanoC₁₋₆ alkyl, aminoC₁₋₆ alkyl, C₁₋₆ alkyloxyC₁₋₆ alkyl, C₁₋₆ alkylthioC₁₋₆ alkyl, aminocarbonylC₁₋₆ alkyl, C₁₋₆alkyloxycarbonylC₁₋₆ alkyl, C₁₋₆ alkylcarbonylC₁₋₆ alkyl, C₁₋₆ alkyloxycarbonyl, mono- or di(C₁₋₆ alkyl)aminoC₁₋₆ alkyl, Ar¹, Ar¹C₁₋₆ alkyloxyC₁₋₆ alkyl; or a radical of formula:
—O—R¹⁰ (a-1),
—S—R¹⁰ (a-2),
—N—R¹¹R¹² (a-3)
wherein
R¹⁰ is hydrogen, C₁₋₆ alkyl, C₁₋₆ alkylcarbonyl, Ar¹, Ar¹ C₁₋₆ alkyl, C₁₋₆ alkyloxycarbonylC₁₋₆ alkyl, or a radical of formula -Alk-OR¹³ or -Alk-NR¹⁴R¹⁵;
R¹¹ is hydrogen, C₁₋₆ alkyl, Ar¹ or Ar¹ C₁₋₆ alkyl;
R¹² is hydrogen, C₁₋₆ alkyl, C₁₋₆ alkylcarbonyl, C₁₋₆ alkyloxycarbonyl, C₁₋₆ alkylaminocarbonyl, Ar¹, Ar¹ C₁₋₆ alkyl, C₁₋₆ alkylcarbonyl-C₁₋₆ alkyl, Ar¹ carbonyl, Ar¹ C₁₋₆ alkylcarbonyl, aminocarbonylcarbonyl, C₁₋₆ alkyloxyC₁₋₆ alkylcarbonyl, hydroxy, C₁₋₆ alkyloxy, aminocarbonyl, di(C₁₋₆ alkyl)aminoC₁₋₆ alkylcarbonyl, amino, C₁₋₆ alkylamino, C₁₋₆ alkylcarbonylamino, or a radical or formula -Alk-OR¹³ or -Alk-NR¹⁴R¹⁵; wherein Alk is C₁₋₆ alkanediyl;
R¹³ is hydrogen, C₁₋₆ alkyl, C₁₋₆ alkylcarbonyl, hydroxyC₁₋₆ alkyl, Ar¹ or Ar¹ C₁₋₆ alkyl;
R¹⁴ is hydrogen, C₁₋₆ alkyl, Ar¹ or Ar¹ C₁₋₆ alkyl;
R¹⁵ is hydrogen, C₁₋₆ alkyl, C₁₋₆ alkylcarbonyl, Ar¹ or Ar¹ C₁₋₆ alkyl;
R⁶ is a radical of formula: 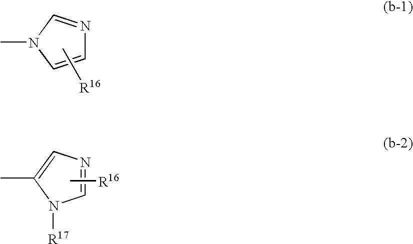
wherein
R¹⁶ is hydrogen, halo, Ar¹, C₁₋₆ alkyl, hydroxyC₁₋₆ alkyl, C₁₋₆ alkyloxyC₁₋₆ alkyl, C₁₋₆ alkyloxy, C₁₋₆ alkylthio, amino, C₁₋₆ alkyloxycarbonyl, C₁₋₆ alkylthioC₁₋₆ alkyl, C₁₋₆ alkylS(O)C₁₋₆ alkyl or C₁₋₆ alkylS(O)₂ C₁₋₆ alkyl;
R¹⁷ is hydrogen, C₁₋₆ alkyl or di(C₁₋₄ alkyl)aminosulfonyl;
R⁷ is hydrogen or C₁₋₆ alkyl provided that the dotted line does not represent a bond;
R⁸ is hydrogen, C₁₋₆ alkyl or Ar²CH₂ or Het¹CH₂;
R⁹ is hydrogen, C₁₋₆ alkyl, C₁₋₆ alkyloxy or halo; or
R⁸ and R⁹ taken together to form a bivalent radical of formula
—CH═CH— (c-1)
—CH₂—CH₂— (c-2)
—CH₂—CH₂—CH₂— (c-3)
—CH₂—O— (c-4), or
—CH₂—CH₂—O— (c-5)
Ar¹ is phenyl; or phenyl substituted with 1 or 2 substituents each independently selected from halo, C₁₋₆ alkyl, C₁₋₆ alkyloxy or trifluoromethyl;
Ar² is phenyl; or phenyl substituted with 1 or 2 substituents each independently selected from halo, C₁₋₆ alkyl, C₁₋₆ alkyloxy or trifluoromethyl; and
Het¹ is pyridinyl; pyridinyl substituted with 1 or 2 substituents each independently selected from halo, C₁₋₆ alkyl, C₁₋₆ alkyloxy or trifluoromethyl;

or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor of the formula

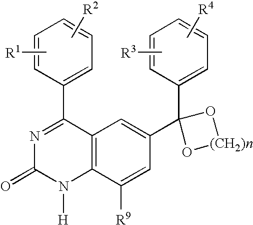

wherein n is 2 or 3 and R¹, R², R³, R⁴ and R⁹ are as defined previously,

or a stereoisomeric form or a pharmaceutically acceptable acid or base addition salt form thereof, at a therapeutically effective dose and frequency.

In another embodiment the invention is a method for treating a synucleinopathic subject comprising administering to the synucleinopathic subject a farnesyl transferase inhibitor of the formula:

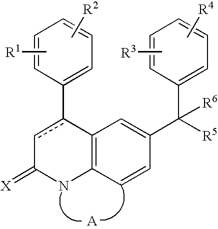

wherein

the dotted line represents an optional bond;

X is oxygen or sulfur;

-A- is a bivalent radical of formula:
—CH═CH— (a-1),
—CH₂—CH₂— (a-2),
—CH₂—CH₂—CH₂— (a-3),
—CH₂—O— (a-4),
—CH₂—CH₂—O— (a-5),
—CH₂—S— (a-6),
—CH₂—CH₂—S— (a-7),
—CH═N— (a-8),
—N═N— (a-9), or
—CO—NH— (a-10);
R¹ and R² each independently are hydrogen, hydroxy, halo, cyano, C₁₋₆ alkyl, trihalomethyl, trihalomethoxy, C₂₋₆ alkenyl, C₁₋₆ alkyloxy, hydroxy C₁₋₆ alkyloxy, C₁₋₆ alkyloxyC₁₋₆ alkyloxy, C₁₋₆ alkyloxycarbonyl, aminoC₁₋₆ alkyloxy, mono- or di(C₁₋₆ alkyl)aminoC₁₋₆ alkyloxy, Ar², Ar²-C₁₋₆ alkyl, Ar²-oxy, Ar²-C₁₋₆ alkyloxy; or
when on adjacent positions R¹ and R² taken together may form a bivalent radical of formula:
—O—CH₂—O— (b-1),
—O—CH₂—CH₂—O— (b-2),
—O—CH═CH— (b-3),
—O—CH₂—CH₂— (b-4),
—O—CH₂—CH₂—CH₂— (b-5), or
—CH═CH—CH═CH— (b-6);

R³ and R⁴ each independently are hydrogen, halo, cyano, C₁₋₆alkyl, C₁₋₆alkoxy, Ar³-oxy, C₁₋₆alkylthio, di(C₁₋₆alkyl)amino, trihalomethyl, trihalomethoxy, or when on adjecent positions R³ and R⁴ taken together may form a bivalent radical of formula:
—O—CH₂—O— (c-1),
—O—CH₂—CH₂—O— (c-2), or
—CH═CH—CH═CH— (c-3);

R⁵ is a radical of formula: 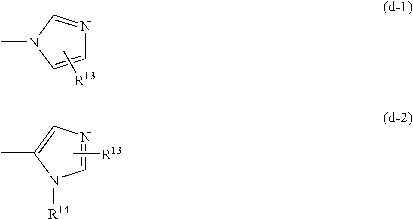
wherein R¹³ is hydrogen, halo, Ar⁴, C₁₋₆ alkyl, hydroxyC₁₋₆ alkyl, C₁₋₆ alkyloxyC₁₋₆ alkyl, C₁₋₆ alkyloxy, C₁₋₆ alkylthio, amino, C₁₋₆ alkyloxycarbonyl, C₁₋₆ alkylS(O)C₁₋₆ alkyl or C₁₋₆ alkylS(O)₂ C₁₋₆ alkyl; R¹⁴ is hydrogen, C₁₋₆ alkyl or di(C₁₋₄ alkyl)aminosulfonyl;

R⁶ is hydrogen, hydroxy, halo, C₁₋₆ alkyl, cyano, haloC₁₋₆ alkyl, hydroxyC₁₋₆ alkyl, cyanoC₁₋₆ alkyl, aminoC₁₋₆ alkyl, C₁₋₆ alkyloxyC₁₋₆ alkyl, C₁₋₆ alkylthioC₁₋₆ alkyl, aminocarbonyl-C₁₋₆ alkyl, C₁₋₆ alkyloxycarbonylC₁₋₆ alkyl, C₁₋₆ alkylcarbonylC₁₋₆ alkyl, C₁₋₆ alkyloxycarbonyl, mono- or di(C₁₋₆ alkyl)aminoC₁₋₆ alkyl, Ar⁵, Ar⁵-C₁₋₆ alkyloxyC₁₋₆ alkyl; or a radical of formula
—O—R⁷ (e-1),
—S—R⁷ (e-2), or
—N—R⁸R⁹ (e-3);

wherein

R⁷ is hydrogen, C₁₋₆ alkyl, C₁₋₆ alkylcarbonyl, Ar⁶, Ar⁶-C₁₋₆ alkyl, C₁₋₆ alkyloxycarbonylC₁₋₆ alkyl, or a radical of formula -Alk-OR¹⁰ or -Alk-NR¹¹R¹²;

R⁸ is hydrogen, C₁₋₆ alkyl, Ar⁷ or Ar⁷-C₁₋₆ alkyl;

R⁹ is hydrogen, C₁₋₆ alkyl, C₁₋₆ alkylcarbonyl, C₁₋₆ alkyloxycarbonyl, C₁₋₆ alkylaminocarbonyl, Ar⁸, Ar⁸-C₁₋₆ alkyl, C₁₋₆ alkylcarbonyl-C₁₋₆ alkyl, Ar⁸-carbonyl, Ar⁸-C₁₋₆ alkylcarbonyl, aminocarbonylcarbonyl, C₁₋₆ alkyloxyC₁₋₆ alkylcarbonyl, hydroxy, C₁₋₆ alkyloxy, aminocarbonyl, di(C₁₋₆ alkyl)aminoC₁₋₆ alkylcarbonyl, amino, C₁₋₆ alkylamino, C₁₋₆ alkylcarbonylamino, or a radical or formula -Alk-OR¹⁰ or -Alk-NR¹¹R¹²;

wherein Alk is C₁₋₆ alkanediyl;

R¹⁰ is hydrogen, C₁₋₆ alkyl, C₁₋₆ alkylcarbonyl, hydroxyC₁₋₆ alkyl, Ar⁹ or Ar⁹-C₁₋₆ alkyl;

R¹¹ is hydrogen, C₁₋₆ alkyl, C₁₋₆ alkylcarbonyl, Ar¹⁰ or Ar¹⁰-C₁₋₆ alkyl;

R¹² is hydrogen, C₁₋₆ alkyl, Ar¹¹ or Ar¹¹-C₁₋₆ alkyl; and

Ar¹ to Ar¹ are each independently selected from phenyl; or phenyl substituted with halo, C₁₋₆ alkyl, C₁₋₆ alkyloxy or trifluoromethyl,

or a stereoisomeric form or a pharmaceutically acceptable acid or base addition salt form thereof, at a therapeutically effective dose and frequency.

In one embodiment, the dotted line represents an optional bond;

X is O or S;

R¹ and R² are each independently selected from hydrogen, halo, C₁₋₆ alkyl, C₁₋₆ alkyloxy, trihalomethyl or trihalomethoxy;

R³ and R⁴ are each independently selected from hydrogen, halo, C₁₋₆ alkyl, C¹⁻⁶ alkyloxy, trihalomethyl or trihalomethoxy;

R⁵ a radical of formula (d-1) wherein R¹³ is hydrogen or R⁵ is a radical of formula (d-2) wherein R¹³ is hydrogen or C₁₋₆ alkyl and R¹⁴ is hydrogen or C₁₋₆ alkyl; and

R⁶ is hydrogen, hydroxy, haloC₁₋₆ alkyl, hydroxyC₁₋₆ alkyl, cyanoC₁₋₆ alkyl, C₁₋₆ alkyloxycarbonylC₁₋₆ alkyl, or a radical of formula —NR⁸R⁹ wherein R⁸ is hydrogen or C₁₋₆ alkyl and R⁹ is hydrogen, C₁₋₆ alkyl, C₁₋₆ alkyloxy or C₁₋₆ alkyloxyC₁₋₆ alkylcarbonyl.

In another embodiment, the invention is a method for treating a synucleinopathic subject comprising administering to the synucleinopathic subject a farnesyl transferase inhibitor of the formula: 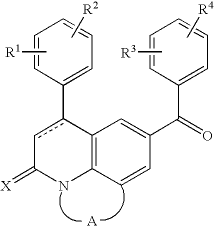
wherein the dotted line represents an optional bond; wherein X, -A-, R¹, R², R³ and R⁴ are as defined previously;

or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor of the formula:

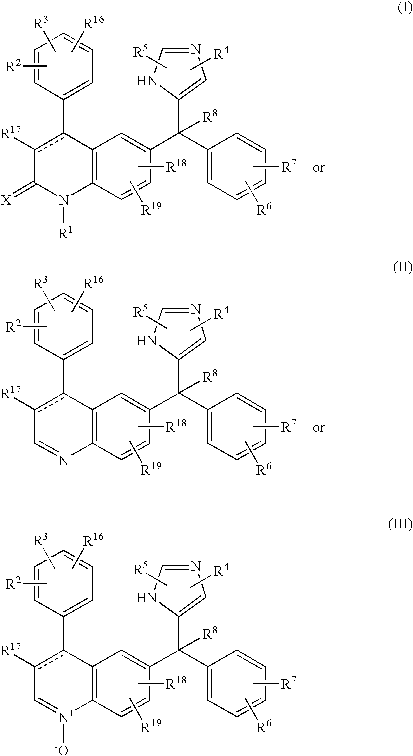

wherein

the dotted line represents an optional bond;

X is oxygen or sulfur;

R¹ is hydrogen, C₁₋₁₂ alkyl, Ar¹, Ar²C₁₋₆ alkyl, quinolinylC₁₋₆ alkyl, pyridylC₁₋₆ alkyl, hydroxyC₁₋₆ alkyl, C₁₋₆ alkyloxyC₁₋₆ alkyl, mono- or di(C₁₋₆ alkyl) aminoC₁₋₆ alkyl, aminoC₁₋₆ alkyl, or a radical of formula -Alk¹-C(═O)—R⁹, -Alk¹-S(O)—R⁹ or -Alk¹-S(O)₂—R⁹,

wherein

Alk¹ is C₁₋₆ alkanediyl,

R⁹ is hydroxy, C₁₋₆ alkyl, C₁₋₆ alkyloxy, amino, C₁₋₈ alkylamino or C₁₋₈ alkylamino substituted with C₁₋₆ alkyloxycarbonyl;

R², R³ and R¹⁶ each independently are hydrogen, hydroxy, halo, cyano, C₁₋₆ alkyl, C₁₋₆ alkyloxy, hydroxyC₁₋₆ alkyloxy, C₁₋₆ alkyloxyC₁₋₆ alkyloxy, aminoC₁₋₆ alkyloxy, mono- or di(C₁₋₆ alkyl)aminoC₁₋₆ alkyloxy, Ar¹, Ar²C₁₋₆ alkyl, Ar² oxy, Ar²C₁₋₆ alkyloxy, hydroxycarbonyl, C₁₋₆ alkyloxycarbonyl, trihalomethyl, trihalomethoxy, C₂₋₆ alkenyl, 4,4-dimethyloxazolyl; or

when on adjacent positions R² and R³ taken together may form a bivalent radical of formula
—O—CH₂—O— (a-1),
—O—CH₂—CH₂—O— (a-2)
—O—CH═CH— (a-3)
—O—CH₂—CH₂— (a-4)
—O—CH₂—CH₂—CH₂— (a-5), or
—CH═CH—CH═CH— (a-6);

R⁴ and R⁵ each independently are hydrogen, halo, Ar¹, C₁₋₆ alkyl, hydroxyC₁₋₆ alkyl, C₁₋₆ alkyloxyC₁₋₆ alkyl, C₁₋₆ alkyloxy, C₁₋₆ alkylthio, amino, hydroxycarbonyl, C₁₋₆ alkyloxycarbonyl, C₁₋₆ alkylS (O) C₁₋₆ alkyl or C₁₋₆ alkylS (O)₂ C₁₋₆ alkyl;

R⁶ and R⁷ each independently are hydrogen, halo, cyano, C₁₋₆ alkyl, C₁₋₆ alkyloxy, Ar² oxy, trihalomethyl, C₁₋₆ alkylthio, di(C₁₋₆ alkyl) amino, or

when on adjacent positions R⁶ and R⁷ taken together may form a bivalent radical of formula
—O—CH₂—O— (c-1), or
—CH═CH—CH═CH— (c-2);
R⁸ is hydrogen, C₁₋₆ alkyl, cyano, hydroxycarbonyl, C₁₋₆ alkyloxycarbonyl, C₁₋₆ alkylcarbonylC₁₋₆ alkyl, cyanocC₁₋₆ alkyl, C₁₋₆ alkyloxycarbonylC₁₋₆ alkyl, carboxyC₁₋₆ alkyl, hydroxyC₁₋₆ alkyl, aminoC₁₋₆ alkyl, mono- or di(C₁₋₆ alkyl)-aminoC₁₋₆ alkyl, imidazolyl, haloC₁₋₆ alkyl, C₁₋₆ alkyloxy-C₁₋₆ alkyl, aminocarbonylC₁₋₆ alkyl, or a radical of formula
—O—R¹⁰ (b-1),
—S—R¹⁰ (b-2),
—N—R¹¹R¹² (b-3),
wherein
R¹⁰ is hydrogen, C₁₋₆ alkyl, C₁₋₆ alkylcarbonyl, Ar¹, Ar²C₁₋₆ alkyl, C₁₋₆ alkyloxycarbonylC₁₋₆ alkyl, a radical or formula -Alk²-OR¹³ or -Alk²-NR¹⁴R¹⁵;
R¹¹ is hydrogen, C₁₋₁₂ alkyl, Ar¹ or Ar²C₁₋₆ alkyl;
R¹² is hydrogen, C₁₋₆ alkyl, C₁₋₆ alkylcarbonyl, C₁₋₆ alkyloxycarbonyl, C₁₋₆ alkylaminocarbonyl, Ar¹, Ar²C₁₋₆ alkyl, C₁₋₆ alkylcarbonylC₁₋₆ alkyl, a natural amino acid, Ar¹ carbonyl, Ar²C₁₋₆ alkylcarbonyl, aminocarbonylcarbonyl, C₁₋₆ alkyloxyC₁₋₆ alkyl-carbonyl, hydroxy, C₁₋₆ alkyloxy, aminocarbonyl, di(C₁₋₆ alkyl)aminoC₁₋₆ alkylcarbonyl, amino, C₁₋₆ alkylamino, C₁₋₆ alkylcarbonylamino, or a radical of formula -Alk²-OR¹³ or -Alk²-NR¹⁴R¹⁵;
wherein
Alk² is C₁₋₆ alkanediyl;
R¹³ is hydrogen, C₁₋₆ alkyl, C₁₋₆ alkylcarbonyl, hydroxyC₁₋₆ alkyl, Ar¹ or Ar²C₁₋₆ alkyl;
R¹⁴ is hydrogen, C₁₋₆ alkyl, Ar¹ or Ar²C₁₋₆ alkyl;
R¹⁵ is hydrogen, C₁₋₆ alkyl, C₁₋₆ alkylcarbonyl, Ar¹ or Ar²C₁₋₆ alkyl;
R¹⁷ is hydrogen, halo, cyano, C₁₋₆ alkyl C₁₋₆-alkyloxycarbonyl, Ar¹;
R¹⁸ is hydrogen, C₁₋₆ alkyl, C₁₋₆ alkyloxy or halo;
R¹⁹ is hydrogen or C₁₋₆ alkyl;
Ar¹ is phenyl or phenyl substituted with C₁₋₆ alkyl, hydroxy, amino, C₁₋₆ alkyloxy or halo; and
Ar² is phenyl or phenyl substituted with C₁₋₆ alkyl, hydroxy, amino, C₁₋₆ alkyloxy or halo;

or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor of the formula: 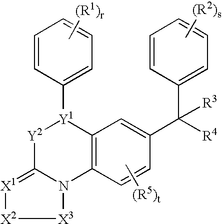
wherein

═X¹—X²—X³— is a trivalent radical of formula
═N—CR⁶═CR⁷— (x-1),
═N—N═CR⁶— (x-2),
═N—NH—C(═O)— (x-3),
═N—N═N— (x-4),
═N—CR⁶═N— (x-5),
═CR⁶—CR⁷═CR⁸— (x-6),
═CR⁶—N═CR⁷— (x-7),
═CR⁶—NH—C(═O)— (x-8), or
═CR⁶—N═N— (x-9);
wherein each R⁶, R⁷ and R⁸ are independently hydrogen, C₁₋₄ alkyl, hydroxy, C₁₋₄ alkyloxy, aryloxy, C₁₋₄ alkyloxycarbonyl, hydroxyC₁₋₆ alkyl, C₁₋₄ alkyloxyC₁₋₄ alkyl, mono- or di(C₁₋₆ alkyl)aminoC₁₋₄ alkyl, cyano, amino, thio, C₁₋₄ alkylthio, arylthio or aryl;

—Y¹—Y² is a trivalent radical of formula
—CH—CHR⁹— (y-1),
—C═N— (y-2),
—CH—NR⁹— (y-3), or
—C═CR⁹— (y-4);
wherein each R⁹ independently is hydrogen, halo, halocarbonyl, aminocarbonyl, hydroxyC₁₋₄ alkyl, cyano, carboxyl, C₁₋₄ alkyl, C₁₋₄ alkyloxy, C₁₋₄ alkyloxyC₁₋₄ alkyl, C₁₋₄ alkyloxycarbonyl, mono- or di(C₁₋₆ alkyl)amino, mono- or di(C₁₋₄ alkyl)aminoC₁₋₄ alkyl, or aryl;

r and s are each independently 0, 1, 2, 3, 4 or 5;

t is 0, 1, 2 or 3;

each R¹ and R² are independently hydroxy, halo, cyano, C₁₋₆ alkyl, trihalomethyl, trihalomethoxy, C₂₋₆ alkenyl, C₁₋₆ alkyloxy, hydroxyC₁₋₆ alkyloxy, C₁₋₆ alkylthio, C₁₋₆ alkyloxyC₁₋₆ alkyloxy, C₁₋₆ alkyloxycarbonyl, aminoC₁₋₆ alkyloxy, mono- or di(C₁₋₆ alkyl)amino, mono- or di(C₁₋₆ alkyl)aminoC₁₋₆ alkyloxy, aryl, arylC₁₋₆ alkyl, aryloxy or arylC₁₋₆ alkyloxy, hydroxycarbonyl, C₁₋₆ alkyloxycarbonyl, aminocarbonyl, aminoC₁₋₆ alkyl, mono- or di(C₁₋₆ alkyl)aminocarbonyl, or mono- or di(C₁₋₆ alkyl)aminoC₁₋₆ alkyl; or

two R¹ or R² substituents adjacent to one another on the phenyl ring independently form together a bivalent radical of formula
—O—CH₂—O— (a-1),
—O—CH₂—CH₂—O— (a-2),
—O═CH═CH— (a-3),
—O—CH₂—CH₂— (a-4),
—O—CH₂—CH₂—CH₂— (a-5), or
—CH═CH—CH═CH— (a-6);

R³ is hydrogen, halo, C₁₋₆ alkyl, cyano, haloC₁₋₆ alkyl, hydroxyC₁₋₆ alkyl, cyanoC₁₋₆ alkyl, aminoC₁₋₆ alkyl, C₁₋₆ alkyloxyC₁₋₆ alkyl, C₁₋₆ alkylthioC₁₋₆ alkyl, aminocarbonyl, C₁₋₆ alkyl, hydroxycarbonyl, hydroxycarbonylC₁₋₆ alkyl, C₁₋₆ alkyloxycarbonylC₁₋₆ alkyl, C₁₋₆ alkylcarbonylC₁₋₆ alkyl, C₁₋₆ alkyloxycarbonyl, aryl, arylC₁₋₆ alkyloxyC₁₋₆alkyl, mono- or di(C₁₋₆ alkyl)aminoC₁₋₆ alkyl; or a radical of formula
—O—R¹⁰ (b-1),
—S—R¹⁰ (b-2), or
—NR¹¹R¹² (b-3),
wherein R¹⁰ is hydrogen, C₁₋₆ alkyl, C₁₋₆ alkylcarbonyl, aryl, arylC₁₋₆ alkyl, C₁₋₆ alkyloxycarbonyl C₁₋₆ alkyl, or a radical of formula -Alk-OR¹³ or -Alk-NR¹⁴R¹⁵;

R¹¹ is hydrogen, C₁₋₆ alkyl, aryl or arylC₁₋₆ alkyl;

R¹² is hydrogen, C₁₋₆ alkyl, aryl, hydroxy, amino, C₁₋₆ alkyloxy, C₁₋₆ alkylcarbonylC₁₋₆ alkyl, arylC₁₋₆ alkyl, C₁₋₆ alkylcarbonylamino, mono- or di(C₁₋₆ alkyl)amino, C₁₋₆ alkylcarbonyl, aminocarbonyl, arylcarbonyl, haloC₁₋₆ alkylcarbonyl, arylC₁₋₆ alkylcarbonyl, C₁₋₆ alkyloxycarbonyl, C₁₋₆ alkyloxyC₁₋₆ alkylcarbonyl, mono- or di(C₁₋₆ alkyl)aminocarbonyl wherein the alkyl moiety may optionally be substituted by one or more substituents independently selected from aryl or C₁₋₃ alkyloxycarbonyl, aminocarbonylcarbonyl, mono- or di(C₁₋₆ alkyl)aminoC₁₋₆ alkylcarbonyl, or a radical of formula -Alk-OR¹³ or -Alk-NR¹⁴R¹⁵;

wherein Alk is C₁₋₆ alkanediyl;

R¹³ is hydrogen, C₁₋₆ alkyl, C₁₋₆ alkylcarbonyl, hydroxyC₁₋₆ alkyl, aryl or arylC₁₋₆ alkyl;

R¹⁴ is hydrogen, C₁₋₆ alkyl, aryl or arylC₁₋₆ alkyl;

R¹⁵ is hydrogen, C₁₋₆ alkyl, C₁₋₆ alkylcarbonyl, aryl or arylC₁₋₆ alkyl;

R⁴ is a radical of formula

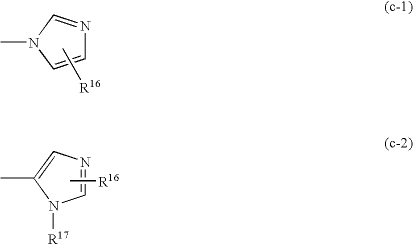

wherein R¹⁶ is hydrogen, halo, aryl, C₁₋₆ alkyl, hydroxyC₁₋₆ alkyl, C₁₋₆ alkyloxyC₁₋₆ alkyl, C₁₋₆ alkyloxy, C₁₋₆ alkylthio, amino, mono- or di(C₁₋₄ alkyl)amino, hydroxycarbonyl, C₁₋₆ alkyloxycarbonyl, C₁₋₆ alkylthioC₁₋₆ alkyl, C₁₋₆ alkylS(O)C₁₋₆ alkyl or C₁₋₆ alkylS(O)₂ C₁₋₆ alkyl;

R¹⁷ is hydrogen, C₁₋₆ alkyl, C₁₋₆ alkyloxyC₁₋₆ alkyl, arylC₁₋₆ alkyl, trifluoromethyl or di(C₁₋₄ alkyl)aminosulfonyl;

R⁵ is C₁₋₆ alkyl, C₁₋₆ alkyloxy or halo; aryl is phenyl, naphthalenyl or phenyl substituted with one or more substituents each independently selected from halo, C₁₋₆ alkyl, C₁₋₆ alkyloxy or trifluoromethyl; with the proviso that that when R¹⁶ is bound to one of the nitrogen atoms in the imidazole ring of formula (c-1) or (c-2), R¹⁶ is hydrogen, aryl, C₁₋₆ alkyl, hydroxyC₁₋₆ alkyl, C₁₋₆ alkyloxyC₁₋₆ alkyl, C₁₋₆ alkyloxycarbonyl, C₁₋₆ alkylS(O)C₁₋₆ alkyl or C₁₋₆ alkylS(O)₂ C₁₋₆ alkyl;

or a stereoisomeric form or a pharmaceutically acceptable acid or base addition salt form thereof, at a therapeutically effective dose and frequency.

In one embodiment, each R¹ and R² are independently hydroxy, halo, cyano, C₁₋₆ alkyl, trihalomethyl, trihalomethoxy, C₂₋₆ alkenyl, C₁₋₆ alkyloxy, hydroxyC₁₋₆ alkyloxy, C₁₋₆ alkylthio, C₁₋₆ alkyloxyC₁₋₆ alkyloxy, C₁₋₆ alkyloxycarbonyl, aminoC₁₋₆ alkyloxy, mono- or di(C₁₋₆ alkyl)amino, mono- or di(C₁₋₆ alkyl)aminoC₁₋₆ alkyloxy, aryl, arylC₁₋₆ alkyl, aryloxy or arylC₁₋₆ alkyloxy, hydroxycarbonyl, or C₁₋₆ alkyloxycarbonyl; or
two R¹ or R² substituents adjacent to one another on the phenyl ring independently form together a bivalent radical of formula
   —O—CH₂—O— (a-1),
   —O—CH₂—CH₂—O— (a-2),
   —O═CH═CH— (a-3),
   —O—CH₂—CH₂— (a-4),
   —O—CH₂—CH₂—CH₂— (a-5), or
   —CH═CH—CH═CH— (a-6);

R¹⁷ is hydrogen, C₁₋₆ alkyl, trifluoromethyl or di(C₁₋₆ alkyl)aminosulfonyl;

with the proviso that that when R¹⁶ is bound to one of the nitrogen atoms in the imidazole ring of formula (c-1), R¹⁶ is hydrogen, aryl, C₁₋₆ alkyl, hydroxyC₁₋₆ alkyl, C₁₋₆ alkyloxyC₁₋₆ alkyl, C₁₋₆ alkyloxycarbonyl, C₁₋₆ alkylS(O)C₁₋₆ alkyl or C₁₋₆ alkylS(O)₂ C₁₋₆ alkyl.

In another embodiment, the invention is a method for treating a synucleinopathic subject comprising administering to the synucleinopathic subject a farnesyl transferase inhibitor of the formula: 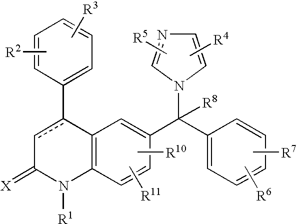
wherein
the dotted line represents an optional bond;
X is oxygen or sulfur;
R¹ is hydrogen, C₁₋₁₂ alkyl, Ar¹, Ar²C₁₋₆ alkyl, quinolinylC₁₋₆ alkyl, pyridylC₁₋₆ alkyl, hydroxyC₁₋₆ alkyl, C₁₋₆ alkyloxyC₁₋₆ alkyl, mono- or di(C₁₋₆ alkyl)aminoC₁₋₆ alkyl, aminoC₁₋₆ alkyl, or a radical of formula -Alk¹-C(═O)—R⁹, -Alk¹-S(O)—R⁹ or -Alk¹-S(O)₂—R⁹, wherein Alk¹ is C₁₋₆ alkanediyl,
R⁹ is hydroxy, C₁₋₆ alkyl, C₁₋₆ alkyloxy, amino, C₁₋₈ alkylamino or C₁₋₈ alkylamino substituted with C₁₋₆ alkyloxycarbonyl;
R² and R³ each independently are hydrogen, hydroxy, halo, cyano, C₁₋₆ alkyl, C₁₋₆ alkyloxy, hydroxyC₁₋₆ alkyloxy, C₁₋₆ alkyloxyC₁₋₆ alkyloxy, aminoC₁₋₆ alkyloxy, mono- or di(C₁₋₆ alkyl)aminoC₁₋₆ alkyloxy, Ar¹, Ar²C₁₋₆ alkyl, Ar² oxy, Ar²C₁₋₆ alkyloxy, hydroxycarbonyl, C₁₋₆ alkyloxycarbonyl, trihalomethyl, trihalomethoxy, C₂₋₆ alkenyl; or
when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);

R⁴ and R⁵ each independently are hydrogen, Ar¹, C₁₋₆ alkyl, C₁₋₆ alkyloxyC₁₋₆ alkyl, C₁₋₆ alkyloxy, C₁₋₆ alkylthio, amino, hydroxycarbonyl, C₁₋₆ alkyloxycarbonyl, C₁₋₆ alkylS(O)C₁₋₆ alkyl or C₁₋₆ alkylS(O)₂ C₁₋₆ alkyl;

R⁶ and R⁷ each independently are hydrogen, halo, cyano, C₁₋₆ alkyl, C₁₋₆ alkyloxy or Ar² oxy;

R⁸ is hydrogen, C₁₋₆ alkyl, cyano, hydroxycarbonyl, C₁₋₆ alkyloxycarbonyl, C₁₋₆ alkylcarbonylC₁₋₆ alkyl, cyanoC₁₋₆ alkyl, C₁₋₆ alkyloxycarbonylC₁₋₆ alkyl, hydroxycarbonylC₁₋₆ alkyl, hydroxyC₁₋₆ alkyl, aminoC₁₋₆ alkyl, mono- or di(C₁₋₆ alkyl)aminoC₁₋₆ alkyl, haloC₁₋₆ alkyl, C₁₋₆ alkyloxyC₁₋₆ alkyl, aminocarbonylC₁₋₆ alkyl, Ar¹, Ar²C₁₋₆ alkyloxyC₁₋₆ alkyl, C₁₋₆ alkylthioC₁₋₆ alkyl;

R¹⁰ is hydrogen, C₁₋₆ alkyl, C₁₋₆ alkyloxy or halo;

R¹¹ is hydrogen or C₁₋₆ alkyl;

Ar¹ is phenyl or phenyl substituted with C₁₋₆ alkyl, hydroxy, amino, C₁₋₆ alkyloxy or halo; and

Ar² is phenyl or phenyl substituted with C₁₋₆ alkyl, hydroxy, amino, C₁₋₆ alkyloxy or halo,

or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor with of formula:

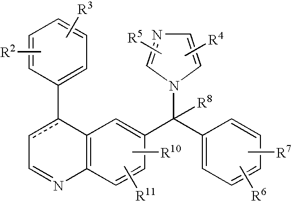

wherein the radicals R₂, R₃, R₄, R₅, R₆, R₇, R₈, R₁₀ and R₁₁ are as defined above, or a stereoisomeric form or a pharmaceutically acceptable acid or base addition salt form thereof, at a therapeutically effective dose and frequency.

In another embodiment, the invention is a method for treating a synucleinopathic subject comprising administering to the synucleinopathic subject a farnesyl transferase inhibitor with the formula:

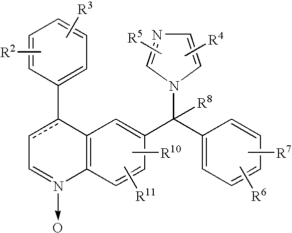

wherein the radicals R₂, R₃, R₄, R₅, R₆, R₇, R₈, R₁₀ and R₁₁ are as defined above, or a stereoisomeric form or a pharmaceutically acceptable acid or base addition salt form thereof, at a therapeutically effective dose and frequency.

In one aspect of the invention is a method of treating a synucleinopathic subject is provided, the method comprising, administering to a synucleinopathic subject a farnesyl transferase inhibitor compound of the formula:

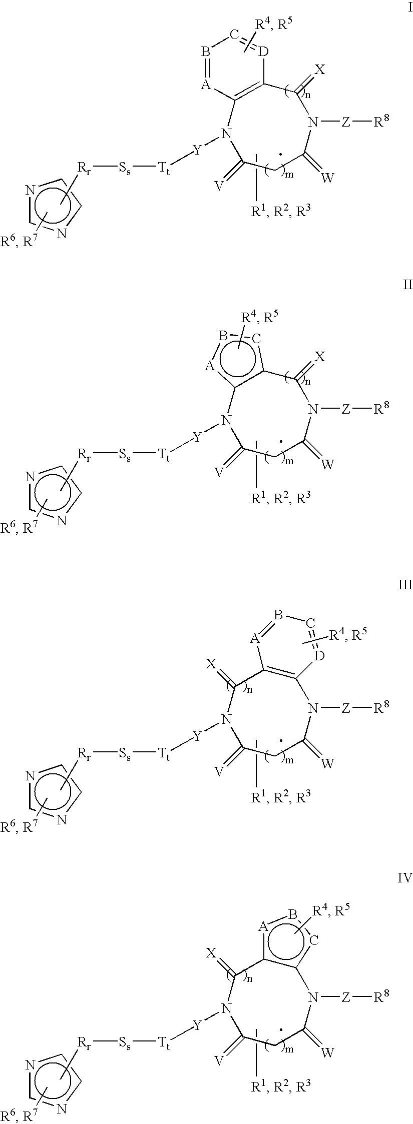

or a stereoisomeric form, or a pharmaceutically acceptable acid or base addition salt form thereof, in a therapeutically effective amount, wherein m,n,r,s and t are 0 or 1; p is 0, 1 or 2; V, W and X are selected from the group consisting of oxygen, hydrogen, R¹, R² or R³; Z and Y are selected from the group consisting of CHR⁹, SO₂, SO₃, CO, CO₂, O, NR¹⁰, SO₂NR¹¹, CONR¹², 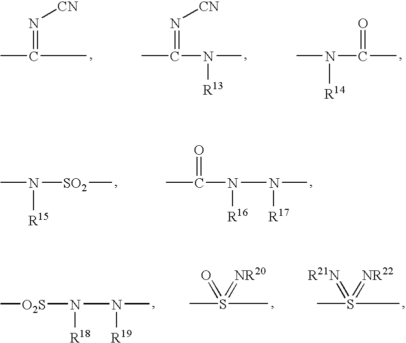
or Z may be absent; R⁶, R⁷, R⁹, R¹⁰, R¹¹, R¹², R¹³, R¹⁴, R¹⁵, R¹⁶, R¹⁷, R¹⁸, R¹⁹, R²⁰, R²¹, R²², R²⁴, R²⁵, R²⁶, R²⁷, R²⁸, R²⁹, R³⁰, R³¹, R³², R³³, R³⁴, R³⁵, R³⁶, R³⁷, and R³⁸ are selected from the group consisting of hydrogen, lower alkyl, substituted alkyl, aryl, or substituted aryl; R⁴, R⁵ are selected from the group consisting of hydrogen, halo, nitro, cyano and U—R²³; U is selected from the group consisting of sulfur, oxygen, NR²⁴, CO, SO, SO₂, CO₂, NR²⁵CO₂, NR²⁶CONR²⁷, NR²⁸SO₂, NR²⁹SO₂NR³⁰, SO₂NR³¹, NR³²CO, CONR³³, PO₂R³⁴ and PO₃R³⁵ or U is absent; R¹, R², and R³ are selected from the group consisting of hydrogen, alkyl, alkoxycarbonyl, substituted alkyl, alkenyl, substituted alkenyl, alkynyl, substituted alkynyl, aralkyl, cycloalkyl, aryl, substituted aryl, heterocyclo, substituted heterocyclo, cyano, carboxy, carbamyl (e.g. CONH₂) or substituted carbamyl further selected from CONH alkyl, CONH aryl, CONH aralkyl or cases where there are two substituents on the nitrogen selected from alkyl, aryl or aralkyl; R⁸ and R²³ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, substituted heterocyclo; any two of R¹, R², and R³ can be joined to form a cycloalkyl group;
R, S and T are selected from the group consisting of CH₂, CO and CH(CH₂)pQ wherein Q is NR³⁶R³⁷, OR³⁸, or CN; and A, B, C and D are carbon, oxygen, sulfur or nitrogen with the provisos that:

1. When m is zero then V and W are not both oxygen or,

2. W and X together can be oxygen only if Z is either absent, O, NR¹⁰, CHR⁹, 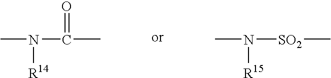
in formulas I and II, and V and X together can be oxygen only if Y is O, NR¹⁰, CHR⁹, 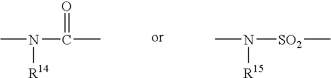
in formulas III and IV or, 3. R²³ may be hydrogen except when U is SO, SO₂, NR²⁵ CO₂ or NR²⁸SO₂, or, 4. R⁸ may be hydrogen except when Z is SO₂, CO₂, or

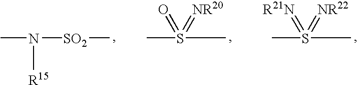

In one embodiment, the invention is a method of treating a synucleinopathic subject, the method comprising, administering to a synucleinopathic subject a farnesyl transferase inhibitor compound of the formula:

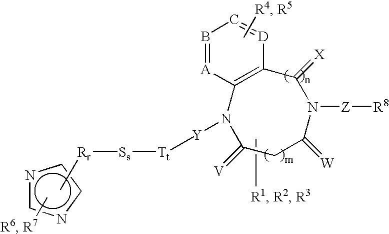

or a stereoisomeric form, or a pharmaceutically acceptable acid or base addition salt form thereof, in a therapeutically effective amount, wherein n is 1; r, s and t are 0 or 1; p is 0, 1 or 2; V, W and X are selected from the group consisting of oxygen, hydrogen, R¹, R² and R³; 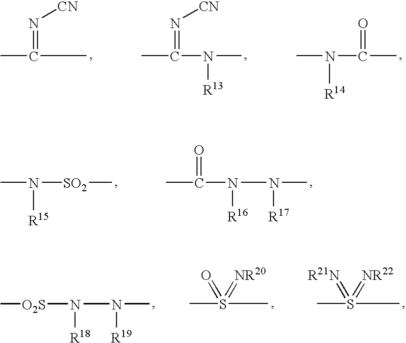
Z and Y are selected from the group consisting of CHR⁹, SO₂, SO₃, CO, CO₂, O, NR¹⁰, SO₂NR¹¹, CONR¹², or Z may be absent; R⁶, R⁷, R⁹, R¹⁰, R¹¹, R¹², R¹³, R¹⁴, R¹⁵, R¹⁶, R¹⁷, R¹⁸, R¹⁹, R²⁰, R²¹, R²², R²⁴, R²⁵, R²⁶, R²⁸, R²⁹, R³⁰, R³¹, R³², R³³, R³⁴, R³⁵, R³⁶, R³⁷, and R³⁸ are selected from the group consisting of hydrogen, lower alkyl, substituted alkyl, aryl and substituted aryl; R⁴ and R⁵ are selected from the group consisting of hydrogen, halo, nitro, cyano and U—R²³; U is selected from the group consisting of sulfur, oxygen, NR²⁴, CO, SO, SO₂, CO₂, NR²⁵CO₂, NR²⁶CONR²⁷, NR²⁸SO₂, NR²⁹SO₂NR³⁰, SO₂NR³¹, NR³²CO, CONR³³, PO₂R³⁴ and PO₃R³⁵ or U is absent; R¹, R² and R³ are selected from the group consisting of hydrogen, alkyl, alkoxycarbonyl, substituted alkyl, alkenyl, substituted alkenyl, alkynyl, substituted alkynyl, aralkyl, cycloalkyl, aryl, substituted aryl, heterocyclo, substituted heterocyclo, cyano, carboxy, carbamyl and substituted carbamyl; R⁸ and R²³ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo and substituted heterocyclo; any two of R¹, R² and R³ may be joined to form a cycloalkyl group; R, S and T are selected from the group consisting of CH₂, CO and CH(CH₂)pQ wherein Q is NR³⁶R³⁷, OR³⁸ or CN; and A, B, C and D are carbon; with the provisos that V and W are not both oxygen; W and X together may be oxygen only if Z is either absent, O, NR¹⁰, CHR⁹, —N(R¹⁴)—C(O)—, —N(R¹⁵)—SO₂—; R²³ may be hydrogen except when U is SO, SO₂, NR²⁵CO₂ or NR²⁸SO₂; and R⁸ may be hydrogen except when Z is SO₂, CO₂, —N(R¹⁵)—SO₂,

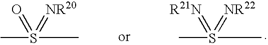

In yet another embodiment of the invention the compound is selected from the group consisting of:
2,3,4,5-Tetrahydro-1-(1H-imidazol-4-ylmethyl)-4-(1-naphthalenylcarbonyl)-1H-1,4-benzodiazepine, hydrochloride;
8-Chloro-2,3,4,5-tetrahydro-1-(1H-imidazol-4-ylmethyl)-4-(1-naphthalenylcarbonyl)-1H-1,4-benzodiazepine, hydrochloride;
2,3,4,5-Tetrahydro-4-(1H-imidazol-4-yl-methyl)-1-(1-1-naphthalenylcarbonyl)-1H-1,4-benzodiazepine, dihydrochloride;
2,3,4,5-Tetrahydro-1-(1H-imidazol-4-yl-methyl)-2-methyl-4-(1-naphthalenylcarbonyl)-1-H-1,4-benzodiazepine, hydrochloride;
2,3,4,5-Tetrahydro-4-(1-naphthalenylcarbonyl)-1-[[1-(phenylmethyl)-1H-imidazol-5-yl]methyl]-1H-1,4-benzodiazepine, hydrochloride;
2,3,4,5-Tetrahydro-(1H-imidazol-4-yl-methyl)-4-(1-naphthalenylsulfonyl)-1H-1,4-benzodiazepine, hydrochloride;
(S)-2,3,4,5-Tetrahydro-1-(1H-imidazol-4-ylmethyl)-3-[2-(methylthio)ethyl]-4-(1-naphthalenylcarbonyl)-1H-1,4-benzodiazepine, hydrochloride;
2,3,4,5-Tetrahydro-1-(1H-imidazol-4-yl-methyl)-N-methyl-N-phenyl-4H-1,4-benzodiazepine-4-carboxamide, hydrochloride;
2-[2,3,4,5-Tetrahydro-1-(1H-imidazol-4-yl-methyl)-1H-1,4-benzodiazepin-4-yl]sulfonyl]benzoic acid, methyl ester, hydrochloride;
7-Bromo-2,3,4,5-tetrahydro-1-(1H-imidazol-4-ylmethyl)-4-(1-naphthalenylcarbonyl)-1H-1,4-benzodiazepine, hydrochloride;
2,3,4,5-Tetrahydro-1-(1H-imidazol-4-ylmethyl)-4-(1-naphthalenenylcarbonyl)-7-phenyl-1H-1,4-benzodiazepine, hydrochloride;
2,3,4,5-Tetrahydro-1-(1H-imidazol-2-ylmethyl)-4-(1-naphthalenylcarbonyl)-1H-1,4-benzodiazepine, dihydrochloride;
2,3,4,5-Tetrahydro-1-[3-(1H-imidazol-2-yl)propyl]-4-(1-naphthalenylcarbonyl)-1H-1,4-benzodiazepine, dihydrochloride;
1-[3-Amino-3-(1H-imidazol-2-yl)propyl]-2,3,4,5-tetrahydro-4-(1-naphthalenylcarbonyl)-1H-1,4-benzodiazepine, trihydrochloride;
2,3,4,5-Tetrahydro-1-(1H-imidazol-4-ylmethyl)-3-methyl-4-(1-napthalenylcarbonyl)-1H-1,4-benzodiazepine, hydrochloride;
(S)-2,3,4,5-Tetrahydro-1-(1H-imidazol-4-ylmethyl)-3-[2-(methylthio)ethyl]-4-(1-naphthalenylmethyl)-1H-1,4-benzodiazepine, hydrochloride;
2,3,4,5-Tetrahydro-1-(1H-imidazol-4-ylmethyl)-9-methyl-4-(1-naphthalenylcarbonyl)-1H-1,4-benzodiazepine, dihydrochloride;
2,3,4,5-Tetrahydro-4-(1H-imidazol-4-ylmethyl)-9-methyl-1-(1-naphthalenylcarbonyl)-1H-1,4-benzodiazepine, dihyrdochloride;
1-[[2-(2-Aminoethyl)-1H-imidazol-4-yl]methyl]-2,3,4,5-tetrahydro-4-(1-naphthalenylcarbonyl)-1H-1,4-benzodiazepine, trihydrochloride;
1-[[2-Aminomethyl)-1H-imidazol-4-yl]methyl]-2,3,4,5-tetrahydro-4-(1-naphthalenylcarbonyl)-1H-1,4-benzodiazepine, trihydrochloride;
N-[2,3,4,5-Tetrahydro-1-(1H-imidazol-4-ylmethyl)-4-(1-naphthalenylcarbonyl)-1H-1,4-benzodiazepin-8-yl]acetamide, dihydrochloride;
2,3,4,5-Tetrahydro-1-(1H-imidazol-4-ylmethyl)-4-(1-naphthalenylcarbonyl)-8-nitro-1H-1,4-benzodiazepine, dihydrochloride;
2,3,4,5-Tetrahydro-1-(1H-imidazol-4-ylmethyl)-4-(1-naphthalenylcarbonyl)-8-amino-1H-1,4-benzodiazepine, dihydrochloride;
N-[2,3,4,5-Tetrahydro-1-(1H-imidazol-4-ylmethyl)-4-(1-naphthalenylcarbonyl)-1H-1,4-benzodiazepin-8-yl]benzamide, dihydrochloride;
N-[2,3,4,5-Tetrahydro-1-(1H-imidazol-4-ylmethyl)-4-(1-naphthalenylcarbonyl)-1H-1,4-benzodiazepin-8-yl]cyclohexanamide, dihydrochloride;
2,3,4,5-Tetrahydro-1-[2-(1H-imidazol-4-yl)ethyl]-4-(1-naphthalenylcarbonyl)-1H-1,4-benzodiazepine, dihydrochloride;
2,3,4,5-Tetrahydro-1-[2-(1H-imidazol-4-yl)ethyl]-4-(1-naphthalenylcarbonyl)-7-phenyl-1H-1,4-benzodiazepine, dihydrochloride;
7-Bromo-2,3,4,5-tetrahydro-1-[2-(1H-imidazol-4-yl)ethyl]-4-(1-naphthalenylcarbonyl)-1H-1,4-benzodiazepine, dihydrochloride;
1-[[1-(2-Aminoethyl)-1H-imidazol-5-yl]methyl]-2,3,4,5-tetrahydro-4-(1-naphthalenylcarbonyl)-7-phenyl-1H-1,4-benzodiazepine, trihydrochloride;
2,3,4,5-Tetrahydro-1-(1H-imidazol-4-ylmethyl)-7-phenyl-1H-1,4-benzodiazepine-4-carboxylic acid, phenylmethyl ester;
2,3,4,5-Tetrahydro-1-(1H-imidazol-4-ylmethyl)-7-phenyl-4-[2-(trifluoromethoxy)benzoyl]-1H-1,4-benzodiazepine;
1,2,3,5-Tetrahydro-1-(1H-imidazol-4-ylmethyl)-N-methyl-N,7-diphenyl-4H-1,4-benzodiazepine-4-carboxamide, dihydrochloride;
2,3,4,5-Tetrahydro-1-(1H-imidazol-4-ylmethyl)-4-(1-naphthaleneylcarbonyl)-7-(1-piperidinylsulfonyl)-1H-1,4-benzodiazepine, monohydrochloride;
2,3,4,5-Tetrahydro-1-(1H-imidazol-4-ylmethyl)-4-(1-naphthalenylcarbonyl)-7-pyridin-2-yl-1H-1,4-benzodiazepine, trihydrochloride;
7-(2-Furanyl)-2,3,4,5-tetrahydro-1-(1H-imidazol-4-ylmethyl)-4-(1-naphthalenylcarbonyl)-1H-1,4-benzodiazepine, dihydrochloride;
2,3,4,5-Tetrahydro-1-(1H-imidazol-4-ylmethyl)-4-(1-naphthalenylcarbonyl)-7-(2-thienyl)-1H-1,4-benzodiazepine, dihydrochloride;
2,3,4,5-Tetrahydro-1-(1H-imidazol-4-ylmethyl)-4-(1-naphthalenylcarbonyl)-7-(4-pyridinyl)-1H -1,4-benzodiazepine, trihydrochloride;
2,3,4,5-Tetrahydro-1-[3-(1H-imidazol-2-yl)propyl]-4-(1-naphthalenylcarbonyl)-7-phenyl-1H-1,4-benzodiazepine, dihydrochloride;
7-Bromo-2,3,4,5-tetrahydro-4-(1H-imidazol-4-ylmethyl)-1-(1-naphthalenylcarbonyl)-1H-1,4-benzodiazepine, dihydrochloride;
8-Chloro-2,3,4,5-tetrahydro-4-(1H-imidazol-4-ylmethyl)-1-(1-naphthalenylcarbonyl)-1H-1,4-benzodiazepine, dihydrochloride;
2,3,4,5-Tetrahydro-4-(1H-imidazol-4-ylmethyl)-1-(1-naphthalenylcarbonyl)-7-phenyl-1H-1,4-benzodiazepine, hydrochloride; 2,3,4,5-Tetrahydro-1,4-bis(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(1-naphthalenylmethyl)-7-phenyl-1H-1,4-benzodiazepine, trifluoroacetate;
2,3,4,5-Tetrahydro-1-(1H-imidazol-4-ylmethyl)-7-methoxy-4-(1-naphthalenylcarbonyl)-1H-1,4-benzodiazepine, dihydrochloride;
2,3,4,5-Tetrahydro-1-(1H-imidazol-4-ylmethyl)-4-(1-naphthalenylcarbonyl)-1H-1,4-benzodiazepine-7-carboxylic acid, dihydrochloride;
2,3,4,5-Tetrahydro-1-(1H-imidazol-5-ylmethyl)-4-(1-naphthalenylcarbonyl)-7-cyclohexyl-1H-1,4-benzodiazepine, 2.5 hydrochloride;
7-Butyl-2,3,4,5-tetrahydro-1-(1H-imidazol-4-ylmethyl)-4-(1-naphthalenylcarbonyl)-1H-1,4-benzodiazepine, dihydrochloride;
1-[[2-(2-Aminoethyl)-1H-imidazol-4-yl]methyl]-2,3,4,5-tetrahydro-4-(1-naphthalenylcarbonyl)-7-phenyl-1H-1,4-benzodiazepine, trihydrochloride;
1-[[2-(Aminomethyl)-1H-imidazol-4-yl]methyl]-2,3,4,5-tetrahydro-4-(1-naphthalenylcarbonyl)-7-phenyl-1H-1,4-benzodiazepine, trihydrochloride;
2,3,4,5-Tetrahydro-1-(1H-imidazol-4-ylmethyl)-4-(1-naphthalenylcarbonyl)-8-[N,N-bis(phenyl-methyl)amino]-1H-1,4-benzodiazepine, trihydrochloride;
N-[2,3,4,5-Tetrahydro-1-(1H-imidazol-4-yl-methyl)-4-(1-naphthalenylcarbonyl)-1H-1,4-benzodiazepin-8-yl]phenylsulfonamide, dihydrochloride;
N-Phenyl-2,3,4,5-tetrahydro-1-(1H-imidazol-4-yl-methyl)-4-(1-naphthalenylcarbonyl)-1H-1,4-benzo-diazepine-7-carboxamide, dihydrochloride;
N-[2,3,4,5-Tetrahydro-1-(1H-imidazol-4-ylmethyl)-4-(1-naphthalenylcarbonyl)-1H-1,4-benzodiazepin-8-yl]-3-methylbenzamide, dihydrochloride;
N-[2,3,4,5-Tetrahydro-1-(1H-imidazol-4-ylmethyl)-4-(1-naphthalenylcarbonyl)-1H-1,4-benzodiazepin-8-yl]-4-methylbenzamide, dihydrochloride;
3-Chloro-N-[2,3,4,5-tetrahydro-1-(1H-imidazol-4-yl-methyl)-4-(1-naphthalenylcarbonyl)-1H-1,4-benzo-diazepin-8-yl]benzamide, dihydrochloride;
7-Bromo-2,3,4,5-tetrahydro-1-[[2-[(dimethylamino)-methyl]-1H-imidazol-4-yl]methyl]-4-(1-naphthalenylcarbonyl)-1H-1,4-benzodiazepine, dihydrochloride;
7-(4-Chlorophenyl)-2,3,4,5-tetrahydro-1-(1H-imidazol-4-ylmethyl)-4-(1-naphthalenylcarbonyl)-1H-1,4-benzodiazepine, dihydrochloride;
7-(3-Aminophenyl)-2,3,4,5-tetrahydro-1-(1H-imidazol-4-ylmethyl)-4-(1-naphthalenylcarbonyl)-1H-1,4-benzodiazepine, trihydrochloride;
1-Methyl-N-[2,3,4,5-tetrahydro-1-(1H-imidazol-4-ylmethyl)-4-(1-naphthalenylcarbonyl)-1H-1,4-benzodiazepin-8-yl]-1H-pyrrole-2-carboxamide, trihydrochloride;
N-[2,3,4,5-Tetrahydro-1-(1H-imidazol-4-ylmethyl)-4-(1-naphthalenylcarbonyl)-1H-1,4-benzodiazepin-8-yl]-3-furancarboxamide, dihydrochloride;
7-(3-Chlorophenyl)-2,3,4,5-tetrahydro-1-(1H-imidazol-4-ylmethyl)-4-(1-naphthalenylcarbonyl)-1H-1,4-benzodiazepine, dihydrochloride;
2-Methyl-N-[2,3,4,5-tetrahydro-1-(1H-imidazol-4-ylmethyl)-4-(1-naphthalenylcarbonyl)-1H-1,4-benzodiazepin-8-yl]benzamide, dihydrochloride;
N-Phenyl-N′-[2,3,4,5-tetrahydro-1-(1H-imidazol-4-ylmethyl)-4-(1-naphthalenylcarbonyl)-1H-1,4-benzodiazepin-8-yl]urea, dihydrochloride;
2,3,4,5-Tetrahydro-1-(1H-imidazol-4-ylmethyl)-4-(1-naphthalenylcarbonyl)-7-(3-pyridinyl)-1H-1,4-benzodiazepine, trihydrochloride;
2,3,4,5-Tetrahydro-1-(1H-imidazol-4-ylmethyl)-9-methoxy-4-(1-naphthalenylcarbonyl)-1H-1,4-diazepine, dihydrochloride;
(R)-2,3,4,5-Tetrahydro-1-(1H-imidazol-4-ylmethyl)-3-[2-(methylthio)ethyl]-4-(1-naphthalenylcarbonyl)-1H-1,4-benzodiazepine, hydrochloride;
2,3,4,5-Tetrahydro-1-(1H-imidazol-4-ylmethyl)-4-(1-naphthalenylcarbonyl)-3-(phenylmethyl)-1H-1,4-benzodiazepine, hydrochloride;
2,3,4,5-Tetrahydro-3-(2-hydroxyethyl)-1-(1H-imidazol-4-ylmethyl)-4-(1-naphthalenylcarbonyl)-1H-1,4-benzodiazepine, trifluoroacetate;
2,3,4,5-Tetrahydro-4-(1H-imidazol-4-ylmethyl)-3-[2-(methylthio)ethyl]-4-(1-naphthalenylcarbonyl)-1H-1,4-benzodiazepine, trifluoroacetate;
(S)-2,3,4,5-Tetrahydro-1-(1H-imidazol-4-ylmethyl)-4-(1-naphthalenylcarbonyl)-3-(phenylmethyl)-1H-1,4-benzodiazepine, trifluoroacetate;
4-Acetyl-7-bromo-2,3,4,5-tetrahydro-1-(1H-imidazol-4-ylmethyl)-3-(phenylmethyl)-1H-1,4-benzodiazepine, hydrochloride;
2,3,4,5-Tetrahydro-4-(1H-imidazol-4-ylmethyl)-1-(1-naphthalenylcarbonyl)-3-(phenylmethyl)-1H-1,4-benzodiazepine, 1.5 hydrochloride;
7-Bromo-1,2,3,5-tetrahydro-1-(1H-imidazol-4-ylmethyl)-3-(phenylmethyl)-4H-1,4-benzodiazepine-4-carboxamide, trifluoroacetate;
7-Bromo-2,3,4,5-tetrahydro-1-(1H-imidazol-4-ylmethyl)-4-(methylsulfonyl)-3-(phenylmethyl)-1H-1,4-benzodiazepine, hydrochloride;
4-Acetyl-2,3,4,5-tetrahydro-1-(1H-imidazol-4-ylmethyl)-7-phenyl-3-(phenylmethyl)-1H-1,4-benzodiazepine, trifluoroacetate;
4-Acetyl-7-bromo-3-[(4-chlorophenyl)methyl]-2,3,4,5-tetrahydro-1-(1H-imidazol-4-ylmethyl)-1H-1,4-benzodiazepine, dihydrochloride;
N-Cyclohexyl-N′-[2,3,4,5-tetrahydro-1-(1H-imidazol-4-ylmethyl)-4-(1-naphthalenylcarbonyl)-1H-1,4-benzodiazepin-8-yl]urea, dihydrochloride;
2,2-Dimethyl-N-[2,3,4,5-tetrahydro-1-(1H-imidazol-4-ylmethyl)-4-(1-naphthalenylcarbonyl)-1H-1,4-benzodiazepin-8-yl]propanamide, dihydrochloride;
2,3,4,5-Tetrahydro-1-(1H-imidazol-4-ylmethyl)-4-(1-naphthalenylsulfonyl)-7-phenyl-1H-1,4-benzodiazepine, monohydrochloride;
4-Acetyl-7-bromo-2,3,4,5-tetrahydro-1-(1H-imidazol-4-ylmethyl)-3-(2-naphthalenylmethyl)-1H-1,4-benzodiazepine, dihydrochloride;
4-Acetyl-7-bromo-2,3,4,5-tetrahydro-1-(1H-imidazol-4-ylmethyl)-3-(1-naphthalenylmethyl)-1H-1,4-benzodiazepine, dihydrochloride;
7-(2-Chlorophenyl)-2,3,4,5-tetrahydro-1-(1H-imidazol-4-ylmethyl)-4-(1-naphthalenylcarbonyl)-1H-1,4-benzodiazepine, dihydrochloride;
2,3,4,5-Tetrahydro-1-(1H-imidazol-4-ylmethyl)-7-phenyl-1H-1,4-benzodiazepine, monohydrochloride;
1-Methyl-N-[2,3,4,5-tetrahydro-1-(1H-imidazol-4-ylmethyl)-4-(1-naphthalenylcarbonyl)-1H-1,4-benzodiazepin-8-yl]-2-piperidinecarboxamide, trihydrochloride;
N-[2,3,4,5-tetrahydro-1-(1H-imidazol-4-ylmethyl)-4-(1-naphthalenylcarbonyl)-1H-1,4-benzodiazepin-8-yl]-4-morpholinecarboxamide, dihydrochloride;
N-[2,3,4,5-Tetrahydro-1-(1H-imidazol-4-ylmethyl)-4-(1-naphthalenylcarbonyl)-1H-1,4-benzodiazepin-8-yl]-3-methylbutanamide, dihydrochloride;
1,2,3,5-Tetrahydro-1-(1H-imidazol-4-ylmethyl)-N,N,7-triphenyl-4H-1,4-2 5 benzodiazepine-4-carboxamide, dihydrochloride;
2,3,4,5-Tetrahydro-1-(1H-imidazol-4-ylmethyl)-7-phenyl-4-[(4-phenyl-1,2,3-thiadiazol-5-yl)carbonyl]-1H-1,4-benzodiazepine, trifluoroacetate;
8-[[(Cyclohexylamino)carbonyl]amino]-2,3,4,5-tetrahydro-1-(1H-imidazol-4-ylmethyl)-3-(phenylmethyl)-1H-1,4-benzodiazepine-4-carboxylic acid, 1,1-dimethylethyl ester;
2,3,4,5-Tetrahydro-1-(1H-imidazol-4-ylmethyl)-8-[[(4-methylphenyl)sulfonyl]amino]-3-(phenylmethyl)-1H-1,4-benzodiazepine-4-carboxylic acid, 1,1-dimethylethylester;
7-Bromo-1,2,3,4-tetrahydro-1-(1H-imidazol-4-ylmethyl)-3-(phenylmethyl)-5H-1,4-benzodiazepin-5-one, dihydrochloride;
2,3,4,5-Tetrahydro-1-(1H-imidazol-4-ylmethyl)-4-[1-oxo-3-(1-piperidinyl)propyl]-7-phenyl-1H-1,4-benzodiazepine, trihydrochloride;
2,3,4,5-Tetrahydro-1-(1H-imidazol-4-ylmethyl)-7-phenyl-4-(4-quinolinylcarbonyl)-1H-1,4-benzodiazepine, trihydrochloride;
4-[(5-Bromo-3-pyridinyl)carbonyl]-2,3,4,5-tetrahydro-1-(1H-imidazol-4-ylmethyl)-7-phenyl-1H-1,4-benzodiazepine, trihydrochloride;
(S)-4-[2-(Dimethylamino)-1-oxo-3-phenylpropyl]-2,3,4,5-tetrahydro-1-(1H-imidazol-4-ylmethyl)-7-phenyl-1H-1,4-benzodiazepine, trihydrochloride;
2,3,4,5-Tetrahydro-4-[4-hydroxy-3-(4-morpholinyl-methyl)benzoyl]-1-(1H-imidazol-4-ylmethyl)-7-phenyl-1H-1,4-benzodiazepine, trihydrochloride;
(S)-2,3,4,5-Tetrahydro-1-(1H-imidazol-4-ylmethyl)-4-[(1-methyl-2-pyrrolidinyl)carbonyl]-7-phenyl-1H-1,4-benzodiazepine, trihydrochloride;
2,3,4,5-Tetrahydro-1-(1H-imidazol-4-ylmethyl)-7-phenyl-4-[[2-(propylthio)-3-pyridinyl]carbonyl]-1H-1,4-benzodiazepine, trihydrochloride;
4-[(2-Chloro-6-methyl-4-pyridinyl)carbonyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-[[2-(phenylthio)-3-pyridinyl]carbonyl]-1H-1,4-benzodiazepine, trihydrochloride;
2,3,4,5-Tetrahydro-1-(1H-imidazol-4-ylmethyl)-4-[[2-(4-methylphenoxy)-3-pyridinyl]carbonyl]-7-phenyl-1H-1,4-benzodiazepine, trihydrochloride;
2,3,4,5-Tetrahydro-1-(1H-imidazol-4-ylmethyl)-4-[(2-methoxy-3-pyridinyl)carbonyl]-7-phenyl-1H-1,4-benzodiazepine, trihydrochloride;
2,3,4,5-Tetrahydro-1-(1H-imidazol-4-ylmethyl)-7-phenyl-4-[(5-phenyl-4-oxazolyl)carbonyl]-1H-1,4-benzodiazepine, dihydrochloride;
4-Acetyl-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-7-phenyl-4-[(tetrahydro-3-furanyl)carbonyl]-1H-1,4-benzodiazepine, dihydrochloride;
2,3,4,5-Tetrahydro-1-(1H-imidazol-4-ylmethyl)-4-[(2-methoxyethoxy)acetyl]-7-phenyl-1H-1,4-benzodiazepine, dihydrochloride;
2,3,4,5-Tetrahydro-1-(1H-imidazol-4-ylmethyl)-4-[4-(4-morpholinylmethyl)benzoyl]-7-phenyl-1H-1,4-benzodiazepine, trihydrochloride;
2,3,4,5-Tetrahydro-1-(1H-imidazol-4-ylmethyl)-4-[4-(methylsulfonyl)benzoyl]-7-phenyl-1H-1,4-benzodiazepine, dihydrochloride;
2,3,4,5-Tetrahydro-1-(1H-imidazol-4-ylmethyl)-4-[1-oxo-3-(phenylsulfonyl)propyl]-7-phenyl-1H-1,4-benzodiazepine, dihydrochloride;
2,3,4,5-Tetrahydro-1-(1H-imidazol-4-ylmethyl)-7-phenyl-4-(3-pyridinylacetyl)-1H-1,4-benzodiazepine, trihydrochloride;
2,3,4,5-Tetrahydro-1-(1H-imidazol-4-ylmethyl)-7-phenyl-4-(2-quinoxalinylcarbonyl)-1H-1,4-benzodiazepine, tetrahydrochloride;
2,3,4,5-Tetrahydro-1-(1H-imidazol-4-ylmethyl)-4-(4-isoquinolinylcarbonyl)-7-phenyl-H-1,4-benzodiazepine, trihydrochloride;
4-[(2-Chloro-3-pyridinyl)carbonyl]-2,3,4,5-tetrahydro-1-(1H-imidazol-4-ylmethyl)-7-phenyl-1H -1,4-benzodiazepine, trihydrochloride;
2,3,4,5-Tetrahydro-1-(1H-imidazol-4-ylmethyl)-7-phenyl-4-(3-pyridinylcarbonyl)-1H-1,4-benzodiazepine, trihydrochloride;
4-[(2,6-Dimethoxy-3-pyridinyl)carbonyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-(2-pyrazinylcarbonyl)-1H-1,4-benzodiazepine, tetrahydrochloride;
4-(2-Ethoxybenzoyl)-2,3,4,5-tetrahydro-1-(1H-imidazol-4-ylmethyl)-7-phenyl-1H-1,4-benzodiazepine, dihydrochloride;
4-[3-(Dimethylamino)benzoyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-[(1-phenylcyclopropyl)carbonyl]-1H-1,4-benzodiazepine, dihydrochloride;
4-[(Bicyclo[4.2.0]octa-1,3,5-trien-7-yl)carbonyl]-2,3,4,5-tetrahydro-1-(1H-imidazol-4-ylmethyl)-7-phenyl-1H-1,4-benzodiazepine, dihydrochloride;
4-Benzoyl-2,3,4,5-tetrahydro-1-(1H-imidazol-4-ylmethyl)-7-phenyl-1H-1,4-benzodiazepine, dihydrochloride;
4-(2-Chlorobenzoyl)-2,3,4,5-tetrahydro-1-(1H-imidazol-4-ylmethyl)-7-phenyl-1H-1,4-benzodiazepine, dihydrochloride;
4-(2,3-Dichlorobenzoyl)-2,3,4,5-tetrahydro-1-(1H-imidazol-4-ylmethyl)-7-phenyl-1H-1,4-benzodiazepine, dihydrochloride;
N-[2-[[2,3,4,5-Tetrahydro-1-(1H-imidazol-4-ylmethyl)-7-phenyl-1H-1,4-benzodiazepin-4-yl]carbonyl]phenyl]-acetamide, dihydrochloride;
2,3,4,5-Tetrahydro-1-(1H-imidazol-4-ylmethyl)-4-(2-phenoxybenzoyl)-7-phenyl-1H-1,4-benzodiazepine, dihydrochloride;
2,3,4,5-Tetrahydro-1-(1H-imidazol-4-ylmethyl)-4-(2-methoxybenzoyl)-7-phenyl-1H-1,4-benzodiazepine, dihydrochloride;
4-(2,3-Dimethoxybenzoyl)-2,3,4,5-tetrahydro-1-(1H-imidazol-4-ylmethyl)-7-phenyl-1H-1,4-benzodiazepine, dihydrochloride;
4-(2,4-Dimethoxybenzoyl)-2,3,4,5-tetrahydro-1-(1H-imidazol-4-ylmethyl)-7-phenyl-1H-1,4-benzodiazepine, dihydrochloride;
4-(2,5-Dimethoxybenzoyl)-2,3,4,5-tetrahydro-1-(1H-imidazol-4-ylmethyl)-7-phenyl-1H-1,4-benzodiazepine, dihydrochloride;
4-(2,6-Dimethoxybenzoyl)-2,3,4,5-tetrahydro-1-(1H-imidazol-4-ylmethyl)-7-phenyl-1H-1,4-benzodiazepine, dihydrochloride;
4-(2,3-Dihydroxybenzoyl)-2,3,4,5-tetrahydro-1-(1H-imidazol-4-ylmethyl)-7-phenyl-1H-1,4-benzodiazepine, dihydrochloride;
4-([1,1′-Biphenyl]-2-ylcarbonyl)-2,3,4,5-tetrahydro-1-(1H-imidazol-4-ylmethyl)-7-phenyl-1H -1,4-benzodiazepine, dihydrochloride;
2,3,4,5-Tetrahydro-1-(1H-imidazol-4-ylmethyl)-4-(2-methylbenzoyl)-7-phenyl-1H-1,4-benzodiazepine, dihydrochloride;
4-(2,3-Dimethylbenzoyl)-2,3,4,5-tetrahydro-1-(1H-imidazol-4-ylmethyl)-7-phenyl-1H-1,4-benzodiazepine, dihydrochloride;
4-(3-Cyanobenzoyl)-2,3,4,5-tetrahydro-1-(1H-imidazol-4-ylmethyl)-7-phenyl-1H-1,4-benzodiazepine, dihydrochloride;
4-(3-Chlorobenzoyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(3-phenoxybenzoyl)-7-phenyl-1H-1,4-benzodiazepine, dihydrochloride;
2,3,4,5-Tetrahydro-1-(1H-imidazol-4-ylmethyl)-4-(3-methoxybenzoyl)-7-phenyl-1H-1,4-benzodiazepine, dihydrochloride;
4-(3,4-Dimethoxybenzoyl)-2,3,4,5-tetrahydro-1-(1H-imidazol-4-ylmethyl)-7-phenyl-1H-1,4-benzodiazepine, dihydrochloride;
4-(3,5-Dimethoxybenzoyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(3-methylbenzoyl)-7-phenyl-1H-1,4-benzodiazepine, dihydrochloride;
4-(1,2-Dioxo-2-phenylethyl)-2,3,4,5-tetrahydro-1-(1H-imidazol-4-ylmethyl)-7-phenyl-1H-1,4-benzodiazepine, dihydrochloride;
4-[(2-Ethoxy-1-naphthalenyl)carbonyl]-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(2-naphthalenylcarbonyl)-7-phenyl-1H-1,4-benzodiazepine, dihydrochloride;
4-(Fluorophenylacetyl)-2,3,4,5-tetrahydro-1-(1H-imidazol-4-ylmethyl)-7-phenyl-1H-1,4-benzodiazepine, dihydrochloride;
4-(Diphenylacetyl)-2,3,4,5-tetrahydro-1-(1H-imidazol-4-ylmethyl)-7-phenyl-1H-1,4-benzodiazepine, dihydrochloride;
2,3,4,5-Tetrahydro-4-(2-hydroxy-1-oxo-2-phenylpropyl)-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(1H-indol-2-ylcarbonyl)-7-phenyl-1H-1,4-benzodiazepine, dihydrochloride;
2,3,4,5-Tetrahydro-1-(1H-imidazol-4-ylmethyl)-4-(1H-indol-3-ylcarbonyl)-7-phenyl-1H-1,4-benzodiazepine, dihydrochloride;
2,3,4,5-Tetrahydro-1-(1H-imidazol-4-ylmethyl)-4-(1H-indol-5-ylcarbonyl)-7-phenyl-1H-1,4-benzodiazepine, dihydrochloride;
2,3,4,5-Tetrahydro-1-(1H-imidazol-4-ylmethyl)-4-[(1-methyl-1H-indol-2-yl)carbonyl]-7-phenyl-1H-1,4-benzodiazepine, dihydrochloride;
4-(2-Benzofuranylcarbonyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-7-phenyl-4-(3-pyridinylcarbonyl)-1H-1,4-benzodiazepine, N-oxide, dihydrochloride;
2,3,4,5-Tetrahydro-1-(1H-imidazol-4-ylmethyl)-7-phenyl-4-(2-pyridinylcarbonyl)-1H-1,4-benzodiazepine, trihydrochloride;
2,3,4,5-Tetrahydro-1-(1H-imidazol-4-ylmethyl)-7-phenyl-4-(2-quinolinylcarbonyl)-1H-1,4-benzodiazepine, trihydrochloride;
2,3,4,5-Tetrahydro-1-(1H-imidazol-4-ylmethyl)-7-phenyl-4-(1-isoquinolinylcarbonyl)-1H-1,4-benzodiazepine, trihydrochloride;
4-(3-Chloro-2-nitrobenzoyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(2-nitrobenzoyl)-7-phenyl-1H-1,4-benzodiazepine, dihydrochloride;
2,3,4,5-Tetrahydro-1-(1H-imidazol-4-ylmethyl)-4-(3-methoxy-2-nitrobenzoyl)-7-phenyl-1H-1,4-benzodiazepine, dihydrochloride;
2,3,4,5-Tetrahydro-1-(1H-imidazol-4-ylmethyl)-4-(1H-indol-4-ylcarbonyl)-7-phenyl-1H-1,4-benzodiazepine, dihydrochloride;
4-[(2,6-Dihydroxy-3-naphthalenyl)carbonyl]-2,3,4,5-tetrahydro-1-(1H-imidazol-4-ylmethyl)-7-phenyl-1H-1,4-benzodiazepine, dihydrochloride;
4-(1H-Benzimidazol-5-ylcarbonyl)-2,3,4,5-tetrahydro-1-(1H-imidazol-4-ylmethyl)-7-phenyl-1H-1,4-benzodiazepine, trihydrochloride;
4-(1H-Benzotriazol-5-ylcarbonyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-[(4-methoxy-2-quinolinyl)carbonyl]-7-phenyl-1H-1,4-benzodiazepine, trihydrochloride;
N-[3-[[2,3,4,5-Tetrahydro-1-(1H-imidazol-4-ylmethyl)-7-phenyl-1H-1,4-benzodiazepin-4-yl]carbonyl]phenyl]-acetamide, dihydrochloride;
2,3,4,5-Tetrahydro-1-(1H-imidazol-4-ylmethyl)-4-(2-methyl-1-oxo-2-phenylpropyl)-7-phenyl-1H-1,4-benzodiazepine, dihydrochloride;
4-[2-(Dimethylamino)benzoyl]-2,3,4,5-tetrahydro-1-(1H-imidazol-4-ylmethyl)-7-phenyl-1H-1,4-benzodiazepine, trihydrochloride;
4-(3-Ethoxybenzoyl)-2,3,4,5-tetrahydro-1-(1H-imidazol-4-ylmethyl)-7-phenyl-1H-1,4-benzodiazepine, dihydrochloride;
2,3,4,5-Tetrahydro-4-(2-hydroxy[1,1′-biphenyl]-3-ylcarbonyl)-1-(1H-imidazol-4-ylmethyl)-7-phenyl-1H-1,4-benzodiazepine, dihydrochloride;
2,3,4,5-Tetrahydro-4-[2-[(2-hydroxyethyl)thio]benzoyl]-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-[(2-methoxy-1-naphthalenyl)carbonyl]-7-phenyl-1H-1,4-benzodiazepine, dihydrochloride;
2,3,4,5-Tetrahydro-4-[(2-hydroxy-4-quinolinyl)-carbonyl]-1-(1H-imidazol-4-ylmethyl)-7-phenyl-1H-1,4-benzodiazepine, dihydrochloride;
2-[[2,3,4,5-Tetrahydro-1-(1H-imidazol-4-ylmethyl)-7-phenyl-1H-1,4-benzodiazepin-4-yl]carbonyl]benzamide, dihydrochloride;
N-(1,1-Dimethylethyl)-2-[[2,3,4,5-tetrahydro-1-(1H-imidazol-4-ylmethyl)-7-phenyl-1H-1,4-benzodiazepin-4-yl]carbonyl]benzamide, dihydrochloride;
N-(4-Fluorophenyl)-N′-[3-[[2,3,4,5-tetrahydro-1-(1H-imidazol-4-ylmethyl)-7-phenyl-1H-1,4-benzodiazepin-4-yl]carbonyl]phenyl]urea, dihydrochloride;
2,3,4,5-Tetrahydro-1-(1H-imidazol-4-ylmethyl)-4-[(3-methyl-4-oxo-2-phenyl-4H-benzopyran-8-yl)carbonyl]-7-phenyl-1H-1,4-benzodiazepine, dihydrochloride;
2,3,4,5-Tetrahydro-1-(1H-imidazol-4-ylmethyl)-7-phenyl-4-[3-(trifluoromethoxy)benzoyl]-1H-1,4-benzodiazepine, dihydrochloride;
4-(2-Cyanobenzoyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-[2-[[(4-methylphenyl)sulfonyl]amino]benzoyl]-7-phenyl-1H-1,4-benzodiazepine, dihydrochloride;
2,3,4,5-Tetrahydro-1-(1H-imidazol-4-ylmethyl)-7-phenyl-4-(6-quinolinylcarbonyl)-1H-1,4-benzodiazepine, trihydrochloride;
2,3,4,5-Tetrahydro-1-(1H-imidazol-4-ylmethyl)-7-phenyl-4-(8-quinolinylcarbonyl)-1H-1,4-benzodiazepine, trihydrochloride;
4-(Benzo[b]thiophen-2-ylcarbonyl)-2,3,4,5-tetrahydro-1-(1H-imidazol-4-ylmethyl)-7-phenyl-1H-1,4-benzodiazepine, dihydrochloride;
4-[[4-(Dimethylamino)-1-naphthalenyl]carbonyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-(1H-purin-6-ylcarbonyl)-1H-1,4-benzodiazepine, trihydrochloride;
2,3,4,5-Tetrahydro-1-(1H-imidazol-4-ylmethyl)-4-(methoxyphenylacetyl)-7-phenyl-1H-1,4-benzodiazepine, dihydrochloride;
2,3,4,5-Tetrahydro-1-(1H-imidazol-4-ylmethyl)-4-[(5-methyl-1-phenyl-1H-pyrazol-4-yl)carbonyl]-7-phenyl-1H-1,4-benzodiazepine, trihydrochloride;
2,3,4,5-Tetrahydro-1-(1H-imidazol-4-ylmethyl)-4-[2-(2-methylphenyl)-1-oxopropyl]-7-phenyl-1H-1,4-benzodiazepine, dihydrochloride;
2,3,4,5-Tetrahydro-1-(1H-imidazol-4-ylmethyl)-7-phenyl-4-[(tetrahydro-4-phenyl-2H-pyran-4-yl)carbonyl]-1H-1,4-benzodiazepine, dihydrochloride;
2,3,4,5-Tetrahydro-1-(1H-imidazol-4-ylmethyl)-4-[2-(methylphenylamino)benzoyl]-7-phenyl-1H-1,4-benzodiazepine, trihydrochloride;
2,3,4,5-Tetrahydro-1-(1H-imidazol-4-ylmethyl)-7-phenyl-4-(4-quinolinylcarbonyl)-1H-1,4-benzodiazepine, N-oxide, dihydrochloride;
N-Methyl-N-(2-pyridinylmethyl)-2-[[2,3,4,5-tetrahydro-1-(1H-imidazol-4-ylmethyl)-7-phenyl-1H-1,4-benzodiazepin-4-yl]carbonyl]benzamide, trihydrochloride;
2,3,4,5-Tetrahydro-1-(1H-imidazol-4-ylmethyl)-4-(3-isoquinolinylcarbonyl)-7-phenyl-1H-1,4-benzodiazepine, trihydrochloride;
2,3,4,5-Tetrahydro-1-(1H-imidazol-4-ylmethyl)-4-[(2-naphthalenylthio)acetyl]-1H-1,4-benzodiazepine, trifluoroacetate (1:2);
4-[3-(3,4-Dimethoxyphenyl)-1-oxopropyl]-2,3,4,5-tetrahydro-1-(1H-imidazol-4-ylmethyl)-1H-1,4-benzodiazepine, trifluoroacetate (1:2);
4-([1,1′-Biphenyl]-4-ylacetyl)-2,3,4,5-tetrahydro-1-(1H-imidazol-4-ylmethyl)-1H-1,4-benzodiazepine, trifluoroacetate (1:2);
2,3,4,5-Tetrahydro-1-(1H-imidazol-4-ylmethyl)-4-(2-naphthalenylacetyl)-1H-1,4-benzodiazepine, trifluoroacetate (1:2);
4-([1,1′-Biphenyl]-2-ylcarbonyl)-2,3,4,5-tetrahydro-1-(1H-imidazol-4-ylmethyl)-1H-1,4-benzodiazepine, trifluoroacetate (1:2);
2,3,4,5-Tetrahydro-1-(1H-imidazol-4-ylmethyl)-4-[(2-phenyl-4-quinolinyl)carbonyl]-1H-1,4-benzodiazepine, trifluoroacetate (1:3);
2,3,4,5-Tetrahydro-1-(1H-imidazol-4-ylmethyl)-4-(3-pyridinylacetyl)-1H-1,4-benzodiazepine, trifluoroacetate (1:3);
4-(9H-Fluoren-9-ylacetyl)-2,3,4,5-tetrahydro-1-(1H-imidazol-4-ylmethyl)-1H-1,4-benzodiazepine, trifluoroacetate (1:2);
(S)-4-[2-(Dimethylamino)-1-oxo-3-phenylpropyl]-2,3,4,5-tetrahydro-1-(1H-imidazol-4-ylmethyl)-1H-1,4-benzodiazepine, trifluoroacetate (1:3);
(S)-2,3,4,5-Tetrahydro-1-(1H-imidazol-4-ylmethyl)-4-[(2-oxo-4-phenyl-3-oxazolidinyl)acetyl]-1H-1,4-benzodiazepine, trifluoroacetate (1:2);
4-(9-Acridinylcarbonyl)-2,3,4,5-tetrahydro-1-(1H-imidazol-4-ylmethyl)-1H-1,4-benzodiazepine, trifluoroacetate (1:3);
2,3,4,5-Tetrahydro-1-(1H-imidazol-4-ylmethyl)-4-(3-phenoxybenzoyl)-1H-1,4-benzodiazepine, trifluoroacetate (1:2);
2,3,4,5-Tetrahydro-1-(1H-imidazol-4-ylmethyl)-4-[[4′-(trifluoromethyl)[1,1′-biphenyl]-2-yl]carbonyl]-1H-1,4-benzodiazepine, trifluoroacetate (1:2);
2,3,4,5-Tetrahydro-1-(1H-imidazol-4-ylmethyl)-4-(4-phenoxybenzoyl)-1H-1,4-benzodiazepine, trifluoroacetate (1:2);
2,3,4,5-Tetrahydro-1-(1H-imidazol-4-ylmethyl)-4-(2-naphthalenylcarbonyl)-1H-1,4-benzodiazepine, trifluoroacetate (1:2);
2,3,4,5-Tetrahydro-1-(1H-imidazol-4-ylmethyl)-4-(1-oxo-4-phenylbutyl)-1H-1,4-benzodiazepine, trifluoroacetate (1:2);
2,3,4,5-Tetrahydro-1-(1H-imidazol-4-ylmethyl)-4-[(2-phenoxyphenyl)acetyl]-1H-1,4-benzodiazepine, trifluoroacetate (1:2);
2,3,4,5-Tetrahydro-1-(1H-imidazol-4-ylmethyl)-4-[2-[(4-methylphenyl)sulfinyl]benzoyl]-1H-1,4-benzodiazepine, trifluoroacetate (1:2);
2,3,4,5-Tetrahydro-1-(1H-imidazol-4-ylmethyl)-4-[2-[(phenylmethyl)amino]benzoyl]-1H-1,4-benzodiazepine, trifluoroacetate (1:3);
1,2,3,5-Tetrahydro-1-(1H-imidazol-4-yl-methyl)-N,N-diphenyl-4H-1,4-benzodiazepine-4-carboxamide, hydrochloride;
1,2,3,5-Tetrahydro-1-(1H-imidazol-4-yl-methyl)-a,7-diphenyl-4H-1,4-benzodiazepine-4-acetic acid, methyl ester, hydrochloride;
4-Acetyl-2,3,4,5-tetrahydro-1-(1H-imidazol-4-ylmethyl)-3-(phenylmethyl)-1H-1,4-benzodiazepine, hydrochloride;
(R)-7-Bromo-2,3,4,5-tetrahydro-1-(1H-imidazol-4-ylmethyl)-4-(methylsulfonyl)-3-(phenylmethyl)-1H-1,4-benzodiazepine, hydrochloride;
(R)-2,3,4,5-Tetrahydro-1-(1H-imidazol-4-ylmethyl)-4-(methylsulfonyl)-3-(phenylmethyl)-1H-1,4-benzodiazepine-7-carbonitrile, monohydrochloride;
(R)-4-Acetyl-2,3,4,5-tetrahydro-1-(1H-imidazol-4-ylmethyl)-7-phenyl-3-(phenylmethyl)-1H-1,4-benzodiazepine, monohydrochloride;
7-Bromo-4-[[2-(dimethylamino)ethyl]sulfonyl]-2,3,4,5-tetrahydro-1-(1H-imidazol-4-ylmethyl)-3-(phenylmethyl)-4H-1,4-benzodiazepine, trifluoroacetate (1:2);
2,3,4,5-Tetrahydro-1-(1H-imidazol-4-ylmethyl)-7-phenyl-4-[(1,2,3,4-tetrahydro-1-quinolinyl)carbonyl]-1H-1,4-benzodiazepine, monohydrochloride;
N-Ethyl-1,2,3,5-tetrahydro-1-(1H-imidazol-4-ylmethyl)-N,7-diphenyl-4H-1,4-benzodiazepine-4-carboxamide, monohydrochloride;
4-[(2,3-Dihydro-1H-indol-1-yl)carbonyl]-2,3,4,5-tetrahydro-1-(1H-imidazol-4-ylmethyl)-7-phenyl-1H-1,4-benzodiazepine, monohydrochloride;
2,3,4,5-Tetrahydro-1-(1H-imidazol-4-ylmethyl)-4-(methylsulfonyl)-3-(phenylmethyl)-7-(4-pyridinyl)-1H-1,4-benzodiazepine, trihydrochloride;
(R)-4-[[2-(Dimethylamino)ethyl]sulfonyl]-2,3,4,5-tetrahydro-1-(1H-imidazol-4-ylmethyl)-7-phenyl-3-(phenylmethyl)-1H-1,4-benzodiazepine, trifluoroacetate (1:1);
[2,3,4,5-Tetrahydro-1-(1H-imidazol-4-ylmethyl)-4-(1-naphthalenylcarbonyl)-1H-1,4-benzodiazepin-8-yl]carbamic acid, cyclohexyl ester, dihydrochloride;
(R)-7-Bromo-2,3,4,5-tetrahydro-1-(1-methyl-1H-imidazol-5-yl)methyl)-4-(methylsulfonyl)-3-(phenylmethyl)-1H-1,4-benzodiazepine, hydrochloride;
(R)-7-Cyano-2,3,4,5-tetrahydro-1-[(1-methyl-1H-imidazol-5-yl)methyl]-4-(methylsulfonyl)-3-(phenylmethyl)-1H-1,4-benzodiazepine, monohydrochloride;
4-[2-(4-Chlorophenyl)-1,2-dioxoethyl]-2,3,4,5-tetrahydro-1-(1H-imidazol-4-ylmethyl)-7-phenyl-1H-1,4-benzodiazepine, hydrochloride;
4-(1,2-Dioxopropyl)-2,3,4,5-tetrahydro-1-(1H-imidazol-4-ylmethyl)-7-phenyl-1H-1,4-benzodiazepine, hydrochloride;
2,3,4,5-Tetrahydro-1-(1H-imidazol-4-ylmethyl)-4-[2-(4-nitrophenyl)-1,2-dioxoethyl]-7-phenyl-1H-1,4-benzodiazepine, hydrochloride;
2,3,4,5-Tetrahydro-1-(1H-imidazol-4-ylmethyl)-4-[2-(4-methoxyphenyl)-1,2-dioxoethyl]-7-phenyl-1H-1,4-benzodiazepine, hydrochloride;
2,3,4,5-Tetrahydro-1-(1H-imidazol-4-ylmethyl)-7-phenyl-4-(3,3,3-trifluoro-1,2-dioxopropyl)-1H-1,4-benzodiazepine, trifluoroacetate (1:2);
(R)-7-Bromo-2,3,4,5-tetrahydro-1-(1H-imidazol-4-ylacetyl)-4-(methylsulfonyl)-3-(phenylmethyl)-1H-1,4-benzodiazepine, monohydrochloride;
(R)-7-Bromo-2,3,4,5-tetrahydro-1-(2-1H-imidazol-4-ylethyl)-4-(methylsulfonyl)-3-(phenylmethyl)-1H-1,4-benzodiazepine, monohydrochloride;
8-[(Cyclohexylcarbonyl)amino]-1,2,3,5-tetrahydro-1-(1H-imidazol-4-ylmethyl)-3-(phenylmethyl)-4H-1,4-benzodiazepine-4-carboxylic acid, methyl ester, dihydrochloride;
N-[2,3,4,5-Tetrahydro-1-(1H-imidazol-4-ylmethyl)-4-(1-naphthalenylcarbonyl)-1H-1,4-benzodiazepin-8-yl-1-piperidinecarboxamide, dihydrochloride;
(R)-7-Cyano-1,2,3,5-tetrahydro-1-(1H-imidazol-4-ylmethyl)-3-(phenylmethyl)-4H-1,4-benzodiazepine-4-carboxylic acid, ethyl ester, hydrochloride;
N-[2,3,4,5-Tetrahydro-1-(1H-imidazol-4-ylmethyl)-4-(methylsulfonyl)-3-(phenylmethyl)-1H-1,4-benzodiazepin-8-yl]cyclohexanecarboxamide, dihydrochloride;
(R)-7-Cyano-4-[[2-(dimethylamino)ethyl]sulfonyl]-2,3,4,5-tetrahydro-1-(1H-imidazol-4-ylmethyl)-3-(phenylmethyl)-4H-1,4-benzodiazepine, dihydrochloride;
(R)-7-Cyano-2,3,4,5-tetrahydro-1-(1H-imidazol-4-ylmethyl)-4-[[2-(4-morpholinyl)ethyl]sulfonyl]-3-(phenylmethyl)-4H-1,4-benzodiazepine, dihydrochloride;
N-[2,3,4,5-Tetrahydro-1-(1H-imidazol-4-ylmethyl)-4-(2-methoxy-3-methylbenzoyl)-1H-1,4-benzodiazepin-8-yl]cyclohexanecarboxamide, dihydrochloride,
8-[(Cyclohexylcarbonyl)amino]-2,3,4,5-tetrahydro-1-(1H-imidazol-4-ylmethyl)-N-phenyl-1H -1,4-benzodiazepine-4-carboxamide, dihydrochloride;
N-[2,3,4,5-Tetrahydro-1-(1H-imidazol-4-ylmethyl)-4-[(2-methylphenyl)sulfonyl]-1H-1,4-benzodiazepin-8-yl]cyclohexanamide, dihydrochloride;
N-[2,3,4,5-Tetrahydro-1-(1H-imidazol-4-ylmethyl)-4-[(2-methoxyphenyl)carbonyl]-1H-1,4-benzodiazepin-8-yl]cyclohexanamide, dihydrochloride;
(R)-7-Cyano-1,2,3,5-tetrahydro-1-(1H-imidazol-4-ylmethyl)-3-(phenylmethyl)-4H-1,4-benzodiazepine-4-sulfonic acid, ethyl ester, hydrochloride;
(3R)-7-Bromo-1-[cyano(1H-imidazol-4-yl)methyl]-2,3,4,5-tetrahydro-4-(methylsulfonyl)-3-(phenylmethyl)-1H-1,4-benzodiazepine, monohydrochloride;
(3R)-1-[2-Amino-1-(1H-imidazol-4-yl)ethyl]-2,3,4,5-tetrahydro-4-(methylsulfonyl)-3-(phenylmethyl)-1H-1,4-benzodiazepine, dihydrochloride;
(3R)-1-[2-(Dimethylamino)-1-(1H-imidazol-4-yl)ethyl]-2,3,4,5-tetrahydro-4-(methylsulfonyl)-3-(phenylmethyl)-1H-1,4-benzodiazepine, dihydrochloride;
(3R)-1-[2-Amino-1-(1H-imidazol-4-yl)ethyl]-7-bromo-2,3,4,5-tetrahydro-4-(methylsulfonyl)-3-(phenylmethyl)-1H-1,4-benzodiazepine, dihydrochloride;
(3R)-1-[2-(Dimethylamino)-1-(1H-imidazol-4-yl)ethyl]-7-bromo-2,3,4,5-tetrahydro-4-(methylsulfonyl)-3-(phenylmethyl)-1H-1,4-benzodiazepine, dihydrochloride;
7-Cyano-1,3,4,5-tetrahydro-1-(1-methyl-1H-imidazol-5-ylmethyl)-3-(phenylmethyl)-4-(phenylsulfonyl)-2H-1,4-benzodiazepin-2-one, monohydrochloride;
7-Cyano-1,3,4,5-tetrahydro-1-(1H-imidazol-4-ylmethyl)-3-(phenylmethyl)-4-(phenylsulfonyl)-2H-1,4-benzodiazepin-2-one, monohydrochloride;
7-Bromo-2,3,4,5-tetrahydro-1-(1H-imidazol-4-ylmethyl)-4-(methylsulfonyl)-3-(2-phenylethyl)-1H-1,4-benzodiazepine, dihydrochloride;
7-Bromo-3-[(3-chlorophenyl)methyl]-2,3,4,5-tetrahydro-1-(1H-imidazol-4-ylmethyl)-4-(methylsulfonyl)-1H-1,4-benzodiazepine, dihydrochloride;
(R)-7-Bromo-3-(cyclohexylmethyl)-2,3,4,5-tetrahydro-1-(1H-imidazol-4-ylmethyl)-4-(methylsulfonyl)-1H-1,4-benzodiazepine, dihydrochloride;
7-Bromo-3-[(2-chlorophenyl)methyl]-2,3,4,5-tetrahydro-1-(1H-imidazol-4-ylmethyl)-4-(methylsulfonyl)-1H-1,4-benzodiazepine, dihydrochloride;
(S)-7-Bromo-3-(cyclohexylmethyl)-2,3,4,5-tetrahydro-1-(1H-imidazol-4-ylmethyl)-4-(methylsulfonyl)-1H-1,4-benzodiazepine, dihydrochloride;
7-Bromo-2,3,4,5-tetrahydro-1-(1H-imidazol-4-ylmethyl)-3-[(4-methoxyphenyl)methyl]-4-(methylsulfonyl)-1H-1,4-benzodiazepine, dihydrochloride;
4-Acetyl-7-bromo-3-[(2-chlorophenyl)methyl]-2,3,4,5-tetrahydro-1-(1H-imidazol-4-ylmethyl)-1H-1,4-benzodiazepine, dihydrochloride;
4-Acetyl-7-bromo-3-[(3-chlorophenyl)methyl]-2,3,4,5-tetrahydro-1-(1H-imidazol-4-ylmethyl)-1H-1,4-benzodiazepine, dihydrochloride;
7-Bromo-2,3,4,5-tetrahydro-1-(1H-imidazol-4-ylmethyl)-3-[(4-hydroxyphenyl)methyl]-4-(methylsulfonyl)-1H-1,4-benzodiazepine, dihydrochloride;
(R)-2,3,4,5-Tetrahydro-1-(1H-imidazol-4-ylmethyl)-4-(methylsulfonyl)-7-phenyl-3-(3-pyridinylmethyl)-1H-1,4-benzodiazepine, dihydrochloride;
2,3,4,5-Tetrahydro-8-(hydroxymethyl)-1-(1H-imidazol-4-ylmethyl)-4-(1-naphthalenylcarbonyl)-1H-1,4-benzodiazepine, dihydrochloride;
2,3,4,5-Tetrahydro-1-(1H-imidazol-4-ylmethyl)-4-(1-naphthalenylcarbonyl)-8-(phenoxymethyl)-1H-1,4-benzodiazepine, dihydrochloride;
N-Cyclohexyl-2,3,4,5-tetrahydro-1-(1H-imidazol-4-ylmethyl)-4-(1-naphthalenylcarbonyl)-1H-1,4-benzodiazepine-8-carboxamide, dihydrochloride;
N-(Cyclohexylmethyl)-2,3,4,5-tetrahydro-1-(1H-imidazol-4-ylmethyl)-4-(1-naphthalenylcarbonyl)-1H-1,4-benzodiazepine-8-carboxamide, dihydrochloride;
2,3,4,5-Tetrahydro-1-(1H-imidazol-4-ylmethyl)-4-(1-naphthalenylcarbonyl)-N-(phenylmethyl)-1H-1,4-benzodiazepine-8-carboxamide, dihydrochloride;
(R)-4-Acetyl-7-[2-[(dimethylamino)methyl]phenyl]-2,3,4,5-tetrahydro-1-(1H-imidazol-4-ylmethyl)-3-(phenylmethyl)-1H-1,4-benzodiazepine, dihydrochloride;
(R)-4-Acetyl-7-cyano-2,3,4,5-tetrahydro-1-(1H-imidazol-4-ylmethyl)-3-(phenylmethyl)-1H-1,4-benzodiazepine, monohydrochloride;
(R)-7-Cyano-2,3,4,5-tetrahydro-1-(1H-imidazol-4-ylmethyl)-4-(1-oxobutyl)-3-(phenylmethyl)-1H-1,4-benzodiazepine, monohydrochloride;
(R)-7-Cyano-2,3,4,5-tetrahydro-1-(1H-imidazol-4-ylmethyl)-4-(2-methyl-1-oxopropyl)-3-(phenylmethyl)-1H-1,4-benzodiazepine, monohydrochloride;
(R)-7-Cyano-2,3,4,5-tetrahydro-1-(1H-imidazol-4-ylmethyl)-3-(phenylmethyl)-4-(2-pyridinylacetyl)-1H-1,4-benzodiazepine, dihydrochloride;
(R)-7-Cyano-2,3,4,5-tetrahydro-1-(1H-imidazol-4-ylmethyl)-3-(phenylmethyl)-4-(2-thienylsulfonyl)-1H-1,4-benzodiazepine, monohydrochloride;
(R)-7-Cyano-2,3,4,5-tetrahydro-1-(1H-imidazol-4-ylmethyl)-4-[(1-methylethyl)sulfonyl]-3-(phenylmethyl)-1H-1,4-benzodiazepine, monohydrochloride;
(R)-7-Cyano-2,3,4,5-tetrahydro-1-(1H-imidazol-4-ylmethyl)-3-(phenylmethyl)-4-[(trifluoromethyl)sulfonyl]-1H-1,4-benzodiazepine, monohydrochloride;
(R)-7-Bromo-2,3,4,5-tetrahydro-1-(1H-imidazol-4-ylmethyl)-3-(phenylmethyl)-4-(propylsulfonyl)-1H-1,4-benzodiazepine, monohydrochloride;
(R)-7-Bromo-2,3,4,5-tetrahydro-1-(1H-imidazol-4-ylmethyl)-3-(phenylmethyl)-4-(phenylsulfonyl)-1H-1,4-benzodiazepine, monohydrochloride;
(R)-2,3,4,5-Tetrahydro-1-(1H-imidazol-4-ylmethyl)-7-phenyl-3-(phenylmethyl)-4-(phenylsulfonyl)-1H-1,4-benzodiazepine, monohydrochloride;
(R)-2,3,4,5-Tetrahydro-1-(1H-imidazol-4-ylmethyl)-7-phenyl-3-(phenylmethyl)-4-(propylsulfonyl)-1H-1,4-benzodiazepine, monohydrochloride;
(R)-7-Cyano-4-[(4-fluorophenyl)sulfonyl]-2,3,4,5-tetrahydro-1-(1H-imidazol-4-ylmethyl)-3-(phenylmethyl)-1H-1,4-benzodiazepine, monohydrochloride;
(R)-7-Cyano-4-[(3-cyanophenyl)sulfonyl]-2,3,4,5-tetrahydro-1-(1H-imidazol-4-ylmethyl)-3-(phenylmethyl)-1H-1,4-benzodiazepine, monohydrochloride;
(R)-7-Cyano-2,3,4,5-tetrahydro-1-(1H-imidazol-4-ylmethyl)-4-[(1-methyl-1H-imidazol-2-yl)sulfonyl]-3-(phenylmethyl)-1H-1,4-benzodiazepine, dihydrochloride;
(R)-4-[(3-Bromophenyl)sulfonyl]-7-cyano-2,3,4,5-tetrahydro-1-(1H-imidazol-4-ylmethyl)-3-(phenylmethyl)-1H-1,4-benzodiazepine, monohydrochloride;
(R)—N-[5-[[7-cyano-2,3,4,5-tetrahydro-1-(1H-imidazol-4-ylmethyl)-3-(phenylmethyl)-1H-1,4-benzodiazepin-4-yl]sulfonyl]-4-methyl-2-thiazolyl]acetamide, dihydrochloride;
4-Acetyl-2,3,4,5-tetrahydro-1-(1H-imidazol-4-ylmethyl)-3-(phenylmethyl)-7-(4-pyridinyl)-1H-1,4-benzodiazepine, trihydrochloride;
2,3,4,5-Tetrahydro-1-(1H-imidazol-4-ylmethyl)-4-(2-phenyl-1,2-dioxoethyl)-7-(4-pyridinyl)-1H-1,4-benzodiazepine, trihydrochloride;
2,3,4,5-Tetrahydro-1-(1H-imidazol-4-ylmethyl)-7-(4-pyridinyl)-4-[2-(trifluoromethoxy)benzolyl]-1H-1,4-benzodiazepine, trihydrochloride;
(R)-2,3,4,5-Tetrahydro-1-[(1-methyl-1H-imidazol-5-yl)methyl]-4-(methylsulfonyl)-7-phenyl-3-(phenylmethyl)-1H-1,4-benzodiazepine;
(R)-7-Cyano-2,3,4,5-tetrahydro-1-(1H-imidazol-4-ylmethyl)-4-(phenylacetyl)-3-(phenylmethyl)-1H-1,4-benzodiazepine, monohydrochloride;
4-(2-Benzothiazolyl)-2,3,4,5-tetrahydro-1-(1H-imidazol-4-ylmethyl)-7-phenyl-1H-1,4-benzazepine, trihydrochloride;
2,3,4,5-Tetrahydro-1-(1H-imidazol-4-ylmethyl)-3-(phenylmethyl)-7-(3-pyridinyl)-4-(trifluoroacetyl)-1H-1,4-benzodiazepine, trihydrochloride;
2,3,4,5-Tetrahydro-1-(1H-imidazol-4-ylmethyl)-4-(methylsulfonyl)-3-(phenylmethyl)-7-(3-pyridinyl)-1H-1,4-benzodiazepine, trihydrochloride;
7-Bromo-3-[(1,1-dimethylethoxy)methyl]-1,2,3,4-tetrahydro-1-(1H-imidazol-4-ylmethyl)-5H-1,4-benzodiazepin-5-one;
7-Bromo-2,3,4,5-tetrahydro-1-(1H-imidazol-4-ylmethyl)-4-(methylsulfonyl)-3-(phenoxymethyl)-1H-1,4-benzodiazepine, dihydrochloride;
7-Bromo-2,3,4,5-tetrahydro-3-(hydroxymethyl)-1-(1H-imidazol-4-ylmethyl)-4-(methylsulfonyl)-1H-1,4-benzodiazepine, monohydrochloride;
7-Bromo-3-[(1,1-dimethylethoxy)methyl]-2,3,4,5-tetrahydro-1-(1H-imidazol-4-ylmethyl)-4-(methylsulfonyl)-1H-1,4-benzodiazepine;
[7-Bromo-2,3,4,5-tetrahydro-1-(1H-imidazol-4-ylmethyl)-3-(phenylmethyl)-1H-1,4-benzodiazepin-8-yl]carbamic acid, 2-methylpropyl ester, trihydrochloride;
[4-Acetyl-7-bromo-2,3,4,5-tetrahydro-1-(1H-imidazol-4-ylmethyl)-3-(phenylmethyl)-1H-1,4-benzodiazepin-8-yl]carbamic acid, 2-methylpropyl ester;
N-[4-Acetyl-2,3,4,5-tetrahydro-1-(1H-imidazol-4-ylmethyl)-3-(phenylmethyl)-1H-1,4-benzodiazepin-8-yl]cyclohexanecarboxamide, dihydrochloride;
[7-Bromo-2,3,4,5-tetrahydro-1-(1H-imidazol-4-ylmethyl)-4-(methylsulfonyl)-3-(phenylmethyl)-1H-1,4-benzodiazepin-8-yl]carbamic acid, 2-methylpropyl ester;
(R)-2,3,4,5-Tetrahydro-1-(1H-imidazol-4-ylmethyl)-4-(phenylsulfonyl)-3-(phenylmethyl)-1H-1,4-benzodiazepine-7-carbonitrile, monohydro chloride;
7-Bromo-1,2,3,5-tetrahydro-1-(1H-imidazol-4-ylmethyl)-3-(phenylmethyl)-4H-1,4-benzodiazepine-4-acetamide;
7-Bromo-4-[(dimethylamino)acetyl]-2,3,4,5-tetrahydro-1-(1H-imidazol-4-ylmethyl)-3-(phenylmethyl)-1H-1,4-benzodiazepine;
(R)-7-Bromo-4-(1,2-dioxopropyl)-2,3,4,5-tetrahydro-1-(1H-imidazol-4-ylmethyl)-3-(phenylmethyl)-1H-1,4-benzodiazepine, trifluoroacetate;
(R)-7-Bromo-4-(cyclopropylcarboonyl)-2,3,4,5-tetrahydro-1-(1H-imidazol-4-ylmethyl)-3-(phenylmethyl)-1H-1,4-benzodiazepine, trifluoroacetate;
(R)-7-Cyano-2,3,4,5-tetrahydro-1-(1H-imidazol-4-ylmethyl)-3-(phenylmethyl)-4-(propylsulfonyl)-1H-1,4-benzodiazepine, monohydrochloride;
7-Bromo-2,3,4,5-tetrahydro-1,4-bis(1H-imidazol-4-ylmethyl)-3-(phenylmethyl)-1H-1,4-benzodiazepine, dihydrochloride;
7-Bromo-1,2,3,5-tetrahydro-1-(1H-imidazol-4-ylmethyl)-N,N-dimethyl-3-(phenylmethyl)-4H-1,4-benzodiazepine-4-sulfonamide, monohydrochloride;
2,3,4,5-Tetrahydro-1-(1H-imidazol-4-ylmethyl)-4-(methylsulfonyl)-3-(phenylmethyl)-1H-1,4-benzodiazepine-7-carbonitrile, monohydrochloride;
(R)-7-Cyano-1,2,3,5-tetrahydro-1-(1H-imidazol-4-ylmethyl)-N,N-dimethyl-3-(phenylmethyl)-4H-1,4-benzodiazepine-4-carboxamide, monohydrochloride;
N,N-Diethyl-2,3,4,5-tetrahydro-1-(1H-imidazol-4-ylmethyl)-4-(methylsulfonyl)-3-(phenylmethyl)-1H-1,4-benzodiazepine-7-carboxamide, monohydrochloride;
2,3,4,5-Tetrahydro-1-(1H-imidazol-4-ylmethyl)-7-phenyl-4-(1-phenyl-1H-tetrazol-5-yl)-1H-1,4-benzodiazepine, monohydrochloride;
(R)-7-Cyano-2,3,4,5-tetrahydro-1-(1H-imidazol-4-ylmethyl)-3-(phenylmethyl)-4-(2-pyrazinylcarbonyl)-4H-1,4-benzodiazepine, monohydrochloride;
(R)-4-[7-Bromo-2,3,4,5-tetrahydro-1-(1H-imidazol-4-ylmethyl)-3-(phenylmethyl)-4H-1,4-benzodiazepin-4-yl]-4-oxobutanoic acid, methyl ester, monohydrochloride;
(R)-7-Cyano-2,3,4,5-tetrahydro-1-(1H-imidazol-4-ylmethyl)-4-(4-morpholinylcarbonyl)-3-(phenylmethyl)-1H-1,4-benzodiazepine, monohydrochloride;
(R)-7-Cyano-2,3,4,5-tetrahydro-1-(1H-imidazol-4-ylmethyl)-3-(phenylmethyl)-4-[[2-(1-pyrrolidinyl)ethyl]sulfonyl]-1H-1,4-benzodiazepine, dihydrochloride;
(S)-2,3,4,5-Tetrahydro-1-(1H-imidazol-4-ylmethyl)-4-(methylsulfonyl)-7-phenyl-3-(3-pyridinylmethyl)-1H-1,4-benzodiazepine, dihydrochloride;
(R)-2,3,4,5-Tetrahydro-1-(1H-imidazol-4-ylmethyl)-7-phenyl-3-(3-pyridinylmethyl)-4-(2-thienylsulfonyl)-1H-1,4-benzodiazepine, dihydrochloride;
(R)-2,3,4,5-Tetrahydro-1-(1H-imidazol-4-ylmethyl)-7-phenyl-4-(propylsulfonyl)-3-(3-pyridinylmethyl)-1H-1,4-benzodiazepine, monohydrochloride;
(R)-7-Bromo-2,3,4,5-tetrahydro-1-(1H-imidazol-4-ylmethyl)-4-(methylsulfonyl)-3-(2-pyridinylmethyl)-1H-1,4-benzodiazepine, monohydrochloride;
2,3,4,5-Tetrahydro-1-(1H-imidazol-4-ylmethyl)-4-(methylsulfonyl)-3-(phenylmethyl)-7-(2-pyrimidinyl)-1H-1,4-benzodiazepine, dihydrochloride;
(R)-7-Bromo-2,3,4,5-tetrahydro-1-(1H-imidazol-4-ylmethyl)-3-(phenylmethyl)-4-[(trifluoromethyl)sulfonyl]-1H-1,4-benzodiazepine, monohydrochloride;
(R)-2,3,4,5-Tetrahydro-1-(1H-imidazol-4-ylmethyl)-7-phenyl-3-(phenylmethyl)-4-(trifluoroacetyl)-1H-1,4-benzodiazepine, monohydrochloride;
(R)-2,3,4,5-Tetrahydro-1-(1H-imidazol-4-ylmethyl)-4-(methylsulfonyl)-3-(phenylmethyl)-7-(4-pyridinyl)-1H-1,4-benzodiazepine, dihydrochloride;
(R)-2,3,4,5-Tetrahydro-1-(1H-imidazol-4-ylmethyl)-3-(phenylmethyl)-7-(4-pyridinyl)-4-(2-thienylsulfonyl)-1H-1,4-benzodiazepine, dihydrochloride;
(R)-2,3,4,5-Tetrahydro-1-(1H-imidazol-4-ylmethyl)-3-(phenylmethyl)-4-(phenylsulfonyl)-7-(4-pyridinyl)-1H-1,4-benzodiazepine, dihydrochloride;
(R)-2,3,4,5-Tetrahydro-1-(1H-imidazol-4-ylmethyl)-3-(phenylmethyl)-4-(propylsulfonyl)-7-(4-pyridinyl)-1H-1,4-benzodiazepine, dihydrochloride;
(R)-7-Cyano-2,3,4,5-tetrahydro-1-(1H-imidazol-4-ylmethyl)-3-(phenylmethyl)-4-[(3,5-dimethyl-isoxazol-4-yl)sulfonyl]-1H-1,4-benzodiazepine, dihydrochloride;
(R)-7-Cyano-4-[(4-cyanophenyl)sulfonyl]-2,3,4,5-tetrahydro-1-(1H-imidazol-4-ylmethyl)-3-(phenylmethyl)-1H-1,4-benzodiazepine, dihydrochloride;
(R)-7-Cyano-2,3,4,5-tetrahydro-1-(1H-imidazol-4-ylmethyl)-3-(phenylmethyl)-4-[(2,2,2-trifluoroethyl)sulfonyl]-1H-1,4-benzodiazepine, dihydrochloride;
(R)-[(5-Bromo-2-thienyl)sulfonyl]-7-cyano-2,3,4,5-tetrahydro-1-(1H-imidazol-4-ylmethyl)-3-(phenylmethyl)-1H-1,4-benzodiazepine, dihydrochloride;
(R)-7-Cyano-2,3,4,5-tetrahydro-1-(1H-imidazol-4-ylmethyl)-4-[(4-methoxyphenyl)sulfonyl]-3-(phenylmethyl)-1H-1,4-benzodiazepine, dihydrochloride;
N-[[7-Bromo-2,3,4,5-tetrahydro-1-(1H-imidazol-4-ylmethyl)-4-(methylsulfonyl)-1H-1,4-benzodiazepin-3-yl]methyl]benzamide, dihydrochloride;
(R)-7-Cyano-1,2,3,5-tetrahydro-1-(1H-imidazol-4-ylmethyl)-N,N-dimethyl-3-(phenylmethyl)-4H-1,4-benzodiazepine-4-sulfonamide, hydrochloride;
(R)-7-Cyano-1,2,3,5-tetrahydro-N,N-dimethyl-1-[(1-methyl-1H-imidazol-5-yl)methyl]-3-(phenylmethyl)-4H-1,4-benzodiazepine-4-sulfonamide, hydrochloride;
(R)-7-Chloro-2,3,4,5-tetrahydro-1-[(1-methyl-1H-imidazol-5-yl)methyl]-4-(methylsulfonyl)-3-(phenylmethyl)-1H-1,4-benzodiazepine, monohydrochloride;
(R)-7-Chloro-2,3,4,5-tetrahydro-1-(1H-imidazol-4-ylmethyl)-4-(methylsulfonyl)-3-(phenylmethyl)-1H-1,4-benzodiazepine, monohydrochloride;
(R)-7-Chloro-2,3,4,5-tetrahydro-1-[(1-methyl-1H-imidazol-5-yl)methyl]-4-(phenylsulfonyl)-3-(phenylmethyl)-1H-1,4-benzodiazepine, monohydrochloride;
(R)-7-Cyano-2,3,4,5-tetrahydro-1-(1H-imidazol-4-ylmethyl)-3-(pyridin-3-ylmethyl)-4-(methylsulfonyl)-1H-1,4-benzodiazepine, tetrahydrochloride;
(R)-7-Bromo-2,3,4,5-tetrahydro-1-(1H-imidazol-2-ylmethyl)-4-(methylsulfonyl)-3-(phenylmethyl)-1H-1,4-benzodiazepine, dihydrochloride;
(R)-7-Bromo-2,3,4,5-tetrahydro-1-(1H-imidazol-4-ylmethyl)-4-[(1-methyl-1H-imidazol-4-yl)sulfonyl]-3-(phenylmethyl)-1H-1,4-benzodiazepine, trihydrochloride;
(R)-7-Chloro-2,3,4,5-tetrahydro-1-(1-methyl-imidazol-5-ylmethyl)-4-[(2-morpholin-4-yl-ethyl)sulfonyl]-3-(phenylmethyl)-1H-1,4-benzodiazepine, dihydrochloride;
(R)-7-Chloro-2,3,4,5-tetrahydro-1-(1H-imidazol-4-ylmethyl)-4-[(2-morpholin-4-yl-ethyl)sulfonyl]-3-(phenylmethyl)-1H-1,4-benzodiazepine, dihydrochloride;
(R)-7-Chloro-4-[(dimethylamino)sulfonyl]-1-[(1-methyl-1H-imidazol-5-yl)methyl]-3-(phenylmethyl)-1H-1,4-benzodiazepine, monohydrochloride;
(R)-7-Chloro-2,3,4,5-tetrahydro-1-(1-methyl-imidazol-5-ylmethyl)-4-[(4-methyl-piperidin-4-yl-ethyl)sulfonyl]-3-(phenylmethyl)-1H-1,4-benzodiazepine, dihydrochloride;
(R)-7-Bromo-2,3,4,5-tetrahydro-1-(1-methyl-imidazol-5-ylmethyl)-4-[(4-methyl-piperidin-4-yl-ethyl)sulfonyl]-3-(phenylmethyl)-1H-1,4-benzodiazepine, dihydrochloride;
(R)-7-Cyano-1,2,3,5-tetrahydro-1-(1H-imidazol-4-ylmethyl)-3-(phenylmethyl)-4H-1,4-benzodiazepine-4-carboxylic acid, isopropyl ester, hydrochloride;
(R)-7-Bromo-2,3,4,5-tetrahydro-4-[[2-(1H-imidazol-1-yl)ethyl]sulfonyl]-1-(1H-imidazol-4-ylmethyl)-3-(phenylmethyl)-1H-1,4-benzodiazepine, dihydrochloride;
(R)-7-Cyano-2,3,4,5-tetrahydro-1-(1H-imidazol-4-ylmethyl)-4-(propylsulfonyl)-3-(3-pyridinylmethyl)-1H-1,4-benzodiazepine, hydrochloride;
7-Bromo-2,3,4,5-tetrahydro-1-(1H-imidazol-4-ylmethyl)-3-(phenylmethyl)-1H-1,4-benzodiazepin-5-one, hydrochloride;
(R)-7-Bromo-2,3,4,5-tetrahydro-1-(1H-imidazol-1-ylacetyl)-4-(methylsulfonyl)-3-(phenylmethyl)-1H-1,4-benzodiazepine, trifluoroacetate;
1,2,4,5-Tetrahydro-1-(1H-imidazol-4-ylmethyl)-2-(2-phenylethyl)-3H-1,4-benzodiazepin-3-one;
2,3,4,5-Tetrahydro-1-(1H-imidazol-4-ylmethyl)-4-(methylsulfonyl)-2-(2-phenylethyl)-1H-1,4-benzodiazepine, monohydrochloride;
(R)-2,3,4,5-Tetrahydro-1-(1H-imidazol-4-ylmethyl)-4-(methylsulfonyl)-7-phenyl-3-(4-pyridinylmethyl)-1H-1,4-benzodiazepine, dihydrochloride;
(R)-2,3,4,5-Tetrahydro-1-(1H-imidazol-2-ylmethyl)-4-(phenylsulfonyl)-3-(phenylmethyl)-1H-1,4-benzodiazepine-7-carbonitrile, hydrochloride;
(R)-7-Cyano-1,2,3,5-tetrahydro-1-(1H-imidazol-4-ylmethyl)-N,N-dimethyl-3-(3-pyridinylmethyl)-4H-1,4-benzodiazepine-4-carboxamide, dihydrochloride;
(R)-7-Cyano-1,2,3,5-tetrahydro-1-(1H-imidazol-4-ylmethyl)-N,N-dimethyl-3-(3-pyridinylmethyl)-4H-1,4-benzodiazepine-4-sulfonamide, dihydrochloride;
(R)-2,3,4,5-Tetrahydro-1-(1-(4-cyanophenylmethyl)-imidazol-5-ylmethyl)-4-(methylsulfonyl)-3-(phenylmethyl)-1H-1,4-benzodiazepine-7-carbonitrile, hydrochloride;
(R)-2,3,4,5-Tetrahydro-1-(1-(4-cyanophenylmethyl)-imidazol-4-ylmethyl)-4-(methylsulfonyl)-3-(phenylmethyl)-1H-1,4-benzodiazepine-7-carbonitrile, hydrochloride;
(R)-4-Benzoyl-7-cyano-2,3,4,5-tetrahydro-1-(1H-imidazol-4-ylmethyl)-3-(phenylmethyl)-1H-1,4-benzodiazepine, monohydrochloride;
(R)-7-Cyano-2,3,4,5-tetrahydro-1-[(1-methyl-1H-imidazol-5-yl)methyl]-3-(pyridin-3-ylmethyl)-4-(methylsulfonyl)-1H-1,4-benzodiazepine, dihydrochloride;
(R)-7-Cyano-2,3,4,5-tetrahydro-1-[(1-methyl-1H-imidazol-5-yl)methyl]-3-(pyridin-3-ylmethyl)-4-(propylsulfonyl)-1H-1,4-benzodiazepine, trihydrochloride;
(R)-7-Cyano-2,3,4,5-tetrahydro-1-[(1H-imidazol-4-yl)methyl]-3-(pyridin-3-ylmethyl)-4-(phenylsulfonyl)-1H-1,4-benzodiazepine, dihydrochloride;
2,3,4,5-Tetrahydro-1-(1H-imidazol-4-ylmethyl)-4-(methylsulfonyl)-7-phenyl-3-(phenylmethyl)-1H-1,4-benzodiazepine;
1,2,3,5-Tetrahydro-1-(1H-imidazol-4-ylmethyl)-N-(1-naphthalenyl)-7-phenyl-4H-1,4-benzodiazepine-4-carboxamide, monohydrochloride;
(S)-7-Bromo-2,3,4,5-tetrahydro-1-(1H-imidazol-4-ylmethyl)-4-(methylsulfonyl)-3-(phenylmethyl)-1H-1,4-benzodiazepine, hydrochloride;
N-[2,3,4,5-Tetrahydro-1-(1H-imidazol-4-ylmethyl)-4-(2,3-dimethylbenzoyl)-1H-1,4-benzodiazepin-8-yl]cyclohexanecarboxamide, dihydrochloride;
(R)-7-Cyano-N-[2-(dimethylamino)ethyl]-2,3,4,5-tetrahydro-1-(1H-imidazol-4-ylmethyl)-N-methyl-3-(phenylmethyl)-1H-1,4-benzodiazepine-4-carboxamide, trifluoroacetate (1:2);
7-Bromo-2,3,4,5-tetrahydro-1-(1H-imidazol-4-ylmethyl)-4-(methylsulfonyl)-2-oxo-3-(phenylmethyl)-1H-1,4-benzodiazepine, trifluoroacetate;
(R)-7-Cyano-4-(2-furanylcarbonyl)-2,3,4,5-tetrahydro-1-(1H-imidazol-4-ylmethyl)-3-(phenylmethyl)-1H-1,4-benzodiazepine, trifluoroacetate (1:1);
(R)-7-Cyano-2,3,4,5-tetrahydro-1-(1H-imidazol-4-ylmethyl)-4-[(4-nitrophenyl)sulfonyl]-3-(phenylmethyl)-1H-1,4-benzodiazepine, trifluoracetate;
(R)-7-Cyano-2,3,4,5-tetrahydro-1-(1H-imidazol-4-ylmethyl)-4-[[4-(4-methyl-1-piperazinyl)phenyl]sulfonyl]-3-(phenylmethyl)-1H-1,4-benzodiazepine, trifluoroacetate;
(R)-7-Cyano-2,3,4,5-tetrahydro-1-(1H-imidazol-4-ylmethyl)-4-[[(4-dimethylamino)phenyl]sulfonyl]-3-(phenylmethyl)-1H-1,4-benzodiazepine, trifluoroacetate;
(R)-7-Bromo-4-[[2-(dimethylamino)ethyl]sulfonyl]-2,3,4,5-tetrahydro-1-(1H-imidazol-4-ylmethyl)-3-(phenylmethyl)-4H-1,4-benzodiazepine, dihydrochloride;
(R)-7-Cyano-2,3,4,5-tetrahydro-1-(1H-imidazol-4-ylmethyl)-3-(phenylmethyl)-4-(3-pyridinylsulfonyl)-1H-1,4-benzodiazepine, trihydrochloride;
2,3,4,5-Tetrahydro-1-(1H-imidazol-4-ylmethyl)-4-(methylsulfonyl)-3-(phenylmethyl)-1H-1,4-benzo-diazepine, dihydrochloride;
(R)-7-Bromo-2,3,4,5-tetrahydro-1-[(1-methyl-1H-imidazol-4-yl)methyl]-4-(methylsulfonyl)-3-(phenylmethyl)-1H-1,4-benzodiazepine, dihydrochloride;
(R)-4-[[3-(Dimethylamino)propyl]sulfonyl]-2,3,4,5-tetrahydro-1-(1H-imidazol-4-ylmethyl)-7-phenyl-3-(phenylmethyl)-1H-1,4-benzodiazepine, dihydrochloride;
(R)-7-Cyano-2,3,4,5-tetrahydro-1-(1H-imidazol-4-ylmethyl)-3-(phenylmethyl)-1H-1,4-benzodiazepine, trihydrochloride;
4-Butyl-2,3,4,5-tetrahydro-1-(1H-imidazol-4-ylmethyl)-3-(phenylmethyl)-1H-1,4-benzodiazepine, trihydrochloride;
(R)-7-Bromo-2,3,4,5-tetrahydro-1-(1H-imidazol-4-ylmethyl)-4-[[2-(4-morpholinyl)ethyl]sulfonyl]-3-(phenylmethyl)-1H-1,4-benzodiazepine, dihydrochloride;
(R)-7-Bromo-2,3,4,5-tetrahydro-1-[(1-methyl-1H-imidazol-5-yl)methyl]-4-[[2-(4-morpholinyl)ethyl]sulfonyl]-3-(phenylmethyl)-1H-1,4-benzodiazepine, dihydrochloride;
(R)-7-Cyano-1-(1H-imidazol-4-ylmethyl)-4-(4-morpholinylsulfonyl)-3-(phenylmethyl)-1H-1,4-benzodiazepine, monohydrochloride;
(R)-7-Cyano-1-[(1-methyl-1H-imidazol-5-yl)methyl]-4-[(4-morpholinyl)sulfonyl]-3-(phenylmethyl)-1H-1,4-benzodiazepine, monohydrochloride;
(R)-7-Cyano-2,3,4,5-tetrahydro-1-(1H-imidazol-4-ylmethyl)-4-[(4-aminophenyl)sulfonyl]-3-(phenylmethyl)-1H-1,4-benzodiazepine, hydrochloride;
2,3,4,5-Tetrahydro-1-(1H-imidazol-4-ylmethyl)-4-[(4-pyridylthio)acetyl]-7-phenyl-1H-1,4-benzodiazepine, dihydrochloride;
N-(4-Chlorophenyl)-N′-cyano-1,2,3,5-tetrahydro-1-(1H-imidazol-4-ylmethyl)-7-phenyl-4H-1,4-benzodiazepine-4-imidamide, monohydrochloride;
4-Acetyl-7-bromo-1,2,4,5,1′,3′-hexahydro-1-(1H-imidazol-4-ylmethyl)spiro[3H-1,4-benzodiazepine-3,2′-[2H]indene], dihydrochloride;
7-Bromo-4-[3-(dimethylamino)-1-oxopropyl]-2,3,4,5-tetrahydro-1-(1H-imidazol-4-ylmethyl)-3-(phenylmethyl)-1H-1,4-benzodiazepine, trifluoroacetate (1:1);
(R)-2,3,4,5-Tetrahydro-1-(1-methyl-1H-imidazol-5-ylmethyl)-4-(phenylsulfonyl)-3-(phenylmethyl)-1H-1,4-benzodiazepine-7-carbonitrile, monohydrochloride;
2,3,4,5-Tetrahydro-1-[(1-methyl-1H-imidazol-5-yl)-methyl]-4-(methyl-sulfonyl)-7-phenyl-3-(pyridin-3-yl-methyl)-1H-1,4-benzodiazepine, hydrochloride (1:1.5), trifluoroacetate (1:0.75) salt;
4-[4-(Fluorophenyl)sulfonyl]-2,3,4,5-tetrahydro-1-(1H-imidazol-4-ylmethyl)-2-(2-phenylethyl)-1H-1,4-benzodiazepine, monohydrochloride;
7-Bromo-2,3,4,5-tetrahydro-1-(1H-imidazol-4-yl-methyl)-4-(methyl-sulfonyl)-2-(2-phenylethyl)-1H-1,4-benzodiazepine, monohydrochloride;
(R)-7-Cyano-2,3,4,5-tetrahydro-1-(1-methyl-1H-imidazol-5-ylmethyl)-4-[[2-(1-morpholinyl)ethyl]sulfonyl]-3-(phenylmethyl)-1H-1,4-benzodiazepine, dihydrochloride;
(R)-7-Bromo-2,3,4,5-tetrahydro-1-(1H-imidazol-4-ylmethyl)-4-(methyl-sulfonyl)-3-(4-bromophenylmethyl)-1H-1,4-benzodiazepine, hydrochloride;
(R)-7-Bromo-2,3,4,5-tetrahydro-1-(1H-imidazol-4-ylmethyl)-4-(methyl-sulfonyl)-3-(thiazol-4-ylmethyl)-1H-1,4-benzodiazepine, hydrochloride;
(R)-7-Bromo-2,3,4,5-tetrahydro-1-(1H-imidazol-4-ylmethyl)-4-(propyl-sulfonyl)-3-(thiazol-4-ylmethyl)-1H-1,4-benzodiazepine, hydrochloride;
(R)-7-Bromo-2,3,4,5-tetrahydro-1-(1H-imidazol-4-ylmethyl)-4-(propylsulfonyl)-3-(4-bromophenylmethyl)-1H-1,4-benzodiazepine, hydrochloride;
(R)-7-Bromo-2,3,4,5-tetrahydro-1-(1H-imidazol-4-ylmethyl)-3-(pyridin-3-ylmethyl)-4-(methylsulfonyl)-1H-1,4-benzodiazepine, trihydrochloride;
(R)-7-Bromo-2,3,4,5-tetrahydro-1-(1H-1-methyl-imidazol-5-ylmethyl)-3-(pyridin-3-ylmethyl)-4-(methylsulfonyl)-1H-1,4-benzodiazepine, dihydrochloride;
(R)-7-Cyano-2,3,4,5-tetrahydro-1-(1H-imidazol-4-ylmethyl)-4-(phenyl-sulfonyl)-3-(4-cyanophenylmethyl)-1H-1,4-benzodiazepine, hydrochloride;
(R)-7-Cyano-4-[(N-methyl-N-phenylmethyl)aminosulfonyl]-1-[(1H-imidazol-4-yl)methyl]-3-(phenylmethyl)-1H-1,4-benzodiazepine, monohydrochloride;
(R)-7-Cyano-4-[N-(tetrahydroisoquinolinyl)sulfonyl]-1-[(1H-imidazol-4-yl)methyl]-3-(phenylmethyl)-1H-1,4-benzodiazepine, monohydrochloride;
(R)-7-Cyano-2,3,4,5-tetrahydro-1-(1H-imidazol-4-ylmethyl)-4-(phenylsulfonyl)-3-(2-thienylmethyl)-1H-1,4-benzodiazepine, hydrochloride;
cis-2,3,4,5-Tetrahydro-1,5-bis(1H-imidazol-4-ylmethyl)-3-(phenylmethyl)-1H-1,5-benzodiazepine-2-carboxylic acid ethyl ester, trifluoroacetate (1:2);
(R)-7-Cyano-4-[(N-piperidinyl)sulfonyl]-1-[(1H-imidazol-4-yl)methyl]-3-(phenylmethyl)-1H-1,4-benzodiazepine, monohydrochloride;
(R)-7-Cyano-2,3,4,5-tetrahydro-1-(1H-1-methyl-imidazol-5-ylmethyl)-3-(phenylmethyl)-4-(2-thienylsulfonyl)-1H-1,4-benzodiazepine, hydrochloride;
(R)-7-Cyano-2,3,4,5-tetrahydro-1-(1H-imidazol-4-ylmethyl)-3-(pyridin-3-ylmethyl)-4-[[2-(dimethylamino)ethyl]sulfonyl]-1H-1,4-benzodiazepine, trihydrochloride;
(R)-7-Cyano-2,3,4,5-tetrahydro-1-(1H-1-methyl-imidazol-5-ylmethyl)-3-(phenylmethyl)-4-(propylsulfonyl)-1H-1,4-benzodiazepine, hydrochloride;
N-(Cyano)-N′-methyl-1,2,3,5-tetrahydro-1-(1H-imidazol-4-ylmethyl)-7-phenyl-4H-1,4-benzodiazepine-4-imidamide, hydrochloride;
(R)-7-Cyano-4-[(2-nitrophenyl)-sulfonyl]-2,3,4,5-tetrahydro-1-(1H-imidazol-4-ylmethyl)-3-(phenyl-methyl)-1H-1,4-benzodiazepine, hydrochloride;

R)-7-Cyano-4-[(4-methyl-phenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-4-(butylsulfonyl)-2,3,4,5-tetrahydro-1-(1H-imidazol-4-ylmethyl)-3-(phenylmethyl)-1H-1,4-benzodiazepine, hydrochloride;
(R)-7-Cyano-4-[(2-trifluoro-methylphenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-4-[(2-trifluoromethoxyphenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-4-[(2-methoxy-carbonylphenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-4-[(2-methyl-sulfonylphenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-2,3,4,5-tetrahydro-1-(1H-imidazol-4-ylmethyl)-3-(phenylmethyl)-4-(((4-methylsulfonyl)-phenyl)-sulfonyl)-1H-1,4-benzodiazepine;
(R)-7-Cyano-2,3,4,5-tetrahydro-1-(1H-imidazol-4-ylmethyl)-3-(phenylmethyl)-4-(((4-trifluoromethyl)-phenyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((3-methoxypropyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((3,4-dimethoxyphenyl)-sulfonyl)-1H-1,4-benzodiazepine;
(R)-7-Cyano-2,3,4,5-tetrahydro-1-(1H-imidazol-4-ylmethyl)-3-((4-fluorophenyl)methyl)-4-(phenylsulfonyl)-1H-1,4-benzodiazepine;
(R)-7-Cyano-4-[(N-cyclopropylmethyl-N-propyl)-aminosulfonyl]-1-[(1H-imidazol-4-yl)methyl]-3-(phenylmethyl)-1H-1,4-benzodiazepine;
(R)-7-Cyano-4-[(N,N-(dibutylamino))-sulfonyl]-1-[(1H-imidazol-4-yl)methyl]-3-(phenylmethyl)-1H-1,4-benzodiazepine;
1,2,3,4-Tetrahydro-7-bromo-4-[(1H-imidazol-4-yl)methyl]-2-phenylmethyl-1-(methylsulfonyl)quinoxaline;
(R)-7-Cyano-2,3,4,5-tetrahydro-1-(1H-imidazol-4-ylmethyl)-3-(phenylmethyl)-4-((imidazol-4-yl)methylsulfonyl)-1H-1,4-benzodiazepine;
(R)-7-Cyano-2,3,4,5-tetrahydro-1-(1H-imidazol-4-ylmethyl)-3-((2-thienyl)methyl)-4-(propylsulfonyl)-1H-1,4-benzodiazepine;
(R)-7-Cyano-2,3,4,5-tetrahydro-1-(1H-imidazol-4-ylmethyl)-3-((2-thienyl)methyl)-4-((2-thienyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((3-methylthiopropyl)-sulfonyl)-1H-1,4-benzodiazepine;
(R)-7-Cyano-2,3,4,5-tetrahydro-1-(1H-imidazol-4-ylmethyl)-3-(phenylmethyl)-4-(((3-methylthioxo)-propyl)-sulfonyl)-1H-1,4-benzodiazepine;
(R)-7-Cyano-2,3,4,5-tetrahydro-1-(1H-imidazol-4-ylmethyl)-3-(phenylmethyl)-4-(((3-methylsulfonyl)-propyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((2-methylpropyl)-sulfonyl)-1H-1,4-benzodiazepine;
(R)-7-Cyano-2,3,4,5-tetrahydro-1-(1H-imidazol-4-ylmethyl)-3-(phenylmethyl)-4-30(cyclopentylsulfonyl)-1H-1,4-benzodiazepine;
(R)-7-Cyano-2,3,4,5-tetrahydro-1-(1H-imidazol-4-ylmethyl)-3-(phenylmethyl)-4-((4,4,4-trifluorobutyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((phenylmethyl)-sulfonyl)-1H-1,4-benzodiazepine;
(R)-7-Cyano-2,3,4,5-tetrahydro-1-(1H-imidazol-4-ylmethyl)-3-(phenylmethyl)-4-[[2-(5-(N-benzoyl)-aminomethyl)-thienyl]-sulfonyl]-1H-1,4-benzodiazepine
(R)-7-Cyano-2,3,4,5-tetrahydro-1-(1H-imidazol-4-ylmethyl)-3-(phenylmethyl)-4-[[2-(1-(3-chloro-5-methyl-pyridin-2-yl))-pyrrolyl]-sulfonyl]-1H-1,4-benzodiazepine;
(R)-7-Cyano-2,3,4,5-tetrahydro-1-(1H-imidazol-4-ylmethyl)-3-(phenylmethyl)-4-((4-carboxyphenyl)-sulfonyl)-1H-1,4-benzodiazepine;
(R)-7-Cyano-2,3,4,5-tetrahydro-1-(1H-imidazol-4-ylmethyl)-3-(phenylmethyl)-4-[((3-methyl-1,2,4-oxadiazol-5-yl)-phenyl)-sulfonyl]-1H-1,4-benzodiazepine;
(R)-7-Cyano-2,3,4,5-tetrahydro-1-(1H-imidazol-4-ylmethyl)-3-(phenylmethyl)-4-((2,5-dimethoxyphenyl)-sulfonyl)-1H-1,4-benzodiazepine;
(R)-7-Cyano-4-[(N-tetrahydroquinolinyl)sulfonyl]-1-[(1H-imidazol-4-yl)methyl]-3-(phenylmethyl)-1H-1,4-benzodiazepine;
(R)-7-Cyano-4-[(N,N-bis-[1-(2-methylpropyl)amino]-sulfonyl]-1-[(1H-imidazol-4-yl)methyl]-3-(phenylmethyl)-1H-1,4-benzodiazepine;
(R)-7-Cyano-4-[(N-methyl-N-phenyl)aminosulfonyl]-1-[(1H-imidazol-4-yl)methyl]-3-(phenylmethyl)-1H-1,4-benzodiazepine;
(R)-7-Cyano-2,3,4,5-tetrahydro-1-(1H-imidazol-4-ylmethyl)-3-(2-(2,6-dimethylphenyl)-ethyl)-4-(methylsulfonyl)-1H-1,4-benzodiazepine;
(R)-7-Cyano-2,3,4,5-tetrahydro-1-(1-(N-phthalimidoethyl)-imidazol-5-ylmethyl)-3-(phenylmethyl)-4-(methylsulfonyl)-1H-1,4-benzodiazepine;
(R)-7-Cyano-2,3,4,5-tetrahydro-1-[(2-(N,N-dimethylamino)-ethyl)-imidazol-5-ylmethyl]-3-(phenylmethyl)-4-(methylsulfonyl)-1H-1,4-benzodiazepine;
(R)-7-Cyano-2,3,4,5-tetrahydro-1-[(2-aminoethyl)-imidazol-5-ylmethyl]-3-(phenylmethyl)-4-(methylsulfonyl)-1H-1,4-benzodiazepine;
(R)-4-(methanesulfonyl)-2,3,4,5-tetrahydro-1-[(1H-imidazol-4-ylmethyl)-3-(phenylmethyl)-1H-8-oxo-pyrimidino[4,5-e]-1,4-diazepine;
(R)-7-Cyano-2,3,4,5-tetrahydro-1-(1H-imidazol-4-ylmethyl)-3-((4-(2-methoxyethoxy)-phenyl)methyl)-4-(phenylsulfonyl)-1H-1,4-benzodiazepine;
(R)-7-Cyano-2,3,4,5-tetrahydro-1-(1H-imidazol-4-ylmethyl)-3-((4-(2-(dimethylamino)-ethoxy)-phenyl)methyl)-4-(phenylsulfonyl)-1H-1,4-benzodiazepine;
(R)-7-Cyano-2,3,4,5-tetrahydro-1-(1H-imidazol-4-ylsulfonyl)-3-(phenylmethyl)-4-(methylsulfonyl)-1H-1,4-benzodiazepine;
(R)-7-Cyano-2,3,4,5-tetrahydro-1-(1H-imidazol-4-ylsulfonyl)-3-(phenylmethyl)-4-(propylsulfonyl)-1H-1,4-benzodiazepine;
(R)-7-Cyano-2,3,4,5-tetrahydro-1-(1H-imidazol-4-ylsulfonyl)-3-(phenylmethyl)-4-(phenylsulfonyl)-1H-1,4-benzodiazepine;
(R)-7-Cyano-2,3,4,5-tetrahydro-1-(1H-imidazol-4-ylsulfonyl)-3-(phenylmethyl)-4-(2-thienylsulfonyl)-1H-1,4-benzodiazepine;
7-Cyano-2,3,4,5-tetrahydro-1-(1H-imidazol-4-ylmethyl)-3-(R)-[(R)-1-phenyl-ethyl]-4-(methylsulfonyl)-1H-1,4-benzodiazepine;
7-Cyano-2,3,4,5-tetrahydro-1-(1H-imidazol-4-ylmethyl)-3-(R)-[(R)-1-phenyl-ethyl]-4-(propylsulfonyl)-1H-1,4-benzodiazepine;
7-Cyano-2,3,4,5-tetrahydro-1-(1H-imidazol-4-ylmethyl)-3-(R)-[(R)-1-phenyl-ethyl]-4-(phenylsulfonyl)-1H-1,4-benzodiazepine;
7-Cyano-2,3,4,5-tetrahydro-1-(1H-imidazol-4-ylmethyl)-3-(R)-[(R)-1-phenyl-ethyl]-4-((2-thienyl)-sulfonyl)-1H-1,4-benzodiazepine;
7-Cyano-2,3,4,5-tetrahydro-1-(1H-imidazol-4-ylmethyl)-3-(S)-[(R)-1-phenyl-ethyl]-4-(methylsulfonyl)-1H-1,4-benzodiazepine;
7-Cyano-2,3,4,5-tetrahydro-1-(1H-imidazol-4-ylmethyl)-3-(S)-[(R)-1-phenyl-ethyl]-4-(propylsulfonyl)-1H-1,4-benzodiazepine;
7-Cyano-2,3,4,5-tetrahydro-1-(1H-imidazol-4-ylmethyl)-3-(S)-[(R)-1-phenyl-ethyl]-4-(phenylsulfonyl)-1H-1,4-benzodiazepine;
7-Cyano-2,3,4,5-tetrahydro-1-(1H-imidazol-4-ylmethyl)-3-(S)-[(R)-1-phenyl-ethyl]-4-((2-thienyl)-sulfonyl)-1H-1,4-benzodiazepine;
7-Cyano-2,3,4,5-tetrahydro-1-(1H-imidazol-4-ylmethyl)-3-(R)-[(S)-1-phenyl-ethyl]-4-(methylsulfonyl)-1H-1,4-benzodiazepine;
7-Cyano-2,3,4,5-tetrahydro-1-(1H-imidazol-4-ylmethyl)-3-(R)-[(S)-1-phenyl-ethyl]-4-(propylsulfonyl)-1H-1,4-benzodiazepine;
7-Cyano-2,3,4,5-tetrahydro-1-(1H-imidazol-4-ylmethyl)-3-(R)-[(S)-1-phenyl-ethyl]-4-(phenylsulfonyl)-1H-1,4-benzodiazepine;
7-Cyano-2,3,4,5-tetrahydro-1-(1H-imidazol-4-ylmethyl)-3-(R)-[(S)-1-phenyl-ethyl]-4-((2-thienyl)-sulfonyl)-1H-1,4-benzodiazepine;
7-Cyano-2,3,4,5-tetrahydro-1-(1H-imidazol-4-ylmethyl)-3-(S)-[(S)-1-phenyl-ethyl]-4-(methylsulfonyl)-1H-1,4-benzodiazepine;
7-Cyano-2,3,4,5-tetrahydro-1-(1H-imidazol-4-ylmethyl)-3-(S)-[(S)-1-phenyl-ethyl]-4-(propylsulfonyl)-1H-1,4-benzodiazepine;
7-Cyano-2,3,4,5-tetrahydro-1-(1H-imidazol-4-ylmethyl)-3-(S)-[(S)-1-phenyl-ethyl]-4-(phenylsulfonyl)-1H-1,4-benzodiazepine;
7-Cyano-2,3,4,5-tetrahydro-1-(1H-imidazol-4-ylmethyl)-3-(S)-[(S)-1-phenyl-ethyl]-4-((2-thienyl)-sulfonyl)-1H-1,4-benzodiazepine;
7-Cyano-2,3,4,5-tetrahydro-1-(1H-imidazol-4-ylmethyl)-3-(R)-[(R)-phenylcyclopropyl)-4-(methylsulfonyl)-1H-1,4-benzodiazepine;
7-Cyano-2,3,4,5-tetrahydro-1-(1H-imidazol-4-ylmethyl)-3-(R)-[(R)-phenylcyclopropyl)-4-(propylsulfonyl)-1H-1,4-benzodiazepine;
7-Cyano-2,3,4,5-tetrahydro-1-(1H-imidazol-4-ylmethyl)-3-(R)-[(R)-phenylcyclopropyl)-4-(phenylsulfonyl)-1H-1,4-benzodiazepine;
7-Cyano-2,3,4,5-tetrahydro-1-(1H-imidazol-4-ylmethyl)-3-(R)-[(R)-phenylcyclopropyl)-4-((2-thienyl)-sulfonyl)-1H-1,4-benzodiazepine;
7-Cyano-2,3,4,5-tetrahydro-1-(1H-imidazol-4-ylmethyl)-3-(R)-[(S)-phenylcyclopropyl)-4-(methylsulfonyl)-1H-1,4-benzodiazepine;
7-Cyano-2,3,4,5-tetrahydro-1-(1H-imidazol-4-ylmethyl)-3-(R)-[(S)-phenylcyclopropyl)-4-(propylsulfonyl)-1H-1,4-benzodiazepine;
7-Cyano-2,3,4,5-tetrahydro-1-(1H-imidazol-4-ylmethyl)-3-(R)-[(S)-phenylcyclopropyl)-4-(phenylsulfonyl)-1H-1,4-benzodiazepine;
7-Cyano-2,3,4,5-tetrahydro-1-(1H-imidazol-4-ylmethyl)-3-(R)-[(S)-phenylcyclopropyl)-4-((2-thienyl)-sulfonyl)-1H-1,4-benzodiazepine;
7-Cyano-2,3,4,5-tetrahydro-1-(1H-imidazol-4-ylmethyl)-3-(S)-[(R)-phenylcyclopropyl)-4-(methylsulfonyl)-1H-1,4-benzodiazepine;
7-Cyano-2,3,4,5-tetrahydro-1-(1H-imidazol-4-ylmethyl)-3-(S)-[(R)-phenylcyclopropyl)-4-(propylsulfonyl)-1H-1,4-benzodiazepine;
7-Cyano-2,3,4,5-tetrahydro-1-(1H-imidazol-4-ylmethyl)-3-(S)-[(R)-phenylcyclopropyl)-4-(phenylsulfonyl)-1H-1,4-benzodiazepine;
7-Cyano-2,3,4,5-tetrahydro-1-(1H-imidazol-4-ylmethyl)-3-(S)-[(R)-phenylcyclopropyl)-4-((2-thienyl)-sulfonyl)-1H-1,4-benzodiazepine;
7-Cyano-2,3,4,5-tetrahydro-1-(1H-imidazol-4-ylmethyl)-3-(S)-[(S)-phenylcyclopropyl)-4-(methylsulfonyl)-1H-1,4-benzodiazepine;
7-Cyano-2,3,4,5-tetrahydro-1-(1H-imidazol-4-ylmethyl)-3-(S)-[(S)-phenylcyclopropyl)-4-(propylsulfonyl)-1H-1,4-benzodiazepine;
7-Cyano-2,3,4,5-tetrahydro-1-(1H-imidazol-4-ylmethyl)-3-(S)-[(S)-phenylcyclopropyl)-4-(phenylsulfonyl)-1H-1,4-benzodiazepine;
7-Cyano-2,3,4,5-tetrahydro-1-(1H-imidazol-4-ylmethyl)-3-(S)-[(S)-phenylcyclopropyl)-4-((2-thienyl)-sulfonyl)-1H-1,4-benzodiazepine;
(R)-7-Cyano-2,3,4,5-tetrahydro-1-(1H-imidazol-4-ylmethyl)-3-(phenylmethyl)-4-[(2-(5-(pyridin-2-yl))-thienyl)-sulfonyl])-1H-1,4-benzodiazepine;
(R)-7-Cyano-2,3,4,5-tetrahydro-1-(1H-imidazol-4-ylmethyl)-3-(phenylmethyl)-4-[(2-(5-(1,2-isoxazol-3-yl))-thienyl)-sulfonyl])-1H-1,4-benzodiazepine;
(R)-7-Cyano-2,3,4,5-tetrahydro-1-(3-(1H-imidazol-2-yl)-propyl)-3-(phenylmethyl)-4-(phenylsulfonyl)-1H-1,4-benzodiazepine;
(R)-7-Cyano-2,3,4,5-tetrahydro-1-(3-(1H-imidazol-2-yl)-propyl)-3-(phenylmethyl)-4-(methylsulfonyl)-1H-1,4-benzodiazepine;
(R)-7-Cyano-2,3,4,5-tetrahydro-1-(3-(1H-imidazol-2-yl)-propyl)-3-(phenylmethyl)-4-(propylsulfonyl)-1H-1,4-benzodiazepine;
(R)-7-Cyano-2,3,4,5-tetrahydro-1-(3-(1H-imidazol-2-yl)-propyl)-3-(phenylmethyl)-4-((2-thienyl)-sulfonyl)-1H-1,4-benzodiazepine;
(R)-7-Cyano-2,3,4,5-tetrahydro-1-(2-(1H-imidazol-2-yl)-ethylsulfonyl)-3-(phenylmethyl)-4-(phenylsulfonyl)-1H-1,4-benzodiazepine;
(R)-7-Cyano-2,3,4,5-tetrahydro-1-(2-(1H-imidazol-2-yl)-ethylsulfonyl)-3-(phenylmethyl)-4-(methylsulfonyl)-1H-1,4-benzodiazepine;
(R)-7-Cyano-2,3,4,5-tetrahydro-1-(2-(1H-imidazol-2-yl)-ethylsulfonyl)-3-(phenylmethyl)-4-(propylsulfonyl)-1H-1,4-benzodiazepine;
(R)-7-Cyano-2,3,4,5-tetrahydro-1-(2-(1H-imidazol-2-yl)-ethylsulfonyl)-3-(phenylmethyl)-4-((2-thienyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((1-oxoethyl)-amino)-1H-1,4-benzodiazepine;
(R)-7-Cyano-2,3,4,5-tetrahydro-1-(1H-imidazol-4-ylmethyl)-3-(phenylmethyl)-4-(methanesulfonylamino)-1H-1,4-benzodiazepine;
(R)-7-Cyano-2,3,4,5-tetrahydro-1-(1H-imidazol-4-ylmethyl)-3-(phenylmethyl)-4-(phenylsulfonylamino)-1H-1,4-benzodiazepine.

In another embodiment of the invention the compound has the formula: 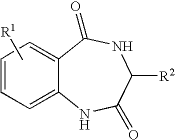
wherein R₁ is selected from Cl, Br, phenyl, pyridyl or cyano and R₂ is selected from substituted aralkyl or substituted heterocycloalkyl.

In yet another embodiment of the invention the compound has the formula 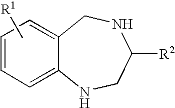
wherein R₁ is selected from Cl, Br, phenyl, pyridyl or cyano and R₂ is selected from substituted aralkyl or substituted heterocycloalkyl.

In another embodiment of the invention wherein the compound has the formula 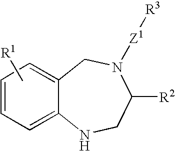
wherein
R₁ is selected from Cl, Br, phenyl, pyridyl or cyano;
R₂ is selected from substituted aralkyl or substituted heterocycloalkyl;
R₃ is selected from substituted alkyl, substituted aryl or substituted heterocyclo;
Z₁ is selected from CO, SO₂, CO₂, CONHR₅, SO₃, SO₂NR₅, or C(NCN)NR₅; R₅ is selected from hydrogen, lower alkyl, substituted alkyl, aryl or substituted aryl.

In one aspect of the invention the compound has the formula 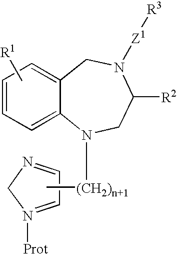
wherein
R₁ is selected from Cl, Br, phenyl, pyridyl or cyano;
R₂ is selected from substituted aralkyl or substituted heterocycloalkyl;
R₃ is selected from substituted alkyl, substituted aryl or substituted heterocyclo;
Z₁ is selected from CO, SO₂, CO₂, CONHR₅, SO₃, SO₂NR₅, or C(NCN)NR₅;
Prot is triphenylmethyl or Boc; and
R₅ is selected from hydrogen, lower alkyl, substituted alkyl, aryl or substituted aryl.

In one aspect the invention provides a method of treating a synucleinopathic subject, the method comprising, administering to a synucleinopathic subject a farnesyl transferase inhibitor compound of the formula:

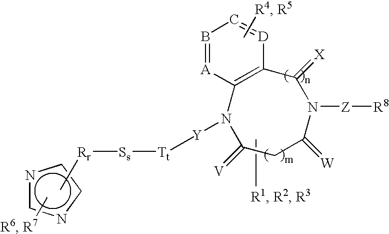

or a stereoisomeric form, or a pharmaceutically acceptable acid or base addition salt form thereof, in a therapeutically effective amount,

wherein

n is 1;
r, s and t are 0 or 1;
p is 0, 1 or 2;
V, W and X are selected from the group consisting of oxygen, hydrogen, R¹, R² and R³; 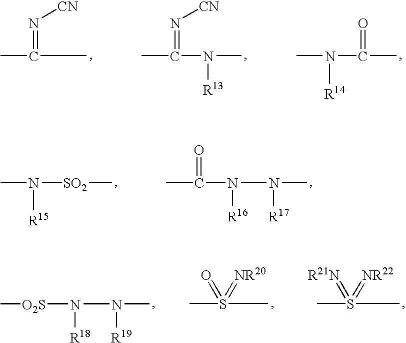
Z and Y are selected from the group consisting of CHR⁹, SO₂, SO₃, CO, CO₂, O, NR¹⁰, SO₂NR¹¹, CONR¹²,
or Z may be absent;
R⁶, R⁷, R⁹, R¹⁰, R¹¹, R¹², R¹³R¹⁴R¹⁵R¹⁶R¹⁷, R¹⁸, R¹⁹, R²⁰, R²¹, R²², R²⁴, R²⁵, R²⁶, R²⁸, R²⁹, R³⁰, R³¹, R³², R³³, R³⁴, R³⁵, R³⁶, R³⁷, and R³⁸ are selected from the group consisting of hydrogen, lower alkyl, substituted alkyl, aryl and substituted aryl;
R⁴ and R⁵ are selected from the group consisting of hydrogen, halo, nitro, cyano and U—R²³;
U is selected from the group consisting of sulfur, oxygen, NR²⁴, CO, SO, SO₂, CO₂, NR²⁵CO₂, NR²⁶CONR²⁷, NR²⁸SO₂, NR²⁹SO₂NR³⁰, SO₂NR³¹, NR³²CO, CONR³³, PO₂R³⁴ and PO₃R³⁵ or U is absent;
R¹, R² and R³ are selected from the group consisting of hydrogen, alkyl, alkoxycarbonyl, substituted alkyl, alkenyl, substituted alkenyl, alkynyl, substituted alkynyl, aralkyl, cycloalkyl, aryl, substituted aryl, heterocyclo, substituted heterocyclo, cyano, carboxy, carbamyl and substituted carbamyl;
R⁸ and R²³ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo and substituted heterocyclo;
any two of R¹, R² and R³ may be joined to form a cycloalkyl group;
R, S and T are selected from the group consisting of CH₂, CO and CH(CH₂)pQ wherein Q is NR³⁶R³⁷, OR³⁸ or CN; and
A, B, C and D are carbon;
with the provisos that
   V and W are not both oxygen;
   W and X together may be oxygen only if Z is either absent, O, NR¹⁰, CHR⁹, —N(R¹⁴)—C(O)—, —N(R¹⁵)—SO₂—;
   R²³ may be hydrogen except when U is SO, SO₂, NR²⁵CO₂ or NR²⁸SO₂; and
   R⁸ may be hydrogen except when Z is SO₂, CO₂, —N(R¹⁵)—SO₂,

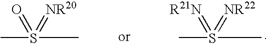

In one embodiment of the invention the pharmaceutically acceptable salt is mesylate. In one embodiment of the invention the compound is (R)-7-cyano-2,3,4,5-tetrahydro-1-(1H-imidazol-4-ylmethyl)-3-(phenylmethyl)-4-(2-thienylsulfonyl)-1H-1,4-benzodiazepine, mesylate salt. In yet another embodiment of the invention the compound is selected from the group consisting of:
2,3,4,5-Tetrahydro-1-(1H-imidazol-4-ylmethyl)-4-(1-naphthalenylcarbonyl)-1H-1,4-benzodiazepine, hydrochloride;
8-Chloro-2,3,4,5-tetrahydro-1-(1H-imidazol-4-ylmethyl)-4-(1-naphthalenylcarbonyl)-1H-1,4-benzodiazepine, hydrochloride;
2,3,4,5-Tetrahydro-1-(1H-imidazol-4-yl-methyl)-2-methyl-4-(1-naphthalenylcarbonyl)-1H-1,4-benzodiazepine, hydrochloride;
2,3,4,5-Tetrahydro-4-(1-naphthalenylcarbonyl)-1-[[1-(phenylmethyl)-1H-imidazol-5-yl]methyl]-1H-1,4-benzodiazepine, hydrochloride;
2,3,4,5-Tetrahydro-(1H-imidazol-4-yl-methyl)-4-(1-naphthalenylsulfonyl)-1H-1,4-benzodiazepine, hydrochloride;
(S)-2,3,4,5-Tetrahydro-1-(1H-imidazol-4-ylmethyl)-3-[2-(methylthio)ethyl]-4-(1-naphthalenylcarbonyl)-1H-1,4-benzodiazepine, hydrochloride;
2,3,4,5-Tetrahydro-1-(1H-imidazol-4-yl-methyl)-N-methyl-N-phenyl-4H-1,4-benzodiazepine-4-carboxamide, hydrochloride;
2-[2,3,4,5-Tetrahydro-1-(1H-imidazol-4-yl-methyl)-1H-1,4-benzodiazepin-4-yl]sulfonyl]benzoic acid, methyl ester, hydrochloride;
7-Bromo-2,3,4,5-tetrahydro-1-(1H-imidazol-4-ylmethyl)-4-(1-naphthalenylcarbonyl)-1H-1,4-benzodiazepine, hydrochloride;
2,3,4,5-Tetrahydro-1-(1H-imidazol-4-ylmethyl)-4-(1-naphthalenenylcarbonyl)-7-phenyl-1H-1,4-benzodiazepine, hydrochloride;
2,3,4,5-Tetrahydro-1-(1H-imidazol-2-ylmethyl)-4-(1-naphthalenylcarbonyl)-1H-1,4-benzodiazepine, dihydrochloride;
2,3,4,5-Tetrahydro-1-[3-(1H-imidazol-2-yl)propyl]-4-(1-naphthalenylcarbonyl)-1H-1,4-benzodiazepine, dihydrochloride;
1-[3-Amino-3-(1H-imidazol-2-yl)propyl]-2,3,4,5-tetrahydro-4-(1-naphthalenylcarbonyl)-1H-1,4-benzodiazepine, trihydrochloride;
2,3,4,5-Tetrahydro-1-(1H-imidazol-4-ylmethyl)-3-methyl-4-(1-napthalenylcarbonyl)-1H-1,4-benzodiazepine, hydrochloride;
(S)-2,3,4,5-Tetrahydro-1-(1H-imidazol-4-ylmethyl)-3-[2-(methylthio)ethyl]-4-(1-naphthalenylmethyl)-1H-1,4-benzodiazepine, hydrochloride;
2,3,4,5-Tetrahydro-1-(1H-imidazol-4-ylmethyl)-9-methyl-4-(1-naphthalenylcarbonyl)-1H-1,4-benzodiazepine, dihydrochloride;
1-[[2-(2-Aminoethyl)-1H-imidazol-4-ylmethyl-2,3,4,5-tetrahydro-4-(1-naphthalenylcarbonyl)-1H -1,4-benzodiazepine, trihydrochloride;
1-[[2-Aminomethyl)-1H-imidazol-4-yl]methyl]-2,3,4,5-tetrahydro-4-(1-naphthalenylcarbonyl)-1H-1,4-benzodiazepine, trihydrochloride;
N-[2,3,4,5-Tetrahydro-1-(1H-imidazol-4-ylmethyl)-4-(1-naphthalenylcarbonyl)-1H-1,4-benzodiazepin-8-yl]acetamide, dihydrochloride;
2,3,4,5-Tetrahydro-1-(1H-imidazol-4-ylmethyl)-4-(1-naphthalenylcarbonyl)-1H-naphtho[2,3-e]-1,4-diazepine, dihydrochloride;
2,3,4,5-Tetrahydro-1-(1H-imidazol-4-ylmethyl)-4-(1-naphthalenylcarbonyl)-8-nitro-1H-1,4-benzodiazepine, dihydrochloride;
2,3,4,5-Tetrahydro-1-(1H-imidazol-4-ylmethyl)-4-(1-naphthalenylcarbonyl)-8-amino-1H-1,4-benzodiazepine, dihydrochloride;
N-[2,3,4,5Tetrahydro-1-(1H-imidazol-4-ylmethyl)-4-(1-naphthalenylcarbonyl)-1H-1,4-benzodiazepin-8-yl]benzamide, dihydrochloride;
N-[2,3,4,5-Tetrahydro-1-(1H-imidazol-4-ylmethyl)-4-(1-naphthalenylcarbonyl)-1H-1,4-benzodiazepin-8-yl]cyclohexanamide, dihydrochloride;
2,3,4,5-Tetrahydro-1-[2-(1H-imidazol-4-yl)ethyl]-4-(1-naphthalenylcarbonyl)-1H-1,4-benzodiazepine, dihydrochloride;
2,3,4,5-Tetrahydro-1-[2-(1H-imidazol-4-yl)ethyl]-4-(1-naphthalenylcarbonyl)-7-phenyl-1H-1,4-benzodiazepine, dihydrochloride;
7-Bromo-2,3,4,5-tetrahydro-1-[2-(1H-imidazol-4-yl)ethyl]-4-(1-naphthalenylcarbonyl)-1H-1,4-benzodiazepine, dihydrochloride;
1-[[1-(2-Aminoethyl)-1H-imidazol-5-yl]methyl]-2,3,4,5-tetrahydro-4-naphthalenylcarbonyl)-7-phenyl-1H-1,4-benzodiazepine, trihydrochloride;
2,3,4,5Tetrahydro-1-(1H-imidazol-4-ylmethyl)-7-phenyl-1H-1,4-benzodiazepine-4-carboxylic acid, phenylmethyl ester;
2,3,4,5-Tetrahydro-1-(1H-imidazol-4-ylmethyl)-7-phenyl-4-[2-(trifluoromethoxy)benzoyl]-1H-1,4-benzodiazepine;
1,2,3,5-Tetrahydro-1-(1H-imidazol-4-ylmethyl)-N-methyl-N,7-diphenyl-4H-1,4-benzodiazepine-4-carboxamide, dihydrochloride;
2,3,4,5-Tetrahydro-1-(1H-imidazol-4-ylmethyl)-4-(1-naphthaleneylcarbonyl)-7-(1-piperidinylsulfonyl)-1H-1,4-benzodiazepine, monohydrochloride;
2,3,4,5-Tetrahydro-1-(1H-imidazol-4-ylmethyl)-4-(1-naphthalenylcarbonyl)-7-pyridin-2-yl-1H-1,4-benzodiazepine, trihydrochloride;
7-(2-Furanyl)-2,3,4,5-tetrahydro-1-(1H-imidazol-4-ylmethyl)-4-(1-naphthalenylcabonyl)-1H-1,4-benzodiazepine, dihydrochloride;
2,3,4,5-Tetrahydro-1-(1H-imidazol-4-ylmethyl)-4-(1-naphthalenylcarbonyl)-7-(2-thienyl)-1H-1,4-benzodiazepine, dihydrochloride;
2,3,4,5-Tetrahydro-1-(1H-imidazol-4-ylmethyl)-4-(1-naphthalenylcarbonyl)-7-(4-pyridinyl)-1H-1,4-benzodiazepine, trihydrochloride;
2,3,4,5-Tetrahydro-1-[3-(1H-imidazol-2-yl)propyl]-4-(1-naphthalenylcarbonyl)-7-phenyl-1H-1,4-benzodiazepine, dihydrochloride;
2,3,4,5-Tetrahydro-1,4-bis(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(1-naphthalenylmethyl)-7-phenyl-1H-1,4-benzodiazepine, trifluoroacetate;
2,3,4,5-Tetrahydro-1-(1H-imidazol-4-ylmethyl)-7-methoxy-4-(1-naphthalenylcarbonyl)-1H-1,4-benzodiazepine, dihydrochloride;
2,3,4,5-Tetrahydro-1-(1H-imidazol-4-ylmethyl)-4-(1-naphthalenylcarbonyl)-1H-1,4-benzodiazepine-7-carboxylic acid, dihydrochloride;
2,3,4,5-Tetrahydro-1-(1H-imidazol-5-ylmethyl)-4-(1-naphthalenylcarbonyl)-7-cyclohexyl-1H-1,4-benzodiazepine, 2,5 hydrochloride;
7-Butyl-2,3,4,5-tetrahydro-1-(1H-imidazol-4-ylmethyl)-4-(1-naphthalenylcarbonyl)-1H-1,4-benzodiazepine, dihydrochloride;
1-[[2-(2-Aminoethyl)-1H-imidazol-4-yl]methyl]-2,3,4,5-tetrahydro-4-(1-naphthalenylcarbonyl)-7-phenyl-1H-1,4-benzodiazepine, trihydrochloride;
1-[[2-(Aminomethyl)-1H-imidazol-4-yl]methyl]-2,3,4,5-tetrahydro-4-(1-naphthalenylcarbonyl)-7-phenyl-1H-1,4-benzodiazepine, trihydrochloride;
2,3,4,5-Tetrahydro-1-(1H-imidazol-4-ylmethyl)-4-(1-naphthalenylcarbonyl)-8-[N,N-bis(phenyl-methyl)amino]-1H-1,4-benzodiazepine, trihydrochloride;
N-[2,3,4,5-Tetrahydro-1-(1H-imidazol-4-yl-methyl)-4-(1-naphthalenylcarbonyl)-1H-1,4-benzodiazepin-8-yl]phenylsulfonamide, dihydrochloride;
N-Phenyl-2,3,4,5-tetrahydro-1-(1H-imidazol-4-yl-methyl)-4-(1-naphthalenylcarbonyl)-1H-1,4-benzo-diazepine-7-carboxamide, dihydrochloride;
N-[2,3,4,5-Tetrahydro-1-(1H-imidazol-4-ylmethyl)-4-(1-naphthalenylcarbonyl)-1H-1,4-benzodiazepin-8-yl]-3-methylbenzamide, dihydrochloride;
N-[2,3,4,5-Tetrahydro-1-(1H-imidazol-4-ylmethyl)-4-(1-naphthalenylcarbonyl)-1H-1,4-benzodiazepin-8-yl]-4-methylbenzamide, dihydrochloride;
3-Chloro-N-[2,3,4,5-tetrahydro-1-(1H-imidazol-4-yl-methyl)-4-(1-naphthalenylcarbonyl)-1H-1,4-benzo-diazepin-8-yl]benzamide, dihydrochloride;
7-Bromo-2,3,4,5-tetrahydro-1-[[2-[(dimethylamino)-methyl]-1H-imidazol-4-yl]methyl]-4-(1-naphthalenylcarbonyl)-1H-1,4-benzodiazepine, dihydrochloride;
7-(4-Chlorophenyl)-2,3,4,5-tetrahydro-1-(1H-imidazol-4-ylmethyl)-4-(1-naphthalenylcarbonyl)-1H-1,4-benzodiazepine, dihydrochloride;
7-(3-Aminophenyl)-2,3,4,5-tetrahydro-1-(1H-imidazol-4-ylmethyl)-4-(1-naphthalenylcarbonyl)-1H-1,4-benzodiazepine, trihydrochloride;
1-Methyl-N-[2,3,4,5-tetrahydro-1-(1H-imidazol-4-ylmethyl)-4-(1-naphthalenylcarbonyl)-1H-1,4-benzodiazepin-8-yl]-1H-pyrrole-2-carboxamide, trihydrochloride;
N-[2,3,4,5-Tetrahydro-1-(1H-imidazol-4-ylmethyl)-4-(1-naphthalenylcarbonyl)-1H-1,4-benzodiazepin-8-yl]-3-furancarboxamide, dihydrochloride;
7-(3-Chlorophenyl)-2,3,4,5-tetrahydro-1-(1H-imidazol-4-ylmethyl)-4-(1-naphthalenylcarbonyl)-1H-1,4-benzodiazepine, dihydrochloride;
2-Methyl-N-[2,3,4,5-tetrahydro-1-(1H-imidazol-4-ylmethyl)-4-(1-naphthalenylcarbonyl)-1H-1,4-benzodiazepin-8-yl]benzamide, dihydrochloride;
N-Phenyl-N′-[2,3,4,5-tetrahydro-1-(1H-imidazol-4-ylmethyl)-4-(1-naphthalenylcarbonyl)-1H-1,4-benzodiazepin-8-yl]urea, dihydrochloride;
2,3,4,5-Tetrahydro-1-(1H-imidazol-4-ylmethyl)-4-(1-naphthalenylcarbonyl)-7-(3-pyridinyl)-1H-1,4-benzodiazepine, trihydrochloride;
2,3,4,5-Tetrahydro-1-(1H-imidazol-4-ylmethyl)-9-methoxy-4-(1-naphthalenylcarbonyl)-1H-1,4-diazepine, dihydrochloride;
(R)-2,3,4,5-Tetrahydro-1-(1H-imidazol-4-ylmethyl)-3-[2-(methylthio)ethyl]-4-(1-naphthalenylcarbonyl)-1H-1,4-benzodiazepine, hydrochloride;
2,3,4,5-Tetrahydro-1-(1H-imidazol-4-ylmethyl)-4-(1-naphthalenylcarbonyl)-3-(phenylmethyl)-1H-1,4-benzodiazepine, hydrochloride;
2,3,4,5-Tetrahydro-3-(2-hydroxyethyl)-1-(1H-imidazol-4-ylmethyl)-4-(1-naphthalenylcarbonyl)-1H-1,4-benzodiazepine, trifluoroacetate;
(S)-2,3,4,5-Tetrahydro-1-(1H-imidazol-4-ylmethyl)-4-(1-naphthalenylcarbonyl)-3-(phenylmethyl)-1H-1,4-benzodiazepine, trifluoroacetate;
4-Acetyl-7-bromo-2,3,4,5-tetrahydro-1-(1H-imidazol-4-ylmethyl)-3-(phenylmethyl)-1H-1,4-benzodiazepine, hydrochloride;
7-Bromo-1,2,3,5-tetrahydro-1-(1H-imidazol-4-ylmethyl)-3-(phenylmethyl)-4H-1,4-benzodiazepine-4-carboxamide, trifluoroacetate;
7-Bromo-2,3,4,5-tetrahydro-1-(1H-imidazol-4-ylmethyl)-4-(methylsulfonyl)-3-(phenylmethyl)-1H-1,4-benzodiazepine, hydrochloride;
4-Acetyl-2,3,4,5-tetrahydro-1-(1H-imidazol-4-ylmethyl)-7-phenyl-3-(phenylmethyl)-1H-1,4-benzodiazepine, trifluoroacetate;
4-Acetyl-7-bromo-3-[(4-chlorophenyl)methyl]-2,3,4,5-tetrahydro-1-(1H-imidazol-4-ylmethyl)-1H -1,4-benzodiazepine, dihydrochloride;
4-Acetyl-2,3,4,5-tetrahydro-1-(1H-imidazol-4-ylmethyl)-3-(phenylmethyl)-1H-naphtho[2,3-e]-1,4-diazepine, monohydrochloride;
N-Cyclohexyl-N′-[2,3,4,5-tetrahydro-1-(1H-imidazol-4-ylmethyl)-4-(1-naphthalenylcarbonyl]-1H-1,4-benzodiazepin-8-yl]urea, dihydrochloride;
2,3,4,5-Tetrahydro-1-(1H-imidazol-4-ylmethyl)-4-(methylsulfonyl)-3-(phenylmethyl)-1H-naphtho[2,3-e]-1,4-diazepine, monohydrochloride;
2,2-Dimethyl-N-[2,3,4,5-tetrahydro-1-(1H-imidazol-4-ylmethyl)-4-(1-naphthalenylcarbonyl)-1H-1,4-benzodiazepin-8-yl]propanamide, dihydrochloride;
2,3,4,5-Tetrahydro-1-(1H-imidazol-4-ylmethyl)-4-(1-naphthalenylsulfonyl)-7-phenyl-1H-1,4-benzodiazepine, monohydrochloride;
4-Acetyl-7-bromo-2,3,4,5-tetrahydro-1-(1H-imidazol-4-ylmethyl)-3-(2-naphthalenylmethyl)-1H-1,4-benzodiazepine, dihydrochloride;
4-Acetyl-7-bromo-2,3,4,5-tetrahydro-1-(1H-imidazol-4-ylmethyl)-3-(1-naphthalenylmethyl)-1H-1,4-benzodiazepine, dihydrochloride;
7-(2-Chlorophenyl)-2,3,4,5-tetrahydro-1-(1H-imidazol-4-ylmethyl)-4-(1-naphthalenylcarbonyl)-1H -1,4-benzodiazepine, dihydrochloride;
2,3,4,5-Tetrahydro-1-(1H-imidazol-4-ylmethyl)-7-phenyl-1H-1,4-benzodiazepine, monohydrochloride;
1-Methyl-N-[2,3,4,5-tetrahydro-1-(1H-imidazol-4-ylmethyl)-4-(1-naphthalenylcarbonyl)-1H-1,4-benzodiazepin-8-yl]-2-piperidinecarboxamide, trihydrochloride;
N-[2,3,4,5-tetrahydro-1-(1H-imidazol-4-ylmethyl)-4-(1-naphthalenylcarbonyl)-1H-1,4-benzodiazepin-8-yl]-4-morpholinecarboxamide, dihydrochloride;
2,3,4,5-Tetrahydro-1-(1H-imidazol-4-ylmethyl)-4-(1-naphthalenylcarbonyl)-1H-1,4-benzodiazepin-8-yl]-3-methylbutanamide, dihydrochloride;
1,2,3,5-Tetrahydro-1-(1H-imidazol-4-ylmethyl)-N,N,7-triphenyl-4H-1,4-benzodiazepin-carboxamide, dihydrochloride;
1,2,3,5-Tetrahydro-1-(1H-imidazol-4-ylmethyl)-3-(phenylmethyl)-4H-naphtho[2,3-e]-1,4-diazepine-4-carboxylic acid, methyl ester, monohydrochloride;
2,3,4,5-Tetrahydro-1-(1H-imidazol-4-ylmethyl)-7-phenyl-4-[(4-phenyl-1,2,3-thiadiazol-5-yl)carbonyl]-1H-1,4-benzodiazepine, trifluoroacetate;
8-[[(Cyclohexylamino)carbonyl]amino]-2,3,4,5-tetrahydro-1-(1H-imidazol-4-ylmethyl)-3-(phenylmethyl)-1H-1,4-benzodiazepine-4-carboxylic acid, 1,1-dimethylethyl ester;
2,3,4,5-Tetrahydro-1-(1H-imidazol-4-ylmethyl)-8-[[(4-methylphenyl)sulfonyl]amino]-3-(phenylmethyl)-1H-1,4-benzodiazepine-4-carboxylic acid, 1,1-dimethylethylester;
7-Bromo-1,2,3,4-tetrahydro-1-(1H-imidazol-4-ylmethyl)-3-(phenylmethyl)-5H-1,4-benzodiazepin-5-one, dihydrochloride;
2,3,4,5-Tetrahydro-1-(1H-imidazol-4-ylmethyl)-4-[1-oxo-3-(1-piperidinyl)propyl]-7-phenyl-1H-1,4-benzodiazepine, trihydrochloride;
2,3,4,5-Tetrahydro-1-(1H-imidazol-4-ylmethyl)-7-phenyl-4-(4-quinolinylcarbonyl)-1H-1,4-benzodiazepine, trihydrochloride;
4-[(5-Bromo-3-pyridinyl)carbonyl]-2,3,4,5-tetrahydro-1-(1H-imidazol-4-ylmethyl)-7-phenyl-1H -1,4-benzodiazepine, trihydrochloride;
(S)-4-[2-(Dimethylamino)-1-oxo-3-phenylpropyl]-2,3,4,5-tetrahydro-1-(1H-imidazol-4-ylmethyl)-7-phenyl-1H-1,4-benzodiazepine, trihydrochloride;
2,3,4,5-Tetrahydro-4-[4-hydroxy-3-(4-morpholinyl-methyl)benzoyl]-1-(1H-imidazol-4-ylmethyl)-7-phenyl-1H-1,4-benzodiazepine, trihydrochloride;
(S)-2,3,4,5-Tetrahydro-1-(1H-imidazol-4-ylmethyl)-4-[(1-methyl-2-pyrrolidinyl)carbonyl]-7-phenyl-1H-1,4-benzodiazepine, trihydrochloride;
2,3,4,5-Tetrahydro-1-(1H-imidazol-4-ylmethyl)-7-phenyl-4-[[2-(propylthio)-3-pyridinyl]carbonyl]-1H-1,4-benzodiazepine, trihydrochloride;
4-[(2-Chloro-6-methyl-4-pyridinyl)carbonyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-[[2-(phenylthio)-3-pyridinyl]carbonyl]-1H-1,4-benzodiazepine, trihydrochloride;
2,3,4,5-Tetrahydro-1-(1H-imidazol-4-ylmethyl)-4-[[2-(4-methylphenoxy)-3-piperidinyl]carbonyl]-7-phenyl-1H-1,4-benzodiazepine, trihydrochloride;
2,3,4,5-Tetrahydro-1-(1H-imidazol-4-ylmethyl)-4-[(2-methoxy-3-pyridinyl)carbonyl]-7-phenyl-1H-1,4-benzodiazepine, trihydrochloride;
2,3,4,5-Tetrahydro-1-(1H-imidazol-4-ylmethyl)-7-phenyl-4-[(5-phenyl-4-oxazolyl)carbonyl]-1H-1,4-benzodiazepine, dihydrochloride;
4-Acetyl-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-7-phenyl-4-[(tetrahydro-3-furanyl)carbonyl]-1H-1,4-benzodiazepine, dihydrochloride;
2,3,4,5-Tetrahydro-1-(1H-imidazol-4-ylmethyl)-4-[(2-methoxyethoxy)acetyl]-7-phenyl-1H-1,4-benzodiazepine, dihydrochloride;
2,3,4,5-Tetrahydro-1-(1H-imidazol-4-ylmethyl)-4-(4-(4-morpholinylmethyl)benzoyl]-7 2,3,4,5-Tetrahydro-1-(1H-imidazol-4-ylmethyl)-4-(4-(4-morpholinylmethyl)benzoyl]-7 2,3,4,5-Tetrahydro-1-(1H-imidazol-4-ylmethyl)-4-[4-(methylsulfonyl)benzoyl]-7-phenyl-1H-1,4-benzodiazepine, dihydrochloride;
2,3,4,5-Tetrahydro-1-(1H-imidazol-4-ylmethyl)-4-[1-oxo-3-(phenylsulfonyl)propyl]-7-phenyl-1H-1,4-benzodiazepine, dihydrochloride;
2,3,4,5-Tetrahydro-1-(1H-imidazol-4-ylmethyl)-7-phenyl-4-(3-pyridinylacetyl)-1H-1,4-benzodiazepine, trihydrochloride;
2,3,4,5-Tetrahydro-1-(1H-imidazol-4-ylmethyl)-7-phenyl-4-(2-quinoxalinylcarbonyl)-1H-1,4-benzodiazepine, tetrahydrochloride;
2,3,4,5-Tetrahydro-1-(1H-imidazol-4-ylmethyl)-4-(4-isoquinolinylcarbonyl)-7-phenyl-1H-1,4-benzodiazepine, trihydrochloride;
4-[(2-Chloro-3-pyridinyl)carbonyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-(3-pyridinylcarbonyl)-1H-1,4-benzodiazepine, trihydrochloride;
4-[(2,6-Dimethoxy-3-pyridinyl)carbonyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-(2-pyrazinylcarbonyl)-1H-1,4-benzodiazepine, tetrahydrochloride;
4-(2-Ethoxybenzoyl)-2,3,4,5-tetrahydro-1-(1H-imidazol-4-ylmethyl)-7-phenyl-1H-1,4-benzodiazepine, dihydrochloride;
4-[3-(Dimethylamino)benzoyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-[(1-phenylcyclopropyl)carbonyl]-1H-1,4-benzodiazepine, dihydrochloride;
4-[(Bicyclo[4,2,0]octa-1,3,5-trien-7-yl)carbonyl]-2,3,4,5-tetrahydro-1-(1H-imidazol-4-ylmethyl)-7-phenyl-1H-1,4-benzodiazepine, dihydrochloride;
4-Benzoyl-2,3,4,5-tetrahydro-1-(1H-imidazol-4-ylmethyl)-7-phenyl-1H-1,4-benzodiazepine, dihydrochloride;
4-(2-Chlorobenzoyl)-2,3,4,5-tetrahydro-1-(1H-imidazol-4-ylmethyl)-7-phenyl-1H-1,4-benzodiazepine, dihydrochloride;
4-(2,3-Dichlorobenzoyl)-2,3,4,5-tetrahydro-1-(1H-imidazol-4-ylmethyl)-7-phenyl-1H-1,4-benzodiazepine, dihydrochloride;
N-[2-[[2,3,4,5-Tetrahydro-1-(1H-imidazol-4-ylmethyl)-7-phenyl-1H-1,4-benzodiazepin-4-yl]carbonyl]phenyl]acetamide, dihydrochloride;
2,3,4,5-Tetrahydro-1-(1H-imidazol-4-ylmethyl)-4-(2-phenoxybenzoyl)-7-phenyl-1H-1,4-benzodiazepine, dihydrochloride;
2,3,4,5-Tetrahydro-1-(1H-imidazol-4-ylmethyl)-4-(2-methoxybenzoyl)-7-phenyl-1H-1,4-benzodiazepine, dihydrochloride;
4-(2,3-Dimethoxybenzoyl)-2,3,4,5-tetrahydro-1-(1H-imidazol-4-ylmethyl)-7-phenyl-1H-1,4-benzodiazepine, dihydrochloride;
4-(2,4-Dimethoxybenzoyl)-2,3,4,5-tetrahydro-1-(1H-imidazol-4-ylmethyl)-7-phenyl-1H-1,4-benzodiazepine, dihydrochloride;
4-(2,5-Dimethoxybenzoyl)-2,3,4,5-tetrahydro-1-(1H-imidazol-4-ylmethyl)-7-phenyl-1H-1,4-benzodiazepine, dihydrochloride;
4-(2,6-Dimethoxybenzoyl)-2,3,4,5-tetrahydro-1-(1H-imidazol-4-ylmethyl)-7-phenyl-1H-1,4-benzodiazepine, dihydrochloride;
4-(2,3-Dihydroxybenzoyl)-2,3,4,5-tetrahydro-1-(1H-imidazol-4-ylmethyl)-7-phenyl-1H-1,4-benzodiazepine, dihydrochloride;
4-([1,1′-Biphenyl]-2-ylcarbonyl)-2,3,4,5-tetrahydro-1-(1H-imidazol-4-ylmethyl)-7-phenyl-1H-1,4-benzodiazepine, dihydrochloride;
2,3,4,5Tetrahydro-1-(1H-imidazol-4-ylmethyl)-4-(2-methylbenzoyl)-7-phenyl-1H-1,4-benzodiazepine, dihydrochloride;
4-(2,3-Dimethylbenzoyl)-2,3,4,5-tetrahydro-1-(1H-imidazol-4-ylmethyl)-7-phenyl-1H-1,4-benzodiazepine, dihydrochloride;
4-(3-Cyanobenzoyl)-2,3,4,5-tetrahydro-1-(1H-imidazol-4-ylmethyl)-7-phenyl-1H-1,4-benzodiazepine, dihydrochloride;
4-(3-Chlorobenzoyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(3-phenoxybenzoyl)-7-phenyl-1H-1,4-benzodiazepine, dihydrochloride;
2,3,4,5-Tetrahydro-1-(1H-imidazol-4-ylmethyl)-4-(3-methoxybenzoyl)-7-phenyl-1H-1,4-benzodiazepine, dihydrochloride;
4-(3,4-Dimethoxybenzoyl)-2,3,4,5-tetrahydro-1-(1H-imidazol-4-ylmethyl)-7-phenyl-1H-1,4-benzodiazepine, dihydrochloride;
4-(3,5-Dimethoxybenzoyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(3-methylbenzoyl)-7-phenyl-1H-1,4-benzodiazepine, dihydrochloride;
4-(1,2-Dioxo-2-phenylethyl)-2,3,4,5-tetrahydro-1-(1H-imidazol-4-ylmethyl)-7-phenyl-1H-1,4-benzodiazepine, dihydrochloride;
4-[(2-Ethoxy-1-naphthalenyl)carbonyl]-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(2-naphthalenylcarbonyl)-7-phenyl-1H-1,4-benzodiazepine, dihydrochloride;
4-(Fluorophenylacetyl)-2,3,4,5-tetrahydro-1-(1H-imidazol-4-ylmethyl)-7-phenyl-1H-1,4-benzodiazepine, dihydrochloride;
4-(Diphenylacetyl)-2,3,4,5-tetrahydro-1-(1H-imidazol-4-ylmethyl)-7-phenyl-1H-1,4-benzodiazepine, dihydrochloride;
2,3,4,5-Tetrahydro-4-(2-hydroxy-1-oxo-2-phenylpropyl)-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(1H-indol-2-ylcarbonyl)-7-phenyl-1H-1,4-benzodiazepine, dihydrochloride;
2,3,4,5-Tetrahydro-1-(1H-imidazol-4-ylmethyl)-4-(1H-indol-3-ylcarbonyl)-7-phenyl-1H-1,4-benzodiazepine, dihydrochloride;
2,3,4,5-Tetrahydro-1-(1H-imidazol-4-ylmethyl)-4-(1H-indol-5-ylcarbonyl)-7-phenyl-1H-1,4-benzodiazepine, dihydrochloride;
2,3,4,5-Tetrahydro-1-(1H-imidazol-4-ylmethyl)-4-[(1-methyl-1H-indol-2-yl)carbonyl]-7-phenyl-1H-1,4-benzodiazepine, dihydrochloride;
4-(2-Benzofuranylcarbonyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-7-phenyl-4-(3-pyridinylcarbonyl)-1H-1,4-benzodiazepine, N-oxide, dihydrochloride;
2,3,4,5-Tetrahydro-1-(1H-imidazol-4-ylmethyl)-7-phenyl-4-(2-pyridinylcarbonyl)-1H-1,4-benzodiazepine, trihydrochloride;
2,3,4,5-Tetrahydro-1-(1H-imidazol-4-ylmethyl)-7-phenyl-4-(2-quinolinylcarbonyl)-1H-1,4-benzodiazepine, trihydrochloride;
2,3,4,5-Tetrahydro-1-(1H-imidazol-4-ylmethyl)-7-phenyl-4-(1-isoquinolinylcarbonyl)-1H-1,4-benzodiazepine, trihydrochloride;
4-(3-Chloro-2-nitrobenzoyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-(2-nitrobenzoyl)-7-phenyl-1H-1,4-benzodiazepine, dihydrochloride;
2,3,4,5-Tetrahydro-1-(1H-imidazol-4-ylmethyl)-4-(3-methoxy-2-nitrobenzoyl)-7-phenyl-1H-1,4-benzodiazepine, dihydrochloride;
2,3,4,5-Tetrahydro-1-(1H-imidazol-4-ylmethyl)-4-(1H-indol-4-ylcarbonyl)-7-phenyl-1H-1,4-benzodiazepine, dihydrochloride;
4-[(2,6-Dihydroxy-3-naphthalenyl)carbonyl]-2,3,4,5-tetrahydro-1-(1H-imidazol-4-ylmethyl)-7-phenyl-1H-1,4-benzodiazepine, dihydrochloride;
4-(1H-Benzimidazol-5-ylcarbonyl)-2,3,4,5-tetrahydro-1-(1H-imidazol-4-ylmethyl)-7-phenyl-1H-1,4-benzodiazepine, trihydrochloride;
4-(1H-Benzotriazol-5-ylcarbonyl)-2,3,4,5-tetrahydro-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-[(4-methoxy-2-quinolinyl)carbonyl]-7-phenyl-1H-1,4-benzodiazepine trihydrochloride;
N-[3-[[2,3,4,5-Tetrahydro-1-(1H-imidazol-4-ylmethyl)-7-phenyl-1H-1,4-benzodiazepin-4-yl]carbonyl]phenyl]-acetamide, dihydrochloride;
2,3,4,5-Tetrahydro-1-(1H-imidazol-4-ylmethyl)-4-(2-methyl-1-oxo-2-phenylpropyl)-7-phenyl-1H-1,4-benzodiazepine, dihydrochloride;
4-[2-(Dimethylamino)benzoyl]-2,3,4,5-tetrahydro-1-(1H-imidazol-4-ylmethyl)-7-phenyl-1H-1,4-benzodiazepine, trihydrochloride;
4-(3-Ethoxybenzoyl)-2,3,4,5-tetrahydro-1-(1H-imidazol-4-ylmethyl)-7-phenyl-1H-1,4-benzodiazepine,dihydrochloride;
2,3,4,5-Tetrahydro-4-(2-hydroxy[1,1′-biphenyl]-3-ylcarbonyl)-1-(1H-imidazol-4-ylmethyl)-7-phenyl-1H-1,4-benzodiazepine, dihydrochloride;
2,3,4,5-Tetrahydro-4-[2-[(2-hydroxyethyl)thio]benzoyl]-1-(1H-imidazol-4-ylmethyl)-7-phenyl-1H-1,4-benzodiazepine, dihydrochloride;
2,3,4,5-Tetrahydro-1-(1H-imidazol-4-ylmethyl)-4-[(2-methoxy-1-naphthalenyl)carbonyl]-7-phenyl-1H-1,4-benzodiazepine, dihydrochloride;
2,3,4,5-Tetrahydro-4-[(2-hydroxy-4-qiunolinyl)-carbonyl]-1-(1H-imidazol-4-ylmethyl)-7-phenyl-1H-1,4-benzodiazepine, dihydrochloride;
2-[[2,3,4,5-Tetrahydro-1-(1H-imidazol-4-ylmethyl)-7-phenyl-1H-1,4-benzodiazepin-4-yl]carbonyl]benzamide, dihydrochloride;
N-(1,1-Dimethylethyl)-2-[[2,3,4,5-tetrahydro-1-(1H-imidazol-4-ylmethyl)-7-phenyl-1H-1,4-benzodiazepin-4-yl]carbonyl]benzamide, dihydrochloride;
N-(4-Fluorophenyl)-N′-[3-[[2,3,4,5-tetrahydro-1-(1H-imidazol-4-ylmethyl)-7-phenyl-1H-1,4-benzodiazepin-4-yl]carbonyl]phenyl]urea, dihydrochloride;
2,3,4,5-Tetrahydro-1-(1H-imidazol-4-ylmethyl)-4-[(3-methyl-4-oxo-2-phenyl-4H-benzopyran-8-yl)carbonyl]-7-phenyl-1H-1,4-benzodiazepine, dihydrochloride;
2,3,4,5-Tetrahydro-1-(1H-imidazol-4-ylmethyl)-7-phenyl-4-[3-(trifluoromethoxy)benzoyl]-1H-1,4-benzodiazepine, dihydrochloride;
4-(2-Cyanobenzoyl)-2,3,4,5-tetrahydro-1-(1H-imidazol-4-ylmethyl)-7-phenyl-1H-1,4-benzodiazepine,dihydrochloride;
2,3,4,5-Tetrahydro-1-(1H-imidazol-4-ylmethyl)-4-[2-[[(4-methophenyl)sulfonyl]amino]benzoyl]-7-phenyl-1H-1,4-benzodiazepine, dihydrochloride;
2,3,4,5-Tetrahydro-1-(1H-imidazol-4-ylmethyl)-7-phenyl-4-(6-quinolinylcarbonyl)-1H-1,4-benzodiazepine, trihydrochloride;
2,3,4,5-Tetrahydro-1-(1H-imidazol-4-ylmethyl)-7-phenyl-4-(8-quinolinylcarbonyl)-1H-1,4-benzodiazepine, trihydrochloride;
4-(Benzo[b]thiophen-2-ylcarbonyl)-2,3,4,5-tetrahydro-1-(1H-imidazol-4-ylmethyl)-7-phenyl-1H-1,4-benzodiazepine, dihydrochloride;
4-[[4-(Dimethylamino)-1-naphthalenyl]-carbonyl]-2,3,4,5-tetrahydro-1-(1H-imidazol-4-ylmethyl)-7-phenyl-1H-1,4-benzodiazepine, trihydrochloride;
2,3,4,5-Tetrahydro-1-(1H-imidazol-4-ylmethyl)-7-phenyl-4-(1H-purin-6-ylcarbonyl)-1H-1,4-benzodiazepine, trihydrochloride;
2,3,4,5-Tetrahydro-1-(1H-imidazol-4-ylmethyl)-4-(methoxyphenylacetyl)-7-phenyl-1H-1,4-benzodiazepine, dihydrochloride;
2,3,4,5-Tetrahydro-1-(1H-imidazol-4-ylmethyl)-4-[(5-methyl-1-phenyl-1H-pyrazol-4-yl)carbonyl]-7-phenyl-1H-1,4-benzodiazepine,trihydrochloride;
2,3,4,5-Tetrahydro-1-(1H-imidazol-4-ylmethyl)-4-[2-(2-methylphenyl)-1-oxopropyl]-7-phenyl-1H-1,4-benzodiazepine, dihydrochloride;
2,3,4,5-Tetrahydro-1-(1H-imidazol-4-ylmethyl)-7-phenyl-4-[(tetrahydro-4-phenyl-2H-pyran-4-yl)carbonyl]-1H-1,4-benzodiazepine, dihydrochloride;
2,3,4,5-Tetrahydro-1-(1H-imidazol-4-ylmethyl)-4-[2-(methylphenylamino)benzoyl]-7-phenyl-1H -1,4-benzodiazepine, trihydrochloride;
2,3,4,5-Tetrahydro-1-(1H-imidazol-4-ylmethyl)-7-phenyl-4-(4quinolinylcarbonyl)-1H-phenyl-1H-1,4-benzodiazepine, trihydrochloride;
2,3,4,5-Tetrahydro-1-(1H-imidazol-4-ylmethyl)-7-phenyl-4-(4-quinolinylcarbonyl)-1H-1,4-benzodiazepine, N-oxide, dihydrochloride;
N-Methyl-N-(2-pyridinylmethyl)-2-[[2,3,4,5-tetrahydro-1-(1H-imidazol-4-ylmethyl)-7-phenyl-1H-1,4-benzodiazepin-4-yl]carbonyl]benzamide, trihydrochloride;
2,3,4,5-Tetrahydro-1-(1H-imidazol-4-ylmethyl)-4-(3-isoquinolinylcarbonyl)-7-phenyl-1H-1,4-bezodiazepine, trihydrochloride;
2,3,4,5-Tetrahydro-1-(1H-imidazol-4-ylmethyl)-4-[(2-naphthalenylthio)acetyl]-1H-1,4-benzodiazepine, trifluoroacetate (1:2);
4-[3-(3,4-Dimethoxyphenyl)-1-oxopropyl]-2,3,4,5-tetrahydro-1-(1H-imidazol-4-ylmethyl)-1H-1,4-benzodiazepine, trifluoroacetate (1:2);
4-([1,1′-Biphenyl]-4-ylacetyl)-2,3,4,5-tetrahydro-1-(1H-imidazol-4-ylmethyl)-1H-1,4-benzodiazepine, trifluoroacetate (1:2);
2,3,4,5-Tetrahydro-1-(1H-imidazol-4-ylmethyl)-4-(2-naphthalenylacetyl)-1H-1,4-benzodiazepine,trifluoroacetate (1:2);
4-([1,1′-Biphenyl]-2-ylcarbonyl)-2,3,4,5-tetrahydro-1-(1H-imidazol-4-ylmethyl)-1H-1,4-benzodiazepine, trifluoroacetate (1:2);
2,3,4,5-Tetrahydro-1-(1H-imidazol-4-ylmethyl)-4-[(2-phenyl-4-quinolinyl)carbonyl]-1H-1,4-benzodiazepine, trifluoroacetate (1:3);
2,3,4,5-Tetrahydro-1-(1H-imidazol-4-ylmethyl)-4-(3-pyridinylacetyl)-1H-1,4-benzodiazepine, trifluoroacetate (1:3);
4-(9H-Fluoren-9-ylacetyl)-2,3,4,5-tetrahydro-1-(1H-imidazol-4-ylmethyl)-1H-1,4-benzodiazepine, trifluoroacetate (1:2);
(S)-4-[2-(Dimethylamino)-1-oxo-3-phenylpropyl]-2,3,4,5-tetrahydro-1-(1H-imidazol-4-ylmethyl)-1H-1,4-benzodiazepine, trifluoroacetate (1:3);
(S)-2,3,4,5-Tetrahydro-1-(1H-imidazol-4-ylmethyl)-4-[(2-oxo-4-phenyl-3-oxazolidinyl)acetyl]-1H-1,4-benzodiazepine, trifluoroacetate (1:2);
4-(9-Acridinylcarbonyl)-2,3,4,5-tetrahydro-1-(1H-imidazol-4-ylmethyl)-1H-1,4-benzodiazepine, trifluoroacetate (1:3);
2,3,4,5-Tetrahydro-1-(1H-imidazol-4-ylmethyl)-4-(3-phenoxybenzoyl)-1H-1,4-benzodiazepine, trifluoroacetate (1:2);
2,3,4,5-Tetrahydro-1-(1H-imidazol-4-ylmethyl)-4-[[4′-(trifluoromethyl)[1,1′-biphenyl]-2-yl]carbonyl]-1H-1,4-benzodiazepine, trifluoroacetate (1:2);
2,3,4,5-Tetrahydro-1-(1H-imidazol-4-ylmethyl)-4-(4-phenoxybenzoyl)-1H-1,4-benzodiazepine, trifluoroacetate (1:2);
2,3,4,5-Tetrahydro-1-(1H-imidazol-4-ylmethyl)-4-(2-naphthalenylcarbonyl)-1H-1,4-benzodiazepine, trifluoroacetate (1:2);
2,3,4,5-Tetrahydro-1-(1H-imidazol-4-ylmethyl)-4-(1-oxo-4-phenylbutyl)-1H-1,4-benzodiazepine, trifluoroacetate (1:2);
2,3,4,5-Tetrahydro-1-(1H-imidazol-4-ylmethyl)-4-[(2-phenoxyphenyl)acetyl]-1H-1,4-benzodiazepine, trifluoroacetate (1:2);
2,3,4,5-Tetrahydro-1-(1H-imidazol-4-ylmethyl)-4-[2-[(4-methylphenyl)sulfinyl]benzoyl]-1H-1,4-benzodiazepine, trifluoroacetate (1:2);
2,3,4,5-Tetrahydro-1-(1H-imidazol-4-ylmethyl)-4-[2-[(phenylmethyl)amino]benzoyl]-1H-1,4-benzodiazepine, trifluoroacetate (1:3);
1,2,3,5-Tetrahydro-1-(1H-imidazol-4-yl-methyl)-N,N-diphenyl-4H-1,4-benzodiazepine-4carboxamide, hydrochloride;
1,2,3,5-Tetrahydro-1-(1H-imidazol-4-yl-methyl)-a,7-diphenyl-4H-1,4-benzodiazepine-4-acetic acid, methyl ester, hydrochloride;
4-Acetyl-2,3,4,5-tetrahydro-1-(1H-imidazol-4-ylmethyl)-3-(phenylmethyl)-1H-1,4-benzodiazepine, hydrochloride;
(R)-7-Bromo-2,3,4,5-tetrahydro-1-(1H-imidazol-4-ylmethyl)-4-(methylsulfonyl)-3-(phenylmethyl)-1H-1,4-benzodiazepine, hydrochloride;
(R)-2,3,4,5-Tetrahydro-1-(1H-imidazol-4-ylmethyl)-4-(methylsulfonyl)-3-(phenylmethyl)-1H-1,4-benzodiazepine-7-carbonitrile, monohydrochloride;
(R)-4-Acetyl-2,3,4,5-tetrahydro-1-(1H-imidazol-4-ylmethyl)-7-phenyl-3-(phenylmethyl)-1H-1,4-benzodiazepine, monohydrochloride;
7-Bromo-4-[[2-(dimethylamino)ethyl]sulfonyl]-2,3,4,5-tetrahydro-1-(1H-imidazol-4-ylmethyl)-3-(phenylmethyl)-4H-1,4-benzodiazepine, trifluoroacetate (1:2);
2,3,4,5-Tetrahydro-1-(1H-imidazol-4-ylmethyl)-7-phenyl-4-[(1,2,3,4-tetrahydro-1-quinolinyl)carbonyl]-1H-1,4-benzodiazepine, monohydrochloride;
N-Ethyl-1,2,3,5-tetrahydro-1-(1H-imidazol-4-ylmethyl)-N,7-diphenyl-4H-1,4-benzodiazepine-4-carboxamide, monohydrochloride;
4-[(2,3-Dihydro-1H-indol-1-yl)carbonyl]-2,3,4,5-tetrahydro-1-(1H-imidazol-4-ylmethyl)-7-phenyl-1H-1,4-benzodiazepine, monohydrochloride;
2,3,4,5-Tetrahydro-1-(1H-imidazol-4-ylmethyl)-4-(methylsulfonyl)-3-(phenylmethyl)-7-(4-pyridinyl)-1H-1,4-benzodiazepine, trihydrochloride;
(R)-4-[[2-(Dimethylamino)ethyl]sulfonyl]-2,3,4,5-tetrahydro-1-(1H-imidazol-4-ylmethyl)-7-phenyl-3-(phenylmethyl)-1H-1,4-benzodiazepine, trifluoroacetate (1:1);
[2,3,4,5-Tetrahydro-1-(1H-imidazol-4-ylmethyl)-4-(1-naphthalenylcarbonyl)-1H-1,4-benzodiazepin-8-yl]carbamic acid, cyclohexyl ester, dihydrochloride;
(R)-7-Bromo-2,3,4,5-tetrahydro-1-(1-methyl-1H-imidazol-5-yl)methyl)-4-(methylsulfonyl)-3-(phenylmethyl)-1H-1,4-benzodiazepine, hydrochloride;
(R)-7-Cyano-2,3,4,5-tetrahydro-1-[(1-methyl-1H-imidazol-5-yl)methyl]-4-(methylsulfonyl)-3-(phenylmethyl)-1H-1,4-benzodiazepine, monohydrochloride;
4-[2-(4-Chlorophenyl)-1,2-dioxoethy]-2,3,4,5-tetrahydro-1-(1H-imidazol-4-ylmethyl)-7-phenyl-1H-1,4-benzodiazepine, hydrochloride;
4-(1,2-Dioxopropyl)-2,3,4,5-tetrahydro-1-(1H-imidazol-4-ylmethyl)-7-phenyl-1H-1,4-benzodiazepine, hydrochloride;
2,3,4,5-Tetrahydro-1-(1H-imidazol-4-ylmethyl)-4-[2-(4-nitrophenyl)-1,2-dioxoethyl]-7-phenyl-1H-1,4-benzodiazepine, hydrochloride;
2,3,4,5-Tetrahydro-1-(1H-imidazol-4-ylmethyl)-4-[2-(4-methoxyphenyl)-1,2-dioxoethyl]-7-phenyl-1H-1,4-benzodiazepine, hydrochloride;
2,3,4,5-Tetrahydro-1-(1H-imidazol-4-ylmethyl)-7-phenyl-4-(3,3,3-trifluoro-1,2-dioxopropyl)-1H-1,4-benzodiazepine, trifluoroacetate (1:2);
(R)-7-Bromo-2,3,4,5-tetrahydro-1-(1H-imidazol-4-ylacetyl)-4-(methylsulfonyl)-3-(phenylmethyl)-1H-1,4-benzodiazepine, monohydrochloride;
(R)-7-Bromo-2,3,4,5-tetrahydro-1-(2-1H-imidazol-4-ylethyl)-4-(methylsulfonyl)-3-(phenylmethyl)-1H-1,4-benzodiazepine, monohydrochloride;
8-[(Cyclohexylcarbonyl)amino]-1,2,3,5-tetrahydro-1-(1H-imidazol-4-ylmethyl)-3-(phenylmethyl)-4H-1,4-benzodiazepine-4-carboxylic acid, methyl ester, dihydrochloride;
N-[2,3,4,5-Tetrahydro-1-(1H-imidazol-4-ylmethyl)-4-(1-naphthalenylcarbonyl)-1H-1,4-benzodiazepin-8-yl]-1-piperidinecarboxamide, dihydrochloride;
(R)-7-Cyano-1,2,3,5-tetrahydro-1-(1H-imidazol-4-ylmethyl)-3-(phenylmethyl)-4H-1,4-benzodiazepine-4-carboxylic acid, ethyl ester, hydrochloride;
N-[2,3,4,5-Tetrahydro-1-(1H-imidazol-4-ylmethyl)-4-(methylsulfonyl)-3-(phenylmethyl)-1H-1,4-benzodiazepin-8-yl]cyclohexanecarboxamide, dihydrochloride;
(R)-7-Cyano-4-[[2-(dimethylamino)ethyl]sulfonyl]-2,3,4,5-tetrahydro-1-(1H-imidazol-4-ylmethyl)-3-(phenylmethyl)-4H-1,4-benzodiazepine, dihydrochloride;
(R)-7-Cyano-2,3,4,5-tetrahydro-1-(1H-imidazol-4-ylmethyl)-4-[[2-(4-morpholinyl)ethyl]sulfonyl]-3-(phenylmethyl)-4H-1,4-benzodiazepine, dihydrochloride;
N-[2,3,4,5-Tetrahydro-1-(1H-imidazol-4-ylmethyl)-4-(2-methoxy-3-methylbenzoyl)-1H-1,4-benzodiazepin-8-yl]cyclohexanecarboxamide, dihydrochloride;
8-[(Cyclohexylcarbonyl)amino]-2,3,4,5-tetrahydro-1-(1H-imidazol-4-ylmethyl)-N-phenyl-1H-1,4-benzodiazepine-4-carboxamide, dihydrochloride;
N-[2,3,4,5-Tetrahydro-1-(1H-imidazol-4-ylmethyl)-4-[(2-methylphenyl)sulfonyl]-1H-1,4-benzodiazepin-8-yl]cyclohexanamide, dihydrochloride;
N-[2,3,4,5-Tetrahydro-1-(1H-imidazol-4-ylmethyl)-4-[(2-methoxyphenyl)carbonyl]-1H-1,4-benzodiazepin-8-yl]cyclohexanamide, dihydrochloride;
(R)-7-Cyano-1,2,3,5-tetrahydro-1-(1H-imidazol-4-ylmethyl)-3-(phenylmethyl)-4H-1,4-benzodiazepine-4-sulfonic acid, ethyl ester, hydrochloride;
(3R)-7-Bromo-1-[cyano(1H-imidazol-4-yl)methyl]-2,3,4,5-tetrahydro-4-(methylsulfonyl)-3-(phenylmethyl)-1H-1,4-benzodiazepine, monohydrochloride;
(3R)-1-[2-Amino-1-(1H-imidazol-4-yl)ethyl]-2,3,4,5-tetrahydro-4-(methylsulfonyl)-3-(phenylmethyl)-1H-1,4-benzodiazepine, dihydrochloride;
(3R)-1-[2-(Dimethylamino)-1-(1H-imidazol-4-yl)ethyl]-2,3,4,5-tetrahydro-4-(methylsulfonyl)-3-(phenylmethyl)-1H-1,4-benzodiazepine, dihydrochloride;
(3R)-1-[2-Amino-1-(1H-imidazol-4-yl)ethyl]-7-bromo-2,3,4,5-tetrahydro-4-(methylsulfonyl)-3-(phenylmethyl)-1H-1,4-benzodiazepine, dihydrochloride;
(3R)-1-[2-(Dimethylamino)-1-(1H-imidazol-4-yl)ethyl]-7-bromo-2,3,4,5-tetrahydro-4-(methylsulfonyl)-3-(phenylmethyl)-1H-1,4-benzodiazepine, dihydrochloride;
7-Cyano-1,3,4,5-tetrahydro-1-(1-methyl-1H-imidazol-5-ylmethyl)-3-(phenylmethyl)-4-(phenylsulfonyl)-2H-1,4-benzodiazepin-2-one, monohydrochloride;
7-Cyano-1,3,4,5-tetrahydro-1-(1H-imidazol-4-ylmethyl)-3-(phenylmethyl)-4-(phenylsulfonyl)-2H-1,4-benzodiazepin-2-one, monohydrochloride;
7-Bromo-2,3,4,5-tetrahydro-1-(1H-imidazol-4-ylmethyl)-4-(methylsulfonyl)-3-(2-phenylethyl)-1H -1,4-benzodiazepine, dihydrochloride;
7-Bromo-3-[(3-chlorophenyl)methyl]-2,3,4,5-tetrahydro-1-(1H-imidazol-4-ylmethyl)-4-(methylsulfonyl)-1H-1,4-benzodiazepine, dihydrochloride;
(R)-7-Bromo-3-(cyclohexylmethyl)-2,3,4,5-tetrahydro-1-(1H-imidazol-4-ylmethyl)-4-(methylsulfonyl)-1H-1,4-benzodiazepine, dihydrochloride;
7-Bromo-3-[(2-chlorophenyl)methyl]-2,3,4,5-tetrahydro-1-(1H-imidazol-4-ylmethyl)-4-(methylsulfonyl)-1H-1,4-benzodiazepine, dihydrochloride;
(S)-7-Bromo-3-(cyclohexylmethyl)-2,3,4,5-tetrahydro-1-(1H-imidazol-4-ylmethyl)-4-(methylsulfonyl)-1H-1,4-benzodiazepine, dihydrochloride;
7-Bromo-2,3,4,5-tetrahydro-1-(1H-imidazol-4-ylmethyl)-3-[(4-methoxyphenyl)methyl]-4-(methylsulfonyl)-1H-1,4-benzodiazepine, dihydrochloride;
4-Acetyl-7-bromo-3-[(2-chlorophenyl)methyl]-2,3,4,5-tetrahydro-1-(1H-imidazol-4-ylmethyl)-1H -1,4-benzodiazepine, dihydrochloride;
4-Acetyl-7-bromo-3-[(3-chlorophenyl)methyl]-2,3,4,5-tetrahydro-1-(1H-imidazol-4-ylmethyl)-1H -1,4-benzodiazepine, dihydrochloride;
7-Bromo-2,3,4,5-tetrahydro-1-(1H-imidazol-4-ylmethyl)-3-[(4-hydroxyphenyl)methyl]-4-(methylsulfonyl)-1H-1,4-benzodiazepine, dihydrochloride;
(R)-2,3,4,5-Tetrahydro-1-(1H-imidazol-4-ylmethyl)-4-(methylsulfonyl)-7-phenyl-3-(3-pyridinylmethyl)-1H-1,4-benzodiazepine, dihydrochloride;
2,3,4,5-Tetrahydro-8-(hydroxymethyl)-1-(1H-imidazol-4-ylmethyl)-4-(1-naphthalenylcarbonyl)-1H -1,4-benzodiazepine, dihydrochloride;
2,3,4,5-Tetrahydro-1-(1H-imidazol-4-ylmethyl)-4-(1-naphthalenylcarbonyl)-8-(phenoxymethyl)-1H-1,4-benzodiazepine, dihydrochloride;
N-Cyclohexyl-2,3,4,5-tetrahydro-1-(1H-imidazol-4-ylmethyl)-4-(1-naphthalenylcarbonyl)-1H-1,4-benzodiazepine-8-carboxamide, dihydrochloride;
N-(Cyclohexylmethyl)-2,3,4,5-tetrahydro-1-(1H-imidazol-4-ylmethyl)-4-(1-naphthalenylcarbonyl)-1H-1,4-benzodiazepine-8-carboxamide, dihydrochloride;
2,3,4,5-Tetrahydro-1-(1H-imidazol-4-ylmethyl)-4-(1-naphthalenylcarbonyl)-N-(phenylmethyl)-1H -1,4-benzodiazepine-8-carboxamide, dihydrochloride;
(R)-4-Acetyl-7-[2-[(dimethylamino)methyl]phenyl]-2,3,4,5-tetrahydro-1-(1H-imidazol-4-ylmethyl)-3-(phenylmethyl)-1H-1,4-benzodiazepine, dihydrochloride;
(R)-4-Acetyl-7-cyano-2,3,4,5-tetrahydro-1-(1H-imidazol-4-ylmethyl)-3-(phenylmethyl)-1H-1,4-benzodiazepine, monohydrochloride;
(R)-7-Cyano-2,3,4,5-tetrahydro-1-(1H-imidazol-4-ylmethyl)-4-(1-oxobutyl)-3-(phenylmethyl)-1H-1,4-benzodiazepine, monohydrochloride;
(R)-7-Cyano-2,3,4,5-tetrahydro-1-(1H-imidazol-4-ylmethyl)-4-(2-methyl-1-oxopropyl)-3-(phenylmethyl)-1H-1,4-benzodiazepine, monohydrochloride;
(R)-7-Cyano-2,3,4,5-tetrahydro-1-(1H-imidazol-4-ylmethyl)-3-(phenylmethyl)-4-(2-pyridinylacetyl)-1H-1,4-benzodiazepine, dihydrochloride;
(R)-7-Cyano-2,3,4,5-tetrahydro-1-(1H-imidazol-4-ylmethyl)-3-(phenylmethyl)-4-(2-thienylsulfonyl)-1H-1,4-benzodiazepine, monohydrochloride;
(R)-7-Cyano-2,3,4,5-tetrahydro-1-(1H-imidazol-4-ylmethyl)-4-[(1-methylethyl)sulfonyl]-3-(phenylmethyl)-1H-1,4-benzodiazepine, monohydrochloride;
(R)-7-Cyano-2,3,4,5-tetrahydro-1-(1H-imidazol-4-ylmethyl)-3-(phenylmethyl)-4-[(trifluoromethyl)sulfonyl]-1H-1,4-benzodiazepine, monohydrochloride;
(R)-7-Bromo-2,3,4,5-tetrahydro-1-(1H-imidazol-4-ylmethyl)-3-(phenylmethyl)-4-(propylsulfonyl)-1H-1,4-benzodiazepine, monohydrochloride;
(R)-7-Bromo-2,3,4,5-tetrahydro-1-(1H-imidazol-4-ylmethyl)-3-(phenylmethyl)-4-(phenylsulfonyl)-1H-1,4-benzodiazepine, monohydrochloride;
(R)-2,3,4,5-Tetrahydro-1-(1H-imidazol-4-ylmethyl)-7-phenyl-3-(phenylmethyl)-4-(phenylsulfonyl)-1H-1,4-benzodiazepine, monohydrochloride;
(R)-2,3,4,5-Tetrahydro-1-(1H-imidazol-4-ylmethyl)-7-phenyl-3-(phenylmethyl)-4-(propylsulfonyl)-1H-1,4-benzodiazepine, monohydrochloride;
(R)-7-Cyano-4-[(4-fluorophenyl)sulfonyl]-2,3,4,5-tetrahydro-1-(1H-imidazol-4-ylmethyl)-3-(phenylmethyl)-1H-1,4-benzodiazepine, monohydrochloride;
(R)-7-Cyano-4-[(3-cyanophenyl)sulfonyl]-2,3,4,5-tetrahydro-1-(1H-imidazol-4-ylmethyl)-3-(phenylmethyl)-1H-1,4-benzodiazepine, monohydrochloride;
(R)-7-Cyano-2,3,4,5-tetrahydro-1-(1H-imidazol-4-ylmethyl)-4-[(1-methyl-1H-imidazol-2-yl)sulfonyl]-3-(phenylmethyl)-1H-1,4-benzodiazepine, dihydrochloride;
(R)-4-[(3-Bromophenyl)sulfonyl]-7cyano-2,3,4,5-tetrahydro-1-(1H-imidazol-4-ylmethyl)-3-(phenylmethyl)-1H-1,4-benzodiazepine, monohydrochloride;
(R)—N-[5-[[7-cyano-2,3,4,5-tetrahydro-1-(1H-imidazol-4-ylmethyl)-3-(phenylmethyl)-1H-1,4-benzodiazepin-4-yl]sulfonyl]-4-methyl-2-thiazolyl]acetamide, dihydrochloride;
4-Acetyl-2,3,4,5-tetrahydro-1-(1H-imidazol-4-ylmethyl)-3-(phenylmethyl)-7-(4-pyridinyl)-1H-1,4-benzodiazepine, trihydrochloride;
2,3,4,5-Tetrahydro-1-(1H-imidazol-4-ylmethyl)-4-(2-phenyl-1,2-dioxoethyl)-7-(4-pyridinyl)-1H-1,4-benzodiazepine, trihydrochloride;
2,3,4,5-Tetrahydro-1-(1H-imidazol-4-ylmethyl)-7-(4-pyridinyl)-4-[2-(trifluoromethoxy)benzoyl]-1H-1,4-benzodiazepine, trihydrochloride;
(R)-2,3,4,5-Tetrahydro-1-[(1-methyl-1H-imidazol-5-yl)methyl]-4-(methylsulfonyl)-7-phenyl-3-(phenylmethyl)-1H-1,4-benzodiazepine;
(R)-7-Cyano-2,3,4,5-tetrahydro-1-(1H-imidazol-4-ylmethyl)-4-(phenylacetyl)-3-(phenylmethyl)-1H-1,4-benzodiazepine, monohydrochloride;
4-(2-Benzothiazolyl)-2,3,4,5-tetrahydro-1-(1H-imidazol-4-ylmethyl)-7-phenyl-1H-1,4-benzazepine, trihydrochloride;
2,3,4,5-Tetrahydro-1-(1H-imidazol-4-ylmethyl)-3-(phenylmethyl)-7-(3-pyridinyl)-4-(trifluoroacetyl)-1H-1,4-benzodiazepine, trihydrochloride;
2,3,4,5-Tetrahydro-1-(1H-imidazol-4-ylmethyl)-4-(methylsulfonyl)-3-(phenylmethyl)-7-(3-pyridinyl)-1H-1,4-benzodiazepine, trihydrochloride;
7-Bromo-3-[(1,1-dimethylethoxy)methyl]-1,2,3,4-tetrahydro-1-(1H-imidazol-4-ylmethyl)-5H-1,4-benzodiazepin-5one;
7-Bromo-2,3,4,5-tetrahydro-1-(1H-imidazol-4-ylmethyl)-4-(methylsulfonyl)-3-(phenoxymethyl)-1H-1,4-benzodiazepine, dihydrochloride;
7-Bromo-2,3,4,5-tetrahydro-3-(hydroxymethyl)-1-(1H-imidazol-4-ylmethyl)-4-(methylsulfonyl)-1H -1,4-benzodiazepine, monohydrochloride;
7-Bromo-3-[(1,1-dimethylethoxy)methyl]-2,3,4,5-tetrahydro-1-(1H-imidazol-4-ylmethyl)-4-(methylsulfonyl)-1H-1,4-benzodiazepine;
[7-Bromo-2,3,4,5-tetrahydro-1-(1H-imidazol-4-ylmethyl)-3-(phenylmethyl)-1H-1,4-benzodiazepin-8-yl]carbamic acid, 2-methylpropyl ester, trihydrochloride;
[4-Acetyl-7-bromo-2,3,4,5-tetrahydro-1-(1H-imidazol-4-ylmethyl)-3-(phenylmethyl)-1H-1,4-benzodiazepin-8-yl]carbamic acid, 2-methylpropyl ester;
N-[4-Acetyl-2,3,4,5-tetrahydro-1-(1H-imidazol-4-ylmethyl)-3-(phenylmethyl)-1H-1,4-benzodiazepin-8-yl]cyclohexanecarboxamide, dihydrochloride;
[7-Bromo-2,3,4,5-tetrahydro-1-(1H-imidazol-4-ylmethyl)-4-(methylsulfonyl)-3-(phenylmethyl)-1H-1,4-benzodiazepin-8-yl]carbamic acid, 2-methylpropyl ester;
(R)-2,3,4,5-Tetrahydro-1-(1H-imidazol-4-ylmethyl)-4-(phenylsulfonyl)-3-(phenylmethyl)-1H-1,4-benzodiazepine-7-carbonitrile, monohydrochloride;
7-Bromo-1,2,3,5-tetrahydro-1-(1H-imidazol-4-ylmethyl)-3-(phenylmethyl)-4H-1,4-benzodiazepine-4-acetamide;
7-Bromo-4-[(dimethylamino)acetyl]-2,3,4,5-tetrahydro-1-(1H-imidazol-4-ylmethyl)-3-(phenylmethyl)-1H-1,4-benzodiazepine;
(R)-7-Bromo-4-(1,2-dioxopropyl)-2,3,4,5-tetrahydro-1-(1H-imidazol-4-ylmethyl)-3-(phenylmethyl)-1H-1,4-benzodiazepine, trifluoroacetate;
(R)-7-Bromo-4-(cyclopropylcarbonyl)-2,3,4,5-tetrahydro-1-(1H-imidazol-4-ylmethyl)-3-(phenylmethyl)-1H-1,4-benzodiazepine, trifluoroacetate;
(R)-7-Cyano-2,3,4,5-tetrahydro-1-(1H-imidazol-4-ylmethyl)-3-(phenylmethyl)-4-(propylsulfonyl)-1H-1,4-benzodiazepine, monohydrochloride;
7-Bromo-2,3,4,5-tetrahydro-1,4-bis(1H-imidazol-4-ylmethyl)-3-(phenylmethyl)-1H-1,4-benzodiazepine, dihydrochloride;
7-Bromo-1,2,3,5-tetrahydro-1-(1H-imidazol-4-ylmethyl)-N,N-dimethyl-3-(phenylmethyl)-4H-1,4-benzodiazepine-4-sulfonamide, monohydrochloride;
2,3,4,5-Tetrahydro-1-(1H-imidazol-4-ylmethyl)-4-(methylsulfonyl)-3-(phenylmethyl)-1H-1,4-benzodiazepine-7-carbonitrile, monohydrochloride;
(R)-7-Cyano-1,2,3,5-tetrahydro-1-(1H-imidazol-4-ylmethyl)-N,N-dimethyl-3-(phenylmethyl)-4H-1,4-benzodiazepine-4-carboxamide monohydrochloride;
N,N-Diethyl-2,3,4,5-tetrahydro-1-(1H-imidazol-4-ylmethyl)-4-(methylsulfonyl)-3-(phenylmethyl)-1H-1,4-benzodiazepine-7-carboxamide, monohydrochloride;
2,3,4,5-Tetrahydro-1-(1H-imidazol-4-ylmethyl)-7-phenyl-4-(1-phenyl-1H-tetrazol-5-yl)-1H-1,4-benzodiazepine, monohydrochloride;
(R)-7-Cyano-2,3,4,5-tetrahydro-1-(1H-imidazol-4-ylmethyl)-3-(phenylmethyl)-4-(2-pyrazinylcarbonyl)-4H-1,4-benzodiazepine, monohydrochloride;
(R)-4-[7-Bromo-2,3,4,5-tetrahydro-1-(1H-imidazol-4-ylmethyl)-3-(phenylmethyl)-4H-1,4-benzodiazepin-4-yl]-4-oxobutanoic acid, methyl ester, monohydrochloride;
(R)-7-Cyano-2,3,4,5-tetrahydro-1-(1H-imidazol-4-ylmethyl)-4-(4-morpholinocarbonyl)-3-(phenylmethyl)-1H-1,4-benzodiazepine, monohydrochloride;
(R)-7-Cyano-2,3,4,5-tetrahydro-1-(1H-imidazol-4-ylmethyl)-3-(phenylmethyl)-4-[[2-(1-pyrrolidinyl)ethyl]sulfonyl]-1H-1,4-benzodiazepine, dihydrochloride;
(S)-2,3,4,5-Tetrahydro-1-(1H-imidazol-4-ylmethyl)-4-(methylsulfonyl)-7-phenyl-3-(3-pyridinylmethyl)-1H-1,4-benzodiazepine, dihydrochloride;
(R)-2,3,4,5-Tetrahydro-1-(1H-imidazol-4-ylmethyl)-7-phenyl-3-(3-pyridinylmethyl)-4-(2-thienylsulfonyl)-1H-1,4-benzodiazepine, dihydrochloride;
(R)-2,3,4,5-Tetrahydro-1-(1H-imidazol-4-ylmethyl)-7-phenyl-4-(propylsulfonyl)-3-(3-pyridinylmethyl)-1H-1,4-benzodiazepine, monohydrochloride;
(R)-7-Bromo-2,3,4,5-tetrahydro-1-(1H-imidazol-4-ylmethyl)-4-(methylsulfonyl)-3-(2-pyridinylmethyl)-1H-1,4-benzodiazepine, monohydrochloride;
2,3,4,5-Tetrahydro-1-(1H-imidazol-4-ylmethyl)-4-(methylsulfonyl)-3-(phenylmethyl)-7-(2-pyrimidinyl)-1H-1,4-benzodiazepine,dihydrochloride;
(R)-7-Bromo-2,3,4,5-tetrahydro-1-(1H-imidazol-4-ylmethyl)-3-(phenylmethyl)-4-[(trifluoromethyl)sulfonyl]-1H-1,4-benzodiazepine, monohydrochloride;
(R)-2,3,4,5-Tetrahydro-1-(1H-imidazol-4-ylmethyl)-7-phenyl-3-(phenylmethyl)-4-(trifluoroacetyl)-1H-1,4-benzodiazepine, monohydrochloride;
(R)-2,3,4,5-Tetrahydro-1-(1H-imidazol-4-ylmethyl)-4-(methylsulfonyl)-3-(phenylmethyl)-7-(4-pyridinyl)-1H-1,4-benzodiazepine, dihydrochloride;
(R)-2,3,4,5-Tetrahydro-1-(1H-imidazol-4-ylmethyl)-3-(phenylmethyl)-7-(4-pyridinyl)-4-(2-thienylsulfonyl)-1H-1,4-benzodiazepine, dihydrochloride;
(R)-2,3,4,5-Tetrahydro 1-(1H-imidazol-4-ylmethyl)-3-(phenylmethyl)-4-(phenylsulfonyl)-7-(4-pyridinyl)-1H-1,4-benzodiazepine, dihydrochloride;
(R)-2,3,4,5-Tetrahydro-1-(1H-imidazol-4-ylmethyl)-3-(phenylmethyl)-4-(propylsulfonyl)-7-(4-pyridinyl-1H-1,4-benzodiazepine, dihydrochloride;
(R)-7-Cyano-2,3,4,5-tetrahydro-1-(1H-imidazol-4-ylmethyl)-3-(phenylmethyl)-4-[(3,5-dimethyl-isoxazol-4-yl)sulfonyl]-1H-1,4-benzodiazepine, dihydrochloride;
(R)-7-Cyano4-[(4-cyanophenyl)sulfonyl]-2,3,4,5-tetrahydro-1-(1H-imidazol-4-ylmethyl)-3-(phenylmethyl)-1H-1,4-benzodiazepine, dihydrochloride;
(R)-7-Cyano-2,3,4,5-tetrahydro-1-(1H-imidazol-4-ylmethyl)-3-(phenylmethyl)-4-[(2,2,2-trifluoroethyl)sulfonyl]-1H-1,4-benzodiazepine, dihydrochloride;
(R)-[(5-Bromo-2-thienyl)sulfonyl]-7-cyano-2,3,4,5-tetrahydro-1-(1H-imidazol-4-ylmethyl)-3-(phenylmethyl)-1H-1,4-benzodiazepine, dihydrochloride;
(R)-7-Cyano-2,3,4,5-tetrahydro-1-(1H-imidazol-4-ylmethyl)-4-[(4-methoxyphenyl)sulfonyl]-3-(phenylmethyl)-1H-1,4-benzodiazepine, dihydrochloride;
N-[[7-Bromo-2,3,4,5-tetrahydro-1-(1H-imidazol-4-ylmethyl)-4-(methylsulfonyl)-1H-1,4-benzodiazepin-3-ylmethyl]benzamide, dihydrochloride;
(R)-7-Cyano-1,2,3,5-tetrahydro-1-(1H-imidazol-4-ylmethyl)-N,N-dimethyl-3-(phenylmethyl)-4H-1,4-benzodiazepine-4-sulfonamide, hydrochloride;
(R)-7-Cyano-1,2,3,5-tetrahydro-N,N-dimethyl-1-[(1-methyl-1H-imidazol-5-yl)methyl]-3-(phenylmethyl)-4H-1,4-benzodiazepine-4-sulfonamide, hydrochloride;
(R)-7-Chloro-2,3,4,5-tetrahydro-1-[(1-methyl-1H-imidazol-5-yl)methyl]-4-(methylsulfonyl)-3-(phenylmethyl)-1H-1,4-benzodiazepine, monohydrochloride;
(R)-7-Chloro-2,3,4,5-tetrahydro-1-(1H-imidazol-4-ylmethyl)-4-(methylsulfonyl)-3-(phenylmethyl)-1H-1,4-benzodiazepine, monohydrochloride;
(R)-7-Chloro-2,3,4,5-tetrahydro-1-[(1-methyl-1H-imidazol-5-yl)methyl]-4-(phenylsulfonyl)-3-phenylmethyl)-1H-1,4-benzodiazepine, monohydrochloride;
(R)-7-Cyano-2,3,4,5-tetrahydro-1-(1H-imidazol-4-ylmethyl)-3-(pyridin-3-ylmethyl)-4-(methylsulfonyl)-1H-1,4-benzodiazepine, tetrahydrochloride;
(R)-7-Bromo-2,3,4,5-tetrahydro-1-(1H-imidazol-2-ylmethyl)-4-(methylsulfonyl)-3-(phenylmethyl)-1H-1,4-benzodiazepine, dihydrochloride;
(R)-7-Bromo-2,3,4,5-tetrahydro-1-(1H-imidazol-4-ylmethyl)-4-[(1-methyl-1H-imidazol-4-yl)sulfonyl]-3-(phenylmethyl)-1H-1,4-benzodiazepine, trihydrochloride;
(R)-7-Chloro-2,3,4,5-tetrahydro-1-(1-methyl-imidazol-5-ylmethyl)-4-[(2-morpholin-4-yl-ethyl)sulfonyl]-3-(phenylmethyl)-1H-1,4-benzodiazepine, dihydrochloride;
(R)-7-Chloro-2,3,4,5-tetrahydro-1-(1H-imidazol-4-ylmethyl)-4-[(2-morpholin-4-yl-ethyl)sulfonyl]-3-(phenylmethyl)-1H-1,4-benzodiazepine, dihydrochloride;
(R)-7-Chloro-4-[(dimethylamino)sulfonyl]-1-[(1-methyl-1H-imidazol-5-yl)methyl]-3-(phenylmethyl)-1H-1,4-benzodiazepine, monohydrochloride;
(R)-7-Chloro-2,3,4,5-tetrahydro-1-(1-methyl-imidazol-5-ylmethyl)-4-[(4-methyl-piperidin-4-yl-ethyl)sulfonyl]-3-(phenylmethyl)-1H-1,4-benzodiazepine, dihydrochloride;
(R)-7-Bromo-2,3,4,5-tetrahydro-1-(1-methyl-imidazol-5-ylmethyl)-4-[(4-methyl-piperidin-4-yl-ethyl)sulfonyl]-3-(phenylmethyl)-1H-1,4-benzodiazepine, dihydrochloride;
(R)-7-Cyano-1,2,3,5-tetrahydro-1-(1H-imidazol-4-ylmethyl)-3-(phenylmethyl)-4H-1,4-benzodiazepine-4-carboxylic acid, isopropyl ester, hydrochloride;
(R)-7-Bromo-2,3,4,5-tetrahydro-4-[[2-(1H-imidazol-1-yl)ethyl]sulfonyl]-1-(1H-imidazol-4-ylmethyl)-3-(phenylmethyl)-1H-1,4-benzodiazepine, dihydrochloride;
(R)-7-Cyano-2,3,4,5-tetrahydro-1-(1H-imidazol-4-ylmethyl)-4-(propylsulfonyl)-3-(3-pyridinylmethyl)-1H-1,4-benzodiazepine, hydrochloride;
7-Bromo-2,3,4,5-tetrahydro-1-(1H-imidazol-4-ylmethyl)-3-(phenylmethyl)-1H-1,4-benzodiazepin-5-one, hydrochloride;
(R)-7-Bromo-2,3,4,5-tetrahydro-1-(1H-imidazol-1-ylacetyl)-4-(methylsulfonyl)-3-(phenylmethyl)-1H-1,4-benzodiazepine, trifluoroacetate;
1,2,4,5-Tetrahydro-1-(1H-imidazol-4-ylmethyl)-2-(2-phenylethyl)-3H-1,4-benzodiazepin-3-one;
2,3,4,5-Tetrahydro-1-(1H-imidazol-4-ylmethyl)-4-(methylsulfonyl)-2-(2-phenylethyl)-1H-1,4-benzodiazepine, monohydrochloride;
(R)-2,3,4,5-Tetrahydro-1-(1H-imidazol-4-ylmethyl)-4-(methylsulfonyl)-7-phenyl-3-(4-pyridinylmethyl)-1H-1,4-benzodiazepine, dihydrochloride;
(R)-2,3,4,5-Tetrahydro-1-(1H-imidazol-2-ylmethyl)-4-(phenylsulfonyl)-3-(phenylmethyl)-1H-1,4-benzodiazepine-7-carbonitrile, hydrochloride;
(R)-7-Cyano-1,2,3,5-tetrahydro-1-(1H-imidazol-4-ylmethyl)-N,N-dimethyl-3-(3-pyridinylmethyl)-4H-1,4-benzodiazepine-4-carboxamide, dihydrochloride;
(R)-7-Cyano-1,2,3,5-tetrahydro-1-(1H-imidazol-4-ylmethyl)-N,N-dimethyl-3-(3-pyridinylmethyl)-4H-1,4-benzodiazepine-4-sulfonamide, dihydrochloride;
(R)-2,3,4,5-Tetrahydro-1-(1-(4cyanophenylmethyl)-imidazol-5-ylmethyl)-4-(methylsulfonyl)-3-(phenylmethyl)-1H-1,4-benzodiazepine-7-carbonitrile, hydrochloride;
(R)-2,3,4,5-Tetrahydro-1-(1-(4-cyanophenylmethyl)-imidazol-4-ylmethyl)-4-(methylsulfonyl)-3phenylmethyl)-1H-1,4-benzodiazepine-7-carbonitrile, hydrochloride;
(R)-4-Benzoyl-7-Cyano-2,3,4,5-tetrahydro-1-(1H-imidazol-4-ylmethyl)-3-(phenylmethyl)-1H-1,4-benzodiazepine, monohydrochloride;
(R)-7-Cyano-2,3,4,5-tetrahydro-1-[(1-methyl-1H-imidazol-5-yl)methyl]-3-(pyridin-3-ylmethyl)-4-(methylsulfonyl)-1H-1,4-benzodiazepine, dihydrochloride;
(R)-7-Cyano-2,3,4,5-tetrahydro-1-[(1-methyl-1H-imidazol-5-yl)methyl]-3-(pyridin-3-ylmethyl)-4-(propylsulfonyl)-1H-1,4-benzodiazepine, trihydrochloride;
(R)-7-Cyano-2,3,4,5-tetrahydro-1-[(1H-imidazol-4-yl)methyl]-3-(pyridin-3-ylmethyl)-4-(phenylsulfonyl)-1H-1,4-benzodiazepine, dihydrochloride;
2,3,4,5-Tetrahydro-1-(1H-imidazol-4-ylmethyl)-4-(methylsulfonyl)-7-phenyl-3-(phenylmethyl)-1H-1,4-benzodiazepine;
1,2,3,5-Tetrahydro-1-(1H-imidazol-4-ylmethyl)-N-(1-naphthalenyl)-7-phenyl-4H-1,4-benzodiazepine-4-carboxamide, monohydrochloride;
(S)-7-Bromo-2,3,4,5-tetrahydro-1-(1H-imidazol-4-ylmethyl)-4-(methylsulfonyl)-3-(phenylmethyl)-1H-1,4-benzodiazepine, hydrochloride;
N-[2,3,4,5-Tetrahydro-1-(1H-imidazol-4-ylmethyl)-4-(2,3-dimethylbenzoyl)-1H-1,4-benzodiazepin-8-yl]cyclohexanecarboxamide, dihydrochloride;
(R)-7-Cyano-N-[2-(dimethylamino)ethyl]-2,3,4,5-tetrahydro-1-(1H-imidazol-4-ylmethyl)-N-methyl-3-(phenylmethyl)-1H-1,4-benzodiazepine-4-carboxamide, trifluoroacetate (1:2);
7-Bromo-2,3,4,5-tetrahydro-1-(1H-imidazol-4-ylmethyl)-4-(methylsulfonyl)-2-oxo-3-(phenylmethyl)-1H-1,4-benzodiazepine, trifluoroacetate;
(R)-7-Cyano-4-(2-furanylcarbonyl)-2,3,4,5-tetrahydro-1-(1H-imidazol-4-ylmethyl)-3-(phenylmethyl)-1H-1,4-benzodiazepine, trifluoroacetate (1:1);
(R)-7-Cyano-2,3,4,5-tetrahydro-1-(1H-imidazol-4-ylmethyl)-4-[(4-nitrophenyl)sulfonyl]-3-(phenylmethyl)-1H-1,4-benzodiazepine, trifluoroacetate;
(R)-7-Cyano-2,3,4,5-tetrahydro-1-(1H-imidazol-4-ylmethyl)-4-[[4-(4-methyl-1-piperazin)phenyl]sulfonyl]-3-(phenylmethyl)-1H-1,4-benzodiazepine, trifluoroacetate;
(R)-7-Cyano-2,3,4,5-tetrahydro-1-(1H-imidazol-4-ylmethyl)-4-[[(4-dimethylamino)phenyl]sulfonyl]-3-(phenylmethyl)-1H-1,4-benzodiazepine, trifluoroacetate;
(R)-7-Bromo-4-[[2-(dimethylamino)ethyl]sulfonyl]-2,3,4,5-tetrahydro-1-(1H-imidazol-4-ylmethyl)-3-(phenylmethyl)-4H-1,4-benzodiazepine, dihydrochloride;
(R)-7-Cyano-2,3,4,5-tetrahydro-1-(1H-imidazol-4-ylmethyl)-3-(phenylmethyl)-4-(3-pyridinylsulfonyl)-1H-1,4-benzodiazepine, trihydrochloride;
2,3,4,5-Tetrahydro-1-(1H-imidazol-4-ylmethyl)-4-(methylsulfonyl)-3-(phenylmethyl)-1H-1,4-benzo-diazepine, dihydrochloride;
(R)-7-Bromo-2,3,4,5-tetrahydro-1-[(1-methyl-1H-imidazol-4-yl)methyl]-4-(methylsulfonyl)-3-(phenylmethyl)-1H-1,4-benzodiazepine, dihydrochloride;
(R)-4-[[3-(Dimethylamino)propyl]sulfonyl]-2,3,4,5-tetrahydro-1-(1H-imidazol-4-ylmethyl)-7-phenyl-3-(phenylmethyl)-1H-1,4-benzodiazepine, dihydrochloride;
(R)-7-Cyano-2,3,4,5-tetrahydro-1-(1H-imidazol-4-ylmethyl)-3-(phenylmethyl)-1H-1,4-benzodiazepine, trihydrochloride;
4-Butyl-2,3,4,5-tetrahydro-1-(1H-imidazol-4-ylmethyl)-3-(phenylmethyl)-1H-1,4-benzodiazepine, trihydrochloride;
(R)-7-Bromo-2,3,4,5-tetrahydro-1-(1H-imidazol-4-ylmethyl)-4-[[2-(4-morpholinyl)ethyl]sulfonyl]-3-(phenylmethyl)-1H-1,4-benzodiazepine, dihydrochloride;
(R)-7-Bromo-2,3,4,5-tetrahydro-1-[(1-methyl-1H-imidazol-5-yl)methyl]-4-[[2-(4-morpholinyl)ethyl]sulfonyl]-3-(phenylmethyl)-1H-1,4-benzodiazepine, dihydrochloride;
(R)-7-Cyano-1-(1H-imidazol-4-ylmethyl)-4-(4-morpholinylsulfonyl)-3-(phenylmethyl)-1H-1,4-benzodiazepine, monohydrochloride;
(R)-7-Cyano-1-[(1-methyl-1H-imidazol-5-yl)methyl]-4-[(4-morpholinyl)sulfonyl-]-3-(phenylmethyl)-1H-1,4-benzodiazepine, monohydrochloride;
(R)-7-Cyano-2,3,4,5-tetrahydro-1-(1H-imidazol-4-ylmethyl)-4-[(4-aminophenyl)sulfonyl]-3-(phenylmethyl)-1H-1,4-benzodiazepine, hydrochloride;
2,3,4,5-Tetrahydro-1-(1H-imidazol-4-ylmethyl)-4-[(4-pyridylthio)acetyl]-7-phenyl-1H-1,4-benzodiazepine, dihydrochloride;
N-(4-Chlorophenyl)-N′-cyano-1,2,3,5-tetrahydro-1-(1H-imidazol-4-ylmethyl)-7-phenyl-4H-1,4-benzodiazepine-4-imidamide, monohydrochloride;
4-Acetyl-7-bromo-1,2,4,5,1′,3′-hexahydro-1-(1H-imidazol-4-ylmethyl)spiro[3H-1,4-benzodiazepine-3,2′-[2H]indene], dihydrochloride;
7-Bromo-4-[3-(dimethylamino)-1-oxopropyl]-2,3,4,5-tetrahydro-1-(1H-imidazol-4-ylmethyl)-3-(phenylmethyl)-1H-1,4-benzodiazepine, trifluoroacetate (1:1);
(R)-2,3,4,5-Tetrahydro-1-(1-methyl-1H-imidazol-5-ylmethyl)-4-(phenylsulfonyl)-3-(phenylmethyl)-1H-1,4-benzodiazepine-7-carbonitrile, monohydrochloride;
2,3,4,5-Tetrahydro-1-[(1-methyl-1H-imidazol-5-yl)-methyl]-4-(methyl-sulfonyl)-7-phenyl-3-(pyridin-3-yl-methyl)-1H-1,4-benzodiazepine, hydrochloride (1:1.5), trifluoroacetate (1:0.75) salt;
4-[4-(Fluorophenyl)sulfonyl]-2,3,4,5-tetrahydro-1-(1H-imidazol-4-ylmethyl)-2-(2-phenylethyl)-1H-1,4-benzodiazepine, monohydrochloride;
7-Bromo-2,3,4,5-tetrahydro-1-(1H-imidazol-4-yl-methyl)-4-(methyl-sulfonyl)-2-(2-phenylethyl)-1H-1,4-benzodiazepine, monohydrochloride;
(R)-7-Cyano-2,3,4,5-tetrahydro-1-(1-methyl-1H-imidazol-5-ylmethyl)-4-[[2-(1-morpholinyl)ethyl]sulfonyl]-3-(phenylmethyl)-1H-1,4-benzodiazepine, dihydrochloride;
(R)-7-Bromo-2,3,4,5-tetrahydro-1-(1H-imidazol-4-ylmethyl)-4-(methyl-sulfonyl)-3-(4-bromophenylmethyl)-1H-1,4-benzodiazepine, hydrochloride;
(R)-7-Bromo-2,3,4,5-tetrahydro-1-(1H-imidazol-4-ylmethyl)-4-(methyl-sulfonyl)-3-(thiazol-4-ylmethyl)-1H-1,4-benzodiazepine, hydrochloride;
(R)-7-Bromo-2,3,4,5-tetrahydro-1-(1H-imidazol-4-ylmethyl)-4-(propyl-sulfonyl)-3-(thiazol-4-ylmethyl)-1H-1,4-benzodiazepine, hydrochloride;
(R)-7-Bromo-2,3,4,5-tetrahydro-1-(1H-imidazol-4-ylmethyl)-4-(propylsulfonyl)-3-(4-bromophenylmethyl)-1H-1,4-benzodiazepine, hydrochloride;
(R)-7-Bromo-2,3,4,5-tetrahydro-1-(1H-imidazol-4-ylmethyl)-3-(pyridin-3-ylmethyl)-4-(methylsulfonyl)-1H-1,4-benzodiazepine, trihydrochloride;
(R)-7-Bromo-2,3,4,5-tetrahydro-1-(1H-1-methyl-imidazol-5-ylmethyl)-3-(pyridin-3-ylmethyl)-4-(methylsulfonyl)-1H-1,4-benzodiazepine, dihydrochloride;
(R)-7-Cyano-2,3,4,5-tetrahydro-1-(1H-imidazol-4-ylmethyl)-4-(phenyl-sulfonyl)-3-(4-cyanophenylmethyl)-1H-1,4-benzodiazepine, hydrochloride;
(R)-7-Cyano-4-[(N-methyl-N-phenylmethyl)aminosulfonyl]-1-[(1H-imidazol-4-yl)methyl]-3-(phenylmethyl)-1H-1,4-benzodiazepine, monohydrochloride;
(R)-7-Cyano4-[N-(tetrahydroisoquinoline)sulfonyl]-1-[(1H-imidazol-4-yl)methyl]-3-(phenylmethyl)-1H-1,4-benzodiazepine, monohydrochloride;
(R)-7-Cyano-2,3,4,5-tetrahydro-1-(1H-imidazol-4-ylmethyl)-4-(phenylsulfonyl)-3-(2-thienylmethyl)-1H-1,4-benzodiazepine, hydrochloride;
cis-2,3,4,5-Tetrahydro-1,5-bis(1H-imidazol-4-ylmethyl)-3-(phenylmethyl)-1H-1,5-benzodiazepine-2-carboxylic acid ethyl ester-trifluoroacetate (1:2);
(R)-7-Cyano4-[(N-piperidinyl)sulfonyl]-1-[(1H-imidazol-4-yl)methyl]-3-(phenylmethyl)-1H-1,4-benzodiazepine, monohydrochloride;
(R)-7-Cyano-2,3,4,5-tetrahydro-1-(1H-1-methyl-imidazol-5-ylmethyl)-3-(phenylmethyl)-4-(2-thienylsulfonyl)-1H-1,4-benzodiazepine, hydrochloride;
(R)-7-Cyano-2,3,4,5-tetrahydro-1-(1H-imidazol-4-ylmethyl)-3-(pyridin-3-ylmethyl)-4-[[2-(dimethylamino)ethyl]sulfonyl]-1H-1,4-benzodiazepine, trihydrochloride;
(R)-7-Cyano-2,3,4,5-tetrahydro-1-(1H-1-methyl-imidazol-5-ylmethyl)-3-(phenylmethyl)-4-(propylsulfonyl)-1H-1,4-benzodiazepine, hydrochloride;
N-(Cyano)-N′-methyl-1,2,3,5-tetrahydro-1-(1H-imidazol-4-ylmethyl)-7-phenyl-4H-1,4-benzodiazepine-4-imidamide, hydrochloride;
(R)-7-Cyano-4-[(2-nitrophenyl)-sulfonyl]-2,3,4,5-tetrahydro-1-(1H-imidazol-4-ylmethyl)-3-(phenyl-methyl)-1H-1,4-benzodiazepine, hydrochloride;
(R)-7-Cyano-4-[(4-methyl-phenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano4-(butylsulfonyl)-2,3,4,5-tetrahydro-1-(1H-imidazol-4-ylmethyl)-3-(phenylmethyl)-1H-1,4-benzodiazepine, hydrochloride;
(R)-7-Cyano-4-[(2-trifluoro-methylphenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-4-[(2-trifluoromethylphenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-4-[(2-methoxy-carbonylphenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-4-[(2-methyl-sulfonylphenyl)sulfonyl]-2,3,4,5-tetrahydro-1-(1H-imidazol-4-ylmethyl)-3-(phenylmethyl)-1H-1,4-benzodiazepine, hydrochloride;
(R)-7-Cyano-2,3,4,5-tetrahydro-1-(1H-imidazol-4-ylmethyl)-3-(phenylmethyl)-4-(((4-methylnonyl)-phenyl)-sulfonyl)-1H-1,4-benzodiazepine;
(R)-7-Cyano-2,3,4,5-tetrahydro-1-(1H-imidazol-4-ylmethyl)-3-(phenylmethyl)-4-(((4-trifluoromethyl)-phenyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((3-methoxypropyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((3,4-dimethoxyphenyl)-sulfonyl)-1H-1,4-benzodiazepine;
(R)-7-Cyano-2,3,4,5-tetrahydro-1-(1H-imidazol-4-ylmethyl)-3-((4-fluorophenyl)methyl)-4-(phenylsulfonyl)-1H-1,4-benzodiazepine;
(R)-7-Cyano-4-(N-cyclopropylmethyl-N-propyl)-aminosulfonyl]-1-[(1H-imidazol-4-yl)methyl]-3-(phenylmethyl)-1H-1,4-benzodiazepine;
(R)-7-Cyano-4-[(N,N-(dibutylamino))-sulfonyl]-1-[(1H-imidazol-4-yl)methyl]-3-(phenylmethyl)-1H-1,4-benzodiazepine;
(R)-7-Chloro-4-(methanesulfonyl)-2,3,4,5-tetrahydro-1-(1H-imidazol-4-ylmethyl)-3-(phenylmethyl)-1H-pyrido[3,4-e]-1,4-diazepine;
1,2,3,4-Tetrahydro-7-bromo-4-[(1H-imidazol-4-yl)methyl]-2-phenylmethyl-1-(methylsulfonyl)quinoxaline;
(R)-7-Cyano-2,3,4,5-tetrahydro-1-(1H-imidazol-4-ylmethyl)-3-(phenylmethyl)-4-((imidazol-4-yl)methylsulfonyl)-1H-1,4-benzodiazepine;
(R)-7-Cyano-2,3,4,5-tetrahydro-1-(1H-imidazol-4-ylmethyl)-3-((2-thienyl)methyl)-4-(propylsulfonyl)-1H-1,4-benzodiazepine;
(R)-7-Cyano-2,3,4,5-tetrahydro-1-(1H-imidazol-4-ylmethyl)-3-((2-thienyl)methyl)-4-((2-thienyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((3-methylthiopropyl)-sulfonyl)-1H-1,4-benzodiazepine;
(R)-7-Cyano-2,3,4,5-tetrahydro-1-(1H-imidazol-4-ylmethyl)-3-(phenylmethyl)-4-(((3-methylthioxo)-propyl)-sulfonyl)-1H-1,4-benzodiazepine;
(R)-7-Cyano-2,3,4,5-tetrahydro-1-(1H-imidazol-4-ylmethyl)-3-(phenylmethyl)-4-(((3-methylsulfonyl)-propyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((2-methylpropyl)-sulfonyl)-1H-1,4-benzodiazepine;
(R)-7-Cyano-2,3,4,5-tetrahydro-1-(1H-imidazol-4-ylmethyl)-3-(phenylmethyl)-4-(cyclopentylsulfonyl)-1H-1,4-benzodiazepine;
(R)-7-Cyano-2,3,4,5-tetrahydro-1-(1H-imidazol-4-ylmethyl)-3-(phenylmethyl)-4-((4,4,4-trifluorobutyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((phenylmethyl)-sulfonyl)-1H-1,4-benzodiazepine;
(R)-7-Cyano-2,3,4,5-tetrahydro-1-(1H-imidazol-4-ylmethyl)-3-(phenylmethyl)-4-[[2-(5-(N-benzoyl)-aminomethyl)-thienyl]-sulfonyl]-1H-1,4-benzodiazepine;
(R)-7-Cyano-2,3,4,5-tetrahydro-1-(1H-imidazol-4-ylmethyl)-3-(phenylmethyl)-4-[[2-(1-(3-chloro-5-methyl-pyridin-2-yl))-pyrrolyl]-sulfonyl]-1H-1,4-benzodiazepine;
(R)-7-Cyano-2,3,4,5-tetrahydro-1-(1H-imidazol-4-ylmethyl)-3-(phenylmethyl)-4-((4-carboxyphenyl)-sulfonyl)-1H-1,4-benzodiazepine;
(R)-7-Cyano-2,3,4,5-tetrahydro-1-(1H-imidazol-4-ylmethyl)-3-(phenylmethyl)-4-[((3-methyl-1,2,4-oxadiazol-5-yl)-phenyl)-sulfonyl]-1H-1,4-benzodiazepine;
(R)-7-Cyano-2,3,4,5-tetrahydro-1-(1H-imidazol-4-ylmethyl)-3-(phenylmethyl)-4-((2,5-dimethoxyphenyl)-sulfonyl)-1H-1,4-benzodiazepine;
(R)-7-Cyano-4-[(N-tetrahydroquinolinyl)sulfonyl]-1-[(1H-imidazol-4-yl)methyl]-3-(phenylmethyl)-1H-1,4-benzodiazepine;
(R)-7-Cyano-4-(N,N-bis-[1-(2-methylpropyl)amino]-sulfonyl]-1-[(1H-imidazol-4-yl)methyl]-3-(phenylmethyl)-1H-1,4-benzodiazepine;
(R)-7-Cyano-4-[(N-methyl-N-phenyl)aminosulfonyl]-1-[(1H-imidazol-4-yl)methyl]-3-(phenylmethyl)-1H-1,4-benzodiazepine;
(R)-7-Cyano-2,3,4,5-tetrahydro-1-(1H-imidazol-4-ylmethyl)-3-(2-(2,6-dimethylphenyl)-ethyl)-4-(methylsulfonyl)-1H-1,4-benzodiazepine;
(R)-7-Cyano-2,3,4,5-tetrahydro-1-(1-(N-phthalimidoethyl)-imidazol-5-ylmethyl)-3-(phenylmethyl)-4-methylsulfonyl)-1H-1,4-benzodiazepine;
(R)-7-Cyano-2,3,4,5-tetrahydro-1-[(2-(N,N-dimethylamino)-ethyl)-imidazol-5-ylmethyl]-3-(phenylmethyl)-4-(methylsulfonyl)-1H-1,4-benzodiazepine;
(R)-7-Cyano-2,3,4,5-tetrahydro-1-[(2-aminoethyl)-imidazol-5-ylmethyl]-3-(phenylmethyl)-4-(methylsulfonyl)-1H-1,4-benzodiazepine;
(R)-7-Bromo-4-(methanesulfonyl)-2,3,4,5-tetrahydro-1-[(1H-imidazol-4-ylmethyl)-3-(phenylmethyl)-1H-thieno[2,3-e]-1,4-diazepine;
(R)-7-Bromo-4-(methanesulfonyl)-2,3,4,5-tetrahydro-1-[(1H-imidazol-4-ylmethyl)-3-(phenylmethyl)-1H-thieno[3,2-e]-1,4-diazepine;
(R)-4-(methanesulfonyl)-2,3,4,5-tetrahydro-1-[(1H-imidazol-4-ylmethyl)-3-(phenylmethyl)-1H-8-oxo-pyrimidino[4,5-e]-1,4-diazepine;
(R)-7-Cyano-2,3,4,5-tetrahydro-1-(1H-imidazol-4-ylmethyl)-3-((4-(2-methoxyethoxy)-phenyl)methyl)-4-(phenylsulfonyl)-1H-1,4-benzodiazepine;
(R)-7-Cyano-2,3,4,5-tetrahydro-1-(1H-imidazol-4-ylmethyl)-3-((4-(2-(dimethylamino)-ethoxy)-phenyl)methyl)-4-(phenylsulfonyl)-1H-1,4-benzodiazepine;
(R)-7-Cyano-2,3,4,5-tetrahydro-1-(1H-imidazol-4-ylsulfonyl)-3-(phenylmethyl)-4-(methylsulfonyl)-1H-1,4-benzodiazepine;
(R)-7-Cyano-2,3,4,5-tetrahydro-1-(1H-imidazol-4-ylsulfonyl)-3-(phenylmethyl)-4-(propylsulfonyl)-1H-1,4-benzodiazepine;
(R)-7-Cyano-2,3,4,5-tetrahydro-1-(1H-imidazol-4-ylsulfonyl)-3-(phenylmethyl)-4-(phenylsulfonyl)-1H-1,4-benzodiazepine;
(R)-7-Cyano-2,3,4,5-tetrahydro-1-(1H-imidazol-4-ylsulfonyl)-3-(phenylmethyl)-4-(2-thienylsulfonyl)-1H-1,4-benzodiazepine;
7-Cyano-2,3,4,5-tetrahydro-1-(1H-imidazol-4-ylmethyl)-3-(R)-[(R)-1-phenyl-ethyl]-4-(methylsulfonyl)-1H-1,4-benzodiazepine;
7-Cyano-2,3,4,5-tetrahydro-1-(1H-imidazol-4-ylmethyl)-3-(R)-[(R)-1-phenyl-ethyl]-4-(propylsulfonyl)-1H-1,4-benzodiazepine;
7-Cyano-2,3,4,5-tetrahydro-1-(1H-imidazol-4-ylmethyl)-3-(R)-[(R)-1-phenyl-ethyl]-4-(phenylsulfonyl)-1H-1,4-benzodiazepine;
7-Cyano-2,3,4,5-tetrahydro-1-(1H-imidazol-4-ylmethyl)-3-(R)-[(R)-1-phenyl-ethyl]-4-((2-thienyl)-sulfonyl)-1H-1,4-benzodiazepine;
7-Cyano-2,3,4,5-tetrahydro-1-(1H-imidazol-4-ylmethyl)-3-(S)-[(R)-1-phenyl-ethyl]-4-(methylsulfonyl)-1H-1,4-benzodiazepine;
7-Cyano-2,3,4,5-tetrahydro-1-(1H-imidazol-4-ylmethyl)-3-(S)-[(R)-1-phenyl-ethyl]-4-(propysulfonyl)-1H-1,4-benzodiazepine;
7-Cyano-2,3,4,5-tetrahydro-1-(1H-imidazol-4-ylmethyl)-3-(S)-[(R)-1-phenyl-ethyl]-4-(phenylsulfonyl)-1H-1,4-benzodiazepine;
7-Cyano-2,3,4,5-tetrahydro-1-(1H-imidazol-4-ylmethyl)-3-(S)-[(R)-1-phenyl-ethyl]-4-((2-thienyl)-sulfonyl)-1H-1,4-benzodiazepine;
7-Cyano-2,3,4,5-tetrahydro-1-(1H-imidazol-4-ylmethyl)-3-(R)-[(S)-1-phenyl-ethyl]-4-(methylsulfonyl)-1H-1,4-benzodiazepine;
7-Cyano-2,3,4,5-tetrahydro-1-(1H-imidazol-4-ylmethyl)-3-(R)-[(S)-1-phenyl-ethyl]-4-(propylsulfonyl)-1H-1,4-benzodiazepine;
7-Cyano-2,3,4,5-tetrahydro-1-(1H-imidazol-4-ylmethyl)-3-(R)-[(S)-1-phenyl-ethyl]-4-(phenylsulfonyl)-1H-1,4-benzodiazepine;
7-Cyano-2,3,4,5-tetrahydro-1-(1H-imidazol-4-ylmethyl)-3-(R)-[(S)-1-phenyl-ethyl]-4-((2-thienyl)-sulfonyl)-1H-1,4-benzodiazepine;
7-Cyano-2,3,4,5-tetrahydro-1-(1H-imidazol-4-ylmethyl)-3-(S)-[(S)-1-phenyl-ethyl]-4-(methylsulfonyl)-1H-1,4-benzodiazepine;
7-Cyano-2,3,4,5-tetrahydro-1-(1H-imidazol-4-ylmethyl)-3-(S)-[(S)-1-phenyl-ethyl]-4-(propylsulfonyl)-1H-1,4-benzodiazepine;
7-Cyano-2,3,4,5-tetrahydro-1-(1H-imidazol-4-ylmethyl)-3-(S)-[(S)-1-phenyl-ethyl]-4-(phenylsulfonyl)-1H-1,4-benzodiazepine;
7-Cyano-2,3,4,5-tetrahydro-1-(1H-imidazol-4-ylmethyl)-3-(S)-[(S)-1-phenyl-ethyl]-4-((2-thienyl)-sulfonyl)-1H-1,4-benzodiazepine;
7-Cyano-2,3,4,5-tetrahydro-1-(1H-imidazol-4-ylmethyl)-3-(R)-[(R)-phenylcyclopropyl)-4-(methylsulfonyl)-1H-1,4-benzodiazepine;
7-Cyano-2,3,4,5-tetrahydro-1-(1H-imidazol-4-ylmethyl)-3-(R)-[(R)-phenylcyclopropyl)-4-propylsulfonyl)-1H-1,4-benzodiazepine;
7-Cyano-2,3,4,5-tetrahydro-1-(1H-imidazol-4-ylmethyl)-3-(R)-[(R)-phenylcyclopropyl)-4-phenylsulfonyl)-1H-1,4-benzodiazepine;
7-Cyano-2,3,4,5-tetrahydro-1-(1H-imidazol-4-ylmethyl)-3-(R)-[(R)-phenylcyclopropyl)-4-((2-thienyl)-sulfonyl)-1H-1,4-benzodiazepine;
7-Cyano-2,3,4,5-tetrahydro-1-(1H-imidazol-4-ylmethyl)-3-(R)-[(S)-phenylcyclopropyl)-4-(methylsulfonyl)-1H-1,4-benzodiazepine;
7-Cyano-2,3,4,5-tetrahydro-1-(1H-imidazol-4-ylmethyl)-3-(R)-[(S)-phenylcyclopropyl)-4-propylsulfonyl)-1H-1,4-benzodiazepine;
7-Cyano-2,3,4,5-tetrahydro-1-(1H-imidazol-4-ylmethyl)-3-(R)-[(S)-phenylcyclopropyl)-4-phenylsulfonyl)-1H-1,4-benzodiazepine;
7-Cyano-2,3,4,5-tetrahydro-1-(1H-imidazol-4-ylmethyl)-3-(R)-[(S)-phenylcyclopropyl)-4-((2-thienyl)-sulfonyl)-1H-1,4-benzodiazepine;
7-Cyano-2,3,4,5-tetrahydro-1-(1H-imidazol-4-ylmethyl)-3-(S)-[(R)-phenylcyclopropyl)-4-(methylsulfonyl)-1H-1,4-benzodiazepine;
7-Cyano-2,3,4,5-tetrahydro-1-(1H-imidazol-4-ylmethyl)-3-(S)-[(R)-phenylcyclopropyl)-4-(propylsulfonyl)-1H-1,4-benzodiazepine;
7-Cyano-2,3,4,5-tetrahydro-1-(1H-imidazol-4-ylmethyl)-3-(S)-[(R)-phenylcyclopropyl)-4-(phenylsulfonyl)-1H-1,4-benzodiazepine;
7-Cyano-2,3,4,5-tetrahydro-1-(1H-imidazol-4-ylmethyl)-3-(S)-[(R)-phenylcyclopropyl)-4-((2-thienyl)-sulfonyl)-1H-1,4-benzodiazepine;
7-Cyano-2,3,4,5-tetrahydro-1-(1H-imidazol-4-ylmethyl)-3-(S)-[(S)-phenylcyclopropyl)-4-(methylsulfonyl)-1H-1,4-benzodiazepine;
7-Cyano-2,3,4,5-tetrahydro-1-(1H-imidazol-4-ylmethyl)-3-(S)-[(S)-phenylcyclopropyl)-4-(propylsulfonyl)-1H-1,4-benzodiazepine;
7-Cyano-2,3,4,5-tetrahydro-1-(1H-imidazol-4-ylmethyl)-3-(S)-[(S)-phenylcyclopropyl)-4-(phenylsulfonyl)-1H-1,4-benzodiazepine;
7-Cyano-2,3,4,5-tetrahydro-1-(1H-imidazol-4-ylmethyl)-3-(S)-[(S)-phenylcyclopropyl)-4-((2-thienyl)-sulfonyl)-1H-1,4-benzodiazepine;
(R)-7-Cyano-2,3,4,5-tetrahydro-1-(1H-imidazol-4-ylmethyl)-3-(phenylmethyl)-4-[(2-(5-(pyridin-2-yl))-thienyl)-sulfonyl])-1H-1,4-benzodiazepine;
(R)-7-Cyano-2,3,4,5-tetrahydro-1-(1H-imidazol-4-ylmethyl)-3-(phenylmethyl)-4-[(2-(5-(−1,2-isoxazol-3-yl))-thienyl)-sulfonyl])-1H-1,4-benzodiazepine;
(R)-7-Cyano-2,3,4,5-tetrahydro-1-(3-(1H-imidazol-2-yl)-propyl)-3-(phenylmethyl)-4-(phenylsulfonyl)-1H-1,4-benzodiazepine;
(R)-7-Cyano-2,3,4,5-tetrahydro-1-(3-(1H-imidazol-2-yl)-propyl)-3-(phenylmethyl)-4-(methylsulfonyl)-1H-1,4-benzodiazepine;
(R)-7-Cyano-2,3,4,5-tetrahydro-1-(3-(1H-imidazol-2-yl)-propyl)-3-(phenylmethyl)-4-(propylsulfonyl)-1H-1,4-benzodiazepine;
(R)-7-Cyano-2,3,4,5-tetrahydro-1-(3-(1H-imidazol-2-yl)-propyl)-3-(phenylmethyl)-4-((2-thienyl)-sulfonyl)-1H-1,4-benzodiazepine;
(R)-7-Cyano-2,3,4,5-tetrahydro-1-(2-(1H-imidazol-2-yl)-ethylsulfonyl)-3-(phenylmethyl)-4-(phenylsulfonyl)-1H-1,4-benzodiazepine;
(R)-7-Cyano-2,3,4,5-tetrahydro-1-(2-(1H-imidazol-2-yl)-ethylsulfonyl)-3-(phenylmethyl-4-(methylsulfonyl)-1H-1,4-benzodiazepine;
(R)-7-Cyano-2,3,4,5-tetrahydro-1-(2-(1H-imidazol-2-yl)-ethylsulfonyl)-3-(phenylmethyl)-4-(propylsulfonyl)-1H-1,4-benzodiazepine;
(R)-7-Cyano-2,3,4,5-tetrahydro-1-(2-(1H-imidazol-2-yl)ethylsulfonyl)-3-(phenylmethyl)-4-((2-thienyl)-sulfonyl)-1H-1,4-benzodiazepine;
(R)-7-Cyano-2,3,4,5-tetrahydro-1-(1H-imidazol-4-ylmethyl)-3-(phenylmethyl)-4-((1-oxoethyl)-amino)-1H-1,4-benzodiazepine;
(R)-7-Cyano-2,3,4,5-tetrahydro-1-(1H-imidazol-4-ylmethyl)-3-(phenylmethyl)-4-(methanesulfonylamino)-1H-1,4-benzodiazepine; and
(R)-7-Cyano-2,3,4,5-tetrahydro-1-(1H-imidazol-4-ylmethyl)-3-(phenylmethyl)-4-(phenylsulfonylamino)-1H-1,4-benzodiazepine.

In one aspect of the invention is a method of treating a synucleinopathic subject, the method comprising, administering to a synucleinopathic subject a farnesyl transferase inhibitor compound of the formula:

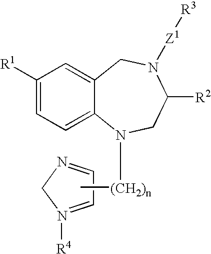

or a stereoisomeric form, or a pharmaceutically acceptable acid or base addition salt form thereof, in a therapeutically effective amount, wherein R₁ is Cl, Br, CN, optionally substituted phenyl, or optionally substituted 2-, 3- or 4-pyridyl; R₂ is optionally substituted lower alkyl, or optionally substituted aralkyl; R₃ and R₅ are each independently optionally substituted lower alkyl, optionally substituted aryl, or optionally substituted heterocyclo; R₄ is hydrogen or lower alkyl; Z₁ is CO, SO₂, CO₂ or SO₂N(R₅)—; and n is 1 or 2. In one embodiment the compound of the invention has the following substituents:
R₁ is Br, or CN;
R₂ is optionally substituted benzyl;
R₃ is optionally substituted lower alkyl, optionally substituted phenyl, optionally substituted
2-thienyl, or optionally substituted 1-piperidinyl;
R₄ is hydrogen, or methyl;
Z₁ is CO, SO₂, or SO₂N(R₅)—;
R₅ is optionally substituted lower alkyl or optionally substituted phenyl;
and n is 1.

In yet another embodiment the compound of the invention has the following substituents:
R₁ is CN;
R₂ is optionally substituted benzyl;
R₃ is optionally substituted lower alkyl, optionally substituted phenyl, optionally substituted 2-thienyl, or optionally substituted 1-piperidinyl;
R₄ is hydrogen, or methyl;
Z is CO, or SO₂; and
n is 1.

In yet another embodiment the compound of the invention has the following substituents:
R₁ is CN;
R₂ is benzyl;
R₃ is n-propyl, n-butyl, 3-methoxypropyl, 2-thienyl, 5-bromo-2-thienyl, phenyl, 4-methoxyphenyl, or 1-piperidinyl;
R₄ is hydrogen;
Z is SO₂; and
n is 1.

In yet another embodiment the compound of the invention is selected from the group consisting of:
(R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile;
(R)-7-cyano-2,3,4,5-tetrahydro-1-(1H-imidazol-4-ylmethyl)-4-(1-oxobutyl)-3-(phenylmethyl)-1H-1,4-benzodiazepine;
(R)-4-[(5-bromo-2-thienyl)sulfonyl]-7-cyano-2,3,4,5-tetrahydro-1-(1H-imidazol-4-ylmethyl)-3-(phenyl methyl)-1H-1,4-benzodiazepine;
(R)-7-cyano-2,3,4,5-tetrahydro-1-(1H-imidazol-4-ylmethyl)-4-[(4-methoxyphenyl)sulfonyl]-3-(phenylmethyl)-1H-1,4-benzodiazepine;
(R)-7-cyano-2,3,4,5-tetrahydro-1-(1H-imidazol-4-ylmethyl)-3-(phenyl methyl)-4-(phenylsulfonyl)-1H-1,4-benzodiazepine;
(R)-7-cyano-2,3,4,5-tetrahydro-1-(1H-imidazol-4-ylmethyl)-3-(phenylmethyl)-4-(propylsulfonyl)-1H-1,4-benzodiazepine;
(R)-4-(butylsulfonyl)-7-cyano-2,3,4,5-tetrahydro-1-(1H-imidazol-4-ylmethyl)-3-(phenylmethyl)-1H-1,4-benzodiazepine;
(R)-7-cyano-2,3,4,5-tetrahydro-1-(1H-imidazol-4-ylmethyl)-3-(phenylmethyl)-4-(1-piperidinylsulfonyl)-1H-1,4-benzodiazepine;
(R)-4-(3-methoxypropylsulfonyl)-7-cyano-2,3,4,5-tetrahydro-1-(1H-imidazol-4-ylmethyl)-3-(phenylmethyl)-1H-1,4-benzodiazepine; and
pharmaceutically acceptable salts thereof.

In certain embodiments of the invention the pharmaceutically acceptable salt is selected from the group consisting of the hydrochloride salt, the methanesulfonic acid salt and the trifluoroacetic acid salt.

In one embodiment of the invention compound of the invention is (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile.

In another embodiment, the invention is a method of treating a synucleinopathic subject, the method comprising, administering to a synucleinopathic subject a farnesyl transferase inhibitor compound of the formula:

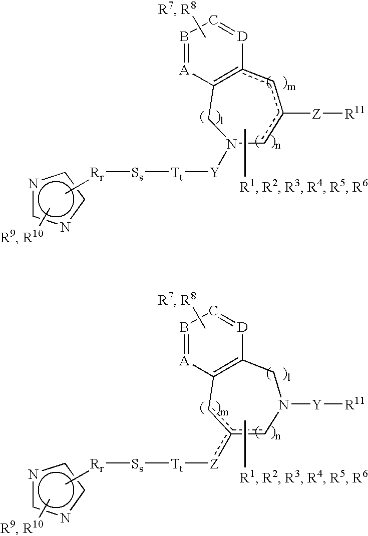

or a stereoisomeric form, or a pharmaceutically acceptable acid or base addition salt form thereof, in a therapeutically effective amount, wherein l, m, r, s and t are 0 or 1; n is 0, 1 or 2; Y is selected from the group consisting of CHR¹², SO₂, SO₃, CO, CO₂, O, NR¹³, SO₂NR¹⁴, CONR¹⁵, C(NCN), C(NCN)NR¹⁶, NR¹⁷CO, NR¹⁸SO₂, CONR¹⁹NR²⁰, SO₂NR²¹NR²², S(O)(NR²³), S(NR²⁴)(NR²⁵), or without Y; Z is selected from the group consisting of CR¹², S, SO, SO₂, SO₃, CO, CO₂, O, NR¹³, SO₂NR¹⁴, CONR¹⁵, NR²⁶NR²⁷, ONR²⁸, NR²⁹O, NR³⁰SO₂NR³¹, NR³²SO₂, NR³³C(NCN), NR³⁴C(NCN)NR³⁵, NR³⁶CO, NR³⁷CONR³⁸, NR³⁹CO₂, OCONR⁴⁰, S(O)(NR⁴¹), S(NR⁴²)(NR⁴³) or CHR¹²; or without Z; R⁷, R⁸ are selected from the group consisting of hydrogen, halo, nitro, cyano and U—R⁴⁴; U is selected from the group consisting of S, O, NR⁴⁵, CO, SO, SO₂, CO₂, NR⁴⁶CO₂, NR⁴⁷CONR⁴⁸, NR⁴⁹SO₂, NR⁵⁰SO₂NR⁵¹, SO₂NR⁵², NR⁵³CO, CONR⁵⁴, PO₂R⁵⁵ and PO₃R⁵⁶ or without U; R⁹, R¹⁰, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸ and R⁵⁹ are selected from the group consisting of hydrogen, lower alkyl, aryl, heterocyclo, substituted alkyl or aryl or substituted heterocyclo; R¹¹ and R⁴⁴ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, substituted heterocyclo; R¹, R², R³, R⁴, R⁵ and R⁶ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, substituted heterocyclo, cyano, carboxy, carbamyl (e.g. CONH₂), substituted carbamyl (where nitrogen may be substituted by groups selected from hydrogen, alkyl, substituted alkyl, aryl or aralkyl, substituted aryl, heterocyclo, substituted heterocyclo), alkoxycarbonyl; any two of R¹, R², R³, R⁴, R⁵ and R⁶ can join to form a cycloalkyl group; any two of R¹, R², R³, R⁴, R⁵ and R⁶ together can be oxo, except when the carbon atom bearing the substituent is part of a double bond; R, S and T are selected from the group consisting of CH₂, CO and CH(CH₂)ₚ Q wherein Q is NR⁵⁷R⁵⁸, OR⁵⁹, or CN; and p is 0, 1 or 2; A, B and C are carbon, oxygen, sulfur or nitrogen; D is carbon, oxygen, sulfur or nitrogen or without D; and with the provisos:
1. When 1 and m are both 0, n is not 0;
2. R¹¹ may be hydrogen except when Z is SO, or when Z is O, NR¹³ or S and the carbon to which it is attached is part of a double bond or when Y is SO₂, CO₂, NR¹⁸SO₂, S(O)(NR²³), or S(NR²⁴)(NR²⁵);
3. R⁴⁴ may be hydrogen except when U is SO, SO₂, NR⁴⁶CO₂ or NR⁴⁹SO₂.
   In one embodiment the compound has the formula: 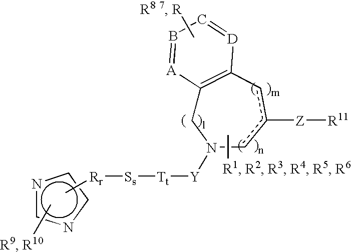
   wherein
r, sand tare 0 or 1;
l is 0; m is 1; n is 1;
Y is selected from the group consisting of CHR¹², SO₂, SO₃, CO₂, O, NR¹³, SO₂NR¹⁴, CONR¹⁵, C(NCN), C(NCN)NR¹⁶, NR¹⁷CO, NR¹⁸SO₂, CONR¹⁹NR²⁰, SO₂NR²¹NR²², S(O)(NR²³), S(NR²⁴)(NR²⁵), or without Y;
Z is selected from the group consisting of S, SO, SO₂, SO₃, CO, CO₂, O, NR¹³, SO₂NR¹⁴, CONR¹⁵, NR²⁶NR²⁷, ONR²⁸, NR²⁹O, NR³⁰SO₂NR³¹NR³²SO₂, NR³³C(NCN), NR³⁴C(NCN)NR³⁵, NR³⁶CO, NR³⁷CONR³⁸, NR³⁹CO₂, OCONR⁴⁰, S(O)(NR⁴¹), or
S(NR⁴²)(NR⁴³);
R⁷, R⁸ are selected from the group consisting of hydrogen, halo, nitro, cyano and U—R⁴⁴;
U is selected from the group consisting of S, O, NR⁴⁵, CO, SO, SO₂, CO₂, NR⁴⁶CO₂, NR⁴⁷CONR⁴⁸, NR⁴⁹SO₂, NR⁵⁰SO₂NR⁵¹, SO₂NR⁵², NR⁵³CO, CONR⁵⁴, PO₂R⁵⁵ and PO₃R⁵⁶ or without U;
R⁹, R¹⁰, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸ and R⁵⁹ are selected from the group consisting of hydrogen, lower alkyl, aryl, heterocyclo, substituted alkyl or aryl;
R¹¹ and R⁴⁴ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, substituted heterocyclo;
R¹, R², R³, R⁴, R⁵ and R⁶ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, substituted heterocyclo, cyano, alkoxycarbonyl, carboxy, carbamyl, substituted carbamyl wherein substituents on the nitrogen of the substituted carbamyl are selected hydrogen, alkyl, substituted alkyl, aryl or aralkyl, substituted aryl, heterocyclo, substituted heterocyclo; any two of R¹, R², R³, R⁴, R⁵ and R⁶ can join to form a cycloalkyl group; any two of R¹, R², R³, R⁴, R⁵ and R⁶ together can be oxo, except when the carbon atom bearing the substituent is part of a double bond;
R, S and T are selected from the group consisting of CH₂, and CH(CH₂)ₚ Q wherein Q is NR⁵⁷, R⁵⁸, OR⁵⁹, or CN;
wherein p is 0, 1 or 2; and
A, B, C and D are carbon; its enantiomers, diastereomers, pharmaceutically acceptable salts and solvates thereof;
with the provisos that:
   1. R¹¹ may be hydrogen except when Z is SO, or when Z is O, NR¹³ or S and the carbon to which it is attached is part of a double bond or when Y is SO₂, CO₂, NR¹⁸SO₂, S(O)(NR²³), or S(NR²⁴)(NR²⁵); and
   2. R⁴⁴ may be hydrogen except when U is SO, SO₂, NR⁴⁶CO₂ or NR⁴⁹SO₂.

In another embodiment the compound has the following substituents:
l, m, r, sand tare 0 or 1; n is 1 or 2;
Y is CHR¹², SO₂, SO₃, CO₂, SO₂NR¹⁴, CONR¹⁵ or without Y;
Z is SO₂, SO₃, CO, CO₂, NR¹³, SO₂NR¹⁴, CONR¹⁵, NR³⁰ SO₂NR³¹, NR³²SO₂, NR³⁶CO, NR³⁷CONR³⁸, or NR³⁹CO₂.

In another embodiment the compound has the following substituents:
l, r, s, and t is 0;
Y is CHR¹², SO₂, SO₂NR¹⁴, or CONR¹⁵ or without Y; and
Z is SO₂, SO₃, CO, CO₂, SO₂NR¹⁴, CONR¹⁵, NR³⁰SO₂NR³¹, NR³²SO₂, NR³⁶CO, NR³⁷ or CONR³⁸, NR³⁹CO₂.

In yet another embodiment the compound has the following substituents:
R⁷, R⁸ is halogen, nitro, cyano or U—R⁴⁴ wherein U is S, O, NR⁴⁶CO₂, NR⁴⁷CONR⁴⁸, R⁴⁴ is hydrogen, alkyl, substituted alkyl, alkenyl, substituted alkenyl, alkynyl, substituted alkynyl, aralkyl, cycloalkyl, aryl, substituted aryl, heterocyclo or substituted heterocyclo, R⁴⁶ and R⁴⁷ is hydrogen, lower alkyl, aryl substituted alkyl or aryl.

In yet another embodiment the salt is of an organic or inorganic acid.

In yet another embodiment the salt is of hydrogen chloride, hydrogen bromide, methanesulfonic acid, hydroxyethanesulfonic acid, sulfuric acid, acetic acid, trifluoroacetic acid, maleic acid, benzenesulfonic acid, toluenesulfonic acid, nitric acid, phosphoric acid, boric acid, tartaric acid, citric acid, succinic acid, benzoic acid, ascorbic acid or salicyclic acid.

In yet another embodiment the compound is:
N-[6-bromo-1,2,3,4-tetrahydro-1-(1H-imidazol-4-ylmethyl)-3-quinolinyl]-1-naphthalenesulfonamide, dihydrochloride;
N-[6-bromo-1,2,3,4-tetrahydro-1-(1H-imidazol-4-ylmethyl)-3-quinolinyl]-1-naphthalenecarboxamide, dihydrochloride;
N-[6-bromo-1,2,3,4-tetrahydro-1-(1H-imidazol-4-ylmethyl)-3-quinolinyl]-N-(phenylmethyl)methanesulfonamide, dihydrochloride;
N-[6-bromo-1,2,3,4-tetrahydro-1-(1H-imidazol-4-ylmethyl)-3-quinolinyl]benzenesulfonamide, dihydrochloride;
N-[6-bromo-1,2,3,4-tetrahydro-1-(1H-imidazol-4-ylmethyl)-3-quinolinyl]-N-(phenylmethyl)acetamide, dihydrochloride;
N-[6-bromo-1,2,3,4-tetrahydro-1-(1H-imidazol-4-ylmethyl)-3-quinolinyl]-N-(4-methoxyphenyl)methyl]methanesulfonamide, monohydrochloride;
N-[6-bromo-1,2,3,4-tetrahydro-1-(1H-imidazol-4-ylmethyl)-3-quinolinyl]-N-[(4-methylphenyl)methyl]methanesulfonamide monohydrochloride;
N-[6-cyano-1,2,3,4-tetrahydro-1-(1H-imidazol-4-ylmethyl)-3-quinolinyl]-N-[(3-methylphenyl)methyl]benzenesulfonamide monohydrochloride;
N-[6-cyano-1,2,3,4-tetrahydro-1-(1H-imidazol-4-ylmethyl)-3-quinolinyl]-N-[(2-methylphenyl)methyl]benzenesulfonamide monohydrochloride;
N-[6-cyano-1,2,3,4-tetrahydro-1-(1H-imidazol-4-ylmethyl)-3-quinolinyl]-N-(phenylethyl)benzenesulfonamide monohydrochloride;
N-[6-Cyano-1,2,3,4-tetrahydro-1-(1H-imidazol-4-ylmethyl)-3-quinolinyl]-N-[(2-ethoxyphenyl)methyl]benzenesulfonamide monohydrochloride;
N-[6-Cyano-1,2,3,4-tetrahydro-1-(1H-imidazol-4-ylmethyl)-3-quinolinyl]-N-(phenylmethyl)benzenesulfonamide monohydrochloride;
N-[6-Cyano-1,2,3,4-tetrahydro-1-(1H-imidazol-4-ylmethyl)-3-quinolinyl]-N-[(2,3-dimethoxyphenyl)methyl]benzenesulfonamide monohydrochloride;
N-[6-Cyano-1,2,3,4-tetrahydro-1-(1H-imidazol-4-ylmethyl)-3-quinolinyl]-N-[(3,5-dimethylphenyl)methyl]benzenesulfonamide monohydrochloride;
N-[6-Cyano-1,2,3,4-tetrahydro-1-(1H-imidazol-4-ylmethyl)-3-quinolinyl]-N-[(1-naphthalenyl)methyl]benzenesulfonamide monohydrochloride;
N-[6-Cyano-1,2,3,4-tetrahydro-1-(1H-imidazol-4-ylmethyl)-3-quinolinyl]-N-[(2-thiophene)methyl]benzenesulfonamide monohydrochloride;
N-[6-Cyano-1,2,3,4-tetrahydro-1-(1H-imidazol-4-ylmethyl)-3-quinolinyl]-N-[(2,5-dimethylphenyl)methyl]benzenesulfonamide monohydrochloride;
N-[6-Cyano-1,2,3,4-tetrahydro-1-(1H-imidazol-4-ylmethyl)-3-quinolinyl]-N-[(3-thiophene)methyl]benzenesulfonamide monohydrochloride;
N-[6-Cyano-1,2,3,4-tetrahydro-1-(1H-imidazol-4-ylmethyl)-3-quinolinyl]-N-[(3-chlorophenyl)methyl]benzenesulfonamide monohydrochloride;
N-[6-Cyano-1,2,3,4-tetrahydro-1-(1H-imidazol-4-ylmethyl)-3-quinolinyl]-N-[(2-fluorophenyl)methyl]benzenesulfonamide monohydrochloride;
N-[6-Cyano-1,2,3,4-tetrahydro-1-(1H-imidazol-4-ylmethyl)-3-quinolinyl]-N-[(3-pyridyl)methyl]benzenesulfonamide monohydrochloride;
N-[6-Cyano-1,2,3,4-tetrahydro-1-[[1-(methyl)-1H-imidazol-5-yl]methyl]-3-quinolinyl]-N-(phenylmethyl)benzenesulfonamide monohydrochloride;
N-[6-Cyano-1,2,3,4-tetrahydro-1-[[1-(methyl)-1H-imidazol-5-yl]methyl]-3-quinolinyl]-N-[(3-thiophenemethyl]benzenesulfonamide monohydrochloride;
N-[6-Cyano-1,2,3,4-tetrahydro-1-(1H-imidazol-4-ylmethyl)-3-quinolinyl]-N-(phenylmethyl)methanesulfonamide monohydrochloride;
N-[6-Cyano-1,2,3,4-tetrahydro-1-[[1-(methyl)-1H-imidazol-5-yl]methyl]-3-quinolinyl]-N-(phenylmethyl)methanesulfonamide monohydrochloride;
(R)—N-[6-Cyano-1,2,3,4-tetrahydro-1-[[1-(methyl)-1H-imidazol-5-yl]methyl]-3-quinolinyl]-N-(phenylmethyl) benzenesulfonamide monohydrochloride.

In yet another embodiment, the invention is a method of treating a synucleinopathic subject, the method comprising, administering to a synucleinopathic subject a farnesyl transferase inhibitor compound of the formula:

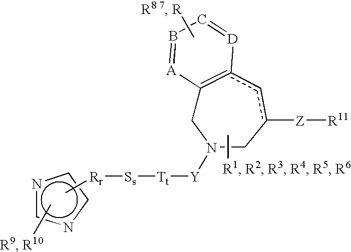

or a stereoisomeric form, or a pharmaceutically acceptable acid or base addition salt form thereof, in a therapeutically effective amount, wherein Y is selected from the group consisting of CHR¹², SO₂, SO₃, CO, CO₂, O, NR¹³, SO₂NR¹⁴, CONR¹⁵, C(NCN), C(NCN)NR¹⁶, NR¹⁷CO, NR¹⁸SO₂, CONR¹⁹NR²⁰, SO₂NR²¹NR²², S(O)(NR²³), and S(R²⁴)(R²⁵), or without Y; Z is selected from the group consisting of S, SO, SO₂, SO₃, CO, CO₂, O, NR¹³, SO₂NR¹⁴, CONR¹⁵, NR²⁶NR²⁷, ONR²⁸, NR²⁹O, NR³⁰SO₂NR³¹, NR³²SO₂, NR³³C(NCN), NR³⁴C(NCN)NR³⁵NR³⁶CO, NR³⁷CONR³⁸, NR³⁹CO2, OCONR⁴⁰, S(O)(NR⁴¹), and S(NR⁴²)(NR⁴³); R⁷ and R⁸ are selected from the group consisting of hydrogen, halo, nitro, cyano and U—R⁴⁴; U is selected from the group consisting of S, O, NR⁴⁵, CO, SO, SO₂, CO₂, NR⁴⁶CO₂, NR⁴⁷CONR⁴⁸, NR⁴⁹SO₂, NR⁵⁰SO₂NR⁵¹, SO₂NR⁵², NR⁵³CO, CONR⁵⁴, PO₂R⁵⁵ and PO₃R⁵⁶ or without U; R⁹, R¹⁰, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, and R⁵⁹ are selected from the group consisting of hydrogen, lower alkyl, aryl, heterocyclo, substituted alkyl and aryl; R¹¹ and R⁴⁴ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, and substituted heterocyclo; R¹, R², R³, R⁴, R⁵ and R⁶ are selected from the group consisting of hydrogen, alkyl, substituted alkyl, alkenyl, substituted alkenyl, alkynyl, substituted alkynyl, aralkyl, cycloalkyl, aryl, substituted aryl, heterocyclo, substituted heterocyclo, cyano, alkoxycarbonyl, carboxy, carbamyl, and substituted carbamyl wherein substituents on the nitrogen of the substituted carbamyl are selected from the group consisting of hydrogen, alkyl, substituted alkyl, aryl, aralkyl, substituted aryl, heterocyclo, and substituted heterocyclo; any two of R¹, R², R³, R⁴, R⁵ and R⁶ can join to form a cycloalkyl group; any two of R¹, R², R³, R⁴, R⁵ and R⁶ together can be oxo, except when the carbon atom bearing the substituent is part of a double bond; R¹, S and T are selected from the group consisting of CH₂ and CH(CH₂)ₚ Q wherein Q is NR⁵⁷R⁵⁸, OR⁵⁹, or CN; p is 0, 1 or 2; and A, B, C and D are carbon; its enantiomer, diastereomer, pharmaceutically acceptable salt or solvate thereof; with the provisos that:
1. R¹¹ may be hydrogen except when Z is SO, or when Z is O, NR¹³ or S and the carbon to which it is attached is part of a double bond or when Y is SO₂, CO₂, NR¹⁸SO₂, S(O)(NR²³), or S(NR²⁴)(NR²⁵); and
2. R⁴⁴ may be hydrogen except when U is SO, SO₂, NR⁴⁶CO₂ or NR⁴⁹SO₂.
   28.

In one embodiment of this aspect of the invention r, s and t are 0 or 1;

Y is CHR², SO₂, SO₃, CO, CO₂, SO₂NR¹⁴, CONR¹⁵ or without Y;

Z is CR¹², SO₂, SO₃, CO, CO₂, NR¹³, SO₂NR¹⁴, CONR¹⁵, NR³⁰SO₂NR³¹, NR³²SO₂, NR³⁶CO, NR³⁷CONR³⁸, NR³⁹CO₂ or without Z.

In yet another embodiment r, s, and t is 0; Y is CHR¹², SO₂, CO, SO₂NR¹⁴, or CONR¹⁵ or without Y; and Z is CR¹², SO₂, SO₃, CO, CO₂, SO₂NR¹⁴, CONR¹⁵, NR³⁰SO₂NR³¹, NR³²SO₂, NR³⁶CO, NR³⁷CONR³⁸, NR³⁹CO₂ or without Z.

In yet another embodiments R⁷, R⁸ is halogen, nitro, cyano or U—R⁴⁴ wherein U is S, O, NR⁴⁶CO₂, NR⁴⁷CONR⁴⁸, R⁴⁴ is hydrogen, alkyl, substituted alkyl, alkenyl, substituted alkenyl, alkynyl, substituted alkynyl, aralkyl, cycloalkyl, aryl, substituted aryl, heterocyclo or substituted heterocyclo, R⁴⁶ and R⁴⁷ is hydrogen, lower alkyl, aryl substituted alkyl or aryl.

In one embodiment the compound of the invention is selected from the group consisting of:
N-[6-bromo-1,2,3,4-tetrahydro-1-(1H-imidazol-4-ylmethyl)-3-quinolinyl]-1-naphthalenesulfonamide, dihydrochloride;
N-[6-bromo-1,2,3,4-tetrahydro-1-(1H-imidazol-4-ylmethyl)-3-quinolinyl]-1-naphthalenecarboxamide, dihydrochloride;
N-[6-bromo-1,2,3,4-tetrahydro-1-(1H-imidazol-4-ylmethyl)-3-quinolinyl]-N-(phenylmethyl)methanesulfonamide, dihydrochloride;
N-[6-bromo-1,2,3,4-tetrahydro-1-(1H-imidazol-4-ylmethyl)-3-quinolinyl]benzenesulfonamide, dihydrochloride;
N-[6-bromo-1,2,3,4-tetrahydro-1-(1H-imidazol-4-ylmethyl)-3-quinolinyl]-N-(phenylmethyl)acetamide, dihydrochloride;
N-[6-bromo-1,2,3,4-tetrahydro-1-(1H-imidazol-4-ylmethyl)-3-quinolinyl]-N-(4-methoxyphenyl)methyl]methanesulfonamide, monohydrochloride;
N-[6-bromo-1,2,3,4-tetrahydro-1-(1H-imidazol-4-ylmethyl)-3-quinolinyl]-N-[(4-methylphenyl)methyl]methanesulfonamide monohydrochloride;
N-[6-cyano-1,2,3,4-tetrahydro-1-(1H-imidazol-4-ylmethyl)-3-quinolinyl]-N-[(3-methylphenyl)methyl]benzenesulfonamide monohydrochloride;
N-[6-cyano-1,2,3,4-tetrahydro-1-(1H-imidazol-4-ylmethyl)-3-quinolinyl]-N-[(2-methylphenyl)methyl]benzenesulfonamide monohydrochloride;
N-[6-cyano-1,2,3,4-tetrahydro-1-(1H-imidazol-4-ylmethyl)-3-quinolinyl]-N-(phenylethyl)benzenesulfonamide monohydrochloride;
N-[6-Cyano-1,2,3,4-tetrahydro-1-(1H-imidazol-4-ylmethyl)-3-quinolinyl]-N-[(2-ethoxyphenyl)methyl]benzenesulfonamide monohydrochloride;
N-[6-Cyano-1,2,3,4-tetrahydro-1-(1H-imidazol-4-ylmethyl)-3-quinolinyl]-N-(phenylmethyl)benzenesulfonamide monohydrochloride;
N-[6-Cyano-1,2,3,4-tetrahydro-1-(1H-imidazol-4-ylmethyl)-3-quinolinyl]-N-[(2,3-dimethoxyphenyl)methyl]benzenesulfonamide monohydrochloride;
N-[6-Cyano-1,2,3,4-tetrahydro-1-(1H-imidazol-4-ylmethyl)-3-quinolinyl]-N-[(3,5-dimethylphenyl)methyl]benzenesulfonamide monohydrochloride;
N-[6-Cyano-1,2,3,4-tetrahydro-1-(1H-imidazol-4-ylmethyl)-3-quinolinyl]-N-[(1-naphthalenyl)methyl]benzenesulfonamide monohydrochloride;
N-[6-Cyano-1,2,3,4-tetrahydro-1-(1H-imidazol-4-ylmethyl)-3-quinolinyl]-N-[(2-thiophene)methyl]benzenesulfonamide monohydrochloride;
N-[6-Cyano-1,2,3,4-tetrahydro-1-(1H-imidazol-4-ylmethyl)-3-quinolinyl]-N-[(2,5-dimethylphenyl)methyl]benzenesulfonamide monohydrochloride;
N-[6-Cyano-1,2,3,4-tetrahydro-1-(1H-imidazol-4-ylmethyl)-3-quinolinyl]-N-[(3-thiophene)methyl]benzenesulfonamide monohydrochloride;
N-[6-Cyano-1,2,3,4-tetrahydro-1-(1H-imidazol-4-ylmethyl)-3-quinolinyl]-N-[(3-chlorophenyl)methyl]benzenesulfonamide monohydrochloride;
N-[6-Cyano-1,2,3,4-tetrahydro-1-(1H-imidazol-4-ylmethyl)-3-quinolinyl]-N-[(2-fluorophenyl)methyl]benzenesulfonamide monohydrochloride;
N-[6-Cyano-1,2,3,4-tetrahydro-1-(1H-imidazol-4-ylmethyl)-3-quinolinyl]-N-[(3-pyridyl)methyl]benzenesulfonamide monohydrochloride;
N-[6-Cyano-1,2,3,4-tetrahydro-1-[[1-(methyl)-1H-imidazol-5-yl]methyl]-3-quinolinyl]-N-(phenylmethyl)benzenesulfonamide monohydrochloride;
N-[6-Cyano-1,2,3,4-tetrahydro-1-[[1-(methyl)-1H-imidazol-5-yl]methyl]-3-quinolinyl]-N-[(3-thiophenemethyl]benzenesulfonamide monohydrochloride;
N-[6-Cyano-1,2,3,4-tetrahydro-1-(1H-imidazol-4-ylmethyl)-3-quinolinyl]-N-(phenylmethyl)methanesulfonamide monohydrochloride;
N-[6-Cyano-1,2,3,4-tetrahydro-1-[[1-(methyl)-1H-imidazol-5-yl]methyl]-3-quinolinyl]-N-(phenylmethyl)methanesulfonamide monohydrochloride;
(R)—N-[6-Cyano-1,2,3,4-tetrahydro-1-[[1-(methyl)-1H-imidazol-5-yl]methyl]-3-quinolinyl]-N-(phenylmethyl)benzenesulfonamide monohydrochloride.

In another embodiment, the invention is a method of treating a synucleinopathic subject, the method comprising, administering to a synucleinopathic subject a farnesyl transferase inhibitor compound of the formula:

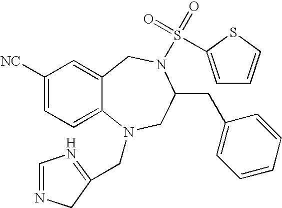

or a stereoisomeric form, or a pharmaceutically acceptable acid or base addition salt form thereof, in a therapeutically effective amount.

In one aspect, the invention provides a method of treating a synucleinopathic subject by administering a farnesyl transferase inhibitor compound of the formula: 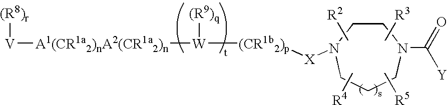
or a stereoisomeric form, or a pharmaceutically acceptable acid or base addition salt form thereof, in a therapeutically effective amount,
wherein:
R^{1a} and R^{1b} are independently selected from:
a) hydrogen,
b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂N—C(O)—, CN, NO₂, (R¹⁰)₂N—C(NR¹⁰)—, R¹⁰C(O)—, R¹⁰C(O)—, N₃, —N(R¹⁰)₂, or R¹¹OC(O)NR¹⁰—,
c) unsubstituted or substituted C₁-C₆ alkyl wherein the substitutent on the substituted C₁-C₆ alkyl is selected from unsubstituted or substituted aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂N—C(O)—, CN, (R¹⁰)₂N—C(NR¹⁰)—, R¹⁰C(O)—, R¹⁰C(O)—, N₃, —N(R¹⁰)₂, and R¹¹OC(O)—NR¹⁰—;
R² and R³ are independently selected from: H; unsubstituted or substituted C₁₋₈ alkyl, unsubstituted or substituted C₂₋₈ alkenyl, unsubstituted or substituted C₂₋₈ alkynyl, unsubstituted or substituted aryl, unsubstituted or substituted heterocycle, 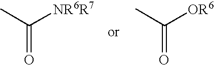
   wherein the substituted group is substituted with one or more of:
1) aryl or heterocycl, unsubstituted or substituted with:
   a) C₁₋₄ alkyl,
   b) (CH₂)ₚ OR⁶,
   c) (CH₂)ₚ NR⁶R⁷,
   d) halogen,
   e) CN,
2) C₃₋₆ cycloalkyl,
3) OR⁶,
4) SR^{6a}, S(O)R^{6a}, SO₂R^{6a},
5) —NR⁶R⁷. 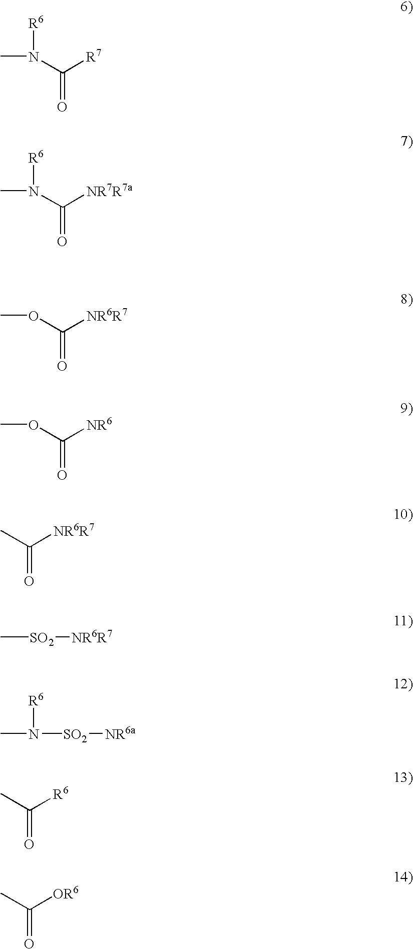
15) N₃ or
16) F; or
R² and R³ are attached to the same C atom and are combined to form —(CH₂)ᵤ—wherein one of the carbon atoms is optionally replaced by a moiety selected from: O, S(O)ₘ, —NC(O)—, and —N(COR¹⁰)—;
R⁴ and R⁵ are independently selected from H and CH₃;
   and any two of R², R³, R⁴ and R⁵ are optionally attached to the same carbon atom;
R⁶, R⁷ and R^{7a} are independently selected from: H; C₁₋₄ alkyl, C₃₋₆ cycloalkyl, heterocycle, aryl, aroyl, heteroaroyl, arylsulfonyl, heteroarylsulfonyl, unsubstituted or substituted with:
   a) C₁₋₄ alkoxy, b) aryl or heterocycle, c) halogen, d) HO,
   e) 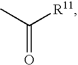
      f) —SO₂R¹¹, or g) N(R¹⁰)₂; or
R⁶ and R⁷ may be joined in a ring;
R⁷ and R^{7a} may be joined in a ring;
R^{6a} is selected from: C₁₋₄ alkyl, C₃₋₆ cycloalkyl, heterocycle, aryl, unsubstituted or substituted with: a) C₁₋₄ alkoxy, b) aryl or heterocycle, c) halogen, d) HO,
e) 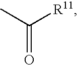
   f) —SO₂R¹¹, or g) N(R¹⁰)₂;
R⁸ is independently selected from:
   a) hydrogen,
   b) aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and
   c) C₁-C₆ alkyl unsubstituted or substituted by aryl, cyanophenyl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NH—, (R¹⁰)₂ NC(O)—, R¹⁰₂N—C(NR¹⁰)—, CN, R¹⁰OC(O)—, R¹⁰OC(O)—, N₃, —N(R¹⁰)₂, or R¹⁰C(O)NH—;
   R⁹ is selected from:
   a) hydrogen,
   b) C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and
   c) C₁-C₆ alkyl unsubstituted or substituted by perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹¹OC(O)NR¹⁰—;
R¹⁰ is independently selected from hydrogen, C₁-C₆ alkyl, benzyl and aryl;
R¹¹ is independently selected from C₁-C₆ alkyl and aryl;
A¹ and A² are independently selected from: a bond, —CH═CH—, —C.tbd.C—,
   —C(O)—, —C(O)NR¹⁰—, —NR¹⁰C(O)—, O, —N(R¹⁰)—,
   —S(O)₂N(R¹⁰)—, —N(R¹⁰)S(O)₂—, or S(O)ₘ;
V is selected from: a) hydrogen, b) heterocycle, c) aryl, d) C₁-C₂₀ alkyl wherein from 0 to 4 carbon atoms are replaced with a heteroatom selected from O, S, and N, and e) C₂-C₂₀ alkenyl,
   provided that V is not hydrogen if A¹ is S(O)ₘ and V is not hydrogen if A¹ is a bond, n is 0 and A² is S(O)ₘ;
W is a heterocycle;
X is —CH₂—, —C(═O)—, or —S(═O)ₘ—;
Y is unsubstituted or substituted aryl or unsubstituted or substituted heterocycle,
   wherein the substituted aryl or substituted heterocycle is substituted with one or more of:
   1) C₁₋₄ alkyl, unsubstituted or substituted with: a) C₁₋₄ alkoxy, b) NR⁶R⁷, c) C₃₋₆ cycloalkyl, d) aryl or heterocycle, e) HO, f) —S(O)ₘR^{6a}, or g) —C(O)NR⁶R⁷, 2) aryl or heterocycle, 3) halogen, 4) OR⁶, 5) NR⁶R⁷, 6) CN, 7) NO₂, 8) CF₃, 9)—S(O)ₘR^{6a}, 10)—C(O)NR⁶R⁷, or 11) C₃-C₆ cycloalkyl
m is 0, 1 or 2; n is 0, 1, 2, 3 or 4; p is 0, 1, 2, 3 or 4; q is 1 or 2; r is to 5, provided that r is 0 when V is hydrogen; s is 0 or 1; t is 0 or 1; and u is 4 or 5.

In one embodiment, the compound may be of the formula: 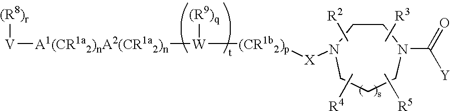
or a stereoisomeric form, or a pharmaceutically acceptable acid or base addition salt form thereof, in a therapeutically effective amount,
wherein:
R^{1a} is independently selected from: hydrogen or C₁-C₆ alkyl;
R^{1b} is independently selected from:
a) hydrogen,
b) aryl, heterocycle, cycloalkyl, R¹⁰O—, —N(R¹⁰)₂ or C₂-C₆ alkenyl,
c) unsubstituted or substituted C₁-C₆ alkyl wherein the substitutent on the substituted C₁-C₆ alkyl is selected from unsubstituted or substituted aryl, heterocycle, cycloalkyl, alkenyl, R¹⁰O— and —N(R¹⁰)₂;
R³, R⁴ and R⁵ are independently selected from H and CH₃;
R² is H; 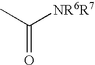
   or C₁₋₅ alkyl, unbranched or branched, unsubstituted or substituted with one or more of:
1) aryl,
2) heterocycle,
3) OR⁶,
4) SR^{6a}, SO₂R^{6a}, or
5) 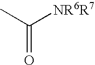
   and any two of R², R³, R⁴, and R⁵ are optionally attached to the same carbon atom;
R⁶, R⁷ and R^{7a} are independently selected from:
H; C₁₋₄ alkyl, C₃₋₆ cycloalkyl, aryl, heterocycle, unsubstituted or substituted with:
   a) C₁₋₄ alkoxy,
   b) halogen, or
   c) aryl or heterocycle;
R^{6a} is selected from:
C₁₋₄ alkyl or C₃₋₆ cycloalkyl, unsubstituted or substituted with:
   a) C₁₋₄ alkoxy,
   b) halogen, or
   c) aryl or heterocycle;
R⁸ is independently selected from:
a) hydrogen,
b) C₁-C₆ alkyl, C₂-C₆ alkenyl, C₂-C₆ alkynyl, C₁-C₆ perfluoroalkyl, F, Cl, R¹⁰O—, R¹⁰C(O)NR¹⁰—, CN, NO₂, (R¹⁰)₂N—C(NR¹⁰)—, R¹⁰OC(O)—, R¹⁰OC(O)—, —N(R¹⁰)₂, or R¹¹OC(O)NR¹⁰—, and
c) C₁-C₆ alkyl substituted by C₁-C₆ perfluoroalkyl, R¹⁰O—, R¹⁰C(O)NR¹⁰—, (R¹⁰)₂ N—C(NR¹⁰)—, R¹⁰C(O)—, R¹⁰OC(O)—, —N(R¹⁰)₂, or R¹¹OC(O)NR¹⁰—;
R⁹ is selected from:
a) hydrogen,
b) C₂-C₆ alkenyl, C₂-C₆ alkynyl, C₁-C₆ perfluoroalkyl, F, Cl, R¹⁰O—, R¹¹S(O)ₘ—, R¹⁰C(O)NR¹⁰—, CN, NO₂, (R¹⁰)₂N—C(NR¹⁰)—, R¹⁰C(O)—, R¹⁰OC(O)—, —N(R¹⁰)₂, or R¹¹OC(O)NR¹⁰—, and
c) C₁-C₆ alkyl unsubstituted or substituted by C₁-C₆ perfluoroalkyl, F, Cl, R¹⁰O—, R¹¹S(O)ₘ—, R¹⁰C(O)NR¹⁰—, CN, (R¹⁰)₂N—C(NR¹⁰)—, R¹⁰C(O)—, R¹⁰OC(O)—, —N(R¹⁰)₂, or R¹¹OC(O)NR¹⁰—;
R¹⁰ is independently selected from hydrogen, C₁-C₆ alkyl, benzyl and aryl;
R¹¹ is independently selected from C₁-C₆ alkyl and aryl;
A¹ and A² are independently selected from: a bond, —CH═CH—, —C.tbd.C—, —C(O)—, —C(O)NR¹⁰—, O, —N(R¹⁰)—, or S(O)ₘ;
V is selected from:
a) hydrogen,
b) heterocycle selected from pyrrolidinyl, imidazolyl, pyridinyl, thiazolyl, pyridonyl, 2-oxopiperidinyl, indolyl, quinolinyl, isoquinolinyl, and thienyl,
c) aryl,
d) C₁-C₂₀ alkyl wherein from 0 to 4 carbon atoms are replaced with a a heteroatom selected from O, S, and N, and
e) C₂-C₂₀ alkenyl, and
provided that V is not hydrogen if A¹ is S(O)ₘ and V is not hydrogen if A¹ is a bond, n is 0 and A² is S(O)ₘ;
W is a heterocycle selected from pyrrolidinyl, imidazolyl, pyridinyl, thiazolyl, pyridonyl, 2-oxopiperidinyl, indolyl, quinolinyl, or isoquinolinyl;
X is —CH₂— or —C(═O)—;
Y is mono- or bicyclic aryl, or mono- or bicyclic heterocycle, unsubstituted or substituted with one or more of: a) C₁₋₄ alkyl, b) C₁₋₄ alkoxy, c) halogen, or d) NR⁶R⁷;
m is 0, 1 or 2; n is 0, 1, 2, 3 or 4; p is 0, 1, 2, 3 or 4; r is 0 to 5, provided that r is 0 when V is hydrogen; s is 0 or 1; and t is 0 or 1.

In another aspect, the invention provides a method of treating a synucleinopathic subject by administering a farnesyl transferase inhibitor compound of the formula: 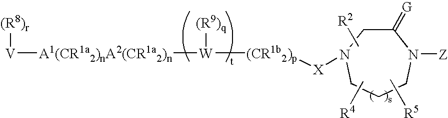
or a stereoisomeric form, or a pharmaceutically acceptable acid or base addition salt form thereof, in a therapeutically effective amount,
wherein:
R^{1a} and R^{1b} are independently selected from:
   a) hydrogen,
   b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, CN(R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—,
   c) unsubstituted or substituted C₁-C₆ alkyl wherein the substitutent on the substituted C₁-C₆ alkyl is selected from unsubstituted or substituted aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, R¹⁰OC(O)—, N₃, —N(R¹⁰)₂, and R¹¹OC(O)—NR¹⁰—;
   R² and R³ are independently selected from: H; unsubstituted or substituted C₁₋₈ alkyl, unsubstituted or substituted C₂₋₈ alkenyl, unsubstituted or substituted C₂₋₈ alkynyl, unsubstituted or substituted aryl, unsubstituted or substituted heterocycle, 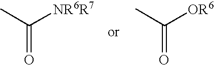
      wherein the substituted group is substituted with one or more of:
1) aryl or heterocycle, unsubstituted or substituted with:
   a) C₁₋₄ alkyl,
   b) (CH₂)ₚ OR⁶,
   c) (CH₂), NR⁶R⁷,
   d) halogen,
   e) CN,
2) C₃₋₆ cycloalkyl,
3) OR⁶,
4) SR^{6a}, S(O)R^{6a} SO₂R^{6a},
5) —NR⁶R⁷. 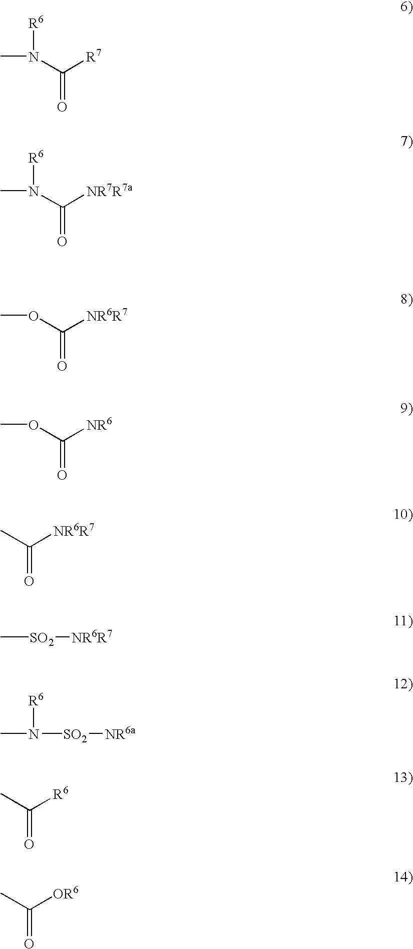
15) N₃ or
16) F; or
R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;
R⁴ is selected from H and CH₃;
and any two of R², R³ and R⁴ are optionally attached to the same carbon atom;
R⁶, R⁷ and R^{7a} are independently selected from: H; C₁₋₄ alkyl, C₃₋₆ cycloalkyl, heterocycle, aryl, aroyl, heteroaroyl, arylsulfonyl, heteroarylsulfonyl, unsubstituted or substituted with:
   a) C₁₋₄ alkoxy,
   b) aryl or heterocycle,
   c) halogen,
   d) HO,
   e) 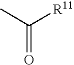
   f) —SO₂R¹¹, or
   g) N(R¹⁰)₂; or
R⁶ and R⁷ may be joined in a ring;
R⁷ and R^{7a} may be joined in a ring;
R^{6a} is selected from: C₁₋₄ alkyl, C₃₋₆ cycloalkyl, heterocycle, aryl, unsubstituted or substituted with:
   a) C₁₋₄ alkoxy,
   b) aryl or heterocycle,
   c) halogen,
   d) HO,
   e) 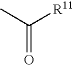
   f) —SO₂R¹¹, or
   g) N(R¹⁰)₂;
R⁸ is independently selected from:
   a) hydrogen,
   b) aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and
   c) C₁-C₆ alkyl unsubstituted or substituted by aryl, cyanophenyl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NH—, (R¹⁰)₂NC(O)—, R¹⁰₂ N—C(NR¹⁰)—, CN, R¹⁰C(O)—, R¹⁰C(O)—, N₃, —N(R¹⁰)₂, or R¹⁰C(O)NH—;
R⁹ is selected from:
   a) hydrogen,
   b) alkenyl, alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and
   c) C₁-C₆ alkyl unsubstituted or substituted by perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹¹OC(O)NR¹⁰—;
R¹⁰ is independently selected from hydrogen, C₁-C₆ alkyl, benzyl and aryl;
R¹¹ is independently selected from C₁-C₆ alkyl and aryl;
A¹ and A² are independently selected from: a bond, —CH═CH—, —C.tbd.C—, —C(O)—, —C(O)NR¹⁰—, —NR¹⁰C(O)—, O, —N(R¹⁰)—, —S(O)₂ N(R¹⁰)—, —N(R¹⁰)S(O)₂—, or S(O)ₘ;
G is H₂ or O;
V is selected from:
   a) hydrogen,
   b) heterocycle,
   c) aryl,
   d) C₁-C₂₀ alkyl wherein from 0 to 4 carbon atoms are replaced with a a heteroatoin selected from O, S, and N, and
   e) C₂-C₂₀ alkenyl,
provided that V is not hydrogen if A¹ is S(O)ₘ and V is not hydrogen if A¹ is a bond, n is 0 and A² is S(O)ₘ;
W is a heterocycle;
X is —CH₂—, —C(═O)—, or —S(═O)ₘ—;
Z is a unsubstituted or substituted group selected from aryl, heteroaryl, arylmethyl, heteroarylmethyl, arylsulfonyl, heteroarylsulfonyl, wherein the substituted group is substituted with one or more of the following:
1) C₁₋₄ alkyl, unsubstituted or substituted with: a) C₁₋₄ alkoxy, b) NR⁶R⁷, c) C₃₋₆ cycloalkyl, d) aryl or heterocycle, e) HO, f) —S(O)ₘR^{6a}, or g) —C(O)NR⁶R⁷, 2) aryl or heterocycle, 3) halogen, 4) OR⁶, 5) NR⁶R⁷, 6) CN, 7) NO₂, 8) CF₃, 9)—S(O)ₘR^{6a}, 10)—C(O)NR⁶R⁷, or 11) C₃-C₆ cycloalkyl;
m is 0, 1 or 2; n is 0, 1, 2, 3 or 4; p is 0, 1, 2, 3 or 4; q is 1 or 2; r is 0 to 5, provided that r is 0 when V is hydrogen; s is 0 or 1; t is 0 or 1; and u is 4 or 5.

In one embodiment, the compound may be of the formula: 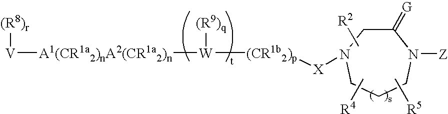
or a stereoisomeric form, or a pharmaceutically acceptable acid or base addition salt form thereof, in a therapeutically effective amount,

wherein:
R^{1a} is independently selected from: hydrogen or C₁-C₆ alkyl;
R^{1b} is independently selected from:
a) hydrogen,
b) aryl, heterocycle, cycloalkyl, R¹⁰O—, —N(R¹⁰)₂ or C₂-C₆ alkenyl,
c) unsubstituted or substituted C₁-C₆ alkyl wherein the substitutent on the substituted C₁-C₆ alkyl is selected from unsubstituted or substituted aryl, heterocycle, cycloalkyl, alkenyl, R¹⁰O— and —N(R¹⁰)₂;
R³ and R⁴ are independently selected from H and CH₃;
R² is H; 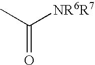
   or C₁₋₅ alkyl, unbranched or branched, unsubstituted or substituted with one or more of:
1) aryl,
2) heterocycle,
3) OR⁶,
4) SR^{6a}, SO₂R^{6a}, or
5) 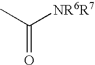
and any two of R², R³, R⁴, and R⁵ are optionally attached to the same carbon atom;
R⁶, R⁷ and R^{7a} are independently selected from:
H; C₁₋₄ alkyl, C₃₋₆ cycloalkyl, aryl, heterocycle, unsubstituted or substituted with:
   a) C₁₋₄ alkoxy,
   b) halogen, or
   c) aryl or heterocycle;
R^{6a} is selected from:
C₁₋₄ alkyl or C₃₋₆ cycloalkyl, unsubstituted or substituted with:
   a) C₁₋₄ alkoxy,
   b) halogen, or
   c) aryl or heterocycle;
R⁸ is independently selected from:
a) hydrogen,
b) C₁-C₆ alkyl, C₂-C₆ alkenyl, C₂-C₆ alkynyl, C₁-C₆ perfluoroalkyl, F, Cl, R¹⁰O—, R¹⁰C(O)NR¹⁰—, CN, NO₂, (R¹⁰)₂ N—C(NR¹⁰)—, R¹⁰C(O)—, R¹⁰OC(O)—, —N(R¹⁰)₂, or R¹¹OC(O)NR¹⁰—, and
c) C₁-C₆ alkyl substituted by C₁-C₆ perfluoroalkyl, R¹⁰O—, R¹⁰C(O)NR¹⁰—, (R¹⁰)₂N—C(NR¹⁰)—, R¹⁰C(O)—, R¹⁰OC(O)—, —N(R¹⁰)₂, or R¹¹OC(O)NR¹⁰—;
R⁹ is selected from:
a) hydrogen,
b) C₂-C₆ alkenyl, C₂-C₆ alkynyl, C₁-C₆ perfluoroalkyl, F, Cl, R¹⁰O—, R¹¹S(O)ₘ—, R¹⁰C(O)NR¹⁰—, CN, NO₂, (R¹⁰)₂N—C(NR¹⁰)—, R¹⁰C(O)—, R¹⁰OC(O)—, —N(R¹⁰)₂, or R¹¹OC(O)NR¹⁰—, and
c) C₁-C₆ alkyl unsubstituted or substituted by C₁-C₆ perfluoroalkyl, F, Cl, R¹⁰O—, R¹¹S(O)ₘ—, R¹⁰C(O)NR¹⁰—, CN, (R¹⁰)₂N—C(NR¹⁰)—, R¹⁰C(O)—, R¹⁰OC(O)—, —N(R¹⁰)₂, or R¹¹OC(O)NR¹⁰—;
R¹⁰ is independently selected from hydrogen, C₁-C₆ alkyl, benzyl and aryl;
R¹¹ is independently selected from C₁-C₆ alkyl and aryl;
A¹ and A² are independently selected from: a bond, —CH═CH—, —C.tbd.C—, —C(O)—, —C(O)NR¹⁰—, O, —N(R¹⁰)—, or S(O)ₘ;
V is selected from:
a) hydrogen,
b) heterocycle selected from pyirolidinyl, imidazolyl, pyridinyl, thiazolyl, pyridonyl, 2-oxopiperidinyl, indolyl, quinolinyl, isoquinolinyl, and thienyl,
c) aryl,
d) C₁-C₂₀ alkyl wherein from 0 to 4 carbon atoms are replaced with a a heteroatom selected from O, S, and N, and
e) C₂-C₂₀ alkenyl, and provided that V is not hydrogen if A¹ is S(O)ₘ and V is not hydrogen if A¹ is a bond, n is 0 and A² is S(O)ₘ;
G is H₂ or O;
W is a heterocycle selected from pyrrolidinyl, imidazolyl, pyridinyl, thiazolyl, pyridonyl, 2-oxopiperidinyl, indolyl, quinolinyl, or isoquinolinyl;
X is —CH₂— or —C(═O)—;
Z is mono- or bicyclic aryl, mono- or bicyclic heteroaryl, mono- or bicyclic arylmethyl, mono- or bicyclic heteroarylmethyl, mono- or bicyclic arylsulfonyl, mono- or bicyclic heteroarylsulfonyl, unsubstituted or substituted with one or two of the following:
1) C₁₋₄ alkyl, unsubstituted or substituted with: a) C₁₋₄ alkoxy, b) NR⁶R⁷, c) C₃₋₆ cycloalkyl, d) aryl or heterocycle, e) HO, f) —S(O)ₘR⁶, or g) —C(O)NR⁶R⁷; 2) aryl or heterocycle, 3) halogen, 4) OR⁶, 5) NR⁶R⁷, 6) CN, 7) NO₂, 8) CF₃, 9) —S(O)ₘR⁶, 10)—C(O)NR⁶R⁷, or 11) C₃-C₆ cycloalkyl;
m is 0, 1 or 2; n is 0, 1, 2, 3 or 4; p is 0, 1, 2, 3 or 4; r is 0 to 5, provided that r is 0 when V is hydrogen; s is 0 or 1; t is 0 or 1; and u is 4 or 5;
provided that when G is H₂ and W is imidazolyl, then the substitutent (R⁸)ᵣ—V— A¹ (CR^{1a}₂)ₙ A² (CR^{1a}₂)ₙ— is not H and
provided that when X is —C(═O)—, or —S(═O)ₘ—, then t is 1 and the substitutent (R⁵)ᵣ—V— A¹ (CR^{1a}₂)ₙ A² (CR^{1a}₂)ₙ— is not H.

In another aspect, the invention provides a method of treating a synucleinopathic subject by administering a farnesyl transferase inhibitor compound of the formula: 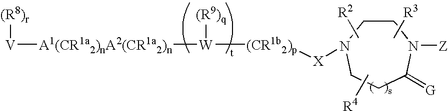
or a stereoisomeric form, or a pharmaceutically acceptable acid or base addition salt form thereof, in a therapeutically effective amount,
wherein:
R^{1a} and R^{1b} are independently selected from:
a) hydrogen,
b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂ or R¹¹OC(O)NR¹⁰—,
c) unsubstituted or substituted C₁-C₆ alkyl wherein the substitutent on the substituted C₁-C₆ alkyl is selected from unsubstituted or substituted aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, R¹⁰OC(O)—, N₃, —N(R¹⁰)₂, and R¹¹OC(O)—NR¹⁰—;
R² and R³ are independently selected from: H; unsubstituted or substituted C₁₋₈ alkyl, unsubstituted or substituted C₂₋₈ alkenyl, unsubstituted or substituted C₂₋₈ alkynyl, unsubstituted or substituted aryl, unsubstituted or substituted heterocycle, 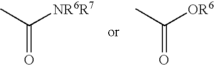
   wherein the substituted group is substituted with one or more of:
1) aryl or heterocycle, unsubstituted or substituted with:
   a) C₁₋₄ alkyl,
   b) (CH₂)ₚ OR⁶,
   c) (CH₂)ₚ NR⁶R⁷,
   d) halogen,
   e) CN,
2) C₃₋₆ cycloalkyl,
3) OR⁶,
4) SR^{6a}, S(O)R^{6a}, SO₂R^{6a},
5) —NR⁶R⁷, 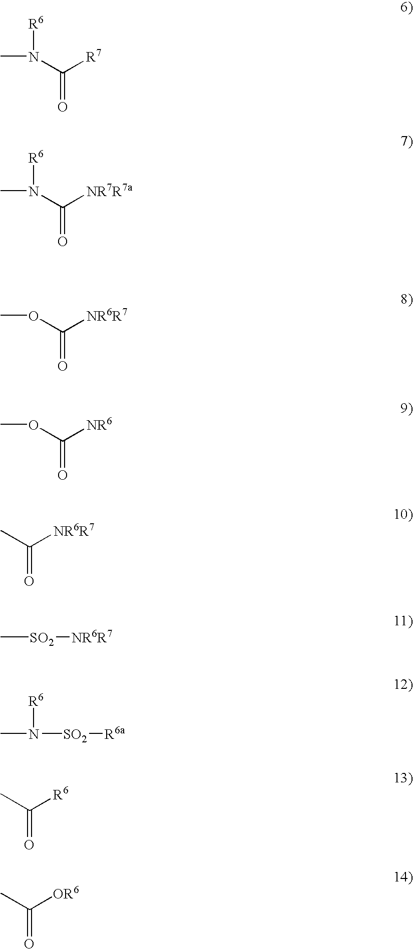
15) N₃ or
16) F; or
R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;
R⁴ is selected from H and CH₃;
and any two of R², R³ and R⁴ are optionally attached to the same carbon atom;
R⁶, R⁷ and R^{7a} are independently selected from: H; C₁₋₄ alkyl, C₃₋₆ cycloalkyl, heterocycle, aryl, aroyl, heteroaroyl, arylsulfonyl, heteroarylsulfonyl, unsubstituted or substituted with:
   a) C₁₋₄ alkoxy,
   b) aryl or heterocycle,
   c) halogen,
   d) HO,
   e) 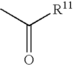
   f) —SO₂R¹¹, or g) N(R¹⁰)₂; or
   R⁶ and R⁷ may be joined in a ring;
   R⁷ and R^{7a} may be joined in a ring;
R^{6a} is selected from: C₁₋₄ alkyl, C₃₋₆ cycloalkyl, heterocycle, aryl, unsubstituted or substituted with:
   a) C₁₋₄ alkoxy,
   b) aryl or heterocycle,
   c) halogen,
   d) HO,
   e) 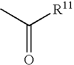
   f) —SO₂R¹¹, or
   g) N(R¹⁰)₂;
R⁸ is independently selected from:
a) hydrogen,
b) aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and
c) C₁-C₆ alkyl unsubstituted or substituted by aryl, cyanophenyl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NH—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹⁰OC(O)NH—;
R⁹ is selected from:
a) hydrogen,
b) C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂ NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and
c) C₁-C₆ alkyl unsubstituted or substituted by perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹¹OC(O)NR¹⁰—;
R¹⁰ is independently selected from hydrogen, C₁-C₆ alkyl, benzyl and aryl;
R¹¹ is independently selected from C₁-C₆ alkyl and aryl;
A¹ and A² are independently selected from: a bond, —CH═CH—, —C.tbd.C—,
—C(O)—, —C(O)NR¹⁰—, —NR¹⁰C(O)—, O, —N(R¹⁰)—,
—S(O)₂ N(R¹⁰)—, —N(R¹⁰)S(O)₂—, or S(O)ₘ;
G is O;
V is selected from:
a) hydrogen,
b) heterocycle,
c) aryl,
d) C₁-C₂₀ alkyl wherein from 0 to 4 carbon atoms are replaced with a a heteroatom selected from O, S, and N, and
e) C₂-C₂₀ alkenyl,
provided that V is not hydrogen if A¹ is S(O)ₘ and V is not hydrogen if A¹ is a bond, n is 0 and A² is S(O)ₘ;
W is a heterocycle;
X is —CH₂—, —C(═O)—, or —S(═O)ₘ—;
Z is a unsubstituted or substituted group selected from aryl, heteroaryl, arylmethyl, heteroarylmethyl, arylsulfonyl, heteroarylsulfonyl, wherein the substituted group is substituted with one or more of the following:
1) C₁₋₄ alkyl, unsubstituted or substituted with: a) C₁₋₄ alkoxy, b) NR⁶R⁷, c) C₃₋₆ cycloalkyl, d) aryl or heterocycle, e) HO, f) —S(O)ₘR^{6a}, or g) —C(O)NR⁶R⁷, 2) aryl or heterocycle, 3) halogen, 4) OR⁶, 5) NR⁶R⁷, 6) CN, 7) NO₂, 8) CF₃, 9)—S(O)ₘR^{6a}, 10) —C(O)NR⁶R⁷, or 11) C₃-C₆ cycloalkyl;
m is 0, 1 or 2; n is 0, 1, 2, 3 or 4; p is 0, 1, 2, 3 or 4; q is 1 or 2; r is 0 to 5, provided that r is 0 when V is hydrogen; s is 1; t is 0 or 1; and u is 4 or 5.

In another aspect, the invention provides a method of treating a synucleinopathic subject by administering one or more of the following farnesyl transferase inhibitor compounds:
2(S)-Butyl-1-(2,3-diaminoprop-1-yl)-4-(1-naphthoyl)piperazine
1-(3-Amino-2-(2-naphthylmethylamino)prop-1-yl)-2(S)-butyl-4-(1-naphthoyl)piperazine
2(S)-Butyl-1-{5-[1-(2-naphthylmethyl)]-4,5-dihydroimidazol}methyl-4-(1-naphthoyl)piperazine
1-[5-(1-Benzylimidazol)methyl]-2(S)-butyl-4-(1-naphthoyl)piperazine
1-{5-[1-(4-Nitrobenzyl)imidazolyl]methyl}-2(S)-butyl-4-(1-naphthoyl)piperazine
1-(3-Acetamidomethylthio-2(R)-aminoprop-1-yl)-2(S)-butyl-4-(1-naphthoyl)piperazine
2(S)-Butyl-1-[2-(1-imidazolyl)ethyl]sulfonyl-4-(1-naphthoyl)piperazine
2(R)-Butyl-1-imidazolyl-4-methyl-4-(1-naphthoyl)piperazine
2(S)-Butyl-4-(1-naphthoyl)-1-(3-pyridylmethyl)piperazine
1-2(S)-butyl-(2(R)-(4-nitrobenzyl)amino-3-hydroxypropyl)-4-(1-naphthoyl)piperazine
1-(2(R)-Amino-3-hydroxyheptadecyl)-2(S)-butyl-4-(1-naphthoyl)piperazine
2(S)-Benzyl-1-imidazolyl-4-methyl-4-(1-naphthoyl)piperazine
1-(2(R)-Amino-3-(3-benzylthio)propyl)-2(S)-butyl-4-(1-naphthoyl)piperazine
1-(2(R)-Amino-3-[3-(4-nitrobenzylthio)propyl]))-2(S)-butyl-4-(1-naphthoyl)piperazine
2(S)-Butyl-1-[(4-imidazolyl)ethyl]-4-(1-naphthoyl)piperazine
2(S)-Butyl-1-[(4-imidazolyl)methyl]-4-(1-naphthoyl)piperazine
2(S)-Butyl-1-[(1-naphth-2-ylmethyl)-1H-imidazol-5-yl)acetyl]-4-(1-naphthoyl)piperazine
2(S)-Butyl-1-[(1-naphth-2-ylmethyl)-1H-imidazol-5-yl)ethyl]-4-(1-naphthoyl)piperazine
1-(2(R)-Amino-3-hydroypropyl)-2(S)-butyl-4-(1-naphthoyl)piperazine
1-(2(R)-Amino-4-hydroxybutyl)-2(S)-butyl-4-(1-naphthoyl)piperazine
1-(2-Amino-3-(2-benzyloxyphenyl)propyl)-2(S)-butyl-4-(1-naphthoyl)piperazine
1-(2-Amino-3-(2-hydroxyphenyl)propyl)-2(S)-butyl-4-(1-naphthoyl)piperazine
1-[3-(4-imidazolyl)propyl]-2(S)-butyl-4-(1-naphthoyl)piperazine
2(S)-n-Butyl-4-(1-naphthoyl)-1-[1-(1-naphthylmethyl)imidazol-5-ylmethyl]-piperazine
2(S)-n-Butyl-4-(1-naphthoyl)-1-[1-(2-naphthylmethyl)imidazol-5-ylmethyl]-piperazine
2(S)-n-Butyl-1-[1-(4-cyanobenzyl)imidazol-5-ylmethyl]-4-(1-naphthoyl)piperazine
2(S)-n-Butyl-1-[1-(4-methoxybenzyl)imidazol-5-ylmethyl]-4-(1-naphthoyl)piperazine
2(S)-n-Butyl-1-[1-(3-methyl-2-butenyl)imidazol-5-ylmethyl]-4-(1-naphthoyl)piperazine
2(S)-n-Butyl-1-[1-(4-fluorobenzyl)imidazol-5-ylmethyl]-4-(1-naphthoyl)piperazine
2(S)-n-Butyl-1-[1-(4-chlorobenzyl)imidazol-5-ylmethyl]-4-(1-naphthoyl)piperazine
1-[1-(4-Bromobenzyl)imidazol-5-ylmethyl]-2(S)-n-butyl-4-(1-naphthoyl)piperazine
1-[1-(4-Bromobenzyl)imidazol-5-ylmethyl]-2(S)-n-butyl-4-(1-naphthoyl)piperazine
2(S)-n-Butyl-4-(1-naphthoyl)-1-[1-(4-trinfluoromethylbenzyl)imidazol-5-ylmethyl]-piperazine
2(S)-n-Butyl-1-[1-(4-methylbenzyl)imidazol-5-ylmethyl]-4-(1-naphthoyl)-piperazine
2(S)-n-Butyl-1-[1-(3-methylbenzyl)imidazol-5-ylmethyl]-4-(1-naphthoyl)-piperazine
1-[1-(4-Phenylbenzyl)imidazol-5-ylmethyl]-2(S)-n-butyl-4-(1-naphthoyl)-piperazine
2(S)-n-Butyl-4-(1-naphthoyl)-1-[1-(2-phenylethyl)imidazol-5-ylmethyl]-piperazine
2(S)-n-Butyl-4-(1-naphthoyl)-1-[1-(4-trifluoromethoxy)-imidazol-5-ylmethyl]piperazine
1-{[1-(4-cyanobenzyl)-1H-imidazol-5-yl]acetyl}-2(S)-n-butyl-4-(1-naphthoyl)piperazine
5(S)-n-Butyl-1-(2,3-dimethylphenyl)-4-(4-imidazolylmethyl)-piperazin-2-one
5(S)-n-Butyl-4-[1-(4-cyanobenzyl)imidazol-5-ylmethyl]-1-(2,3-dimethylphenyl)piperazin-2-one
4-[1-(4-Cyanobenzyl)imidazol-5-ylmethyl]-1-(2,3-dimethylphenyl)-5(S)-(2-methoxyethyl)piperazin-2-one
(S)-1-(3-Chlorophenyl)-4-[1-(4-cyanobenzyl)-5-imidazolylmethyl]-5-[2-(methanesulfonyl)ethyl]-2-piperazinone
(S)-1-(3-Chlorophenyl)-4-[1-(4-cyanobenzyl)-5-imidazolylmethyl]-5-[2-(ethanesulfonyl)ethyl]-2-piperazinone
(R)-1-(3-Chlorophenyl)-4-[1-(4-cyanobenzyl)-5-imidazolylmethyl]-5-[2-(ethanesulfonyl)methyl]-2-piperazinone
(S)-1-(3-Chlorophenyl)-4-[1-(4-cyanobenzyl)-5-imidazolylmethyl]-5-[N-ethyl-2-acetamido]-2-piperazinone
(±)-5-(2-Butynyl)-1-(3-chlorophenyl)-4-[1-(4-cyanobenzyl)-5-imidazolylmethyl]-2-piperazinone
1-(3-Chlorophenyl)-4-[1-(4-cyanobenzyl)-5-imidazolylmethyl]-2-piperazinone
5(S)-Butyl-4-[1-(4-cyanobenzyl-2-methyl)-5-imidazolylmethyl]-1-(2,3-dimethylphenyl)-piperazin-2-one
4-[1-(2-(4-Cyanophenyl)-2-propyl)-5-imidazolylmethyl]-1-(3-chlorophenyl)-5(S)-(2-methylsulfonylethyl)piperazin-2-one
5(S)-n-Butyl-4-[1-(4-cyanobenzyl)-5-imidazolylmethyl]-1-(2-methylphenyl)piperazin-2-one
4-[1-(4-Cyanobenzyl)-5-imidazolylmethyl]-5(S)-(2-fluoroethyl)-1-(3-chlorophenyl)piperazin-2-one
4-[3-(4-Cyanobenzyl)pyridin-4-yl]-1-(3-chlorophenyl)-5(S)-(2-methylsulfonylethyl)-piperazin-2-one
4-[5-(4-Cyanobenzyl)-1-imidazolylethyl]-1-(3-chlorophenyl)piperazin-2-one or a stereoisomeric form, or a pharmaceutically acceptable acid or base addition salt form thereof, in a therapeutically effective amount.

In another aspect, the invention provides a method of treating a synucleinopathic subject by administering one or more of the following farnesyl transferase inhibitor compounds:
1-{5-[1-(4-Nitrobenzyl)imidazolyl]methyl}-2(S)-butyl-4-(1-naphthoyl)piperazine 1-[5-(1-Benzylimidazol)methyl]-2(S)-butyl-4-(1-naphthoyl)piperazine
1-(2(R)-Amino-3-(3-benzylthio)propyl)-2(S)-butyl-4-(1-naphthoyl)piperazine
1-(2(R)-Amino-3-[3-(4-nitrobenzylthio)propyl])-2(S)-butyl-4-(1-naphthoyl)piperazine
2(S)-n-Butyl-1-[1-(4-cyanobenzyl)imidazol-5-ylmethyl]-4-(1-naphthoyl)piperazine
2(S)-n-Butyl-1-[1-(4-cyanobenzyl)imidazol-5-ylmethyl]-4-(2,3 dimethylphenyl)piperazin-5-one
2(S)-n-Butyl-1-[1-(4-chlorobenzyl)imidazol-5-ylmethyl]-4-(1-naphthoyl)piperazine
1-{[1-(4-Cyanobenzyl)-1H-imidazol-5-yl]acetyl}-2(S)-n-butyl-4-(1-naphthoyl)piperazine
1-[1-(4-Cyanobenzyl)imidazol-5-ylmethyl]-4-(2,3-dimethylphenyl)-2(S)-(2-methoxyethyl)piperazin-5-one
5(S)-n-Butyl-4-[1-(4-cyanobenzyl)-5-imidazolylmethyl]-1-(2-methylphenyl)piperazin-2-one
(S)-1-(3-Chlorophenyl)-4-[1-(4-cyanobenzyl)-5-imidazolylmethyl]-5-[2-(methanesulfonyl)ethyl]-2-piperazinone
(S)-1-(3-Chlorophenyl)-4-[1-(4-cyanobetizyl)-5-imidazolylmethyl]-5-[2-(ethanesulfonyl)ethyl]-2-piperazinone
(R)-1-(3-Chlorophenyl)-4-[1-(4-cyanobenzyl)-5-imidazolylmethyl]-5-[2-(ethanesulfonyl)methyl]-2-piperazinone
1-(3-Chlorophenyl)-4-[1-(4-cyanobenzyl)-5-imidazolyl-methyl]-2-piperazinone
   or a stereoisomeric form, or a pharmaceutically acceptable acid or base addition salt form thereof, in a therapeutically effective amount.

In one embodiment, the compound may be 1-(3-Chlorophenyl)-4-[1-(4-cyanobenzyl)-5-imidazolyl-methyl]-2-piperazinone or a stereoisomeric form, or a pharmaceutically acceptable acid or base addition salt form thereof.

In another aspect, the invention provides a method of treating a synucleinopathic subject by administering one or more of the following farnesyl transferase inhibitor compounds:
5(S)-n-Butyl-1-(2,3-dimethylphenyl)-4-(4-imidazolylmethyl)-piperazin-2-one
5 (S)-n-Butyl-4-[1-(4-cyanobenzyl)imidazol-5-ylmethyl]-1-(2,3-dimethylphenyl)piperazin-2-one
4-[1-(4-Cyanobenzyl)imidazol-5-ylmethyl]-1-(2,3-dimethylphenyl)-5(S)-(2-methoxyethyl)piperazin-2-one
(S)-1-(3-Chlorophenyl)-4-[1-(4-cyanobenzyl)-5-imidazolylmethyl]-5-[2-(methanesulfonyl)ethyl]-2-piperazinone
(S)-1-(3-Chlorophenyl)-4-[1-(4-cyanobenzyl)-5-imidazolylmethyl]-5-[2-(ethanesulfonyl)ethyl]-2-piperazinone
(R)-1-(3-Chlorophenyl)-4-[1-(4-cyanobenzyl)-5-imidazolylmethyl]-5-[2-(ethanesulfonyl)methyl]-2-piperazinone
(S)-1-(3-Chlorophenyl)-4-[1-(4-cyanobenzyl)-5-imidazolylmethyl]-5-[N-ethyl-2-acetamido]-2-piperazinone
(±)-5-(2-Butynyl)-1-(3-chlorophenyl)-4-[1-(4-cyanobenzyl)-5-imidazolylmethyl]-2-piperazinone
1-(3-Chlorophenyl)-4-[1-(4-cyanobenzyl)-5-imidazolylmethyl]-2-piperazinone
5(S)-Butyl-4-[1-(4-cyanobenzyl-2-methyl)-5-imidazolylmethyl]-1-(2,3-dimethylphenyl)-piperazin-2-one
4-[1-(2-(4-Cyanophenyl)-2-propyl)-5-imidazolylmethyl]-1-(3-chlorophenyl)-5(S)-(2-methylsulfonylethyl)piperazin-2-one
5(S)-n-Butyl-4-[1-(4-cyanobenzyl)-5-imidazolylmethyl]-1-(2-methylphenyl)piperazin-2-one
4-[1-(4-Cyanobenzyl)-5-imidazolylmethyl]-5(S)-(2-fluoroethyl)-1-(3-chlorophenyl)piperazin-2-one
4-[5-(4-Cyanobenzyl)-1-imidazolylethyl]-1-(3-chlorophenyl)piperazin-2-one.

In another aspect, the invention provides a method of treating a synucleinopathic subject by administering one or more of the following farnesyl transferase inhibitor compounds:
1-(3-Trifluoromethoxyphenyl)-4-[1-(4-cyanobenzyl)imidazolylmethyl]-2-piperazinone
1-(2,5-Dimethylphenyl)-4-[1-(4-cyanobenzyl)imidazolylmethyl]-2-piperazinone
1-(3-Methylphenyl)-4-[1-(4-cyanobenzyl)imidazolylmethyl]-2-piperazinone
1-(3-Iodophenyl)-4-[1-(4-cyanobenzyl)imidazolylmethyl]-2-piperazinone
1-(3-Chlorophenyl)-4-[1-(3-methoxy-4-cyanobenzyl)imidazolylmethyl]-2-piperazinone
1-(3-Trifluoromethoxyphenyl)-4-[1-(3-methoxy-4-cyanobenzylimidazo)ylmethyl]-2-piperazinone
(R)-5-[(Benzyloxy)methyl]-1-(3-chlorophenyl)-4-[1-(4-cyanobenzyl)-imidazolylmethyl]-2-piperazinone
1-(3-Chlorophenyl)-4-[1-(2-fluoro-4-cyanobenzyl)-1H-imidazol-5-ylmethyl]piperazin-2-one
4-[1-(4-Cyanobenzyl)-1H-imidazol-5-ylmethyl]-1-(3-methylthiophenyl)piperazin-2-one
4-[1-(4-Cyanobenzyl)-1H-imidazol-5-ylmethyl]-1-(3,5-dichlorophenyl)piperazin-2-one
1-(3-Chlorophenyl)-4-{[1-(4-cyanophenyl)-1-ethyl]-1H-imidazol-5-ylmethyl)piperazin-2-one
1-(3-Chloro-4-fluorophenyl)-4-1-(4-cyanobenzyl)-1H-imidazol-5-ylmethyl]-piperazin-2-one
4-[1-(4-Cyanobenzyl)-1H-imidazol-5-ylmethyl]-1-(3,5-dimethylphenyl)piperazin-2-one
(S)-5-Benzyl-4-[3-(4-cyanobenzyl-1-imidazol-5-yl)prop-1-yl)-1-phenyl-2-piperazinone
1-(3-Chlorophenyl)-4-[1-(4-nitrobenzyl)-1H-imidazol-5-ylmethyl]piperazin-2-one
4-[1-(4-Cyanobenzyl)-1H-imidazol-5-ylmethyl]-1-(3,5-difluorophenyl)piperazin-2-one
4-[1-(4-Cyanobenzyl)-1H-imidazol-5-ylmethyl]-1-(3,4-difluorophenyl)piperazin-2-one.

In another aspect, the invention provides a method of treating a synucleinopathic subject by administering a farnesyl transferase inhibitor compound of the formula: 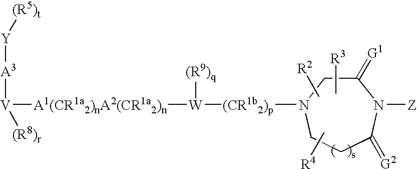
or a stereoisomeric form, or a pharmaceutically acceptable acid or base addition salt form thereof, in a therapeutically effective amount,

wherein:
R^{1a} and R^{1b} are independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, R¹⁰O—, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O), R¹⁰OC(O, —N(R¹⁰)₂, or R¹¹ OC(O)NR¹⁰—, or
c) unsubstituted or substituted C₁-C₆ alkyl wherein the substitutent on the substituted C₁-C₆ alkyl is selected from unsubstituted or substituted aryl, unsubstituted or substituted heterocycle, unsubstituted or substituted C₃-C₁₀ cycloalkyl, C₂-C₈ alkenyl, C₂-C₈ alkynyl, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰OC(O)—, R¹⁰OC(O)—, —N(R¹⁰)₂, and R¹¹OC(O)NR¹⁰—;
R² and R³ are independently selected from: H, unsubstituted or substituted C₁₋₆ alkyl, unsubstituted or substituted C₂₋₈ alkenyl, unsubstituted or substituted C₂₋₈ alkynyl, unsubstituted or substituted aryl, unsubstituted or substituted heterocycle, 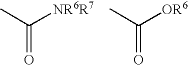
   wherein the substituted group is substituted with one or more of:
1) aryl or heterocycle, unsubstituted or substituted with:
   a) C₁₋₆ alkyl,
   b) (CH₂)pOR⁶⁷,
   c) (CH₂)pNR⁶R⁷,
   d) halogen,
   e) CN,
2) C₃₋₆ cycloalkyl,
3) OR⁶,
4) SR^{6a}, S(O)R^{6a}, SO₂R
5) —NR⁶R⁷, 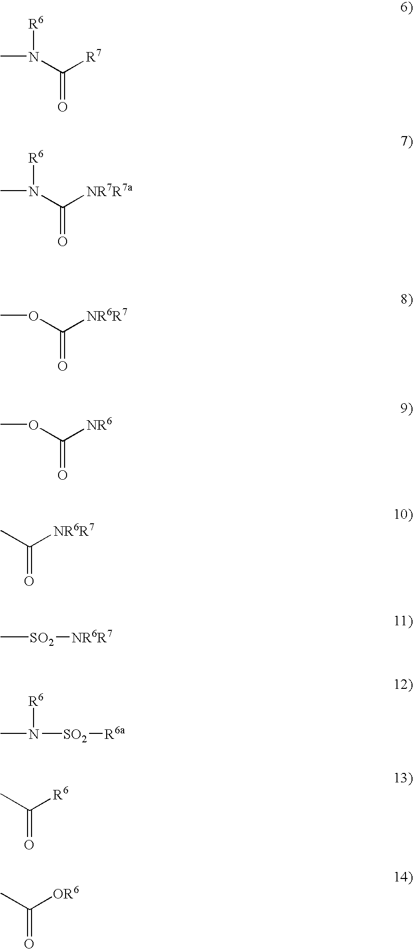
15) N₃ or
16) F; or
R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;
R⁴ is selected from H and unsubstituted or substituted C₁-C₆ alkyl; and any two of R², R³ or R⁴ are optionally attached to the same carbon atom;
R⁵ is independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰OC(O)NR¹⁰—, (R¹⁰)₂NC(OH, (R¹⁰)₂NC(O)NR¹⁰—, CN, NO₂, R¹⁰OC(O)—, R¹⁰OC(O), —N(R¹⁰)₂, or R¹¹OC(O)NR¹⁰—, and
c) C₁-C₆ alkyl, unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O), R¹⁰OC(O), —N(R¹⁰)₂, or R¹¹OC(O)NR¹⁰—;
R⁶, R⁷ and R^{7a} are independently selected from: H, C₁-C₆ alkyl, C₃₋₆ cycloalkyl, heterocycle, aryl, aroyl, heteroaroyl, arylsulfonyl, heteroarylsulfonyl, unsubstituted or substituted with:
a) C₁₋₆ alkoxy,
b) C₁-C₂₀ alkyl
c) aryl or heterocycle,
d) halogen,
e) HO,
f) —C(O)R¹¹,
g) —SO₂R¹¹, or
h) N(R¹⁰)₂; or
R⁶ and R⁷ may be joined in a ring;
R⁷ and R⁷a may be joined in a ring;
R^{6a} is selected from: C₁-C₆ alkyl, C₃₋₆ cycloalkyl, heterocycle, aryl, aroyl, heteroaroyl, arylsulfonyl, heteroarylsulfonyl, unsubstituted or substituted with:
a) C₁₋₄ alkoxy,
b) C₁-C₂₀ alkyl
c) aryl or heterocycle,
d) halogen,
e) HO,
f) —C(O)R¹¹,
g) —SO₂R¹¹, or
h) N(R¹⁰)₂;
R⁸ is independently selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O), R¹⁰OC(O)—, —N(R¹⁰)₂, or R¹¹OC(O)NR¹⁰—, and
c) C₁-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹C(O)NR¹⁰—;
R⁹ is selected from:
a) hydrogen,
b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ—, R¹⁰C(O)NR¹⁰—, (R¹⁰)₂NC(OH)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and
c) C₁-C₆ alkyl unsubstituted or substituted by aryl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—,
R¹⁰ is independently selected from hydrogen, unsubstituted or substituted C₁-C₆ alkyl, perfluoroalkyl, unsubstituted or substituted aralkyl, and unsubstituted or substituted aryl; R¹¹ is independently selected from unsubstituted or substituted C₁-C₆ alkyl and unsubstituted or substituted aryl;
A¹ and A² are independently selected from: a bond, —CH═CH—, —C≡C—, —C(O)—, —C(O)NR¹⁰—, —NR¹⁰C(O)—, O, —N(R¹⁰)—, —S(O)₂N(R¹⁰), —N(R¹⁰)S(O)₂—, or S(O)ₘ;
A³ is selected from —C(O)—, —C(R^{1a})₂—, O, —N(R¹⁰)— and S(O)ₘ;
G¹ or G² is selected from H₂ or O, provided that if G¹ is O then G² is H₂ and if G² is O, then G¹ is H₂;
V is selected from:
a) heterocycle, and
b) aryl,
W is a heterocycle;
Y is heteroaryl;
Z is a unsubstituted or substituted group selected from aryl, heteroaryl, arylmethyl, heteroarylmethyl, arylsulfonyl, heteroarylsulfonyl, wherein the substituted group is substituted with one or more of the following:
1. C₁-C₆ alkyl, unsubstituted or substituted with:
a) C₁₋₆ alkoxy,
b) NR⁶R⁷,
c) C₃₋₆ cycloalkyl,
d) aryl or heterocycle,
e) HO,
f) —S(O)ₘR^{6a}, or
g) —C(O)NR⁶R⁷,
2. unsubstituted or substituted aryl or unsubstituted or substituted heterocycle,
3. halogen,
4. OR⁶,
5. NR⁶R⁷,
6. CN,
7. NO₂;
8. CF₃,
9. —S(O)ₘR^{6a},
10. —C(O)NR⁶R⁷,
11. —OCF₃,
12. unsubstituted or substituted C₁₋₆ alkoxy,
13. C₂-C₈alkenyl,
14. C₂-C₈ alkynyl, or
15. C₃-C₁₀cycloalkyl;
m is 0, 1 or 2;
n is 0, 1, 2, 3 or 4;
p is 0, 1, 2, 3 or 4;
q is 0, 1 or 2;
r is 0 to 5;
s is 0 or 1;
t is 0 to 5;
u is 4 or 5; and
x is 0, 1, 2, 3 or 4.

In another aspect, the invention provides a method of treating a synucleinopathic subject by administering a farnesyl transferase inhibitor compound of the formula: 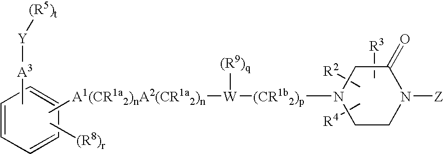
or a stereoisomeric form, or a pharmaceutically acceptable acid or base addition salt form thereof, in a therapeutically effective amount,

wherein:
R^{1a} and R^{1b} are independently selected from:
a) hydrogen,
b) unsubstituted or substituted aryl, unsubstituted or substituted heterocycle, unsubstituted or substituted C₃-C₁₀ cycloalkyl, R¹⁰O—, —N(R¹⁰)₂, or, C₂-C₈ alkenyl, or
c) unsubstituted or substituted C₁-C₆ alkyl wherein the substitutent on the substituted C₁-C₆ alkyl is selected from unsubstituted or substituted aryl, unsubstituted or substituted heterocycle, unsubstituted or substituted C₃-C₁₀ cycloalkyl, C₂-C₈ alkenyl, R¹⁰O—, or —N(R¹⁰)₂;
R² and R³ are independently selected from: H, unsubstituted or substituted C₁₋₆ 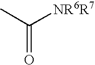
   wherein the substituted group is substituted with one or more of:
1) aryl or heterocycle, unsubstituted or substituted with:
   a) C₁-C₆alkyl,
   b) (CH₂)ₚOR⁶,
   c) (CH₂)ₚNR⁶R⁷.
   d) halogen,
   e) CN;
2. C₃₋₆ cycloalkyl;
3. OR⁶;
4. SR^{6a}, S(O)R^{6a}, SO₂R^{6a},
5) —NR⁶R⁷, 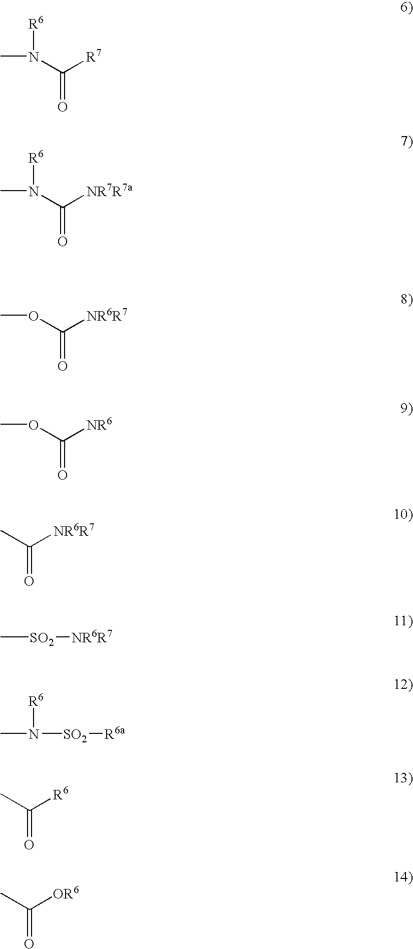
15) N₃ or
16) F; or
   R² and R³ are attached to the same C atom and are combined to form —(CH₂)ᵤ— wherein one of the carbon atoms is optionally replaced by a moiety selected from: O, S(O)ₘ, —NC(O), and —N(COR¹⁰)—;
R⁴ is selected from H and unsubstituted or substituted C₁-C₆ alkyl; and any two of R², R³ or R⁴ are optionally attached to the same carbon atom;
R⁵ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O), —N(R¹⁰)₂, or R¹¹OC(O)NR¹⁰—;

R⁶, R⁷ and R^{7a} are independently selected from: H, C₁-C₆ alkyl, C₃₋₆ cycloalkyl, heterocycle, aryl, aroyl, heteroaroyl, arylsulfonyl, heteroarylsulfonyl, unsubstituted or substituted with:

a) C₁₋₆ alkoxy,

b) C₁-C₂₀ alkyl

c) aryl or heterocycle,

d) halogen,

e) HO,

f) —C(O)R¹¹,

g) —SO₂R¹¹, or

h) N(R¹⁰)₂; or

R⁶ and R⁷ may be joined in a ring;

R⁷ and R^{7a} may be joined in a ring;

R⁶a is selected from: C₁-C₆ alkyl, C₃₋₆ cycloalkyl, heterocycle, aryl, aroyl, heteroaroyl, arylsulfonyl, heteroarylsulfonyl, unsubstituted or substituted with:

a) C₁₋₆ alkoxy,

b) C₁-C₂₀ alkyl

c) aryl or heterocycle,

d) halogen,

e) HO,

f) —C(O)R¹¹,

g) —SO₂R¹¹, or

h) N(R¹⁰)₂; or

R⁸ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰, CN, NO₂, R¹⁰C(O)—, R¹⁰OC(O), —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, C₂-C₈ alkenyl, C₂-C₈ alkynyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O), —N(R¹⁰)₂, or R¹¹OC(O)NR¹⁰—;

R⁹ is selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ—,

R¹⁰C(O)NR¹⁰—, (R¹⁰)₂NC(OH)—, (R¹⁰)₂NC(O)NR¹⁰—, R¹⁰2N—C(NR¹⁰)—, CN, NO₂, R¹⁰C(O)—, R¹⁰OC(O), N₃, —N(R¹⁰)₂, or R¹¹OC(O)NR¹⁰—, and

c) C₁-C₆ alkyl unsubstituted or substituted by aryl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;

R¹⁰ is independently selected from hydrogen, unsubstituted or substituted C₁-C₆ alkyl, perfluoroalkyl, unsubstituted or substituted aralkyl, and unsubstituted or substituted aryl;

R¹¹ is independently selected from unsubstituted or substituted C₁-C₆ alkyl and unsubstituted or substituted aryl;

A¹ and A² are independently selected from: a bond, —CH═CH—, —C≡C—, —C(O—, C(O)NR¹⁰—, —NR¹⁰OC(O)—, O, —N(R¹⁰)—, —S(O)₂N(R¹⁰)—, —N(R¹⁰)S(O)₂—, or S(O)ₘ;

A³ is selected from —C(O)—, —C(R^{1a})₂—, O, —N(R¹⁰)— and S(O)ₘ;

W is a heterocycle selected from imidazolyl, pyridyl, thiazolyl, indolyl, quinolinyl, isoquinolinyl and thienyl;

Y is heteroaryl;

Z is a unsubstituted or substituted group selected from aryl, heteroaryl, arylmethyl, heteroarylmethyl, arylsulfonyl, heteroarylsulfonyl, wherein the substituted group is substituted with one or more of the following:

1. C₁-C₆ alkyl, unsubstituted or substituted with:

a) C₁₋₆ alkoxy,

b) NR⁶R⁷,

c) C₃₋₆ cycloalkyl,

d) aryl or heterocycle,

e) HO,

f) —S(O)ₘR^{6a}, or

g) —C(O)NR⁶R⁷,

2. unsubstituted or substituted aryl or unsubstituted or substituted heterocycle,

3. halogen,

4. OR,

5. NR⁶R⁷,

6. CN,

7. NO₂,

8. CF₃;

9. —S(O)ₘR^{6a},

10. —C(O)NR⁶R⁷,

11. C₃-C₆ cycloalkyl,

12. —OCF₃, or

13. unsubstituted or substituted C₁₋₆ alkoxy;

m is 0, 1 or 2;

n is 0, 1, 2, 3 or 4;

p is 0, 1, 2, 3 or 4;

q is 0, 1 or 2;

r is 0 to 5;

t is 0 to 5;

u is 4 or 5; and

x is 0, 1, 2, 3 or 4.

In another aspect, the invention provides a method of treating a synucleinopathic subject by administering a farnesyl transferase inhibitor compound of the formula: 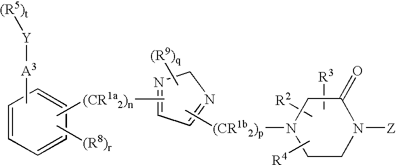
or a stereoisomeric form, or a pharmaceutically acceptable acid or base addition salt form thereof, in a therapeutically effective amount,

wherein: R^{1a} and R^{1b} are independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, R¹⁰O—, or —N(R¹⁰)₂, or

c) unsubstituted or substituted C₁-C₆ alkyl wherein the substitutent on the substituted C₁-C₆ alkyl is selected from unsubstituted or substituted aryl, unsubstituted or substituted heterocycle, unsubstituted or substituted C₃-C₁₀ cycloalkyl, C₂-C₈ alkenyl, R¹⁰O—, or —N(R¹⁰)₂;

R² is H, unsubstituted or substituted C₁₋₆ alkyl, or 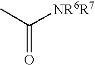
wherein the substituted group is substituted with one or more of:

1) aryl,

2) heterocycle,

3) OR⁶,

4) SR^{6a}, SO₂R^{6a}, or

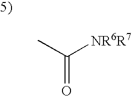

R³ and R⁴ are independently selected from H and unsubstituted or substituted C₁-C₆ alkyl; and any two of R², R³ or R⁴ are optionally attached to the same carbon atom;

R⁵ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, perfluoroalkyl, F, Cl, Br, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹1OC(O)NR¹⁰—;

R⁶ and R⁷ are independently selected from: H, C₁-C₆ alkyl, C₃₋₆ cycloalkyl, heterocycle, aryl, unsubstituted or substituted with:

a) C₁₋₆ alkoxy,

b) C₁-C₂₀ alkyl

c) aryl or heterocycle,

d) halogen, or

e) HO;

R⁶ and R⁷ may be joined in a ring;

R^{6a} is selected from: C₁-C₆ alkyl, C₃₋₆ cycloalkyl, heterocycle, aryl, aroyl, heteroaroyl, arylsulfonyl, heteroarylsulfonyl, unsubstituted or substituted with:

a) C₁₋₆ alkoxy,

b) C₁-C₂₀ alkyl

c) aryl or heterocycle,

d) halogen, or

e) HO;

R⁸ is independently selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, unsubstituted or substituted C₁-C₆ alkoxy, R¹¹S(O)ₘ—, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹⁰OC(O)NR¹⁰—;

R⁹ is selected from:

a) hydrogen,

b) unsubstituted or substituted aryl, unsubstituted or substituted heterocycle, unsubstituted or substituted C₃-C₁₀ cycloalkyl, unsubstituted or substituted C₂-C₈ alkenyl, unsubstituted or substituted C₂-C₈ alkynyl, perfluoroalkyl, halo, R¹⁰O—, R¹¹S(O)ₘ, R¹⁰C(O)NR¹⁰—, (R¹⁰)₂NC(O)—, (R¹⁰)₂NC(O)NR¹⁰—, CN, NO₂, R¹⁰C(O)—, R¹⁰OC(O)—, —N(R¹⁰)₂, or R¹¹OC(O)NR¹⁰—, and

c) C₁-C₆ alkyl unsubstituted or substituted by aryl, heterocycle, C₃-C₁₀ cycloalkyl, perfluoroalkyl, halo, R¹⁰O—, R¹¹S(O)ₘ—, R¹⁰C(O)NR¹⁰—, (R¹⁰)₂NC(O)—, (R¹⁰)₂NC(O)NR¹⁰—, CN, R¹⁰C(O)—, R¹⁰OC(O)—, —N(R¹⁰)₂, or R¹¹OC(O)NR¹⁰—;

R¹⁰ is independently selected from hydrogen, unsubstituted or substituted C₁-C₆ alkyl, perfluoroalkyl, unsubstituted or substituted aralkyl, and unsubstituted or substituted aryl;

R¹¹ is independently selected from unsubstituted or substituted C₁-C₆ alkyl and unsubstituted or substituted aryl;

A³ is selected from —C(O)—, —C(R^{1a})₂—, O, —N(R¹⁰)— and S(O)ₘ;

Y is heteroaryl;

Z is a unsubstituted or substituted group selected from aryl, heteroaryl, arylmethyl, heteroarylmethyl, wherein the substituted group is substituted with one or more of the following:

1. C₁-C₆ alkyl, unsubstituted or substituted with: a) C₁₋₆ alkoxy, b) NR⁶R⁷, c) C₃₋₆ cycloalkyl, d) aryl or heterocycle, e) HO, f) —S(O)ₘR^{6a}, or g) —C(O)NR⁶R⁷, 2. unsubstituted or substituted aryl or unsubstituted or substituted heterocycle, 3. halogen, 4. OR⁶, 5. NR⁶R⁷, 6. CN, 7. NO₂, 8. CF₃; 9. —S(O)ₘR^{6a}, 10. —C(O)NR⁶R⁷, 11. C₃-C₆cycloalkyl, 12. —OCF₃, or 13. unsubstituted or substituted C₁₋₆ alkoxy;

m is 0, 1 or 2; n is 0, 1, 2, 3 or 4; p is 0, 1, 2, 3 or 4; q is 0, 1 or 2; r is 0 to 5; t is 0 to 5; and u is 4 or 5.

In another aspect, the invention provides a method of treating a synucleinopathic subject by administering a farnesyl transferase inhibitor compound of the list comprising of: (3-chlorophenyl)-4-[1-(3-(3-pyridyloxy)-4-cyanobenzyl)-5-imidazolylmethyl]-2-piperazinone; and 1-(2-(n-Butyloxy)phenyl)-4-[1-(3-((6-methyl-2-pyridyl)oxy)-4-cyanobenzyl)-2-methyl-5-imidazolylmethyl]-2-piperazinone; or a stereoisomeric form, or a pharmaceutically acceptable acid or base addition salt form thereof, in a therapeutically effective amount.

In another aspect, the invention provides a method of treating a synucleinopathic by administering one or more of the following a farnesyl transferase inhibitor compounds: 1-(3-chlorophenyl)-4-[1-(3-((2-chlorophenyl)oxy)-4-cyanobenzyl)-5-imidazolylmethyl]-2-piperazinone; 1-(3-chlorophenyl)-4-[1-(3-((3-chlorophenyl)oxy)-4-cyanobenzyl)-5-imidazolylmethyl]-2-piperazinone; 1-(3-chlorophenyl)-4-[1-(3-((4-chlorophenyl)oxy)-4-cyanobenzyl)-5-imidazolylmethyl]-2-piperazinone; 1-(3-chlorophenyl)-4-[1-(³-((4-biphenylyl)oxy)-4-cyanobenzyl)-5-imidazolylmethyl]-2-piperazinone; 1-(3-chlorophenyl)-4-[1-(3-((3-(2-hydroxy-1-ethoxy)phenyl)oxy)-4-cyanobenzyl)-5-imidazolylmethyl]-2-piperazinone; 1-(3-chlorophenyl)-4-[1-(3-((4-(benzyloxy)phenyl)oxy)-4-cyanobenzyl)-5-imidazolylmethyl]-2-piperazinone; and 1-(2-(n-Butyloxy)phenyl)-4-[1-(3-((3-(2-hydroxy-1-ethoxy)phenyl)oxy)-4-cyanobenzyl)-2-methyl-5-imidazolylmethyl]-2-piperazinone, or a stereoisomeric form, or a pharmaceutically acceptable acid or base addition salt form thereof, in a therapeutically effective amount.

In one embodiment, the compound may be 1-(3-chlorophenyl)-4-[1-(3-((2-chlorophenyl)oxy)-4-cyanobenzyl)-5-imidazolylmethyl]-2-piperazinone. In another embodiment, the compound may be 1-(3-chlorophenyl)-4-[1-(3-((3-chlorophenyl)oxy)-4-cyanobenzyl)-5-imidazolylmethyl]-2-piperazinone. In another embodiment, the compound may be 1-(3-chlorophenyl)-4-[1-(3-((4-chlorophenyl)oxy)-4-cyanobenzyl)-5-imidazolylmethyl]-2-piperazinone. In another embodiment, the compound may be 1-(3-chlorophenyl)-4-[1-(3-((4-biphenylyl)oxy)-4-cyanobenzyl)-5-imidazolylmethyl]-2-piperazinone. In another embodiment, the compound may be 1-(3-chlorophenyl)-4-[1-(3-((3-(2-hydroxy-1-ethoxy)phenyl)oxy)-4-cyanobenzyl)-5-imidazolylmethyl]-2-piperazinone. In another embodiment, the compound may be 1-(3-chlorophenyl)-4-[1-(3-((4-(benzyloxy)phenyl)oxy)-4-cyanobenzyl)-5-imidazolylmethyl]-2-piperazinone. In another embodiment, the compound may be 1-(2-(n-Butyloxy)phenyl)-4-[1-(3-((3-(2-hydroxy-1-ethoxy)phenyl)oxy)-4-cyanobenzyl)-2-methyl-5-midazolylmethyl]-2-piperazinone.

In another aspect, the invention provides a method of treating a synucleinopathic subject by administering one or more of the following farnesyl transferase inhibitor compounds: 2(S)-Butyl-1-(2,3-diaminoprop-1-yl)-1-(1-naphthoyl)piperazine; 1-(3-Amino-2-(2-naphthylmethylamino)prop-1-yl)-2(S)-butyl-4-(1-naphthoyl)piperazine; 2(S)-Butyl-1-{5-[1-(2-naphthylmethyl)]-4,5-dihydroimidazol}methyl-4-(1-naphthoyl)piperazine; 1-[5-(1-Benzylimidazol)methyl]-2(S)-butyl-4-(1-naphthoyl)piperazine; 1-{(5-[1-(4-nitrobenzyl)]imidazolylmethyl}-2(S)-butyl-4-(1-naphthoyl)piperazine; 1-(3-Acetamidomethylthio-2(R)-aminoprop-1-yl)-2(S)-butyl-4-(1-naphthoyl)piperazine; 2(S)-Butyl-1-[2-(1-imidazolyl)ethyl]sulfonyl-4-(1-naphthoyl)piperazine; 2(R)-Butyl-1-imidazolyl-4-methyl-4-(1-naphthoyl)piperazine; 2(S)-Butyl-4-(1-naphthoyl)-1-(3-pyridylmethyl)piperazine; 1-2(S)-butyl-(2(R) -(4-nitrobenzyl)amino-3-hydroxypropyl)-4-(1-naphthoyl)piperazine; 1-(2(R)-Amino-3-hydroxyheptadecyl)-2(S)-butyl-4-(1-naphthoyl)-piperazine; 2(S)-Benzyl-1-imidazolyl-4-methyl-4-(1-naphthoyl)piperazine; 1-(2(R)-Amino-3-(3-benzylthio)propyl)-2(S)-butyl-4-(1-naphthoyl)piperazine; 1-(2(R)-Amino-3-[3-(4-nitrobenzylthio)propyl])-2(S)-butyl-4-(1-naphthoyl)piperazine; 2(S)-Butyl-1-[(4-imidazolyl)ethyl]-4-(1-naphthoyl)piperazine; 2(S)-Butyl-1-[(4-imidazolyl)methyl]-4-(1-naphthoyl)piperazine; 2(S)-Butyl-1-[(1-naphth-2-ylmethyl)-1H-imidazol-5-yl)acetyl]-4-(1-naphthoyl)piperazine; 2(S)-Butyl-1-[(1-naphth-2-ylmethyl)-1H-imidazol-5-yl)ethyl]-4-(1-naphthoyl)piperazine; 1-(2(R)-Amino-3-hydroypropyl)-2(S)-butyl-4-(1-naphthoyl)piperazine; 1-(2(R)-Amino-4-hydroxybutyl)-2(S)-butyl-4-(1-naphthoyl)piperazine; 1-(2-Amino-3-(2-benzyloxyphenyl)propyl)-2(S)-butyl-4-(1-naphthoyl)piperazine; 1-(2-Amino-3-(2-hydroxyphenyl)propyl)-2(S)-butyl-4-(1-naphthoyl)piperazine; 1-[3-(4-imidazolyl)propyl]-2(S)-butyl-4-(1-naphthoyl)-piperazine; 2(S)-n-Butyl-4-(2,3-dimethylphenyl)-1-(4-imidazolylmethyl)-piperazin-5-one; 2(S)-n-Butyl-1-[1-(4-cyanobenzyl)imidazol-5-ylmethyl]-4-(2,3-dimethylphenyl)piperazin-5-one; 1-[1-(4-Cyanobenzyl)imidazol-5-ylmethyl]-4-(2,3-dimethylphenyl)-2(S)-(2-methoxyethyl)piperazin-5-one; 2(S)-n-Butyl-4-(1-naphthoyl)-1-[1-(1-naphthylmethyl)imidazol-5-ylmethyl]-piperazine; 2(S)-n-Butyl-4-(1-naphthoyl)-1-[1-(2-naphthylmethyl)imidazol-5-ylmethyl]-piperazine; 2(S)-n-Butyl-1-[1-(4-cyanobenzyl)imidazol-5-ylmethyl]-4-(1-naphthoyl)piperazine; 2(S)-n-Butyl-1-[1-(4-methoxybenzyl)imidazol-5-ylmethyl]-4-(1-naphthoyl)piperazine; 2(S)-n-Butyl-1-[1-(3-methyl-2-butenyl)imidazol-5-ylmethyl-4-(1-naphthoyl)piperazine; 2(S)-n-Butyl-1-[1-(4-fluorobenzyl)imidazol-5-ylmethyl]-4-(1-naphthoyl)piperazine; 2(S)-n-Butyl-1-[1-(4-chlorobenzyl)imidazol-5-ylmethyl]-4-(1-naphthoyl)piperazine; 1-[1-(4-Bromobenzyl)imidazol-5-ylmethyl]-2(S)-n-butyl-4-(1-naphthoyl)piperazine; 2(S)-n-Butyl-4-(1-naphthoyl)-1-[1-(4-trifluoromethylbenzyl)imidazol-5-ylmethyl]-piperazine; 2(S)-n-Butyl-1-[1-(4-methylbenzyl)imidazol-5-ylmethyl]-4-(1-naphthoyl)-piperazine; 2(S)-n-Butyl-1-[1-(3-methylbenzyl)imidazol-5-ylmethyl]-4-(1-naphthoyl)-piperazine; 1-[1-(4-Phenylbenzyl)imidazol-5-ylmethyl]-2(S)-n-butyl-4-(1-naphthoyl)-piperazine; 2(S)-n-Butyl-4-(1-naphthoyl)-1-[1-(2-phenylethyl)imidazol-5-ylmethyl]-piperazine; 2(S)-n-Butyl-4-(1-naphthoyl)-1-[1-(4-trifluoromethoxy)imidazol-5-ylmethyl]piperazine; 1-1 [1-(4-cyanobenzyl)-1H-imidazol-5-yl]acetyl]-2(S)-n-butyl-4-(1-naphthoyl)piperazine; (S)-1-(3-Chlorophenyl)-4-[1-(4-cyanobenzyl)-5-imidazolylmethyl]-5-[2-(methanesulfonyl)ethyl]-2-piperazinone; (S)-1-(3-Chlorophenyl)-4-[1-(4-cyanobenzyl)-5-imidazolylmethyl]-5-[2-(ethanesulfonyl)ethyl]-2-piperazinone; (R)-1-(3-Chlorophenyl)-4-[1-(4-cyanobenzyl)-5-imidazolylmethyl]-5-[2-(ethanesulfonyl)methyl]-2-piperazinone; (S)-1-(3-Chlorophenyl)-4-[1-(4-cyanobenzyl)-5-imidazolylmethyl]-5-[N-ethyl-2-acetamido]-2-piperazinone; (±)-5-(2-Butynyl)-1-(3-chlorophenyl)-4-[1-(4-cyanobenzyl)-5-imidazolylmethyl]-2-piperazinone; 1-(3-Chlorophenyl)-4-[1-(4-cyanobenzyl)-5-imidazolylmethyl]-2-piperazinone; 5(S)-Butyl-4-[1-(4-cyanobenzyl-2-methyl)-5-imidazolylmethyl]-1-(2,3-dimethylphenyl)-piperazin-2-one; 4-[1-(2-(4-Cyanophenyl)-2-propyl)-5-imidazolylmethyl]-1-(3-chlorophenyl)-5(S)-(2-methylsulfonylethyl)piperazin-2-one; 5(S)-n-Butyl-4-[1-(4-cyanobenzyl)-5-imidazolylmethyl]-(2-methylphenyl)piperazin-2-one; 4-[1-(4-Cyanobenzyl)-5-imidazolylmethyl]-5(S)-(2-fluoroethyl)-1-(3-chlorophenyl)piperazin-2-one; 4-[3-(4-Cyanobenzyl)pyridin-4-yl]-1-(3-chlorophenyl)-5(S)-(2-methylsulfonylethyl)-piperazin-2-one; 4-[5-(4-Cyanobenzyl)-1-imidazolylethyl]-1-(3-chlorophenyl)piperazin-2-one; 4-{3-[4-(−2-Oxo-2-H-pyridin-1-yl)benzyl-3-H-imidazol-4-ylmethyl]benzonitrile; 4-{3-[4-3-Methyl-2-oxo-2-H-pyridin-1-yl)benzyl]-3-H-imidazol-4-ylmethyl]benzonitrile; 4-{3-[4-(−2-Oxo-piperidin-1-yl)benzyl]-3-H-imidazol-4-ylmethyl]benzonitrile; 4-{3-[3-Methyl-4-(2-oxopiperidin-1-yl)-benzyl]-3-H-imidizol-4-ylmethyl}-benzonitrile; (4-{3-[4-(2-Oxo-pyrrolidin-1-yl)-benzyl]-3H-imidizol-4-ylmethyl}-benzonitrile; 4-13-[4-(3-Methyl-2-oxo-2-H-pyrazin-1-yl)-benzyl-3-H-imidizol-4-ylmethyl}-benzonitrile; 4-{3-[2-Methoxy-4-(2-oxo-2-H-pyridin-1-yl)-benzyl]-3-H-imidizol-4-ylmethyl}-benzonitrile; 4-{1-[4-(5-Chloro-2-oxo-2H-pyridin-1-yl)-benzyl]-1H-pyrrol-2-ylmethyl}-benzonitrile; 4-[1-(2-Oxo-2H-[1,2′]bipyridinyl-5′-ylmethyl)-1H-pyrrol-2-ylmethyl]-benzonitrile; 4-[1-(5-Chloro-2-oxo-2H-[1,2′]bipyridinyl-5′-ylmethyl)-1H-pyrrol-2-ylmethyl]-benzonitrile; 4-[3-(2-Oxo-1-phenyl-1,2-dihydropyridin-4-ylmethyl)-3H-imidazol-4-ylmethyl]benzonitrile; 4-{3-[1-(3-Chloro-phenyl)-2-oxo-1,2-dihydropyridin-4-ylmethyl]-3H-imidazol-4-ylmethyl}benzonitrile; 19,20-Dihydro-19-oxo-5H,17H-18,21-ethano-6, 10:12,16-dimetheno-22H -imidazo[3,4-h][1,8,11,14]oxatriazacycloeicosine-9-carbonitrile; 19-Chloro-22,23-dihydro-22-oxo-5H-21,24-ethano-6,10-metheno-25H-dibenzo[b,e]imidazo[4,3-1][1, 4,7,10,13]dioxatriazacyclononadecine-9-carbonitrile; 22,23-Dihydro-22-oxo-5H-21,24-ethano-6,10-metheno-25H-dibenzo[b,e]imidazo[4,3-1][1,4,7,10,13]dioxatriazacyclononadecine-9-carbonitrile; 20-Chloro-23,24-dihydro-23-oxo-5H-22′,25-ethano-6, 10:12,16-dimetheno-12H, 26H-benzo[b]imidazo[4,3-i][1,17,4,7,10]dioxatriazacyclohemicosine-9-carbonitrile; (S)-20-Chloro-23,24-dihydro-27-[2-(methylsulfonyl)ethyl]-23-oxo-5H-22,25-ethano-6, 10:12,16-dimetheno-12H,26H-benzo[b]imidazo[4,3-i][1,17,4,7,10]dioxatriazacyclohemicosine-9-carbonitrile; (±)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile; (+)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile; (−)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile; 5H,17H,20H-18,21-Ethano-6, 10:12,16-dimetheno-22H-imidazo[3,4-h][1,8,11,14]oxatriazacycloeicosin-20-one; (±)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile; (+) or (−)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile; (Enantiomer A) (−) or (+)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile; (Enantiomer B) (±)-19,20-Dihydro-19,22-dioxo-5H-18,21-ethano-12,14-etheno-6,10-metheno-22H-benzo[d]imidazo[4,3-k][1,6,9,12]oxatriazacyclooctadecine-9-carbonitrile; 325 18,19-dihydro-19-oxo-5H,17H-6, 10:12, 16-dimetheno-1H-imidazo[4,3-c][1,11,4]dioxaazacyclononadecine-9-carbonitrile; 17,18-dihydro-18-oxo-5H-6, 10:12,16-dimetheno-12H,20H-imidazo[4,3-c][1,11,4]dioxaazacyclooctadecine-9-carbonitrile; (±)-17,18,19,20-tetrahydro-19-phenyl-5H-6,10:12,16-dimetheno-21H-imidazo[3,4-h][1,8,11]oxadiazacyclononadecine-9-carbonitrile; 21,22-dihydro-5H-6,10:12,16-dimetheno-23H-benzo[g]imidazo[4,3-1][1,8,11]oxadiazacyclononadecine-9-carbonitrile; 22,23-dihydro-23-oxo-5H,21H-6,10:12,16-dimetheno-24H-benzo g]imidazo[4,3-m][1,8,12]oxadiazaeicosine-9-carbonitrile; 22,23-dihydro-5H, 21H-6, 10:12,16-dimetheno-24H-benzo[g]imidazo[4,3-m][1,8,11]oxadiazaeicosine-9-carbonitrile; 1-(3-trifluoromethoxyphenyl)-4-[1-(4-cyano-3-methoxybenzyl)-5-imidazolyl methyl]-2-piperazinone; or a pharmaceutically acceptable salt, stereoisomer or optical isomer thereof. Specific examples of a farnesyl-protein transferase inhibitor are 1-(3-Chlorophenyl)-4-[1-(4-cyanobenzyl)-5-imidazolylmethyl]-2-piperazinone; (R)-1-(3-Chlorophenyl)-4-[1-(4-cyanobenzyl)-5-imidazolylmethyl]-5-[2-(ethanesulfonyl)methyl]-2-piperazinone; 4-[1-(5-Chloro-2-oxo-2H-[1,2′]bipyridinyl-5′-ylmethyl)-1H-pyrrol-2-ylmethyl]-benzonitrile; and 1-[N-(1-(4-cyanobenzyl)-5-imidazolylmethyl)-N-(4-cyanobenzyl)amino]-4-(phenoxy)benzene; (±)-19,20-Dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile; 1-(3-trifluoromethoxyphenyl)-4-[1-(4-cyano-3-methoxybenzyl)-5-imidazolyl methyl]-2-piperazinone; 3-(biphenyl-4-ylmethoxy)-4-imidazol-1-ylmethyl-benzonitrile; 3-(biphenyl-4-yl-2-ethoxy)-4-imidazol-1-ylmethylbenzonitrile; 3-(biphenyl-3-ylmethoxy)-4-imidazol-1-ylmethyl-benzonitrile; 2-(biphenyl-4-ylmethoxy)-4-imidazol-1-ylmethyl-benzonitrile; 2-(biphenyl-4-yl-2-ethoxy)-4-imidazol-1-ylmethyl-benzonitrile; 1-tert-butoxycarbonyl-4-(3-chlorophenyl)-2(S)-[2-(2-cyano-5-imidazol-1-ylmethyl-phenoxy)ethyl]piperazine; 2-(3-chlorophenoxy)-4-imidazol-1-ylmethyl-benzonitrile; 2-(4-chlorophenyl-2-ethoxy)-4-imidazol-1-ylmethyl-benzonitrile; 2-(3-chlorophenyl-2-ethoxy)-4-imidazol-1-ylmethyl-benzonitrile; 2-(2-chlorophenyl-2-ethoxy)-4-imidazol-1-ylmethyl-benzonitrile; 2-(phenyl-2-ethoxy)-4-imidazol-1-ylmethyl-benzonitrile; 2-(3-chlorobenzyloxy)-4-imidazol-1-ylmethyl-benzonitrile; 2-(4-chlorobenzyloxy)-4-imidazol-1-ylmethyl-benzonitrile; 2-(2,4-dichlorobenzyloxy)-4-imidazol-1-ylmethyl-benzonitrile; 2-(benzyloxy)-4-imidazol-1-ylmethyl-benzonitrile; 2-(biphenyl-2-ylmethoxy)-4-imidazol-1-ylmethyl-benzonitrile; 2-(phenyl-4-butoxy)-4-imidazol-1-ylmethyl-benzonitrile; 2-(phenyl-3-propoxy)-4-imidazol-1-ylmethyl-benzonitrile; 2-(biphenyl-4-yl-2-ethoxy)-4-(1,2,4-triazol-1-yl)methyl-benzonitrile; 2-(biphenyl-4-yl-2-ethoxy)-4-(2-methyl-imidazol-1-yl)methyl-benzonitrile; 2-(biphenyl-4-yl-2-ethoxy)-4-benzimidazol-1-yl)methyl-benzonitrile; 4-imidazol-1-ylmethyl-2-(naphthalen-2-yloxy)-benzonitrile; 2-(3-cyanophenoxy)-4-imidazol-1-ylmethyl-benzonitrile; 2-(3-bromophenoxy)-4-imidazol-1-ylmethyl-benzonitrile; 2-(biphen-3-yloxy)-4-imidazol-1-ylmethyl-benzonitrile; 2-(biphen-4-yloxy)-4-imidazol-1-ylmethyl-benzonitrile; 2-(3-acetylphenoxy)-4-imidazol-1-ylmethyl-benzonitrile; 2-(2-acetylphenoxy)-4-imidazol-1-ylmethyl-benzonitrile; 2-(3-trifluoromethylphenoxy)-4-imidazol-1-ylmethyl-benzonitrile; 2-(3-methylphenoxy)-4-imidazol-1-ylmethyl-benzonitrile; 2-(2-methylphenoxy)-4-imidazol-1-ylmethyl-benzonitrile; 2-(4-methylphenoxy)-4-imidazol-1-ylmethyl-benzonitrile; 2-(3-methoxyphenoxy)-4-imidazol-1-ylmethyl-benzonitrile; 2-(2-methoxyphenoxy)-4-imidazol-1-ylmethyl-benzonitrile; 2-(4-methoxyphenoxy)-4-imidazol-1-ylmethyl-benzonitrile; 2-(3,5-dimethylphenoxy)-4-imidazol-1-ylmethyl-benzonitrile; 2-(3,4-dimethylphenoxy)-4-imidazol-1-ylmethyl-benzonitrile; 2-(3,5-dimethoxyphenoxy)-4-imidazol-1-ylmethyl-benzonitrile; 2-(1-naphthyloxy)-4-imidazol-1-ylmethyl-benzonitrile; 2-(2,4-dichlorophenoxy)-4-imidazol-1-ylmethyl-benzonitrile; 2-(3-fluorophenoxy)-4-imidazol-1-ylmethyl-benzonitrile; 2-(3-t-butylphenoxy)-4-imidazol-1-ylmethyl-benzonitrile; 2-[3-(N,N-diethylamino)phenoxy]-4-imidazol-1-ylmethyl-benzonitrile; 2-(3-n-propylphenoxy)-4-imidazol-1-ylmethyl-benzonitrile; 2-(2,3-dimethoxyphenoxy)-4-imidazol-1-ylmethyl-benzonitrile; 2-(2,3-dimethylphenoxy)-4-imidazol-1-ylmethyl-benzonitrile; 2-(3,4-dimethoxyphenoxy)-4-imidazol-1-ylmethyl-benzonitrile; 2-(2,5-dimethoxyphenoxy)-4-imidazol-1-ylmethyl-benzonitrile; 2-(3,4-dichlorophenoxy)-4-imidazol-1-ylmethyl-benzonitrile; 2-(2,4-dimethylphenoxy)-4-imidazol-1-ylmethyl-benzonitrile; 2-(4-chloro-2-methylphenoxy)-4-imidazol-1-ylmethyl-benzonitrile; 2-(5-chloro-2-methylphenoxy)-4-imidazol-1-ylmethyl-benzonitrile; 2-(2-chloro-4,5-dimethylphenoxy)-4-imidazol-1-ylmethyl-benzonitrile; 2-(5-hydroxymethyl-2-methoxyphenoxy)-4-imidazol-1-ylmethyl-benzonitrile; 4-imidazol-1-ylmethyl-2-(3-phenylamino-phenoxy)-benzonitrile; 4-imidazol-1-ylmethyl-2-[3-(2-methylphenylamino)-phenoxy]-benzonitrile; 4-imidazol-1-ylmethyl-2-(3-phenoxy-phenoxy)-benzonitrile; 2-(2-benzoyl-phenoxy)-4-imidazol-1-ylmethyl-benzonitrile; 1-(5-chloro-2-methoxy-phenyl)-3-[3-(2-cyano-5-imidazol-1-ylmethyl-phenoxy)-phenyl]-urea; 1-(2,5-dimethoxy-phenyl)-3-[3-(2-cyano-5-imidazol-1-ylmethyl-phenoxy)-phenyl]-urea; 2-(3-benzyloxy-phenoxy)-4-imidazol-1-ylmethyl-benzonitrile; 2-(4-benzyloxy-phenoxy)-4-imidazol-1-ylmethyl-benzonitrile; 2-(2-benzyl-phenoxy)-4-imidazol-1-ylmethyl-benzonitrile; 2-(3-ethynyl-phenoxy)-4-imidazol-1-ylmethyl-benzonitrile; 2-(4-acetyl-3-methyl-phenoxy)-4-imidazol-1-ylmethyl-benzonitrile; 4-imidazol-1-ylmethyl-2-(1H-indazol-6-yloxy)-benzonitrile; 4-imidazol-1-ylmethyl-2-(5,6,7,8-tetrahydro-naphthalen-1-yloxy)-benzonitrile; 4-imidazol-1-ylmethyl-2-(8-oxo-5,6,7,8-tetrahydro-naphthalen-1-yloxy)-benzonitrile; 4-imidazol-1-ylmethyl-2-(1H-indol-7-yloxy)-benzonitrile; 4-imidazol-1-ylmethyl-2-(3-oxo-indan-4-yloxy)-benzonitrile; 4-imidazol-1-ylmethyl-2-(1H-indol-4-yloxy)-benzonitrile; 2-[3-(2-hydroxy-ethoxy)-phenoxy]-4-imidazol-1-ylmethyl-benzonitrile; 4-imidazol-1-ylmethyl-2-(4-imidazol-1-yl-phenoxy)-benzonitrile; 4-(2-cyano-5-imidazol-1-ylmethyl-phenoxy)-biphenyl-4-carbonitrile; N-[3-(2-cyano-5-imidazol-1-ylmethyl-phenoxy)-phenyl]-acetamide; 4-imidazol-1-ylmethyl-2-(9-oxo-9H-fluoren-4-yloxy)-benzonitrile; 3-(2-cyano-5-imidazol-1-ylmethyl-phenoxy)-Nphenyl-benzamide; 3-(2-cyano-5-imidazol-1-ylmethyl-phenoxy)-N-ethyl-N-phenyl-benzamide; 3-(2-cyano-5-imidazol-1-ylmethyl-phenoxy)-N-cyclopropylmethyl-N-phenyl-benzamide; 2-(5-chloro-pyridin-3-yloxy)-4-imidazol-1-ylmethyl-benzonitrile; N-[3-(2-cyano-5-imidazol-1-ylmethyl-phenoxy)-phenyl]-benzenesulfonamide; 4-imidazol-1-ylmethyl-2-(indan-5-yloxy)-benzonitrile; 3-(9H-carbazol-2-yloxy)-4-imidazol-1-ylmethyl-benzonitrile; 4-imidazol-1-ylmethyl-2-(5,6,7,8-tetrahydro-naphthalen-2-yloxy)-benzonitrile; 4-imidazol-1-ylmethyl-2-(2-methoxy-4-propenyl-phenoxy)-benzonitrile; 4-imidazol-1-ylmethyl-2-[4-(3-oxo-butyl)-phenoxy]-benzonitrile; 2-(3-chlorophenoxy)-5-imidazol-1-ylmethyl-benzonitrile; 2-(4-chlorophenoxy)-4-imidazol-1-ylmethyl-benzonitrile; 2-(3,5-dichlorophenoxy)-4-imidazol-1-ylmethyl-benzonitrile; 2-(pyridin-3-yloxy)-4-imidazol-1-ylmethyl-benzonitrile; 2-(2-chlorophenoxy)-4-imidazol-1-ylmethyl-benzonitrile; 2-(3-chlorophenoxy)-5-(4-phenyl-imidazol-1-ylmethyl)-benzonitrile; 2-(biphen-2-yloxy)-4-imidazol-1-ylmethyl-benzonitrile; 2-(phenoxy)-4-imidazol-1-ylmethyl-benzonitrile; 2-(2-chloro-4-methoxyphenoxy)-4-imidazol-1-ylmethyl-benzonitrile; 2-(2-chlorophenylsulfanyl)-4-imidazol-1-ylmethyl-benzonitrile; 4-imidazol-1-ylmethyl-2-(naphthalen-2-ylsulfanyl)-benzonitrile; 2-(2,4-dichlorophenylsulfanyl)-4-imidazol-1-ylmethyl-benzonitrile; 2-(2,4-dichloro-benzenesulfinyl)-4-imidazol-1-ylmethyl-benzonitrile; 2-(2,4-dichloro-benzenesulfonyl)-4-imidazol-1-ylmethyl-benzonitrile; 2-(2-methyl-pyridin-3-yloxy)-4-imidazol-1-ylmethyl-benzonitrile; 2-(2,4-dimethyl-pyridin-3-yloxy)-4-imidazol-1-ylmethyl-benzonitrile; 2-(4-chloro-2-methoxyphenoxy)-4-imidazol-1-ylmethyl-benzonitrile; 2-(2-chlorophenoxy)-4-(5-methyl-imidazol-1-ylmethyl)-benzonitrile; 2-(2-chlorophenoxy)-4-(4-methyl-imidazol-1-ylmethyl)-benzonitrile; 2-(3-chloro-5-trifluoromethyl-pyridin-2-yloxy)-4-imidazol-1-ylmethyl-benzonitrile; 2-(2,4-dichlorophenoxy)-4-(2-methyl-imidazol-1-ylmethyl)-benzonitrile; N-[3-(2-cyano-5-imidazol-1-ylmethyl-phenoxy)-phenyl]-benzamide; 2-[3-(2-cyano-5-imidazol-1-ylmethyl-phenoxy)-phenyl]-N-phenyl-acetamide; 4-imidazol-1-ylmethyl-2-(quinolin-6-yloxy)-benzonitrile; 4-imidazol-1-ylmethyl-2-(2-oxo-1,2-dihydro-quinolin-6-yloxy)-benzonitrile; N-[3-(2-cyano-5-imidazol-1-ylmethyl-phenoxy)-phenyl]-2-phenyl-acetamide; 5-(2-cyano-5-imidazol-1-ylmethyl-phenoxy)-N-cyclohexyl-nicotinamide; N-(3-chloro-phenyl)-5-(2-cyano-5-imidazol-1-ylmethyl-phenoxy)-nicotinamide; 2-(2,3-dimethoxyphenoxy)-4-(2,4-dimethyl-imidazol-1-ylmethyl)-benzonitrile; 4-(2-methyl-imidazol-1-ylmethyl)-2-(naphthalen-2-yloxy)-benzonitrile; 4-(1-imidazol-1-yl-1-methyl-ethyl)-2-(naphthalen-2-yloxy)-benzonitrile; 1-[4-iodo-3-(naphthalen-2-yloxy)-benzyl]-1H-imidazole; acetic acid 3-[3-(2-chloro-phenoxy)-4-cyano-benzyl]-3H-imidazol-4-ylmethyl ester; 2-(2-chloro-phenoxy)-4-(5-hydroxymethyl-imidazol-1-ylmethyl)-benzonitrile; 4-(5-aminomethyl-imidazol-1-ylmethyl)-2-(2-chloro-phenoxy)-benzonitrile; N-{3-[4-cyano-3-(2,3-dimethoxy-phenoxy)-benzyl]-3H-imidazol-4-ylmethyl}-2-cyclohexyl-acetamide; 2-(3-chloro-phenoxy)-4-[(4-chloro-phenyl)-imidazol-1-yl-methyl]-benzonitrile; 2-(3-chloro-phenoxy)-4-[1-(4-chloro-phenyl)-2-hydroxy-1-imidazol-1-yl-ethyl]-benzonitrile; 2-(3-chloro-phenoxy)-4-[(4-chloro-phenyl)-hydroxy-(3H-imidazol-4-yl)-methyl]-benzonitrile; 2-(2,4-dichloro-phenylsulfanyl)-4-[5-(2-morpholin-4-yl-ethyl)-imidazol-1-ylmethyl]-benzonitrile; 2-(2,4-dichloro-phenoxy)-4-[5-(2-morpholin-4-yl-ethyl)-imidazol-1-ylmethyl]-benzonitrile; 4-[hydroxy-(3-methyl-3H-imidazol-4-yl)-methyl]-2-(naphthalen-2-yloxy)-benzonitrile; 4-[amino-(3-methyl-3H-imidazol-4-yl)-methyl]-2-(naphthalen-2-yloxy)-benzonitrile; 4-[1-hydroxy-1-(3-methyl-3H-imidazol-4-yl)-ethyl]-2-(naphthalen-2-yloxy)-benzonitrile; 4-[1-amino-1-(3-methyl-3H-imidazol-4-yl)-ethyl]-2-(naphthalen-2-yloxy)-benzonitrile hydrochloride; 3-{2-cyano-5-[1-amino-1-(3-methyl-3H-imidazol-4-yl)-ethyl]-phenoxy}-N-ethyl-N-phenyl-benzamide; 3-{2-cyano-5-[1-hydroxy-1-(3-methyl-3H-imidazol-4-yl)-ethyl]-phenoxy}-N-ethyl-N-phenyl-benzamide; 4-[1-hydroxy-1-(3-methyl-3H-imidazol-4-yl)-ethyl]-2-(3-phenylamino-phenoxy)-benzonitrile; 4-[1-hydroxy-1-(3-methyl-3H-imidazol-4-yl)-ethyl]-2-(3-phenoxy-phenoxy)-benzonitrile; 2-(3-benzoyl-phenoxy)-4-[1-hydroxy-1-(3-methyl-3H-imidazol-4-yl)-ethyl]-benzonitrile; 2-(3-tert-butyl-phenoxy)-4-[1-hydroxy-1-(3-methyl-3H-imidazol-4-yl)-ethyl]-benzonitrile; 2-(3-diethylamino-phenoxy)-4-[1-hydroxy-1-(3-methyl-3H-imidazol-4-yl)-ethyl]-benzonitrile; 2-(5-chloro-2-oxo-2H-[1,2′]bipyridinyl-5′-ylmethoxy)-4-imidazol-1-ylmethyl-benzonitrile; 4-Imidazol-1-ylmethyl-2-[2-(2-oxo-2H-pyridin-1-yl)-phenoxy]-benzonitrile; 4-Imidazol-1-ylmethyl-2-[3-(2-oxo-2H-pyridin-1-yl)-phenoxy]-benzonitrile; 4-Imidazol-1-ylmethyl-2-[4-(2-oxo-2H-pyridin-1-yl)-phenoxy]-benzonitrile; 4-imidazol-1-ylmethyl-2-[3-(2-oxo-piperidin-1-yl)-phenoxy]-benzonitrile; 4-imidazol-1-ylmethyl-2-[4-(2-oxo-piperidin-1-yl)-phenoxy]-benzonitrile; 4-imidazol-1-ylmethyl-2-[2-(3-methyl-2-oxo-piperidin-1-yl)-phenoxy]-benzonitrile; 4-imidazol-1-ylmethyl-2-(3-morpholin-4-yl-phenoxy)-benzonitrile; 4-imidazol-1-ylmethyl-2-(3-piperidin-1-ylmethyl-phenoxy)-benzonitrile; 2-[2-(3,3-dimethyl-2-oxo-piperidin-1-yl)-phenoxy]-4-imidazol-1-ylmethyl-benzonitrile; 2-[3-(3-ethyl-1-methyl-2-oxo-azepan-3-yl)-phenoxy]-4-imidazol-1-ylmethyl-benzonitrile; 2-[3-(3-ethyl-1-methyl-2-oxo-azepan-3-yl)-phenoxy]-4-(2-methyl-imidazol-1-yl)methyl-benzonitrile; 2-[3-(3-ethyl-1-methyl-2-oxo-azepan-3-yl)-phenoxy]-4-(5-methyl-imidazol-1-yl)methyl-benzonitrile; 2-[3-(3-ethyl-1-methyl-2-oxo-azepan-3-yl)-phenoxy]-4-(2,5-dimethyl-imidazol-1-yl)methyl-benzonitrile; 2-[3-(3-ethyl-1-methyl-2-oxo-azepan-3-yl)-phenoxy]-4-[1,2,4]triazol-4-ylmethyl-benzonitrile; 2-[3-(3-ethyl-1-methyl-2-oxo-azepan-3-yl)-phenoxy]-4-[1,2,4]triazol-1-ylmethyl-benzonitrile; 4-imidazol-1-ylmethyl-2-[3-(1-methyl-2-oxo-azepan-3-yl)-phenoxy]-benzonitrile; 4-imidazol-1-ylmethyl-2-[3-(1-methyl-2-oxo-azocan-3-yl)-phenoxy]-benzonitrile; 4-imidazol-1-ylmethyl-2-[3-(1-methyl-2-oxo-piperidin-3-yl)-phenoxy]-benzonitrile; 4-imidazol-1-ylmethyl-2-[3-(3-ethyl-1-methyl-2-oxo-piperidin-3-yl)-phenoxy]-benzonitrile; 4-imidazol-1-ylmethyl-2-[3-(2-oxo-azepan-3-yl)-phenoxy]-benzonitrile; 2-[3-(3-hydroxymethyl-1-methyl-2-oxo-azepan-3-yl)-phenoxy]-4-imidazol-1-ylmethyl-benzonitrile; 2-[3-(3-cyclopropylmethyl-1-methyl-2-oxo-azepan-3-yl)-phenoxy]-4-imidazol-1-ylmethyl-benzonitrile; 2-[4-bromo-3-(3-cyclopropylmethyl-1-methyl-2-oxo-azepan-3-yl)-phenoxy]-4-imidazol-1-ylmethyl-benzonitrile; 2-[3-(3-methoxymethyl-1-methyl-2-oxo-azepan-3-yl)-phenoxy]-4-imidazol-1-ylmethyl-benzonitrile; 2-[3-(3-ethyl-2-oxo-azepan-3-yl)-phenoxy]-4-imidazol-1-ylmethyl-benzonitrile; 2-[3-(3-ethyl-azepan-3-yl)-phenoxy]-4-imidazol-1-ylmethyl-benzonitrile; 2-[3-(1-acetyl-3-ethyl-azepan-3-yl)-phenoxy]-4-imidazol-1-ylmethyl-benzonitrile; 3-[3-(2-cyano-5-imidazol-1-ylmethyl-phenoxy)-phenyl]-3-ethyl-azepane-1-carboxylic acid-tert-butyl ester; 4-[5-(2-amino-ethyl)-2-methyl-imidazol-1-ylmethyl]-2-[3-(3-ethyl-1-methyl-2-oxo-azepan-3-yl)-phenoxy]-benzonitrile; 2-[3-(3-ethyl-1-methyl-2-oxo-azepan-3-yl)-phenoxy]-4-[2-methyl-5-(2-morpholin-4-yl-ethyl)-imidazol-1-ylmethyl]-benzonitrile; N-[2-(3-{4-cyano-3-[3-(3-ethyl-1-methyl-2-oxo-azepan-3-yl)-phenoxy]-benzyl}-2-methyl-3H-imidazol-4-yl)-ethyl]-acetamide; 3-ethyl-3-[3-(3-imidazol-1-ylmethyl-phenoxy)-phenyl]-1-methyl-azepan-2-one; 2-[3-(3-ethyl-1-methyl-2-oxo-azepan-3-yl)-phenoxy]-4-(3-methyl-3-H-imidazol-4-ylmethyl)-benzonitrile; 2-[3-(3-ethyl-1-methyl-2-oxo-azepan-3-yl)-phenoxy]-4-(3H-imidazol-4-ylmethyl)-benzonitrile; 2-[3-(3-ethyl-1-methyl-2-oxo-azepan-3-yl)-phenoxy]-4-[hydroxy-(3-methyl-3-H-imidazol-4-yl)-methyl]-benzonitrile; 4-[amino-(3-methyl-3-H-imidazol-4-yl)-methyl]-2-[3-(3-ethyl-1-methyl-2-oxo-azepan-3-yl)-phenoxy]-benzonitrile; 2-[3-(3-ethyl-1-methyl-2-oxo-azepan-3-yl)-benzyl]-4-(3-methyl-3H-imidazole-4-carbonyl)-benzonitrile; 2-[3-(3-ethyl-1-methyl-2-oxo-azepan-3-yl)-phenoxy]-4-(hydroxy-pyridin-3-yl-methyl)-benzonitrile; 2-[3-(3-ethyl-1-methyl-2-oxo-azepan-3-yl)-phenoxy]-4-pyridin-3-ylmethyl-benzonitrile; 2-[3-(3-ethyl-1-methyl-2-oxo-azepan-3-yl)-phenoxy]-4-pyridin-2-ylmethyl-benzonitrile; 2-[3-(3-ethyl-1-methyl-2-oxo-azepan-3-yl)-phenoxy]-4-[1-hydroxy-1-(3-methyl-3H-imidazol-4-yl)-ethyl]-benzonitrile; 2-[3-(3-ethyl-1-methyl-2-oxo-azepan-3-yl)-phenoxy]-4-[1]-amino-1-(3-methyl-3H-imidazol-4-yl)-ethyl]-benzonitrile; 1-[1-(4′-Cyanobenzyl) imidazol-5-ylmethyl]-4-[1-phenyl-1-cyclopentylcarbonyl]piperazine; 1-[1-(4′-Cyanobenzyl) imidazol-5-ylmethyl]-4-[Cyclohexylphenylacetyl]piperazine; 1-[1-(4′-Cyanobenzyl)imidazol-5-ylmethyl]-4-[1-(3-methoxyphenyl)-1-cyclopentylcarbonyl]piperazine; 1-[1-(4′-Cyanobenzyl) imidazol-5-ylmethyl]-4-[1-(3-phenoxyphenyl)-1-cyclopentylcarbonyl]piperazine; 1-[1-(4′-Cyano-3-fluorobenzyl)imidazol-5-ylmethyl]-4-[1-(3-hydroxyphenyl)-1-cyclohexylcarbonyl]piperazine; 1-[1-(4′-Cyanobenzyl)imidazol-5-ylmethyl]piperazine-4-carboxylic acid-(2,6-dimethoxy)benzyl ester; 1-[1-(4′-Cyanobenzyl)imidazol-5-ylmethyl]piperazine-4-(DL-2-hydroxy-2-(o-methoxyphenyl)) acetamide; 1-[1-(4′-Cyanobenzyl) imidazol-5-ylmethyl]-4-[1-(2,6-dimethylbenzyloxycarbonyl]piperazine; 1-[1-(4′-Cyanobenzyl) imidazol-5-ylmethyl]-4-[1-(2-methoxyphenyl)-1-cyclopentylcarbonyl]piperazine; (+/−) 1-[1-(4′-Cyanobenzyl)imidazol-5-ylmethyl]-4-[1-(bicyclo[3.1.0]hex-3-yl)-1-(3-methoxyphenyl)-carbonyl]piperazine; (R/S) 2[4-((Phenyl)methyloxycarbonyl-1-piperazine)]-2-[1-(4′-cyanobenzyl)-2-methyl-5-imidazol]acetonitrile; 1-[1-(4′-methylbenzyl)imidazol-5-ylmethyl]-4-[1-(2,6-dimethylbenzyloxycarbonyl]piperazine; 1-[1-(4′-Cyanobenzyl)imidazol-5-ylmethyl]piperazine-4-carboxylic acid-(4-nitro)phenyl ester; 1-[1-(4-Cyanobenzyl)imidazol-5-ylmethyl]-4-[3-(4-fluorophenyl)-3-(tricyclo[3.3.1.1^{3 7}]dec-2-yl)-propionyl]piperazine; 2-(1-(4′-cyanobenzyl)imidazol-5-yl-2-[4-(phenylmethyloxy carbonyl)piperazin-1-yl]acetamide; 1-[1-(4′-cyanobenzyl)imidazol-5-ylmethyl]-4-[1-(2-methoxy-5-chlorobenzyloxycarbonyl]piperazine; 1-[1-(4′-cyanobenzyl)imidazol-5-ylmethyl]-4-[1-(pentafluororobenzyloxycarbonyl]piperazine; 1-[1-(4′-cyanobenzyl)imidazol-5-ylmethyl]-4-[1-(2-ethoxybenzyloxycarbonyl]piperazine; 1-[1-(4′-cyanobenzyl)imidazol-5-ylmethyl]-4-{1-[(2-methoxypyridin-3-yl)methyloxycarbonyl]}piperazine; 1-[1-(4′-cyanobenzyl)imidazol-5-ylmethyl]-4-[1-(2-trifluoromethoxybenzyloxycarbonyl]piperazine; 1-[1-(4′-cyanobenzyl)imidazol-5-ylmethyl]-4-[1-(2,3-methylenedioxybenzyloxycarbonyl]piperazine; 1-[1-(4′-Cyanobenzyl)imidazol-5-ylmethyl]piperazine-4-carboxylic acid benzyl ester; 1-[1-(4′-Cyanobenzyl)imidazol-5-ylmethyl]-piperazine-3-carboxylic acid-4-carboxylic acid benzyl ester; 1-[1-(4′-Cyanobenzyl)imidazol-5-ylmethyl]-3-methyl carboxy-piperazine-4-carboxylic acid, or a stereoisomeric form, or a pharmaceutically acceptable acid or base addition salt form thereof, in a therapeutically effective amount.

In one embodiment, the compound may be one or more of the following: 1-(3-Chlorophenyl)-4-[1-(4-cyanobenzyl)-5-imidazolylmethyl]-2-piperazinone; (R)-1-(3-Chlorophenyl)-4-[1]-(4-cyanobenzyl)-5-imidazolylmethyl]-5-[2-(ethanesulfonyl)methyl]-2-piperazinone; 4-[1-(5-Chloro-2-oxo-2H-[1,2′]bipyridinyl-5′-ylmethyl)-1H-pyrrol-2-ylmethyl]-benzonitrile and 1-[N-(1-(4-cyanobenzyl)-5-imidazolylmethyl)-N-(4-cyanobenzyl)amino]-4-(phenoxy)benzene, or a stereoisomeric form, or a pharmaceutically acceptable acid or base addition salt form thereof, in a therapeutically effective amount.

In another aspect, the invention provides a method of treating a synucleinopathic subject by administering a one or more farnesyl transferase inhibitor compounds described in U.S. Pat. No. 5,919,785 and U.S. Pat. No. 5,859,012 (the disclosures of which are incorporated herein by reference) or a stereoisomeric form, or a pharmaceutically acceptable acid or base addition salt form thereof, in a therapeutically effective amount.

In another aspect, the invention provides a method of treating a synucleinopathic subject by administering a farnesyl transferase inhibitor compound of the formula: 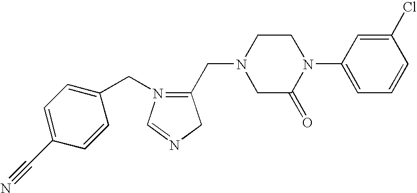
or a stereoisomeric form, or a pharmaceutically acceptable acid or base addition salt form thereof, in a therapeutically effective amount.

Accordingly, in one aspect, the invention provides a method of treating a synucleinopathic subject by administering a farnesyl transferase inhibitor compound of the formula: 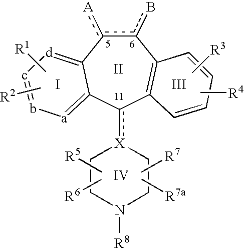
or a stereoisomeric form, or a pharmaceutically acceptable acid or base addition salt form thereof, in a therapeutically effective amount, wherein:

one of a, b, c and d represents N or N⁺O⁻, and the remaining a, b, c, and d groups represent carbon, wherein each carbon has an R¹ or R² group bound to said carbon; or

each of a, b, c, and d is carbon, wherein each carbon has an R¹ or R² group bound to said carbon;

the dotted line (- - -) represents optional bonds;

X represents N or CH when the optional bond to C11 is absent, and represents C when the optional bond to C11 is present;

when the optional bond is present between carbon atom 5 and carbon atom 6 then there is only one A substituent bound to C-5 and there is only one B substituent bound to C-6 and A or B is other than H;

when the optional bond is not present between carbon atom 5 and carbon atom 6 then there are two A substituents bound to C-5, wherein each A substituent is independently selected, and two B substituents bound to C-6, wherein each B substituent is independently selected, and wherein at least one of the two A substituents or one of the two B substituents are H, and wherein at least one of the two A substituents or one of the two B substituents is other than H;

A and B are independently selected from the group consisting of: (1) H; (2) —R⁹; (3) —R⁹—C(O)—R⁹; (4) —R⁹—CO₂—R^{9a}; (5) —(CH₂)ₚR²⁶; (6) —C(O)N(R⁹)₂, wherein each R⁹ is the same or different; (7) —C(O)NHR⁹; (8) —C(O)NH—CH₂—C(O)—NH₂; (9) —C(O)NHR²⁶; (10) —(CH₂)ₚC(R⁹)—O—R^{9a}; (11) —(CH₂)ₚ(R⁹)₂, wherein each R⁹ is the same or different; (12) —(CH₂)ₚC(O)R⁹; (13) —(CH₂)ₚC(O)R²⁷; (14) —(CH₂)ₚC(O)N(R⁹)₂, wherein each R⁹ is the same or different; (15) —(CH₂)ₚC(O)NH(R⁹); (16) —(CH₂)ₚC(O)N(R²⁶)₂, wherein each R²⁶ is the same or different; (17) —(CH₂)ₚN(R⁹)—R^{9a}; (18) —(CH₂)ₚN(R²⁶)₂, wherein R²⁶ is the same or different; (19) —(CH₂)ₚNHC(O)R⁵; (20) —(CH₂)ₚNHC(O)₂R⁵⁰; (21) —(CH₂)ₚN(C(O)R^{27a})₂ wherein each R^{27a} is the same or different; (22) —(CH₂)ₚNR⁵¹C(O)R²⁷; (23) —(CH₂)ₚNR⁵¹C(O)R²⁷ wherein R⁵¹ is not H, and R⁵¹ and R²⁷ taken together with the atoms to which they are bound form a 5 or 6 membered heterocycloalkyl ring consisting; (24) —(CH₂)ₚNR⁵¹C(O)NR²⁷; (25) —(CH₂)ₚNR⁵¹C(O)NR²⁷ wherein R⁵¹ is not H, and R⁵¹ and R²⁷ taken together with the atoms to which they are bound form a 5 or 6 membered heterocycloalkyl ring; (26) —(CH₂)ₚNR⁵¹C(O)N(R^{27a})₂, wherein each R^{27a} is the same or different; (27) —(CH₂)ₚNHSO₂N(R⁵¹)₂, wherein each R⁵¹ is the same or different; (28) —(CH₂)ₚNHCO₂R⁵⁰; (29) —(CH₂)ₚNC(O)NHR⁵¹; (30) —(CH₂)ₚCO₂R⁵¹; (31) —NHR⁹; (32) 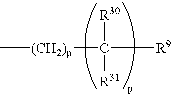
wherein R³⁰ and R³¹ are the same or different, and each p is independently selected; (33)

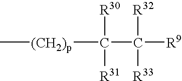

wherein R³⁰, R³¹, R³² and R³³ are the same or different; (34) -alkenyl-CO₂R^{9a}; (35) -alkenyl-C(O)R^{9a}; (36) -alkenyl-CO₂R⁵¹; (37) -alkenyl-C(O)—R^{27a}; (38) (CH₂)ₚ-alkenyl-CO₂—R⁵¹; (37) —(CH₂)ₚC═NOR⁵¹; and (39) —(CH₂)ₚ-phthalimid;

p is 0, 1, 2, 3 or 4;

each R¹ and R² is independently selected from the group consisting of: (1) H; (2) Halo; (3) —CF₃, (4) —OR¹⁰; (5) —COR¹⁰; (6) —SR¹⁰; (7) —S(O)ₜR¹⁵ wherein t is 0, 1 or 2; (8) —N(R¹⁰)₂; (9) —NO₂; (10) —OC(O)R¹⁰; (11) —CO₂R¹⁰; (12) —OCO₂R¹⁵; (13) —CN; (14) —NR¹⁰COOR¹⁵; (15) —SR¹⁵C(O)OR¹⁵; (16) —SR¹⁵N(R¹³)₂ provided that R¹⁵ in —SR¹⁵N(R³)₂ is not —CH₂ and wherein each R is independently selected from the group consisting of: H and —C(O)OR¹⁵; (17) benzotriazol-1-yloxy; (18) tetrazol-5-ylthio; (19) substituted tetrazol-5-ylthio; (20) alkynyl; (21) alkenyl; and (22) alkyl, said alkyl or alkenyl group optionally being substituted with halogen, —OR¹⁰ or —CO₂R¹⁰;

R³ and R⁴ are the same or different and each independently represent H, and any of the substituents of R¹ and R²;

R⁵, R⁶, R⁷ and R^{7a} each independently represent: H, —CF₃, —COR¹⁰, alkyl or aryl, said alkyl or aryl optionally being substituted with —S(O)ₜR¹⁵, —NR¹⁰COOR¹⁵, —C(O)R¹⁰; or —CO₂R¹⁰, or R⁵ is combined with R⁶ to represent ═O or ═S;

R⁸ is selected from the group consisting of:

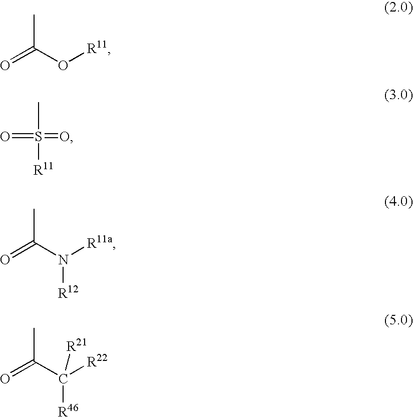

R⁹ is selected from the group consisting of: (1) unsubstituted heteroaryl; (2) substituted heteroaryl; (3) arylalkoxy; (4) substituted arylalkoxy; (5) heterocycloalkyl; (6) substituted heterocycloalkyl; (7) heterocycloalkylalkyl; (8) substituted heterocycloalkylalkyl; (9) unsubstituted heteroarylalkyl; (10) substituted heteroarylalkyl; (11) unsubstituted heteroarylalkenyl; (12) substituted heteroarylalkenyl; (13) unsubstituted heteroarylalkynyl and (14) substituted heteroarylalkynyl;

wherein said substituted R⁹ groups are substituted with one or more substituents selected from the group consisting of: (1) —OH; (2) —CO₂R¹⁴; (3) —CH₂OR¹⁴; (4) halogen; (5) alkyl; (6) amino; (7) trityl; (8) heterocycloalkyl; (9) cycloalkyl; (10) arylalkyl; (11) heteroaryl; (12) heteroarylalkyl and

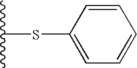

wherein R¹⁴ is independently selected from the group consisting of: H; alkyl; aryl, arylalkyl, heteroaryl and heteroarylalkyl;

R^{9a} is selected from the group consisting of: alky and arylalkyl;

R¹⁰ is selected from the group consisting of: H; alkyl; aryl and arylalkyl;

R¹¹ is selected from the group consisting of: (1) alkyl; (2) substituted alkyl; (3) unsubstituted aryl; (4) substituted aryl; (5) unsubstituted cycloalkyl; (6) substituted cycloalkyl; (7) unsubstituted heteroaryl; (8) substituted heteroaryl; (9) heterocycloalkyl; and (10) substituted heterocycloalkyl; wherein said substituted alkyl, substituted cycloalkyl, and substituted heterocycloalkyl R¹¹ groups are substituted with one or more substituents selected from the group consisting of: (1) —OH; (2) fluoro; and (3) alkyl; and wherein said substituted aryl and substituted heteroaryl R¹¹ groups are substituted with one or more substituents independently selected from the group consisting of: (1) —OH; (2) halogen; and (3) alkyl;

R^{11a} is selected from the group consisting of: (1) H; (2) OH; (3) alkyl; (4) substituted alkyl; (5) unsubstituted aryl; (6) substituted aryl; (7) unsubstituted cycloalkyl; (8) substituted cycloalkyl; (9) unsubstituted heteroaryl; (10) substituted heteroaryl; (11) heterocycloalkyl; and (12) substituted heterocycloalkyl; wherein said substituted alkyl, substituted cycloalkyl, and substituted heterocycloalkyl R^{11a} groups are substituted with one or more substituents independently selected from the group consisting of: (1) —OH; (2) —CN; (3) —CF₃; (4) fluoro; (5) alkyl; (6) cycloalkyl; (7) heterocycloalkyl; (8) arylalkyl; (9) heteroarylalkyl; (10) alkenyl and (11) heteroalkenyl; and wherein said substituted aryl and substituted heteroaryl R^{11a} groups have one or more substituents independently selected from the group consisting of: (1) —OH; (2) —CN; (3) —CF₃; (4) halogen; (5) alkyl; (6) cycloalkyl; (7) heterocycloalkyl; (8) arylalkyl; (9) heteroarylalkyl; (10) alkenyl; and (11) heteroalkenyl;

R¹² is selected from the group consisting of: H, alkyl, piperidine Ring V, cycloalkyl, and -alkyl-(piperidine Ring V);

R¹⁵ is selected from the group consisting of: alkyl and aryl;

R²¹, R²² and R⁴⁶ are independently selected from the group consisting of: (1) —H; (2) alkyl; (3) unsubstituted aryl; (4) substituted aryl substituted with one or more substituents independently selected from the group consisting of: alkyl, halogen, CF₃ and OH; (5) unsubstituted cycloalkyl; (6) substituted cycloalkyl substituted with one or more substituents independently selected from the group consisting of: alkyl, halogen, CF₃ and OH; (7) heteroaryl of the formula, 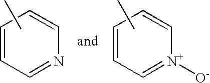
and (8) heterocycloalkyl of the formula: 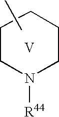
wherein R⁴⁴ is selected from the group consisting of: (a) —H, (b) alkyl; (c) alkylcarbonyl; (d) alkyloxy carbonyl; (e) haloalkyl; and (f) —C(O)NH(R⁵¹);

R²⁶ is selected from the group consisting of: (1) H; (2) alkyl; (3) alkoxyl; (4) —CH₂—CN; (5) R⁹; (6) —CH₂CO₂H; (7) —C(O)alkyl; and (8) CH₂CO₂alkyl;

R²⁷ is selected from the group consisting of: (1) —H; (2) —OH; (3) alkyl; and (4) alkoxy;

R^{27a} is selected from the group consisting of: (1) alkyl; and (2) alkoxy;

R³⁰, R³¹, R³² and R³³ are independently selected from the group consisting of: (1) —H; (2) —OH; (3) ═O; (4) alkyl; (5) aryl (e.g. phenyl); (6) arylalkyl (e.g. benzyl); (7) —OR^{9a}; (8) —NH₂; (9) —NHR^{9a}; and (10) —N(R^{9a})₂ wherein each R^{9a} is independently selected;

R⁵⁰ is selected from the group consisting of: (1) alkyl; (2) unsubstituted heteroaryl; (3)

substituted heteroary; and (4) amino; wherein said substituents on said substituted R⁵⁰ groups are independently selected from the group consisting of: alkyl, halogen, and —OH;

R⁵¹ is selected from the group consisting of: H, and alkyl;

provided that a ring carbon atom adjacent to a ring heteroatom in a substituted heterocycloalkyl moiety is not substituted with a heteroatom or a halo atom; and provided that a ring carbon atom, that is not adjacent to a ring heteroatom, in a substituted heterocycloalkyl moiety, is not substituted with more than one heteroatom; and provided that a ring carbon atom, that is not adjacent to a ring heteroatom, in a substituted heterocycloalkyl moiety, is not substituted with a heteroatom and a halo atom; and provided that a ring carbon in a substituted cycloalkyl moiety is not substituted with more than one heteroatom; and provided that a carbon atom in a substituted alkyl moiety is not substituted with more than one heteroatom; and provided that the same carbon atom in a substituted alkyl moiety is not substituted with both heteroatoms and halo atoms.

In one embodiment, the compound has the formula: 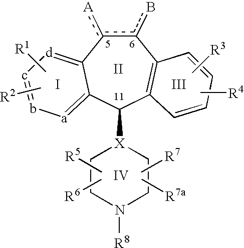
X═CH or N; B is H when the optional bond is present between C-5 and C-6, and when the optional bond between C-5 and C-6 is absent then each B is H.

In another embodiment, the compound has the formula: 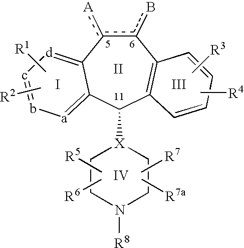
X═CH or N; A is H when the optional bond is present between C-5 and C-6, and when the optional bond between C-5 and C-6 is absent then each A is H.

In any embodiment of this aspect of the invention, R¹ to R⁴ each may be independently selected from H or halo. R⁵ to R⁷ may be H. In one embodiment, a may be N and the remaining b, c and d substituents may be carbon. In another embodiment, a, b, c, and d may be carbon. The optional bond between C-5 and C-6 may be present. Alternatively, the optional bond between C-5 and C-6 may be absent. R⁸ may be group 2.0, or 4.0. One of A and B may be H and the other may be R⁹. R⁹ may be selected from the group consisting of: (1) heterocycloalkylalkyl of the formula —(CH₂)ₙ-heterocycloalkyl; (2) substituted heterocycloalkylalkyl of the formula —(CH₂)ₙ-substituted heterocycloalkyl; (3) unsubstituted heteroarylalkyl of the formula —(CH₂)ₙ-heteroaryl; and (4) substituted heteroarylalkyl of the formula —(CH₂)ₙ-substituted heteroaryl; wherein n is 1, 2, or 3 and the substituents for said substituted R⁹ groups are each independently selected from the group consisting of: (1) —OH; (2) —CO₂R¹⁴; (3) —CH₂OR¹⁴, (3) halo, (4) alkyl; (5) amino; (6) trityl; (7) heterocycloalkyl; (8) arylalkyl; (9) heteroaryl and (10) heteroarylalkyl. wherein R¹⁴ is independently selected from the group consisting of: H and alkyl. In another embodiment, R⁹ may be selected from the group consisting of: (1) —(CH₂)ₙ-imidazolyl; (2) —(CH₂)ₙ-substituted imidazolyl; (3) —(CH₂)ₙ-morpholinyl; (4) —(CH₂)ₙ-substituted morpholinyl, (5) —(CH₂)ₙ-piperazinyl, and (6) —(CH₂)ₙ-substituted piperazinyl, wherein n is 1, 2, or 3. R¹¹ may be selected from the group consisting of: alkyl, cycloalkyl and substituted cycloalkyl wherein the substituents are selected from the group consisting of: halo, alkyl and amino; and R^{11a} may be selected from: alkyl, unsubstituted aryl, 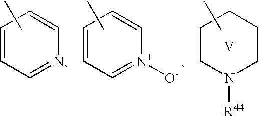
and substituted aryl, cycloalkyl or substituted cycloalkyl, wherein the substituents on said substituted groups are selected from the group consisting of: halo, —CN or CF₃; (3) R², R², and R²² are H; and (4) R⁴⁶ is selected from the group consisting of: unsubstituted aryl, 2247 substituted aryl wherein the substituents are selected from the group consisting of: alkyl, alkylcarbonyl and haloalkyl, and wherein R⁴⁴ is selected from the group consisting of: H or —C(O)NH₂. In another embodiment, R⁸ may be selected from the group consisting of: (1) group 2.0 wherein R¹¹ is selected from the group consisting of: t-butyl and cyclohexyl; (2) group 3.0 wherein R¹¹ is selected from the group consisting of: methyl and t-butyl; (3) group 4.0 wherein, R¹² is H, and R^{11a} is selected from the group consisting of: t-butyl, cyanophenyl, chlorophenyl, fluorophenyl and cyclohexyl; (4) group 5.0 wherein R²¹ and R²² are H, and R⁴⁶ is selected from the group consisting of: 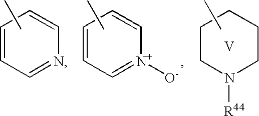
wherein R⁴⁴ is —C(O)NH₂. R⁸ may be group 4.0.

In one embodiment, the optional bond between C5 and C6 may be present and A is H and B is R⁹.

In one embodiment, (1) R¹ to R⁴ each may be independently selected from the group consisting of: H and halo; (2) R⁵, R⁶, R⁷, and R^{7a} are H; (3) a is N and the remaining b, c and d substituents are carbon; (4) the optional bond between C5 and C6 is present; (5) A is H; (6) B is R⁹; (7) R⁸ is group 2.0 or 4.0; (8) R¹¹ is selected from the group consisting of: alkyl, cycloalkyl and substituted cycloalkyl wherein the substituents are selected from the group consisting of: halo, alkyl and amino; (9) R^{11a} is selected from the group consisting of: alkyl, unsubstituted aryl, substituted aryl, cycloalkyl or substituted cycloalkyl, wherein the substituents on said substituted groups are are selected from the group consisting of: halo, —CN and CF₃; (10) R¹² is H; (11) R⁹ is selected from the group consisting of: (a) —(CH₂)ₙ-heterocycloalkyl; (b) —(CH₂)ₙ-substituted heterocycloalkyl; (c) —(CH₂)ₙ-heteroaryl, and (d) —(CH₂)ₙ-substituted heteroaryl; wherein n is 1, 2, or 3 and the substituents for said substituted R⁹ groups are each independently selected from the group consisting of: (1) —OH; (2) —CO₂R¹⁴; (3) —CH₂OR¹⁴, (4) halo, (5) alkyl; (6) amino; (7) trityl; (8) heterocycloalkyl; (9) arylalkyl; (10) heteroaryl and (11) heteroarylalkyl; wherein R¹⁴ is independently selected from the group consisting of: H and alkyl; and (12) X is N or CH.

In another embodiment, (1) R¹ to R⁴ each may be independently selected from H, Br or Cl; (2) R⁹ is selected from the group consisting of: (a) —(CH₂)ₙ-imidazolyl; (b) —(CH₂)ₙ-substituted imidazolyl; (c) —(CH₂)ₙ-morpholinyl; (d) —(CH₂)ₙ-substituted morpholinyl, (e) —(CH₂)ₙ-piperazinyl, or (f) —(CH₂)ₙ-substituted piperazinyl, wherein n is 1, 2, or 3; (3) R¹¹ is selected from the group consisting of: t-butyl and cyclohexyl; (4) R¹² is H; and (5) R^{11a} is selected from the group consisting of: t-butyl, cyanophenyl, chlorophenyl, fluorophenyl and cyclohexy.

In yet another embodiment, (1) R¹ and R² are H; (2) R³ is H; (3) R⁴ is Cl; (5) R⁸ is 4.0 wherein R^{11a} is cyanophenyl; and R¹² is H; and (6) R⁹ is selected from the group consisting of: —CH₂-imidazolyl, and —CH₂-imidazolyl wherein said imidazolyl moiety is substituted with a methyl group.

In one embodiment, the farnesyl transferase inhibitor compound may have the formula:

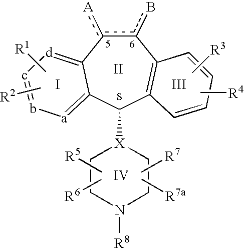

X may be N.

In one embodiment, the farnesyl transferase inhibitor compound may have the formula:

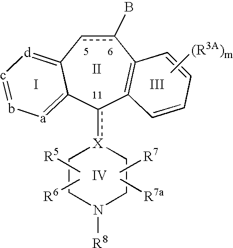

wherein:

(A) one of a, b, c and d represents N or N⁺O⁻, and the remaining a, b, c, and d groups represent CR¹ wherein each R¹ group on each carbon is the same or different; or

(B) each a, b, c, and d group represents CR¹ wherein each R¹ group on each carbon is the same or different;

(C) the dotted lines (- - -) represent optional bonds;

(D) X represents N or CH when the optional bond to C11 is absent, and represents C when the optional bond to C11 is present;

(E) R¹ is selected from the group consisting of: (1) H; (2) halo; (3) —CF₃; (4) —OR¹⁰; (5) COR¹⁰; (6) —SR¹⁰; (7) —S(O)ₜR¹⁵; (8) —N(R¹⁰)₂; (9) —NO₂; (10) —OC(O)R¹⁰; (11) CO₂R¹⁰; (12) —OCO₂R¹⁰; (13) —CN; (14) —NR¹⁰COOR¹⁵; (15) —SR¹⁵C(O)OR¹⁵; (16) —SR¹⁵N(R¹³)₂ wherein each R¹³ is independently selected from the group consisting of: H and —C(O)OR¹⁵, and provided that R¹⁵ in —SR¹⁵N(R¹³)₂ is not —CH₂; (17) benzotriazol-1-yloxy; (18) tetrazol-5-ylthio; (19) substituted tetrazol-5-ylthio; (20) alkynyl; (21) alkenyl; (22) alkyl; (23) alkyl substituted with one or more substitutents independently selected from the group consisting of: halogen, —OR¹⁰ and —CO₂R¹⁰; (24) alkenyl substituted with one or more substitutents independently selected from the group consisting of: halogen, —OR¹⁰ and —CO₂R¹⁰;
(F) Each R is independently selected from the group consisting of: (1) halo; (2) —CF₃; (3) —OR¹⁰; (4) COR¹⁰; (5) —SR¹⁰; (6) —S(O)ₜR¹⁵; (7) —N(R¹⁰)₂; (8) —NO₂; (9) —OC(O)R¹⁰; (10) CO₂R¹⁰; (11) —OCO₂R¹⁰; (12) —CN; (13) —NR¹⁰COOR¹⁵; (14) —SR¹⁵C(O)OR¹⁵; (15) —SR¹⁵N(R¹³)₂ wherein each R¹³ is independently selected from the group consisting of: H and —C(O)OR¹⁵, and provided that R¹⁵ in —SR¹⁵N(R¹³)₂ is not —CH₂; (16) benzotriazol-1-yloxy; (17) tetrazol-5-ylthio; (18) substituted tetrazol-5-ylthio; (19) alkynyl; (20) alkenyl; (21) alkyl; (22) alkyl substituted with one or more substitutents independently selected from the group consisting of: halogen, —OR¹⁰ and —CO₂R¹⁰; and (23) alkenyl substituted with one or more substitutents independently selected from the group consisting of: halogen, —OR¹⁰ and —CO₂R¹⁰;

(G) m is 0, 1 or 2;

(H) t is 0, 1 or 2

(I) R⁵, R⁶, R⁷ and R^{7a} are each independently selected from the group consisting of: (1) H; (2) —CF₃; (3) —COR¹⁰; (4) alkyl; (5) unsubstituted aryl; (6) alkyl substituted with one or more groups selected from the group consisting of: —OR¹⁰, —SR¹⁰, —S(O)ₜR¹⁵, —NR¹⁰COOR¹⁵, —N(R¹⁰)₂, —NO₂, —C(O)R¹⁰; —OCOR¹⁰, —OCO₂R¹⁵, CO₂R¹⁰, and OPO₃R¹⁰; and (7) aryl substituted with one or more groups selected from the group consisting of: —OR¹⁰, —SR¹⁰, —S(O)ₜR¹⁵, —NR¹⁰COOR¹⁵, —N(R¹⁰)2′-NO₂, —C(O)R¹⁰; —OCOR¹⁰, —OCO₂R¹⁵, —CO₂R¹⁰, and OPO₃R¹⁰; or

(J) R⁵ together with R⁶ represents ═O or ═S;

(K) R⁸ is selected from the group consisting

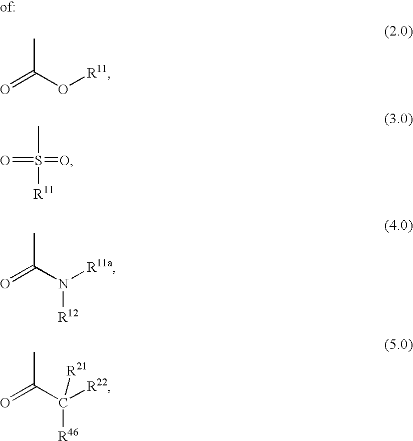

(L) R¹⁰ is selected from the group consisting of: H; alkyl; aryl and arylalkyl;

(M) R¹¹ is selected from: (1) alkyl; (2) substituted alkyl; (3) unsubstituted aryl; (4) substituted aryl; (5) unsubstituted cycloalkyl; (6) substituted cycloalkyl; (7) unsubstituted heteroaryl; (8) substituted heteroaryl; (9) heterocycloalkyl; and (10) substituted heterocycloalkyl; wherein said substituted alkyl, substituted cycloalkyl, and substituted heterocycloalkyl R¹¹ groups are substituted with one or more substituents selected from the group consisting of: (1) —OH; (2) fluoro; and (3) alkyl; and wherein said substituted aryl and substituted heteroaryl R¹¹ groups are substituted with one or more substituents selected from the group consisting of: (1) —OH; (2) halogen; and (3) alkyl;

(N) R^{11a} is selected from the group consisting of: (1) H; (2) OH; (3) alkyl; (4) substituted alkyl; (5) unsubstituted aryl; (6) substituted aryl; (7) unsubstituted cycloalkyl; (8) substituted cycloalkyl; (9) unsubstituted heteroaryl; (10) substituted heteroaryl; (11) heterocycloalkyl; and (12) substituted heterocycloalkyl; wherein said substituted alkyl, substituted cycloalkyl, and substituted heterocycloalkyl R^{11a} groups are substituted with one or more substituents selected from the group consisting of: (1) —OH; (2) —CN; (3) —CF₃; (4) fluoro; (5) alkyl; (6) cycloalkyl; (7) heterocycloalkyl; (8) arylalkyl; (9) heteroarylalkyl; (10) alkenyl and (11) heteroalkenyl; and wherein said substituted aryl and substituted heteroaryl R^{11a} groups are substituted with one or more substituents selected from the group consisting of: (1) —OH; (2) —CN; (3) —CF₃; (4) halogen; (5) alkyl; (6) cycloalkyl; (7) heterocycloalkyl; (8) arylalkyl; (9) heteroarylalkyl; (10) alkenyl and (11) heteroalkenyl; (O)R¹² is selected from the group consisting of: H, alkyl, piperidine Ring V, cycloalkyl, and -alkyl-(piperidine Ring V);

(P) R¹⁵ is selected from the group consisting of: alkyl and aryl;

(Q) R²¹, R²² and R⁴⁶ are independently selected from the group consisting of: (1) H; (2) alkyl; (3) unsubstituted aryl; (4) substituted aryl substituted with one or more substituents selected from the group consisting of: alkyl, halogen, CF₃ or OH; (5) unsubstituted cycloalkyl; (6) substituted cycloalkyl substituted with one or more substituents selected from the group consisting of: alkyl, halogen, CF₃ or OH; (7) heteroaryl of the formula, 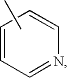 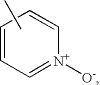
(8) piperidine Ring V: 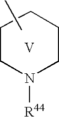
wherein R⁴⁴ is selected from the group consisting of: (a) H, (b) alkyl; (c) alkylcarbonyl; (d) alkyloxy carbonyl; (e) haloalkyl and (f) —C(O)NH(R⁵¹);

(R) R⁵¹ is selected from the group consisting of: —H and alkyl (e.g., methyl, ethyl, propyl, butyl and t-butyl);

(S) B is the group:

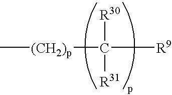

(T) in said B group: (1) p of the —(CH₂)ₚ— moiety is 0; (2) p of the 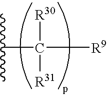
moiety is 1 to 3; (3) when p is one for the moiety 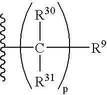
then R³⁰ is selected from the group consisting of: —OH and —NH₂, and R³¹ is alkyl; (4) when p is 2 or 3 for the moiety 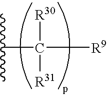
then: (1) for one —CR³⁰R³¹— moiety, R³⁰ is selected from the group consisting of: —OH and —NH₂, and R³¹ is alkyl; and (2) for the remaining —CR³⁰R³¹— moieties R³⁰ and R³¹ are hydrogen; and (5) R⁹ is unsubstituted heteroaryl or substituted heteroaryl, provided that when said heteroaryl group contains nitrogen in the ring, then said heteroaryl group is not bound by a ring nitrogen to the adjacent —CR³⁰R³¹— moiety when R³⁰ is —OH or —NH₂.

In one embodiment, (4) a is N; (5) b, c and d are CR¹ groups wherein all of said R¹ substituents are H, or one R¹ substituent is halo and the remaining two R¹ substituents are hydrogen; (6) m is 1, and R^{3A} is halo, or m is 2 and each R^{3A} is the same or different halo (e.g., Br or Cl); and (7) R⁵, R⁶, R⁷, and R^{7a} are H.

In one embodiment, the farnesyl transferase inhibitor compound may have the formula:

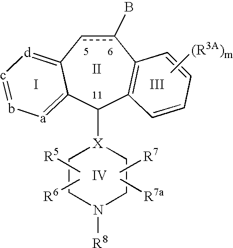

wherein:

(A) B is the group:

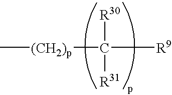

(B) in said B group: (1) p of the —(CH₂)ₚ— moiety is 0; (2) p of the 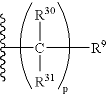
moiety is 1 to 3; (3) when p is one for the moiety 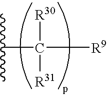
then R³⁰ is selected from the group consisting of: —OH and —NH₂, and R³¹ is alkyl; (d) when p is 2 or 3 for the moiety 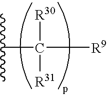
then: (1) for one —CR³⁰R³¹— moiety, R³⁰ is selected from the group consisting of: —OH and —NH₂, and R³¹ is alkyl; and (2) for the remaining —CR³⁰R³¹-moieties R³⁰ and R³¹ are hydrogen; and (e) R⁹ is unsubstituted heteroaryl or substituted heteroaryl, provided that when said heteroaryl group contains nitrogen in the ring, then said heteroaryl group is not bound by a ring nitrogen to the adjacent —CR³⁰R³¹— moiety when R³⁰ is —OH or —NH₂;

(C) a is N;

(D) b, c and d are CR¹ groups wherein all of said R¹ substituents are H, or one R¹ substituent is halo and the remaining two R¹ substituents are hydrogen;

(E) m is 1, and R^{3A} is halo, or m is 2 and each R^{3A} is the same or different halo;

(F) X is N or CH;

(G) R⁵, R⁶, R⁷, and R^{a} are H;

(H) R⁸ is selected from the group consisting of:

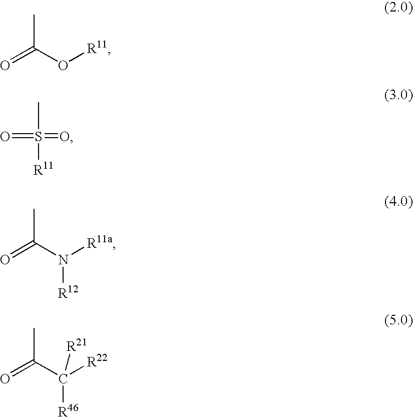

(I) R¹¹ is selected from: (1) alkyl; (2) substituted alkyl; (3) unsubstituted aryl; (4) substituted aryl; (5) unsubstituted cycloalkyl; (6) substituted cycloalkyl; (7) unsubstituted heteroaryl; (8) substituted heteroaryl; (9) heterocycloalkyl; and (10) substituted heterocycloalkyl; wherein said substituted alkyl, substituted cycloalkyl, and substituted heterocycloalkyl R¹¹ groups are substituted with one or more substituents selected from the group consisting of: (1) —OH; (2) fluoro; and (3) alkyl; and wherein said substituted aryl and substituted heteroaryl R¹¹ groups are substituted with one or more substituents selected from the group consisting of: (1) —OH; (2) halogen; and (3) alkyl;

(J) R^{11a} is selected from the group consisting of: (1) H; (2) OH; (3) alkyl; (4) substituted alkyl; (5) unsubstituted aryl; (6) substituted aryl; (7) unsubstituted cycloalkyl; (8) substituted cycloalkyl; (9) unsubstituted heteroaryl; (10) substituted heteroaryl; (11) heterocycloalkyl; and (12) substituted heterocycloalkyl; wherein said substituted alkyl, substituted cycloalkyl, and substituted heterocycloalkyl R^{11a} groups are substituted with one or more substituents selected from the group consisting of: (1) —OH; (2) —CN; (3) —CF₃; (4) fluoro; (5) alkyl; (6) cycloalkyl; (7) heterocycloalkyl; (8) arylalkyl; (9) heteroarylalkyl; (10) alkenyl and (11) heteroalkenyl; and wherein said substituted aryl and substituted heteroaryl R^{11a} groups are substituted with one or more substituents selected from the group consisting of: (1) —OH; (2) —CN; (3) —CF₃; (4) halogen; (5) alkyl; (6) cycloalkyl; (7) heterocycloalkyl; (8) arylalkyl; (9) heteroarylalkyl; (10) alkenyl and (11) heteroalkenyl;

(K) R¹² is selected from the group consisting of: H, alkyl, piperidine Ring V, cycloalkyl, and -alkyl-(piperidine Ring V);

(L) R²¹, R²² and R⁴⁶ are independently selected from the group consisting of: (1) H; (2) alkyl; (3) unsubstituted aryl; (4) substituted aryl substituted with one or more substituents selected from the group consisting of: alkyl, halogen, CF₃ or OH; (5) unsubstituted cycloalkyl; (6) substituted cycloalkyl substituted with one or more substituents selected from the group consisting of: alkyl, halogen, CF₃ or OH; (7) heteroaryl of the formula, 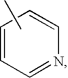 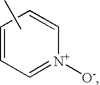
(8) piperidine Ring V: 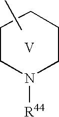
wherein R⁴⁴ is selected from the group consisting of: (a) H, (b) alkyl; (c) alkylcarbonyl; (d) alkyloxy carbonyl; (e) haloalkyl and (f) —C(O)NH(R⁵¹); and

(M) R⁵¹ is selected from the group consisting of: H and alkyl (e.g., methyl, ethyl, propyl, butyl and t-butyl).

In one embodiment, (A) in the B group: (1) p of the 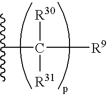
moiety is 0; (2) p of the 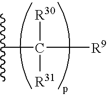
moiety is 1 to 2; (3) when p is one for the moiety 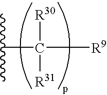
then R³⁰ is selected from the group consisting of: —OH and —NH₂, and R³¹ is C₁-C₂ alkyl; (4) when p is 2 or 3 for the moiety 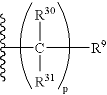
then: (1) for one —CR³⁰R³¹— moiety, R³⁰ is selected from the group consisting of: —OH and —NH₂, and R³¹ is C₁-C₂ alkyl; and (2) for the remaining —CR³⁰R³¹— moieties R³⁰ and R³¹ are hydrogen; and (5) R⁹ is imidazolyl or substituted imidazolyl, provided that said imidazolyl group is not bound by a ring nitrogen to the adjacent —CR³⁰R³¹— moiety when R³⁰ is —OH or —NH₂;

(B) R⁸ is 2.0;

(C) R¹¹ is alkyl;

(D) X is N;

(E) b, c and d are CR¹ groups wherein all of said R¹ substituents are H;

(F) m is 1, and R^{3A} is halo; and

(G) X is N.

In one embodiment, in the B group: (1) p of the —(CH₂)ₚ— moiety is 0; (2) p of the 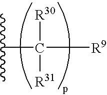
moiety is 1; (3) R³⁰ is selected from the group consisting of: —OH and —NH₂, and R³¹ is C₁-C₂ alkyl; and (4) R⁹ is substituted imidazolyl wherein said the substituent is an alkyl group, provided that said imidazolyl group is not bound by a ring nitrogen to the adjacent —CR³⁰R¹³— moiety.

In another embodiment, (A) in said B group: (1) p of the —(CH₂)ₚ— moiety is 0; (2) p of the 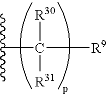
moiety is 1; (3) R³⁰ is —OH, and R³¹ is methyl; and (4) R⁹ is substituted imidazolyl wherein the substituent is a methyl group, provided that said imidazolyl group is not bound by a ring nitrogen to the adjacent —CR³⁰R³¹— moiety; and (B) R^{3A} is Cl; and (C) R¹¹ is alkyl.

R⁹ may be

R¹¹ may be t-butyl.

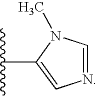

In one embodiment, the farnesyl transferase inhibitor compound may have the formula:

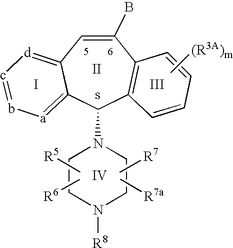

wherein all substituents may be as defined above.

In one embodiment, the farnesyl transferase inhibitor compound may have the formula:

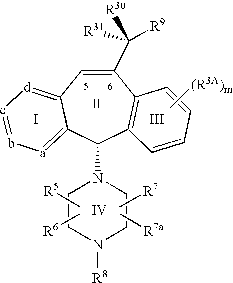

wherein all substituents may be as defined above.

In one embodiment, the farnesyl transferase inhibitor compound may have the formula:

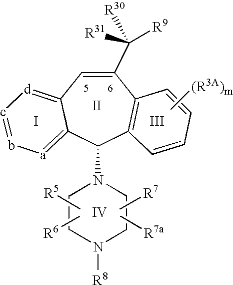

wherein all substituents may be as defined above.

In one embodiment, (A) in the B group: (1) p of the —(CH₂)ₚ— moiety is 0; (2) p of the 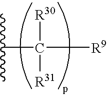
moiety is 1; (3) R³⁰ is —OH, and R³¹ is methyl; and (4) R⁹ is substituted imidazolyl wherein the substituent is a methyl group, provided that said imidazolyl group is not bound by a ring nitrogen to the adjacent —CR³⁰R³¹— moiety; and (B) R^{3A} is Cl; and (C) R¹¹ is alkyl.

R⁹ may be

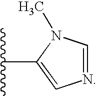

R¹¹ may be t-butyl.

In one embodiment, (A) in the B group: (1) p of the —(CH₂)ₚ— moiety is 0; (2) p of the 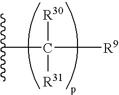
moiety is 1; (3) R³⁰ is OH, and R³¹ is methyl; and (4) R⁹ is substituted imidazolyl wherein the substituent is a methyl group, provided that said imidazolyl group is not bound by a ring nitrogen to the adjacent —CR³⁰R³¹— moiety; and (B) R^{3A} is Cl; and (C) R¹¹ is alkyl

R⁹ may be

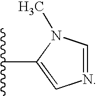

R¹¹ may be t-butyl.

In another aspect, the invention provides a method of treating a synucleinopathic subject by administering a farnesyl transferase inhibitor compound of the formula:

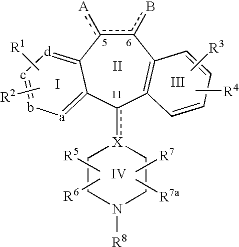

or a stereoisomeric form, or a pharmaceutically acceptable acid or base addition salt form thereof, in a therapeutically effective amount,

wherein:

one of a, b, c and d represents N or N⁺O⁻, and the remaining a, b, c, and d groups represent carbon, wherein each carbon has an R¹ or R² group bound to said carbon; or

each of a, b, c, and d is carbon, wherein each carbon has an R¹ or R² group bound to said carbon; the dotted lines (—) represent optional bonds;

X represents N or CH when the optional bond is absent, and represents C when the optional bond is present;
when the optional bond is present between carbon atom 5 and carbon atom 6 then there is only one A substituent bound to carbon atom 5 and there is only one B substituent bound to carbon atom 6 and A or B is other than H;
   when the optional bond is not present between carbon atom 5 and carbon atom 6, then there are two A substituents bound to carbon atom 5 and two B substituents bound to carbon atom 6, wherein each A and B substituent is independently selected from the group consisting of:

(1) —H; (2) —R⁹; (3) —R⁹—C(O)R⁹; (4) —R⁹—CO₂—R^{9a}; (5) —(CH₂)pR²⁶; (6) —C(O)N(R⁹)₂, wherein each R⁹ is the same or different; (7) —C(O)NHR⁹; (8) —C(O)NH—CH₂—C(O)—NH₂; (9) —C(O)NHR²⁶; (10) —(CH₂)pC(R⁹)—O—R^{9a}; (11) —(CH₂)p(R⁹)₂, wherein each R⁹ is the same or different; (12) —(CH₂)pC(O)R⁹; (13) —(CH₂)pC(O)R^{27a}; (14) —(CH₂)pC(O)N(R⁹)₂, wherein each R⁹ is the same or different; (15) —(CH₂)pC(O)NH(R⁹); (16) —(CH₂)pC(O)N(R²⁶)₂, wherein each R²⁶ is the same or different; (17) —(CH₂)pN(R⁹)—R^{9a}; (18) —(CH₂)pN(R²⁶)₂, wherein R²⁶ is the same or different; (19) —(CH₂)pNHC(O)R⁵⁰; (20) —(CH₂)pNHC(O)₂R⁵⁰; (21) —(CH₂)pN(C(O)R^{27a})₂ wherein each R^{27a} is the same or different; (22) —(CH₂)pNR⁵¹C(O)R²⁷, or R⁵¹ and R²⁷ taken together with the atoms to which they are bound form a heterocycloalkyl ring consisting of, 5 or 6 members, provided that when R⁵¹ and R²⁷ form a ring, R⁵¹ is not H; (23) —(CH₂)pNR⁵¹C(O)NR²⁷, or

R⁵¹ and R²⁷ taken together with the atoms to which they are bound form a heterocycloalkyl ring consisting or 5 or 6 members, provided that when R⁵¹ and R²⁷ form a ring, R⁵¹ is not H; (24) —(CH₂)pNR⁵¹C(O)N(R^{27a})₂, wherein each R^{27a} is the same or different; (25) —(CH₂)pNHSO₂N(R⁵¹)₂, wherein each R⁵¹ is the same or different; (26) —(CH₂)pNHCO₂R⁵⁰; (27) —(CH₂)pNC(O)NHR⁵¹; (28) —(CH₂)pCO₂R⁵¹; (29) —NHR⁹; (30) 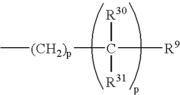
wherein R³⁰ and R³¹ are the same or different; (31) 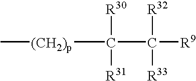
wherein R³⁰, R³¹, R³² and R³³ are the same or different; (32) -alkenyl-CO₂R^{9a}; (33)-alkenyl-C(O)R^{9a}; (34)-alkenyl-CO₂R⁵¹; (35)-alkenyl-C(O)—R^{27a}; (36) (CH₂)p-alkenyl-CO₂—R⁵¹; (37) —(CH₂)pC═NOR⁵¹ and (38) —(CH₂)p-Phthalimid;
p is 0, 1, 2, 3 or 4;
each R¹ and R² is independently selected from H, Halogen, —CF₃, —OR¹⁰, COR¹⁰, —SR¹⁰, —S(O)ₜ¹⁵ wherein t is 0, 1 or 2, —N(R¹⁰)₂, —NO₂, —OC(O)R¹⁰, CO₂R¹⁰, —OCO₂R¹⁵, —CN, —NR¹⁰COOR¹⁵, —SR¹⁵C(O)OR¹⁵—SR¹⁵N(R¹³)₂ provided that R¹⁵ in —SR¹⁵N(R¹³)₂ is not —CH₂, and wherein each R¹³ is independently selected from H or —C(O)OR¹⁵, benzotriazol-1-yloxy, tetrazol-5-ylthio, or substituted tetrazol-5-ylthio, alkynyl, alkenyl or alkyl, said alkyl or alkenyl group optionally being substituted with halogen, —OR¹⁰ or —CO₂R¹⁰;
R³ and R⁴ are the same or different and each independently represent H, or any of the substituents of R¹ and R²;
R⁵, R⁶, R⁷ and R^{7a} each independently represent H, —CF₃, —COR¹⁰, alkyl or aryl, said alkyl or aryl optionally being substituted with —OR¹⁰, —SR¹⁰, —S(O)ₜR¹⁵, —NR¹⁰COOR¹⁵, —N(R¹⁰)₂, —NO₂, —C(O)R¹⁰, —OCOR¹⁰, —OCO₂R¹⁵, —CO₂R¹⁰, OPO₃R¹⁰, or R⁵ is combined with R⁶ to represent ═O or ═S;
R⁸ is selected from the group consisting of: 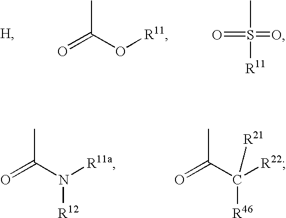
R⁹ is selected from the group consisting of: (1) heteroaryl; (2) substituted heteroaryl; (3) arylalkoxy; (4) substituted arylalkoxy; (5) heterocycloalkyl; (6) substituted heterocycloalkyl; (7) heterocycloalkylalkyl; (8) substituted heterocycloalkylalkyl; (9) heteroarylalkyl; (10) substituted heteroarylalkyl; (11) heteroarylalkenyl; (12) substituted heteroarylalkenyl; (13) heteroarylalkynyl; (14) substituted heteroarylalkynyl; (15) arylalkyl; (16) substituted arylalkyl; (17) alkenyl, and (18) substituted alkenyl; wherein said substituted R⁹ groups are substituted with one or more substituents selected from the group consisting of: (1) —OH; (2) —CO₂R¹⁴; (3) —CH₂OR¹⁴, (4) halogen; (5) alkyl; (6) amino; (7) trityl; (8) heterocycloalkyl; (9) cycloalkyl; (10) arylalkyl; (11) heteroaryl; (12) heteroarylalkyl and (13) 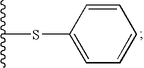
wherein
R¹⁴ is independently selected from the group consisting of: H; alkyl; aryl, arylalkyl, heteroaryl and heteroarylalkyl;
R^{9a} is selected from the group consisting of: alky and arylalkyl;
R¹⁰ is selected from the group consisting of: H; alkyl; aryl and arylalkyl;
R¹¹ is selected from the group consisting of: (1) alkyl; (2) substituted alkyl; (3) aryl; (4) substituted aryl; (5) cycloalkyl; (6) substituted cycloalkyl; (7) heteroaryl; (8) substituted heteroaryl; (9) heterocycloalkyl; and (10) substituted heterocycloalkyl; wherein said substituted R¹¹ groups have 1, 2 or 3 substituents selected from the group consisting of: (1) —OH; (2) halogen and (3) alkyl;
R^{11a} is selected from the group consisting of: (1) H; (2) OH; (3) alkyl; (4) substituted alkyl; (5) aryl; (6) substituted aryl; (7) cycloalkyl; (8) substituted cycloalkyl; (9) heteroaryl; (10) substituted heteroaryl; (11) heterocycloalkyl; and (12) substituted heterocycloalkyl; wherein said substituted R^{11a} groups have one or more substituents selected from the group consisting of: (1) —OH; (2) —CN; (3) —CF₃; (4) halogen; (5) alkyl; (6) cycloalkyl; (7) heterocycloalkyl, (8) arylalkyl; (9) heteroarylalkyl; (10) alkenyl and (11) heteroalkenyl;
R¹² is selected from the group consisting of: H, and alkyl;
R¹⁵ is selected from the group consisting of: alkyl and aryl;
R²¹, R²² and R⁴⁶ are independently selected from the group consisting of: (1) —H; (2) alkyl; (3) aryl; (4) substituted aryl, optionally substituted with one or more substituents selected from the group consisting of: alkyl, halogen, CF₃ and OH; (5) cycloalkyl; (6) substituted cycloalkyl; optionally substituted with one or more substituents selected from the group consisting of: alkyl, halogen, CF₃ and OH; (7) heteroaryl of the formula, 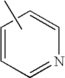 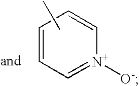
and (8) heterocycloalkyl of the formula: 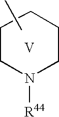
wherein R⁴⁴ is selected from the group consisting of: (1) —H; (2) alkyl; (3) alkylcarbonyl; (4) alkyloxy carbonyl; (5) haloalkyl and (6) —C(O)NH(R⁵¹); when R²¹, R²² or R⁴⁶ is the heterocycloalkyl of the formula above, Ring V is selected from the group consisting of: 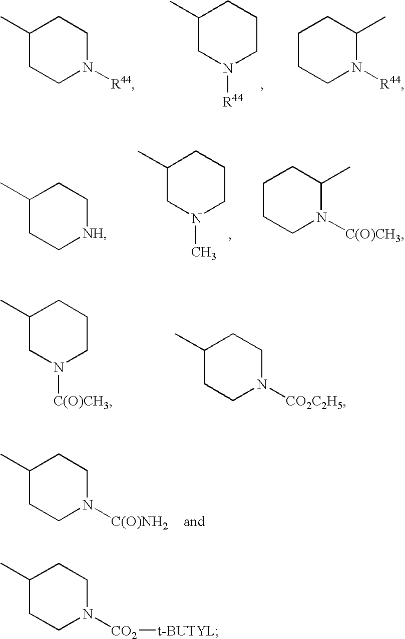
R²⁶ is selected from the group consisting of: (1) —H; (2) alkyl; (3) alkoxyl; (4) —CH₂—CN; (5) R⁹; (6) —CH₂CO₂H; (7) —C(O)alkyl and (8) CH₂CO₂alkyl;
R²⁷ is selected from the group consisting of: (1) —H; (2) —OH; (3) alkyl and (4) alkoxy; R^{27a} is selected from the group consisting of: (1) alkyl and (2) alkoxy;
R³⁰ through R³³ are independently selected from the group consisting of: (1) —H; (2) —OH; (3) ═O; (4) alkyl; (5) aryl and (6) arylalkyl;
R⁵⁰ is selected from the group consisting of: (1) alkyl; (2) heteroaryl; (3) substituted heteroaryl and (4) amino; wherein said substituents on said substituted R⁵⁰ groups are independently selected from the group consisting of: alkyl; halogen; and —OH;
R^{50a} is selected from the group consisting of: (1) heteroaryl; (2) substituted heteroaryl and (3) amino; R⁵¹ is selected from the group consisting of: —H, and alkyl.

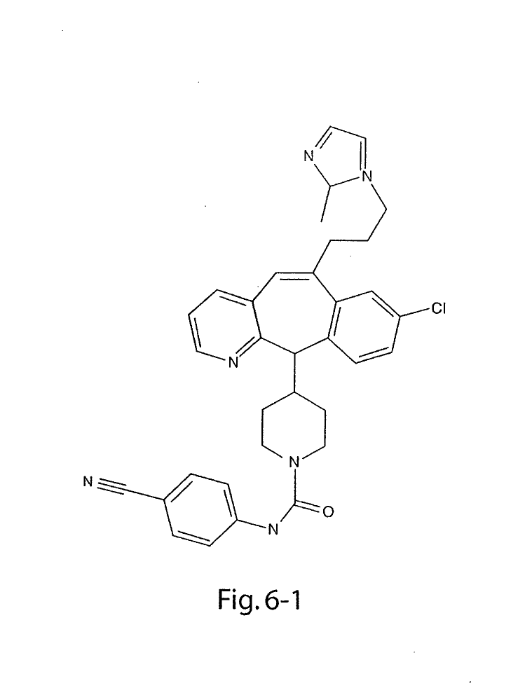

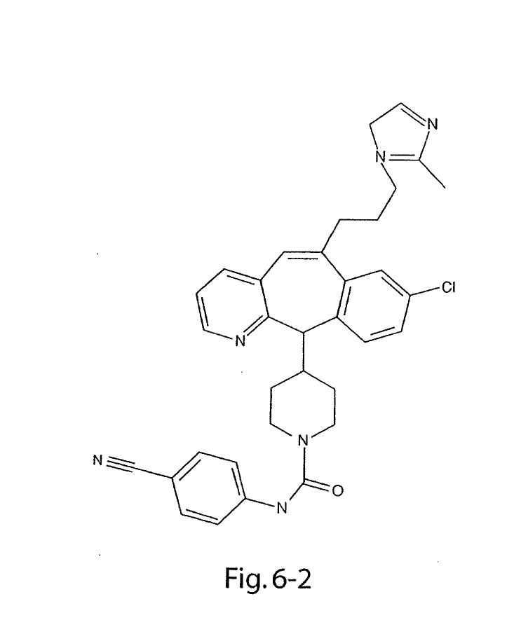

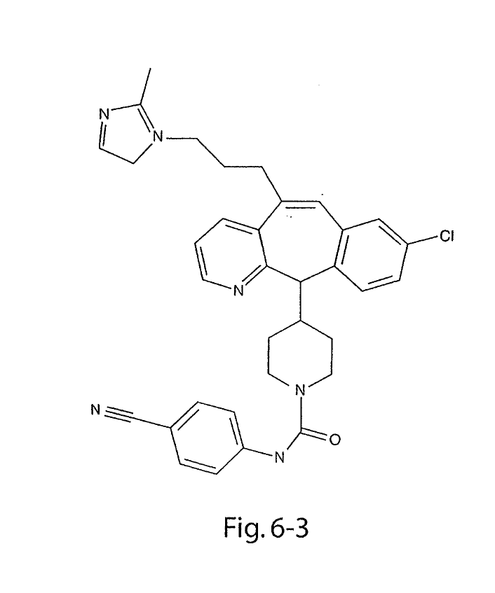

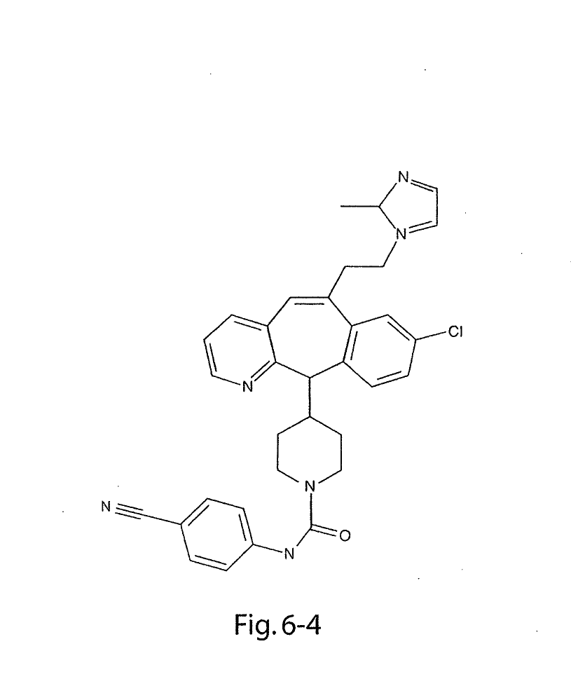

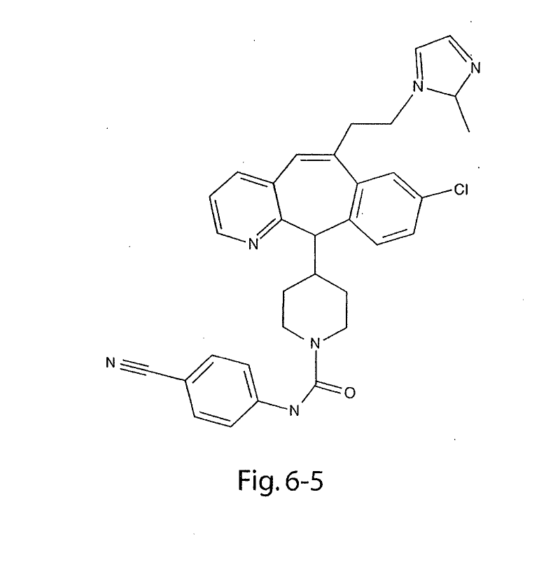

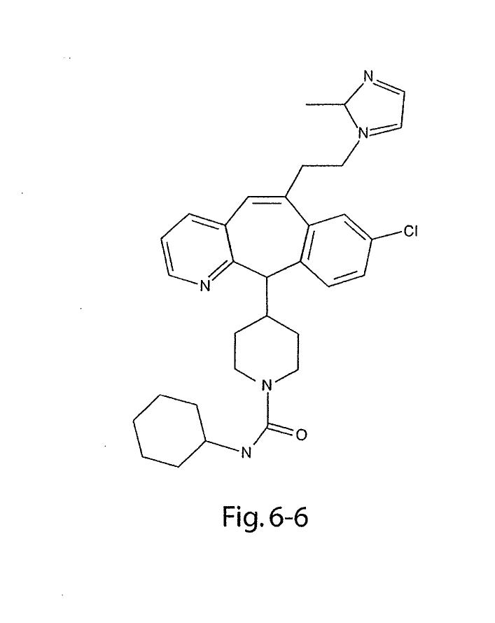

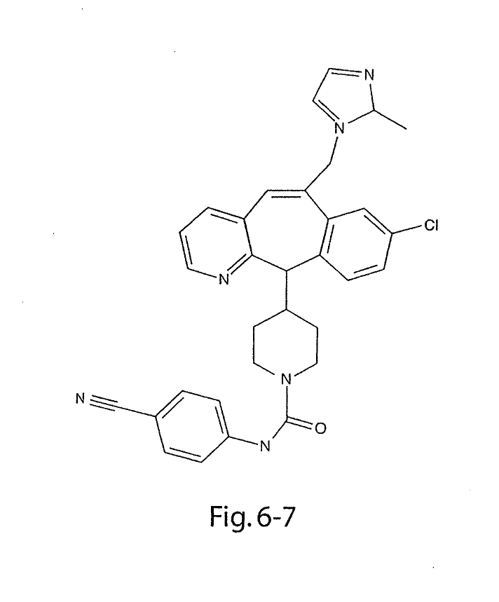

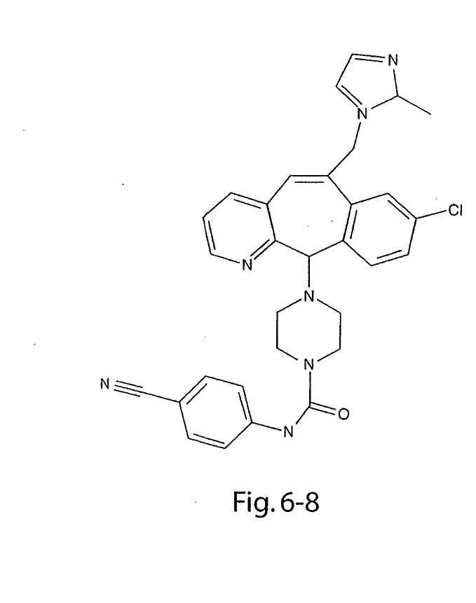

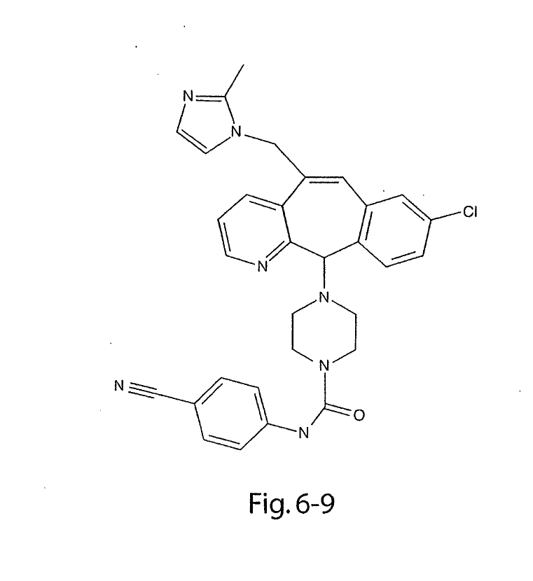

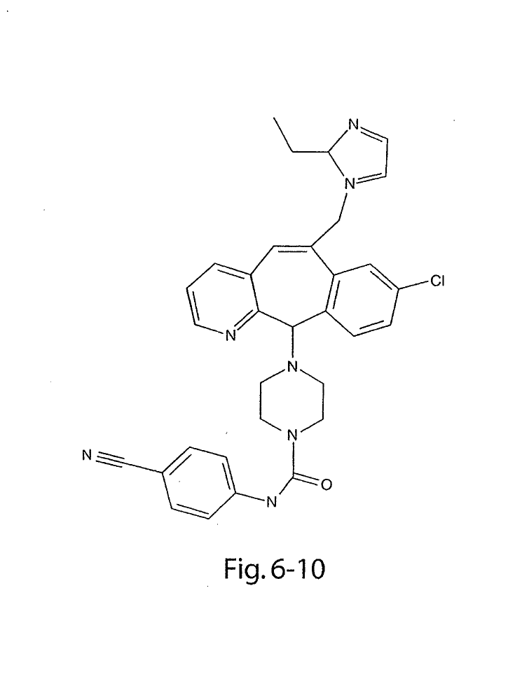

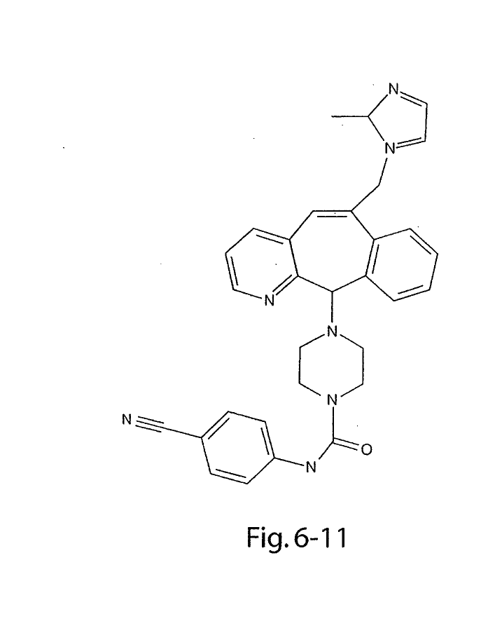

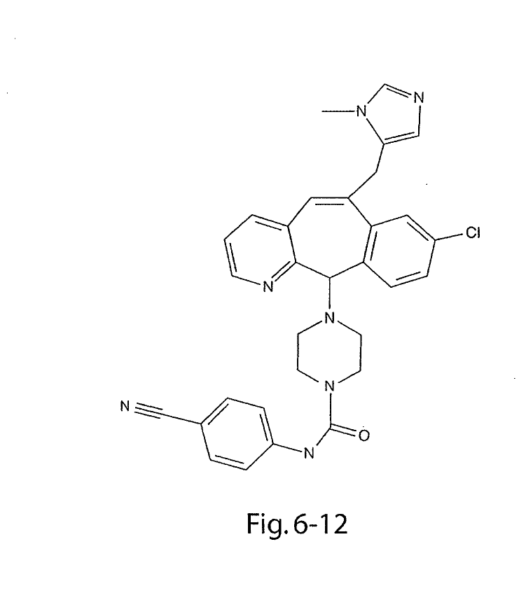

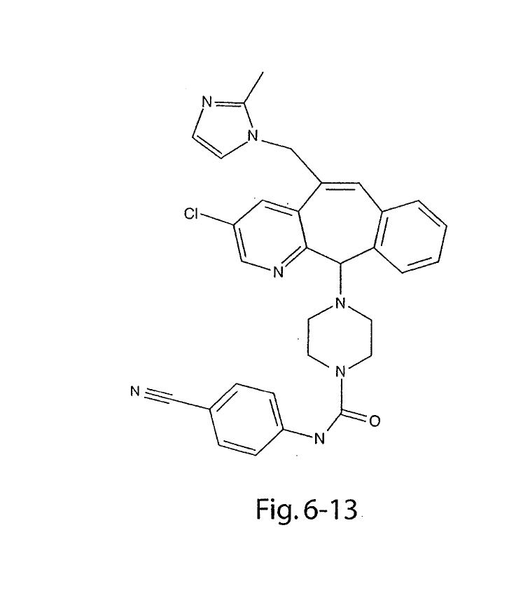

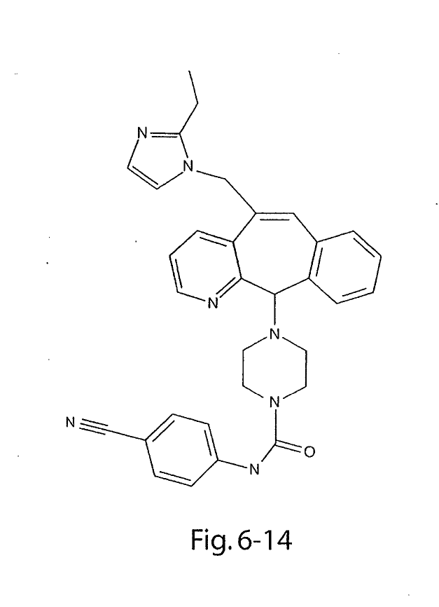

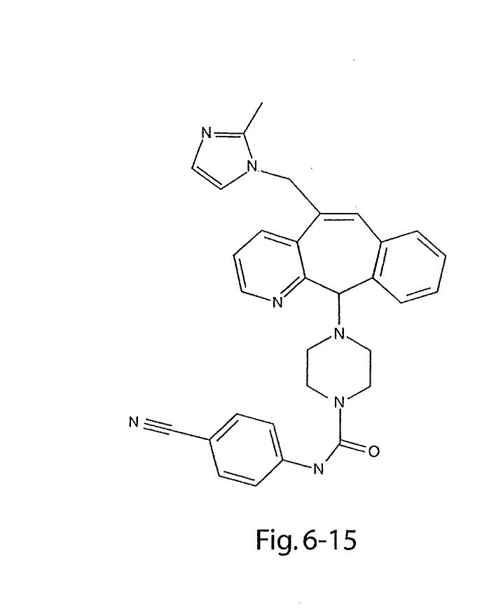

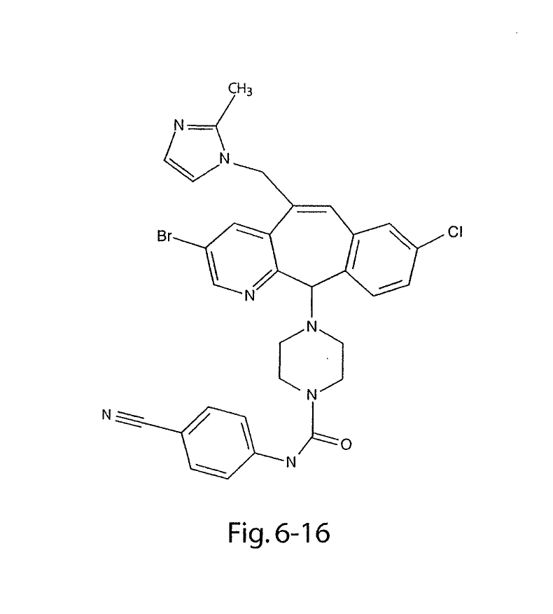

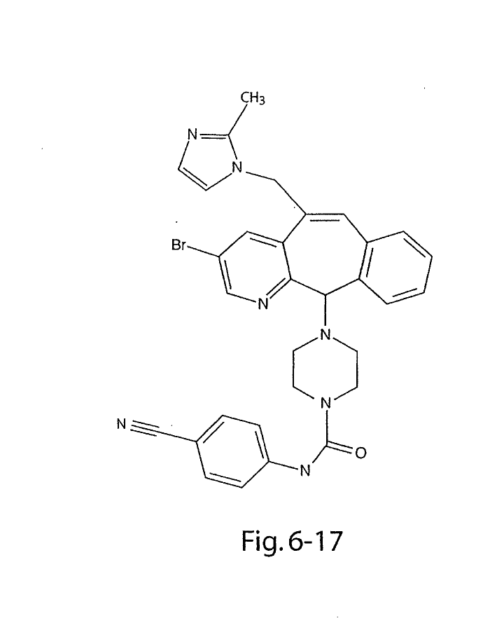

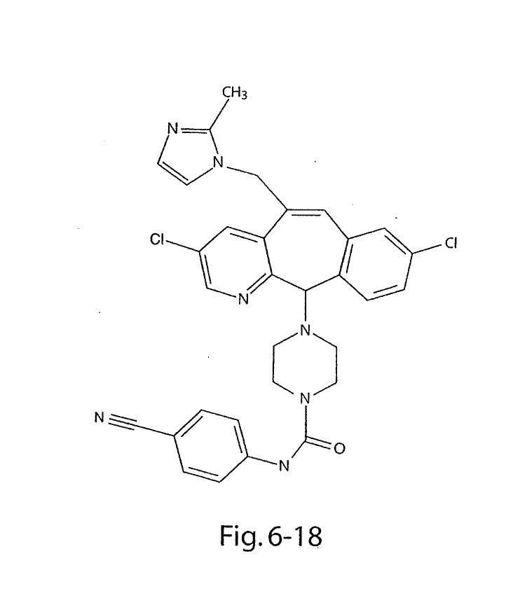

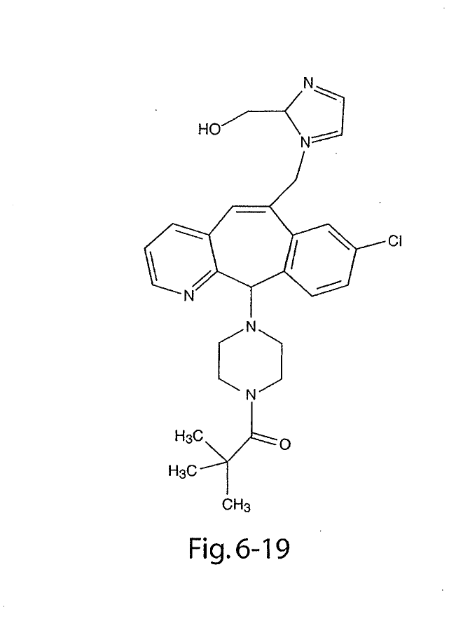

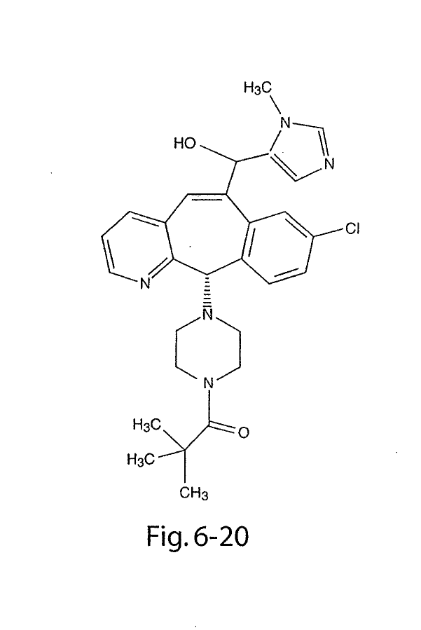

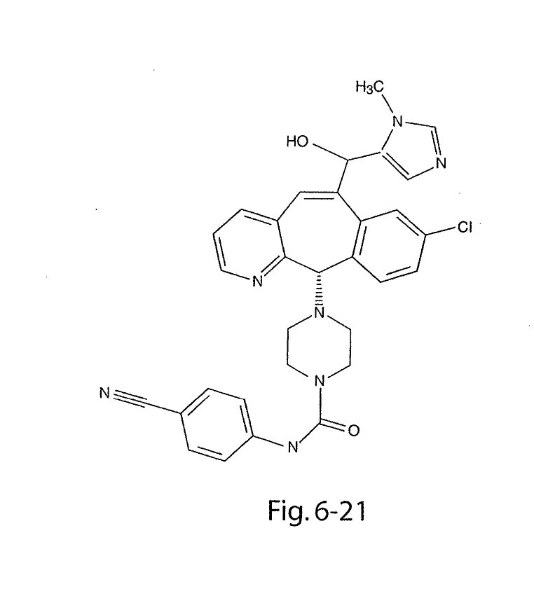

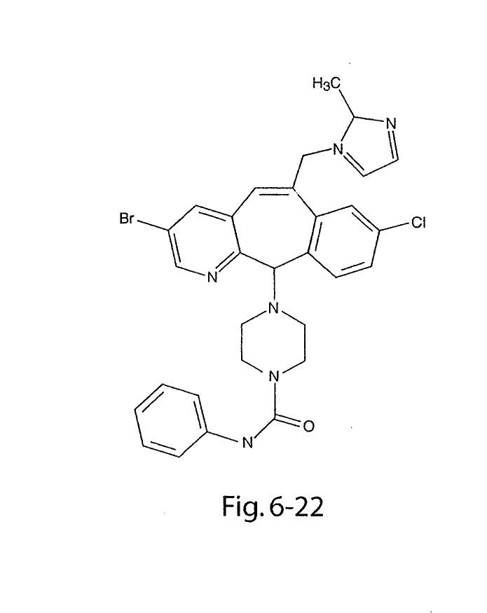

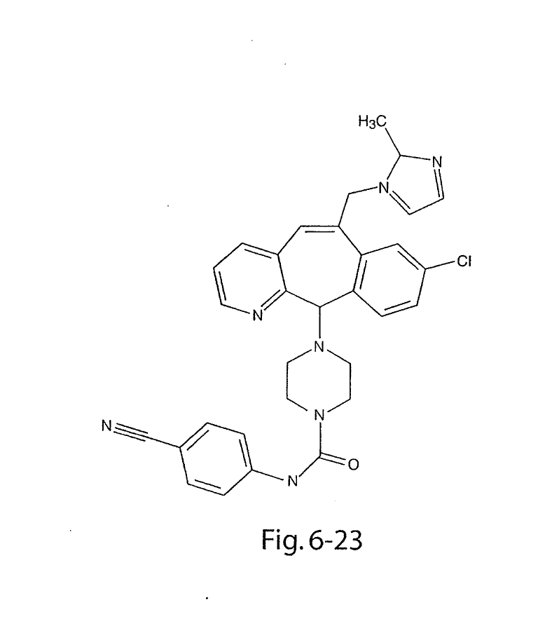

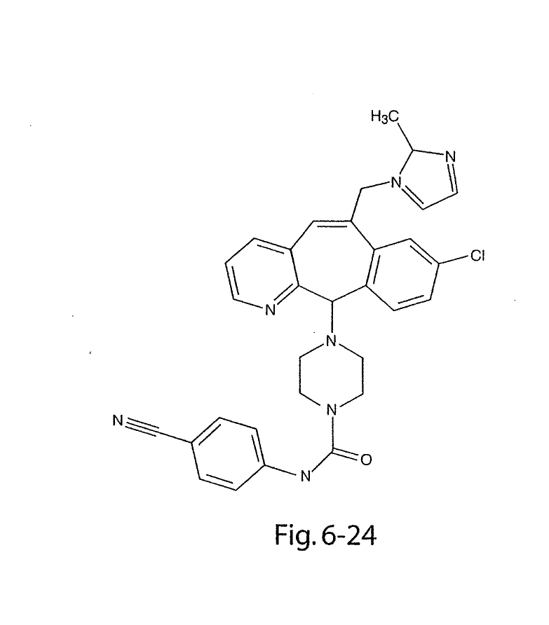

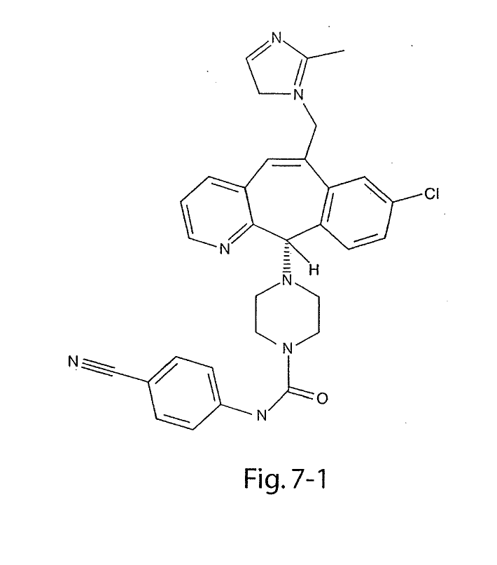

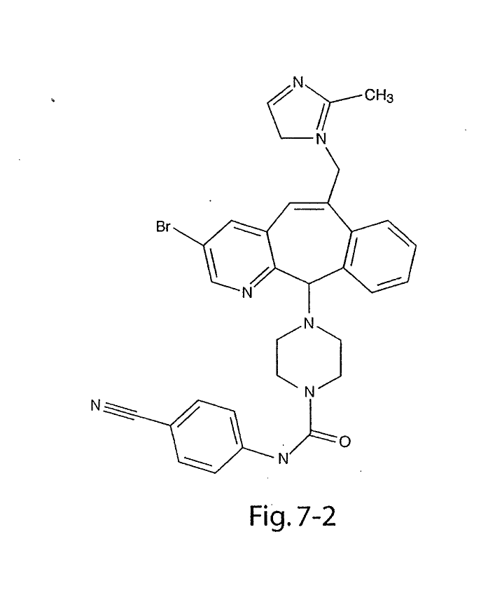

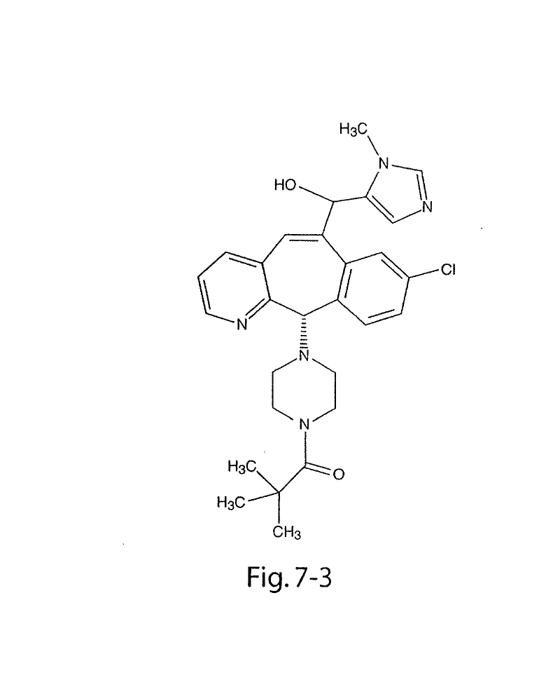

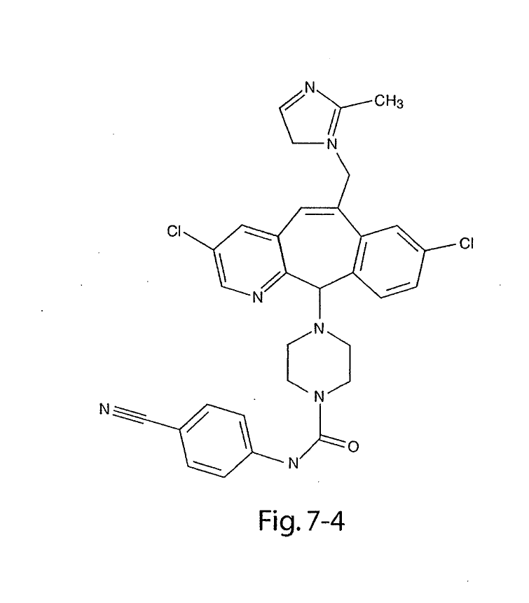

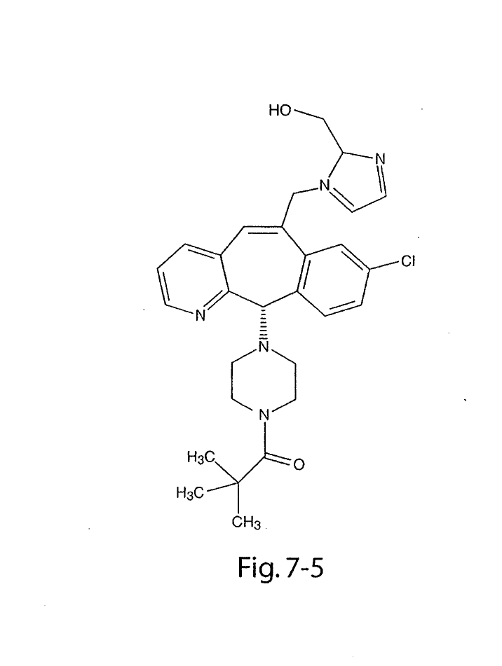

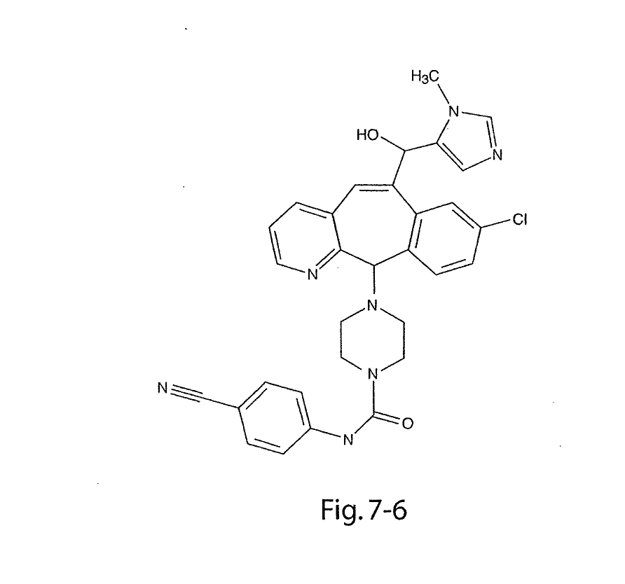

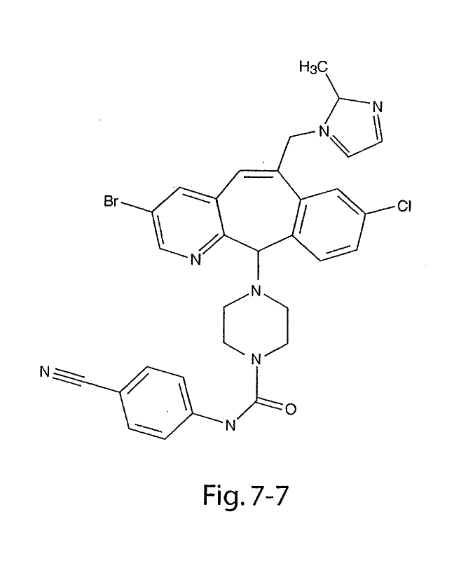

In one embodiment, the compound may have any of the structures shown in FIG. 6. In another embodiment, the compound may have any of the structures shown in FIG. 7.

In another aspect, the invention provides a method of treating a synucleinopathic subject by administering a farnesyl transferase inhibitor compound of the formula:

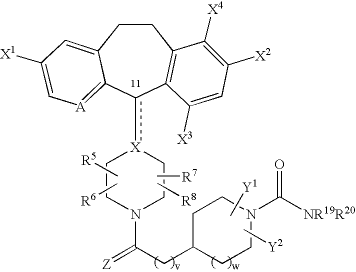

or a stereoisomeric form, or a pharmaceutically acceptable acid or base addition salt form thereof, in a therapeutically effective amount,

wherein:

A represents N or N-oxide;
X represents N, CH or C, such that when X is N or CH, there is a single bond to carbon atom 11 as represented by the solid line; or when X is C, there is a double bond to carbon atom 11, as represented by the solid and dotted lines;
X¹ and X² are independently selected from bromo or chloro, and X³ and X⁴ are independently selected from hydrogen, bromo or chloro provided that at least one of X³ and X⁴ is hydrogen;
Y¹ and Y² are independently selected from hydrogen or alkyl;
Z is ═O or ═S;
R⁵, R⁶, R⁷ and R⁸ each independently represents hydrogen, —CF₃, —COR¹⁰, alkyl or aryl, and further wherein R⁵ may be combined with R⁶ to represent ═O or ═S and/or R⁷ may be combined with R⁸ to represent ═O or ═S;
R¹⁰, R¹⁹ and R²⁰ independently represent hydrogen, alkyl, alkoxy, aryl, aralkyl, heteroaryl, heteroarylalkyl, cycloalkyl, cycloalkylalkyl, heterocycloalkyl and heterocycloalkylalkyl, with the proviso that R¹⁹ and R²⁰ are not both hydrogen;
v is zero, 1, 2 or 3; and
w is zero or 1.

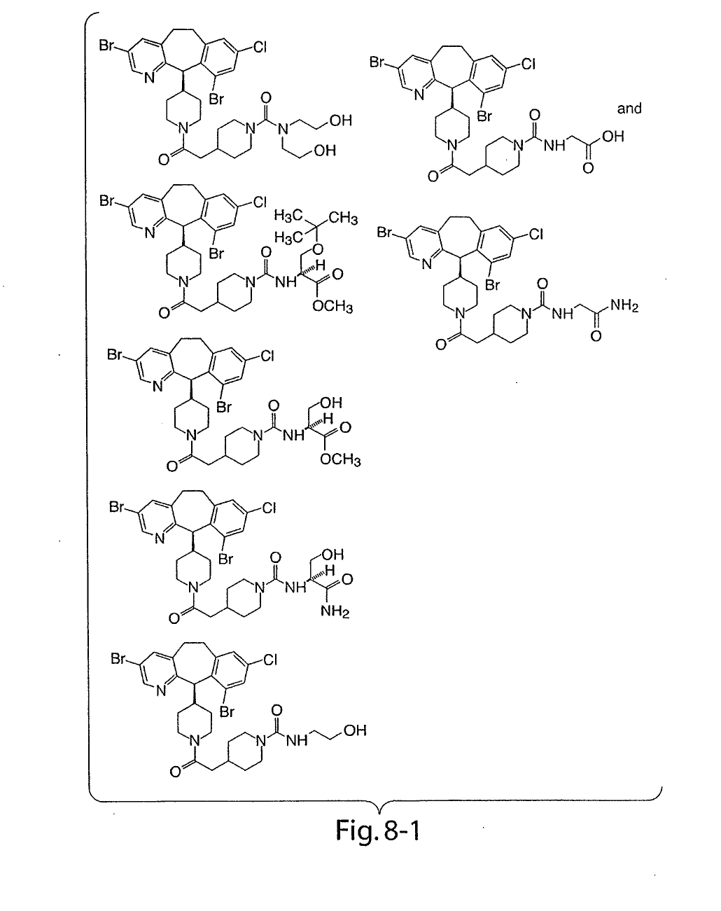

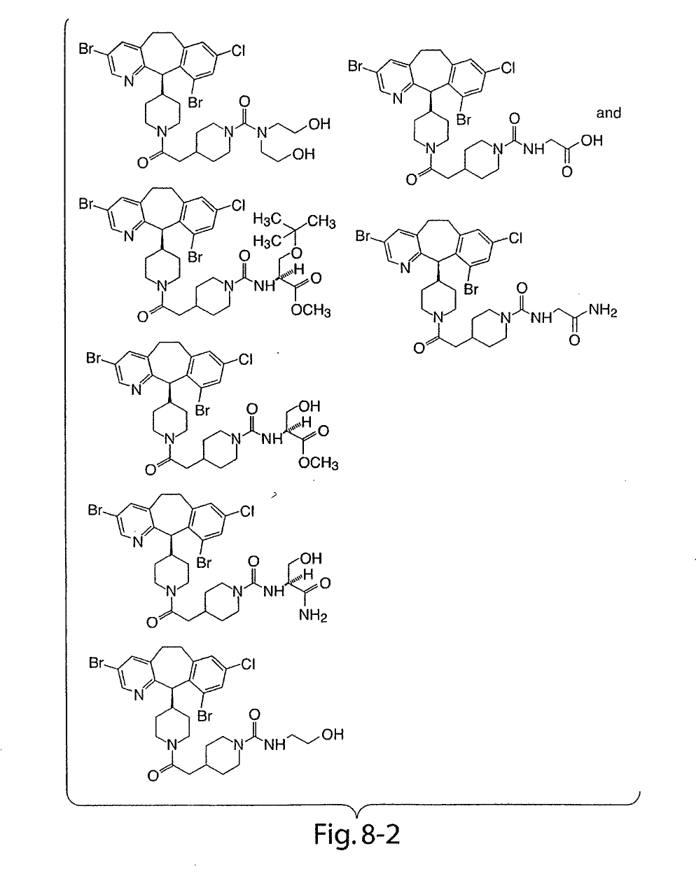

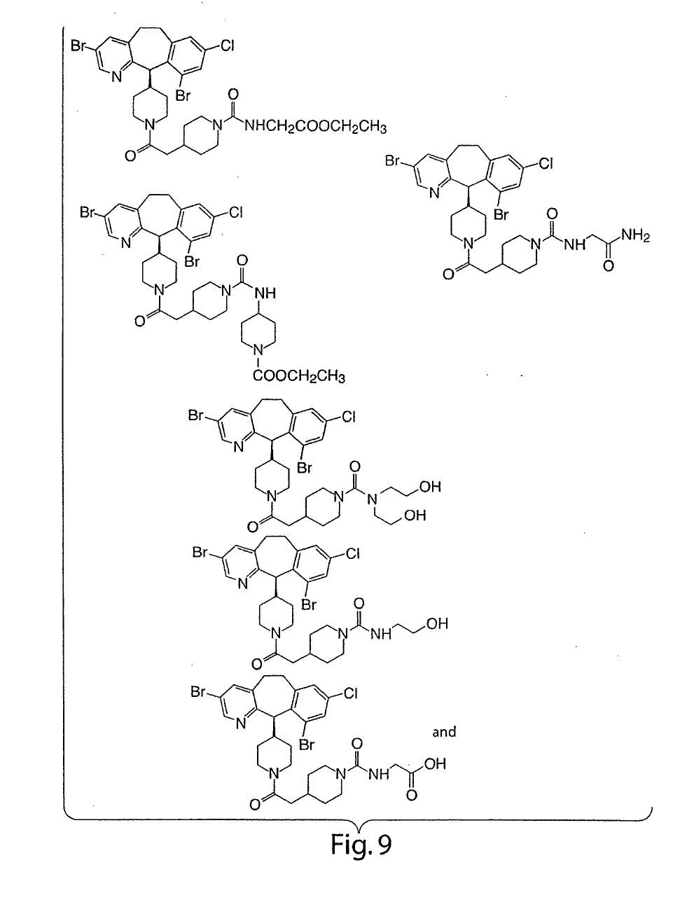

In one embodiment, there may be a single bond at carbon atom 11, X is CH, Z is ═O and R⁵, R⁶, R⁷ and R⁸ are hydrogen. In one embodiment, X¹ is bromo, X² is chloro, X³ is bromo and X⁴ is hydrogen. In one embodiment, Z is ═O; v is 1, w is 1, and Y¹ and Y² are hydrogen. In one embodiment, R¹⁹ and R²⁰ are independently selected from hydrogen, aryl and heterocycloalkyl with h the proviso that R¹⁹ and R²⁰ are not both hydrogen. In one embodiment, the aryl group is substituted with alkoxy; and the heterocycloalkyl group is substituted with —COOR¹⁰ wherein R¹⁰ is hydrogen or alkyl. In one embodiment, there is a single bond at carbon atom 11, X is CH, Z is ═O, R⁵, R⁶, R⁷ and R⁸ are hydrogen, X¹ is bromo, X² is chloro, X³ is bromo and X⁴ is hydrogen, v is 1, w is 1, and Y¹ and Y² are hydrogen, R¹⁹ and R²⁰ are independently selected from hydrogen, aryl and heterocycloalkyl; wherein the aryl group is substituted with alkoxy; and the heterocycloalkyl group is substituted with —COOR¹⁰ wherein R¹⁰ is hydrogen or alkyl, with the proviso that R¹⁹ and R²⁰ are not both hydrogen. In one embodiment, the compound may be any of the compounds shown in FIG. 8. In another embodiment, the compound may be any of the compounds shown in FIG. 9. In one embodiment, there is a single bond at carbon atom 11, X is CH, Z is ═O and R⁵, R⁶, R⁷ and R⁸ are hydrogen. In one embodiment, X¹ is bromo, X² is chloro, X³ is bromo and X⁴ is hydrogen. In one embodiment, Z is ═O; v is 1, w is 1, and Y¹ and Y² are hydrogen. In one embodiment, R¹⁹ and R²⁰ are independently selected from hydrogen, alkyl, aryl and heterocycloalkyl with the proviso that R¹⁹ and R²⁰ are not both hydrogen. In one embodiment, the alkyl group is substituted with —OR¹⁰, alkoxy, —OCOR¹⁰, —CONR¹⁰R¹² or —COOR¹⁰, wherein R¹⁰ and R¹² are independently selected from hydrogen, alkyl or alkoxy; the aryl group is substituted with alkoxy; and the heterocycloalkyl group is substituted with —COOR¹⁰ wherein R¹⁰ is hydrogen or alkyl. In one embodiment, there is a single bond at carbon atom 11, X is CH, Z is ═O, R⁵, R⁶, R⁷ and R⁸ are hydrogen, X¹ is bromo, X² is chloro, X³ is bromo and X⁴ is hydrogen, v is 1, w is 1, and Y¹ and Y² are hydrogen, R¹⁹ and R²⁰ are independently selected from hydrogen, alkyl, aryl and heterocycloalkyl, wherein the alkyl group is substituted with —OR¹⁰, alkoxy, —OCOR¹⁰, —CONR¹⁰R¹² or —COOR¹⁰, wherein R¹⁰ and R¹² are independently selected from hydrogen, alkyl or alkoxy; the aryl group is substituted with alkoxy; the heterocycloalkyl group is substituted with —COOR¹⁰ wherein R¹⁰ is hydrogen or alkyl, with the proviso that R¹⁹ and R²⁰ are not both hydrogen.

In another aspect, the invention provides a method of treating a synucleinopathic subject by administering a farnesyl transferase inhibitor compound of the formula:

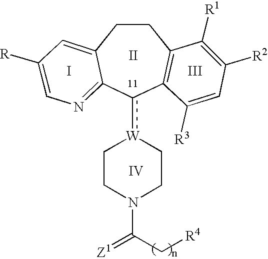

or a stereoisomeric form, or a pharmaceutically acceptable acid or base addition salt form thereof, in a therapeutically effective amount,

wherein:
R and R² are independently selected from halo;
R¹ and R³ are independently selected from the group consisting of H and halo, provided that at least one of R¹ and R³ is H;
W is N, CH or C, when the double bond is present at the C-11 position;
R⁴ is 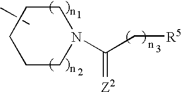
or R⁵; R⁵ is 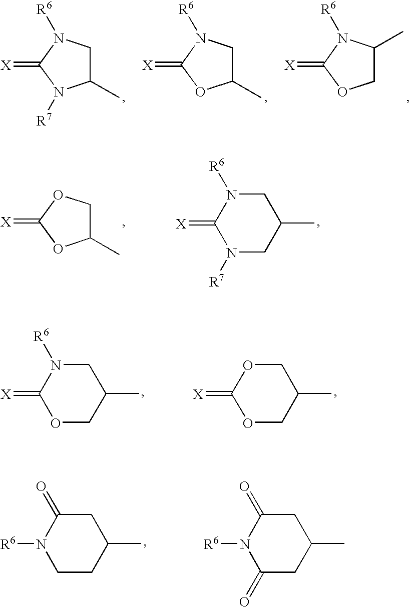
R⁶ and R⁷ are independently selected from the group consisting of H, alkyl, substituted alkyl, acyl, aryl, aralkyl, heterocycloalkyl and heteroaryl;
X is ═O or ═S;
Z¹ and Z² are independently ═O or ═S;
n and n₃ are independently 0, 1 or 2; and
n₁ and n₂ are independently 0 or 1.

In one embodiment, X is ═O and R⁶ and R⁷ are each hydrogen. In one embodiment, n is 1 and n₃ is 0 or 1. In one embodiment, R is bromo and R² is chloro or bromo. In one embodiment, R is bromo, R² is chloro or bromo, R¹ is H, and R³ is chloro or bromo. In one embodiment, R is bromo, R² is chloro or bromo, R³ is H, and R¹ is chloro or bromo. In one embodiment, the compound may any one of the following:

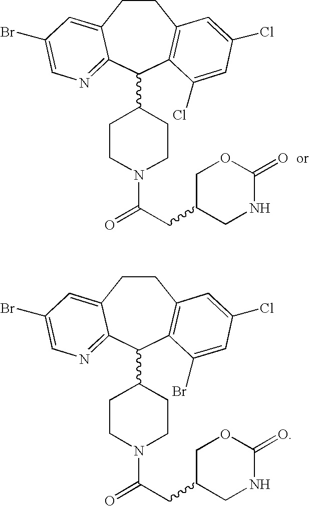

In another aspect, the invention provides a method of treating a synucleinopathic subject by administering a farnesyl transferase inhibitor compound of the formula:

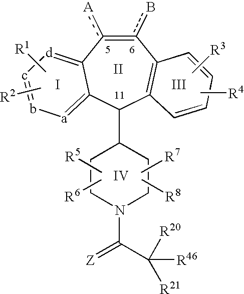

or a stereoisomeric form, or a pharmaceutically acceptable acid or base addition salt form thereof, in a therapeutically effective amount,

wherein:
a represents N and the remaining b, c and d groups represent CR¹ or CR²;
R¹ is selected from H or halo;
R² is selected from NO₂, Br, Cl or I;
R³ is Cl;
R⁴ is H or halo;
R⁵, R⁶, R⁷ and R⁸ are H;
the dotted line between carbon atoms 5 and 6 represents an optional double bond, such that when a double bond is present, A and B independently represent H, and when no double bond is present between carbon atoms 5 and 6, A and B each independently represent H₂;
R²⁰ and R²¹ are independently selected from H or alkyl;
R⁴⁶ is selected from: pyridyl, pyridyl N-oxide or piperidine Ring V: 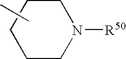
wherein R⁵⁰ represents alkyl, alkylcarbonyl, alkyloxycarbonyl, haloalkyl, or —C(O)NH(R¹⁰) wherein R¹⁰ is H or alkyl; and

Z represents O.

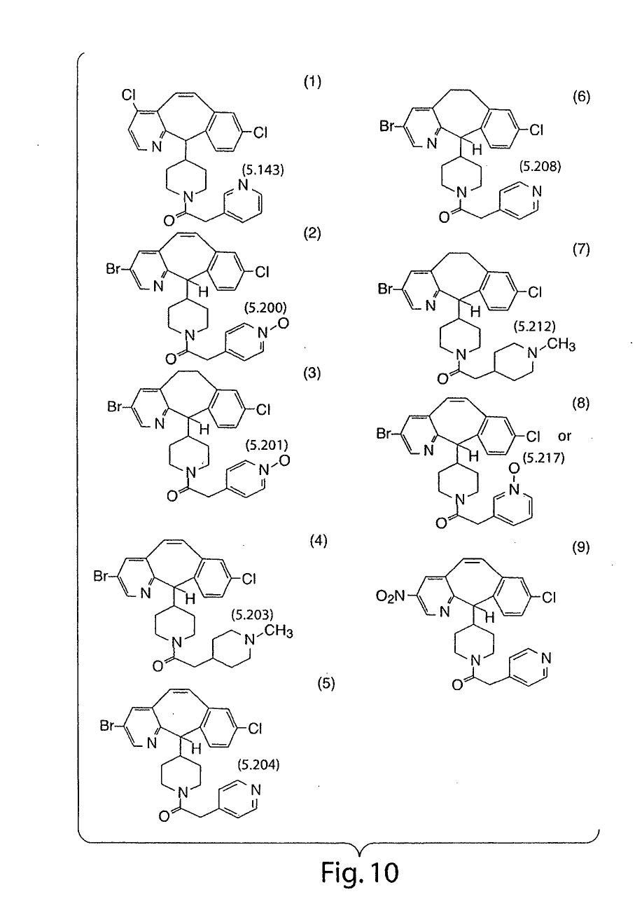

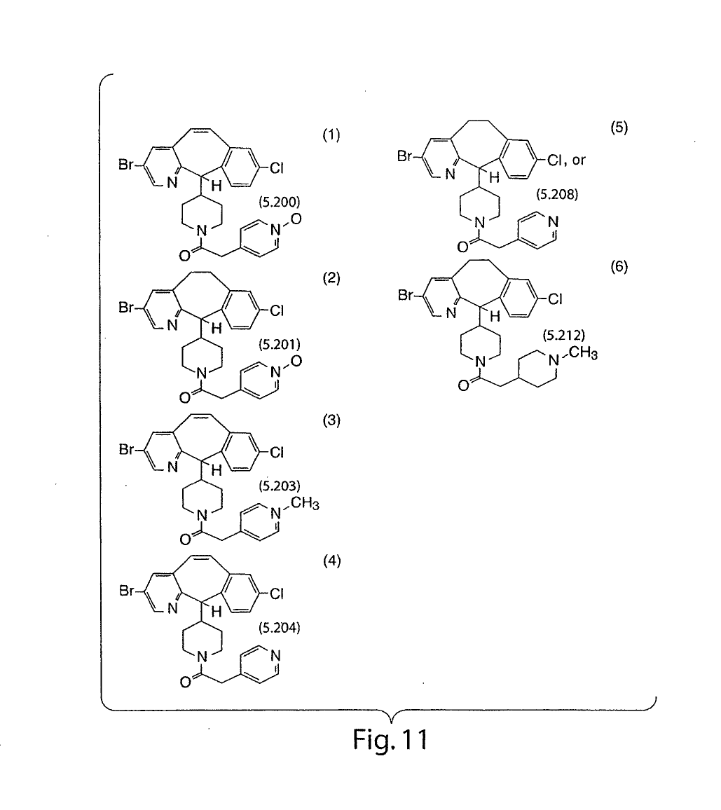

In one embodiment, R¹ is H. In one embodiment, R² is selected from Br, Cl or I. In one embodiment, R² is Br at the C-3 position. In one embodiment, R² is Br at the C-3 position and R³ is at the C-8 position. In one embodiment, both R²⁰ and R²¹ are hydrogen, or both R²⁰ and R²¹ are alkyl. In one embodiment, both R²⁰ and R²¹ are hydrogen. In one embodiment, R⁴⁶ is 3-pyridyl, 4-pyridyl, 3-pyridyl N-oxide, 4-pyridyl N-oxide, 4-N-methyl piperidinyl, 3-N-methylpiperidinyl, 4-N-acetylpiperidinyl or 3-N-acetylpiperidinyl. In one embodiment, R⁴⁶ is 3-pyridyl, 4-pyridyl, 3-pyridyl N-oxide, or 4-pyridyl N-oxide. In one embodiment, R⁴⁶ is 4-pyridyl or 4-pyridyl N-oxide. In one embodiment, the compound may be any of the compounds shown in FIG. 10. In another embodiment, the compound may be any of the compounds shown in FIG. 11. In one embodiment, the compound is of the formula: 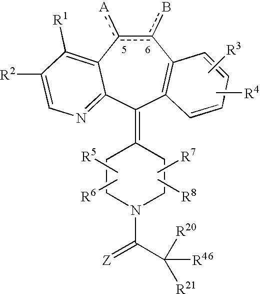
wherein:
R¹ is selected from H or halo;
R² is selected from —CH₃, Br, or I;
R³ is Cl;
R⁴ is H or halo;
R⁵, R⁶, R⁷ and R⁸ are H;
the dotted line between carbon atoms 5 and 6 represents an optional double bond, such that when a double bond is present, A and B independently represent H, and when no double bond is present between carbon atoms 5 and 6, A and B each independently represent H₂;
R²⁰ and R²¹ are H;
R⁴⁶ is selected from: pyridyl, pyridyl N-oxide, triazolyl, 1-N-methylpiperazinyl, 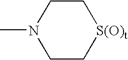
   wherein t is 0, 1 or 2, or piperidine Ring V: 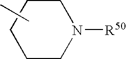
wherein R⁵⁰ represents alkyl, alkylcarbonyl, alkoxycarbonyl, haloalkyl, or —C(O)NH(R¹⁰) wherein R¹⁰ is H or alkyl; and
Z represents O.

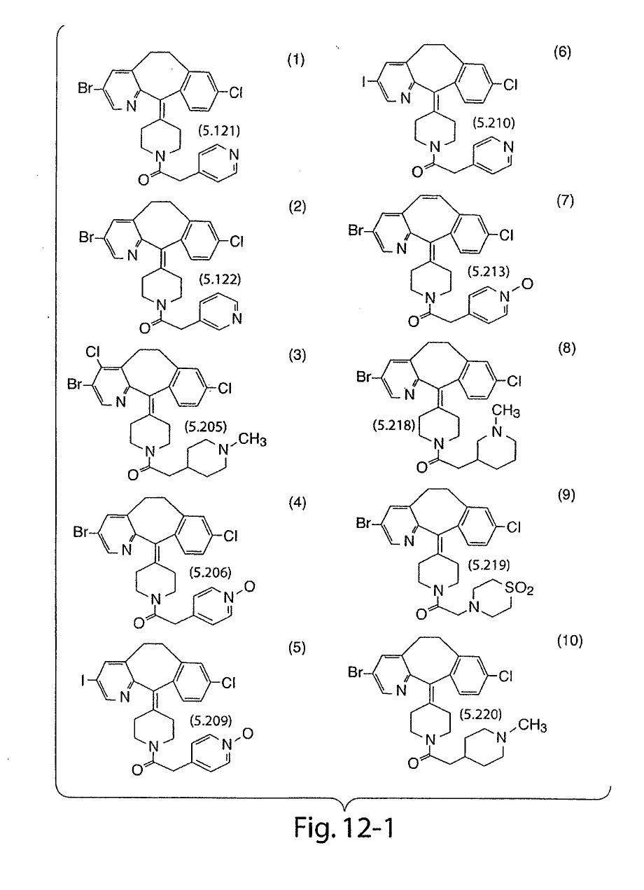

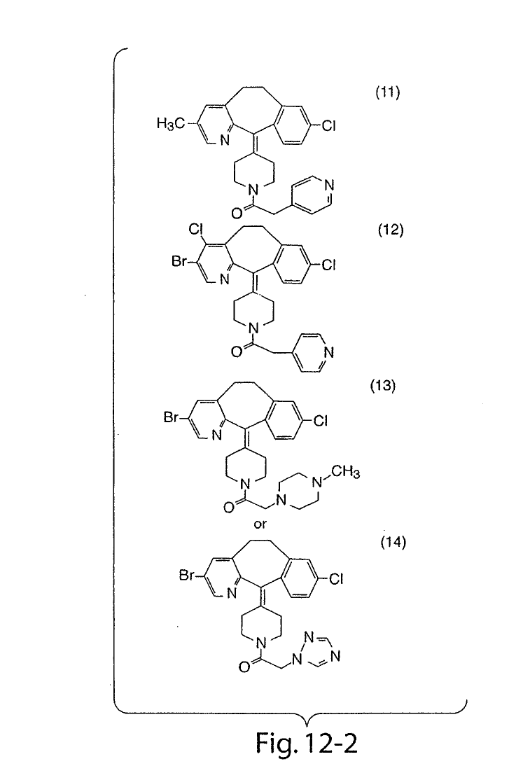

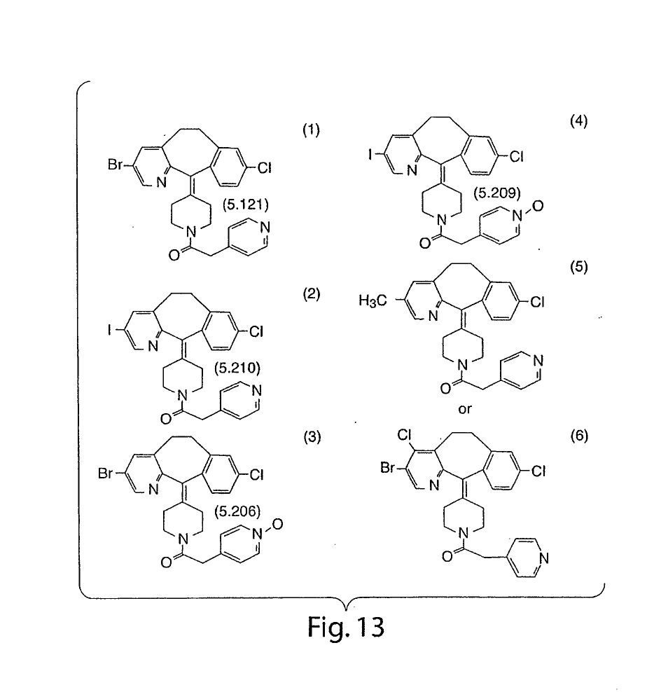

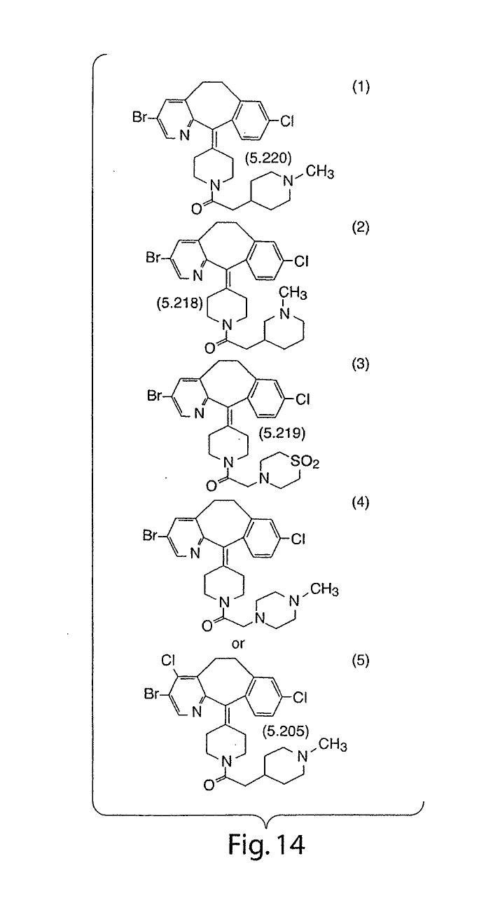

In one embodiment, R¹ is H. In one embodiment, R² is selected from Br. In one embodiment, R² is Br and R³ is at the C-8 position. In one embodiment, R⁴⁶ is selected from 3-pyridyl, 4-pyridyl, 3-pyridyl N-oxide, 4-pyridyl N-oxide, 4-N-methyl piperidinyl, 3-N-methylpiperidinyl, 4-N-acetylpiperidinyl or 3-N-acetylpiperidinyl. In one embodiment, R⁴⁶ is selected from: 3-pyridyl, 4-pyridyl, 3-pyridyl N-oxide, or 4-pyridyl N-oxide. In one embodiment, R⁴⁶ is selected from 4-pyridyl or 4-pyridyl N-oxide. In one embodiment, the compound may be any of the compounds shown in FIG. 12, FIG. 13, or FIG. 14.

In one aspect, the compound may have the formula: 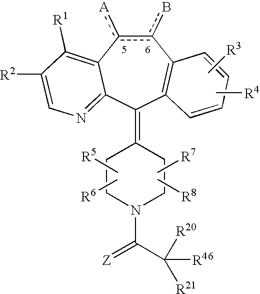
wherein:
R¹ is selected from H or halo;
R² is Cl;
R³ is Cl;
R⁴ is H or halo;
R⁵, R⁶, R⁷ and R⁸ are H;
the dotted line between carbon atoms 5 and 6 represents an optional double bond, such that when a double bond is present, A and B independently represent H, and when no double bond is present between carbon atoms 5 and 6, A and B each independently represent H₂;
R²⁰ and R²¹ are H;
R⁴⁶ is selected from: 4-pyridyl N-oxide, 4-pyridyl or piperidine Ring V:
wherein R⁵⁰ represents alkyl, alkylcarbonyl, alkyloxycarbonyl, haloalkyl, or —C(O)NH(R¹⁰) wherein R¹⁰ is H or alkyl; and
Z represents O.

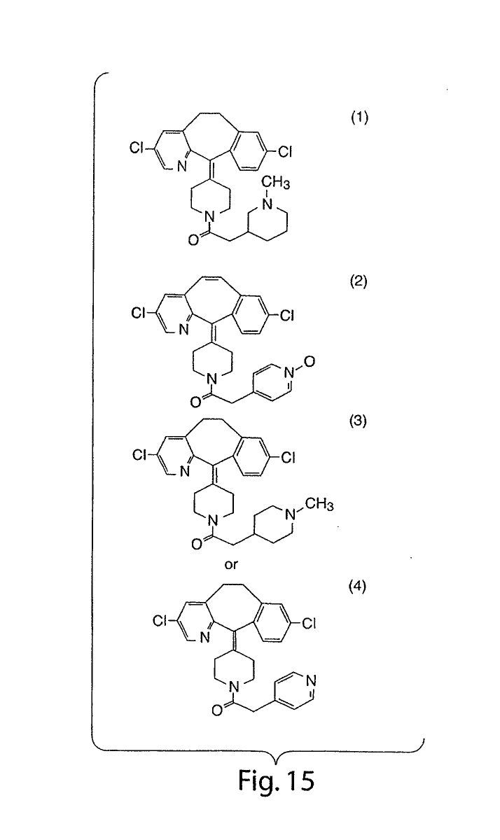

In one embodiment, R¹ is H. In one embodiment, R³ is at the C-8 position. In one embodiment, R⁴⁶ is 4-pyridyl N-oxide, 4-N-methyl piperidinyl, or 3-N-methylpiperidinyl. In one embodiment, the compound may have any structure shown in FIG. 15.

In one aspect, the compound may be of the formula: 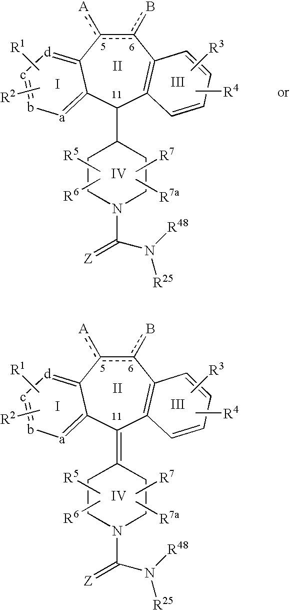
wherein: a represents N and the remaining b, c and d groups represent CR¹ or CR²;
R¹ and R² are independently selected from H, halo, —CF₃, lower alkyl or benzotriazol-1-yloxy;
R³ and R⁴ are independently selected from H or halo;
R⁵, R⁶, R⁷ and R⁸ are H;
the dotted line between carbon atoms 5 and 6 represents an optional double bond, such that when a double bond is present, A and B independently represent H, and when no double bond is present between carbon atoms 5 and 6, A and B each independently represent H₂;
R²⁵ represents pyridyl, pyridyl N-oxide, N-methyl-piperidinyl or phenyl;
R⁴⁸ represents H or alkyl; and
Z represents O.

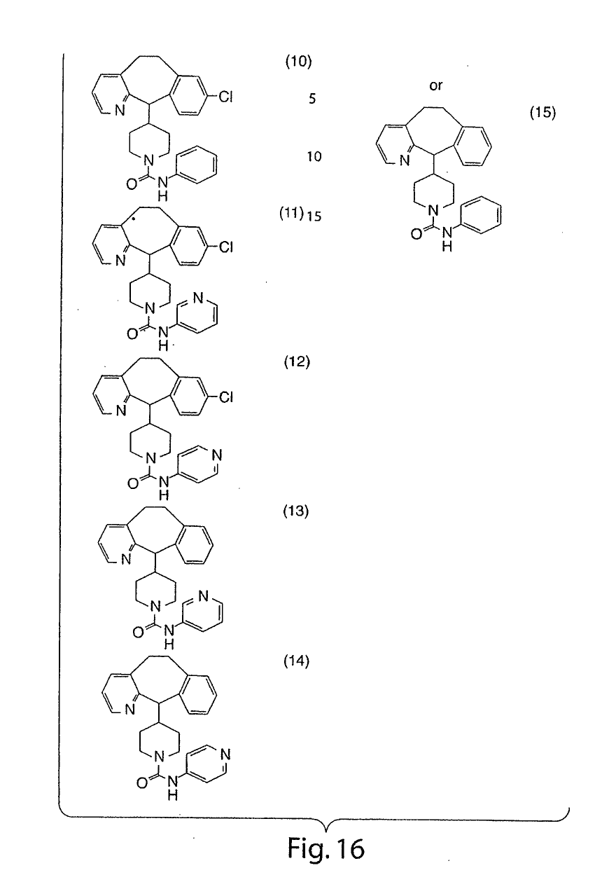

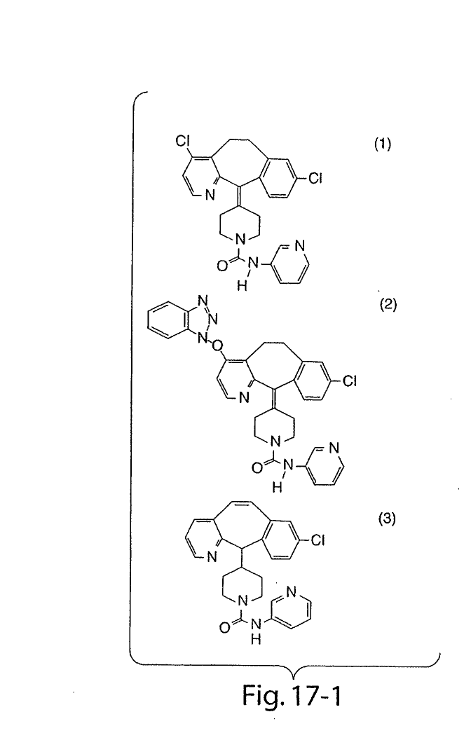

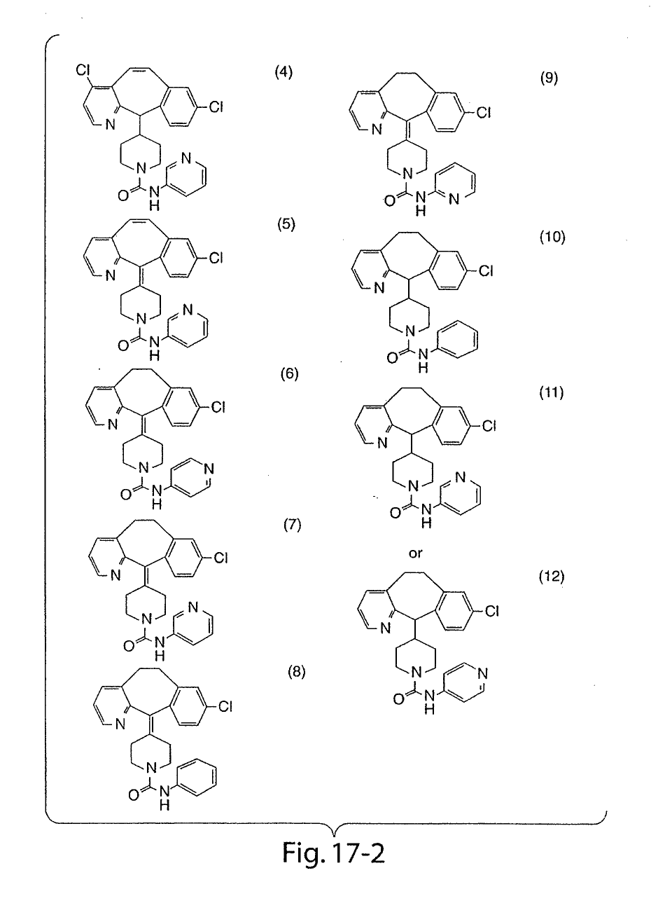

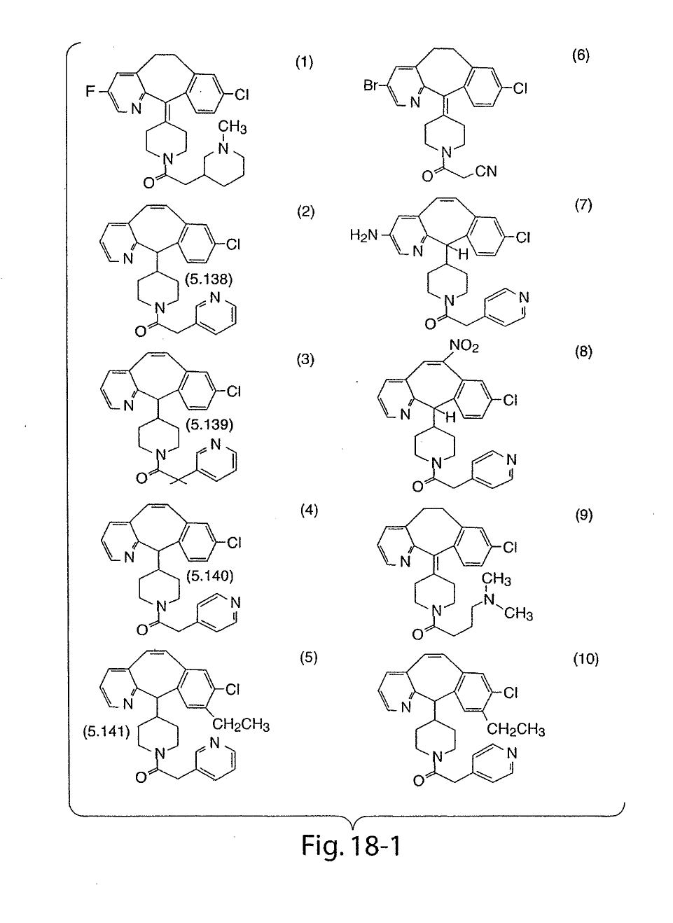

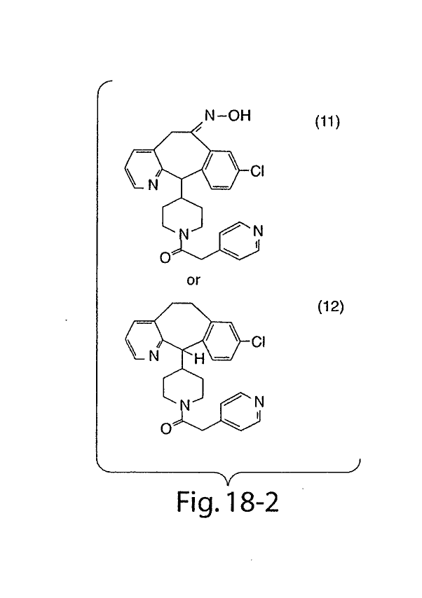

In one embodiment, R¹ is Cl or H; and R² is H, Cl or Br. In one embodiment, R³ is Cl. In one embodiment, R²⁵ represents phenyl, 2-pyridyl, 3-pyridyl, 4-pyridyl, 2-pyridyl N-oxide, 3-pyridyl N-oxide, or 4-pyridyl N-oxide. In one embodiment, R⁴⁸ represents H or methyl. In one embodiment, R²⁵ represents phenyl, 2-pyridyl, 3-pyridyl, 4-pyridyl, 2-pyridyl N-oxide, 3-pyridyl N-oxide, or 4-pyridyl N-oxide; and R⁴⁸ represents H or methyl. In one embodiment, R¹ is Cl or H; R² is Br, Cl, or I; R³ and R⁴ independently represent H or halo; R²⁵ represents phenyl, 2-pyridyl, 3-pyridyl, 4-pyridyl, 2-pyridyl N-oxide, 3-pyridyl N-oxide, or 4-pyridyl N-oxide; and R⁴⁸ represents H or methyl. In one embodiment, R³ is Cl at the C-8 position and R⁴ is H. In one embodiment, the compound may have any structure shown in FIG. 16, FIG. 17, or FIG. 18.

In one aspect, the compound may be of the formula: 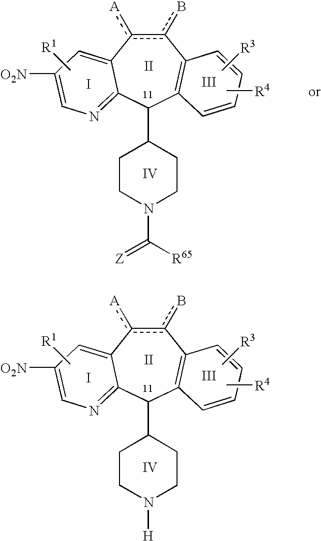
wherein:
R¹ is selected from H or halo;
R³ is Cl;
R⁴ is H or halo;
the dotted line between carbon atoms 5 and 6 represents an optional double bond, such that when a double bond is present, A and B independently represent H, and when no double bond is present between carbon atoms 5 and 6, A and B each independently represent H₂; and
R⁶⁵ represents H or —OR⁶⁶ wherein R⁶⁶ represents alkyl.

In one embodiment, the compound is:

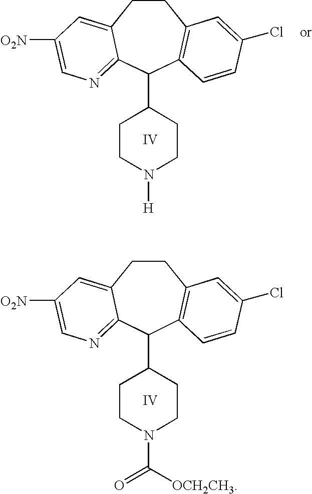

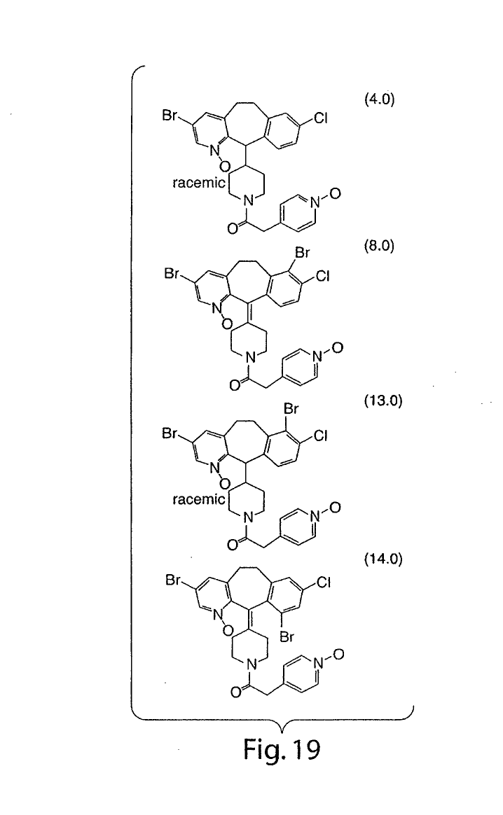

In another aspect, the invention provides a method of treating a synucleinopathic subject by administering a farnesyl transferase inhibitor compound having a formula shown in FIG. 19, or a stereoisomeric form, or a pharmaceutically acceptable acid or base addition salt form thereof, in a therapeutically effective amount.

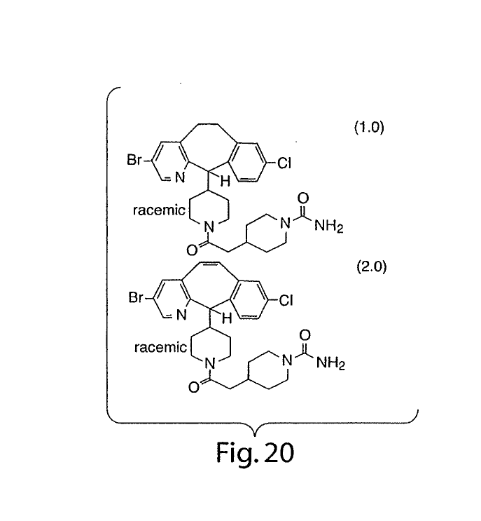

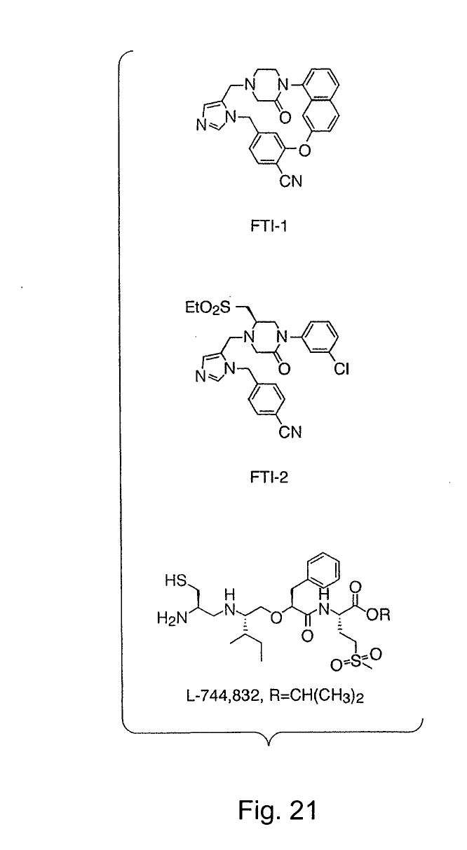

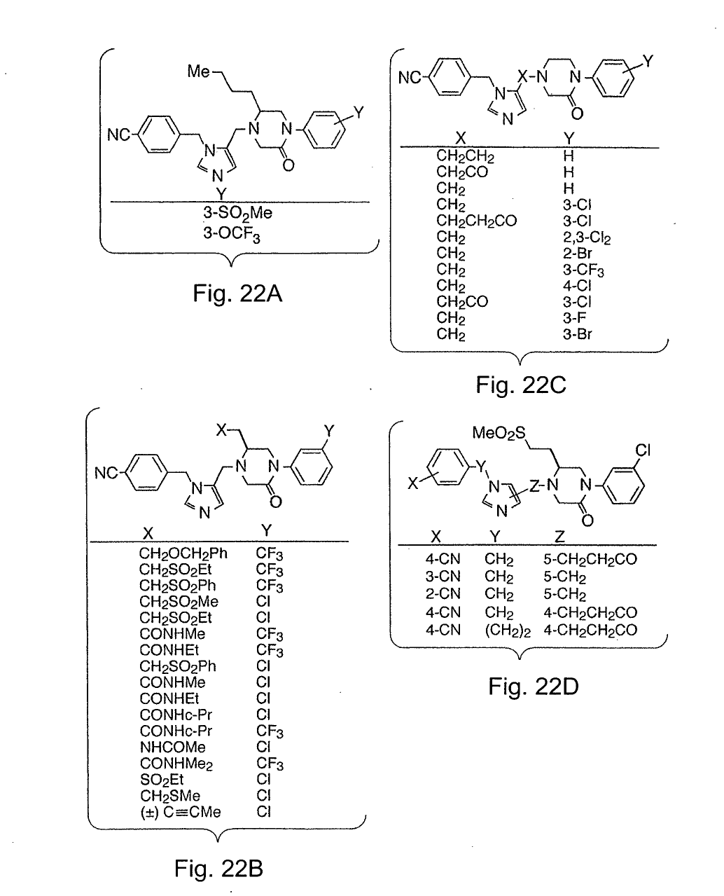

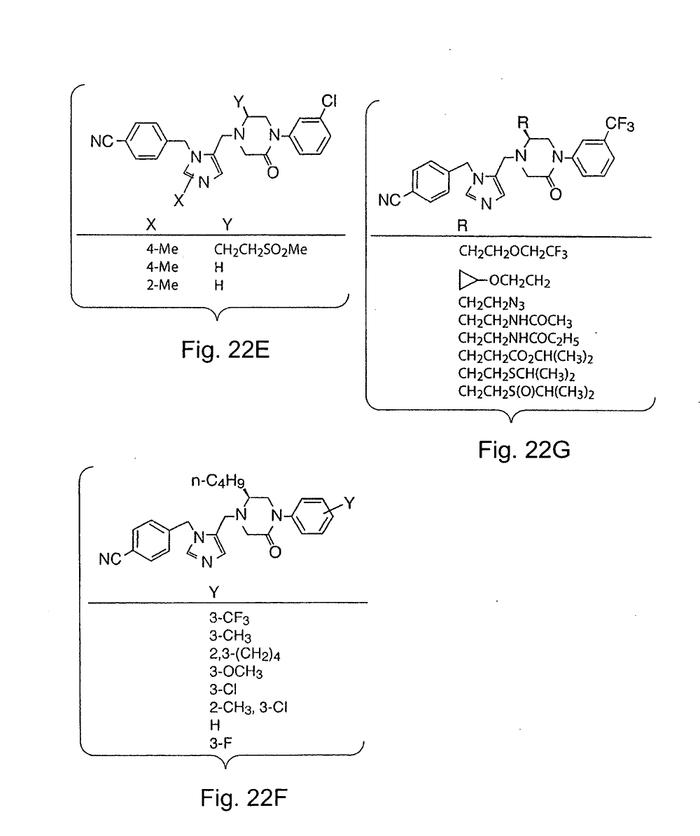

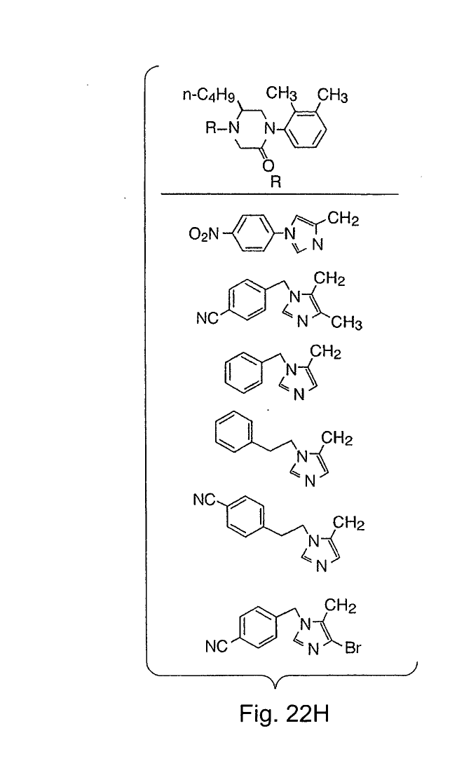

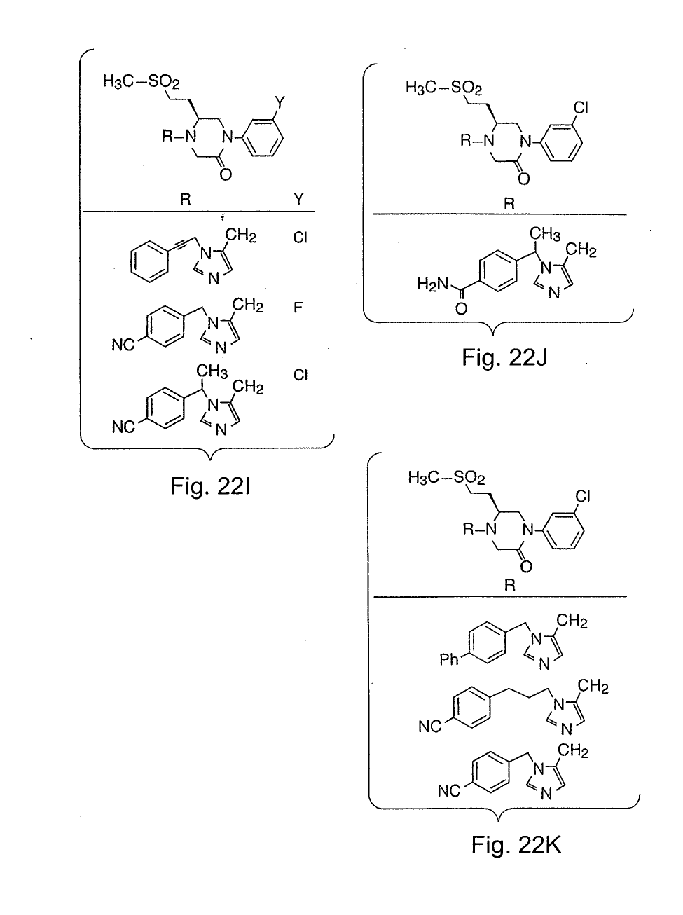

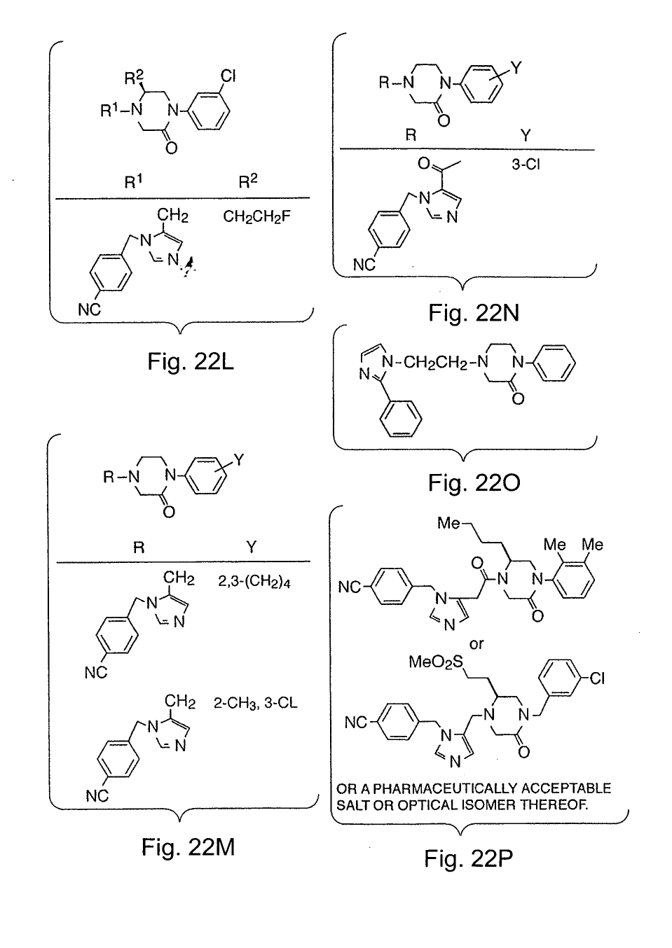

In another aspect, the invention provides a method of treating a synucleinopathic subject by administering a farnesyl transferase inhibitor having a formula shown in FIG. 20, or a stereoisomeric form, or a pharmaceutically acceptable acid or base addition salt form thereof, in a therapeutically effective amount. In one embodiment, the compound is:

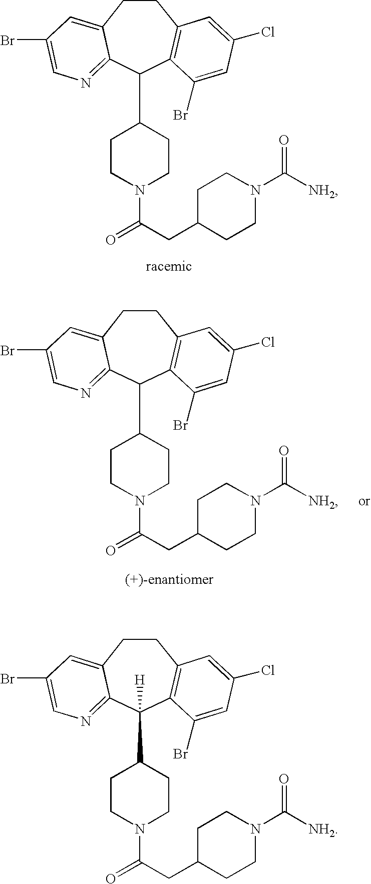

In another aspect, the invention provides a method of treating a synucleinopathic subject by administering a therapeutically effective amount of a stereoisomeric form, or a pharmaceutically acceptable acid or base addition salt form of a farnesyl transferase inhibitor compound of the formula:

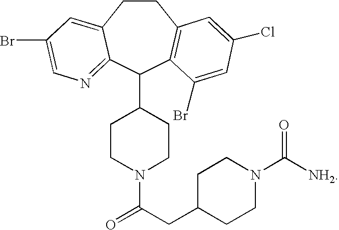

In another aspect, the invention provides a method of treating a synucleinopathic subject, by administering a therapeutically effective amount of a stereoisomeric form, or a pharmaceutically acceptable acid or base addition salt form of a farnesyl transferase inhibitor compound of the formula:

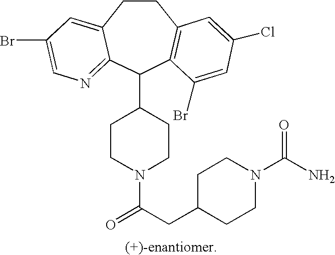

In another aspect, the invention provides a method of treating a synucleinopathic subject by administering a therapeutically effective amount of a stereoisomeric form, or a pharmaceutically acceptable acid or base addition salt form of a farnesyl transferase inhibitor compound of the formula:

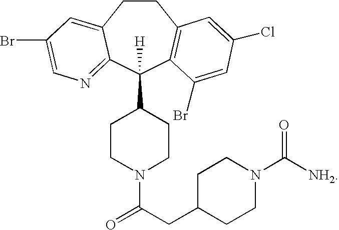

It should be appreciated that in any of the aspects or embodiments described herein, the farnesyl transferase inhibitor compound(s) may be provided in any suitable stereoisomeric form, and/or pharmaceutically acceptable acid or base addition salt form, and in a therapeutically effective amount. Also, in any one of the aspects or embodiments described herein, the synucleinopathic subject may have a synucleinopathy selected from the group consisting of: Parkinson's disease, diffuse Lewy body disease, and multiple system atrophy disorder. The subject may be human. The effective amount of any one or more compounds may be from about 10 ng/kg of body weight to about 1000 mg/kg of body weight, and the frequency of administration may range from once a day to once a month. However, other dosage amounts and frequencies also may be used as the invention is not limited in this respect. In addition, a subject may be administered one or more non-farnesyl transferase inhibitor compounds in an amount effective to treat a neurological disorder. In one embodiment, the non-farnesyl transferase inhibitor compound(s) may be one or more of the following: dopamine agonist, DOPA decarboxylase inhibitor, dopamine precursor, monoamine oxidase blocker, cathechol O-methyl transferase inhibitor, anticholinergic, and/or NMDA antagonist. For example, the non-farnesyl trasferase inhibitor compound(s) may be Memantine, Aricept, and/or other acetylcholinesterase inhibitors.

Another aspect of the invention provides an article of manufacture comprising packaging material and a farnesyl transferase inhibitor compound, wherein the article of manufacture further comprises a label or package insert indicating that the farnesyl transferase inhibitor compound can be administered to a subject for treating a synucleinopathy. In a preferred embodiment the subject is a human. In one embodiment the invention comprises further administering to the subject an amount of one or more non-farnesyl transferase inhibitor compounds effective to treat a neurological disorder. In one embodiment of the invention each non-farnesyl transferase inhibitor compound is selected from the group consisting of: dopamine agonist, DOPA decarboxylase inhibitor, dopamine precursor, monoamine oxidase blocker, cathechol O-methyl transferase inhibitor, anticholinergic, and NMDA antagonist. In one embodiment of the invention each non-farnesyl trasferase inhibitor compound is selected from the group consisting of Memantine, Aricept, and other acetylcholinesterase inhibitors.

In methods of the invention, the term “synucleinopathic subject” refers to a subject that is affected by or at risk of developing a synucleinopathy (e.g. predisposed, for example genetically predisposed, to developing a synucleinopathy) and/or any neurodegenerative disorders characterized by pathological synuclein aggregations. Several neurodegenerative disorders including Parkinson's Disease, Diffuse Lewy Body disease (DLBD) and Multiple System Atrophy (MSA) are collectively grouped as synucleinopathies.

Synucleins are small proteins (123 to 143 amino acids) characterized by repetitive imperfect repeats SEQ ID NO: 8 (KTKEGV) distributed throughout most of the amino terminal half of the polypeptide in the acidic carboxy-terminal region. There are three human synuclein proteins termed α, β, and γ, and they are encoded by separate genes mapped to chromosomes 4221.3-q22, 5q23 and 10q23.2-q23.3, respectively. The most recently cloned synuclein protein synoretin, has a close homology to y synuclein and is predominantly expressed within the retina. α-synuclein, also referred to as non-amyloid component of senile plaques precursor protein (NACP), SYN1 or synelfin, is a heat-stable, “natively unfolded” protein of poorly defined function. It is predominantly expressed in the central nervous system (CNS) neurons where it is localized to presynaptic terminals. Electron microscopy studies have localized α-synuclein in close proximity to synaptic vesicles at axonal termini, suggesting a role for α-synuclein in neurotransmission or synaptic organization, and biochemical analysis has revealed that a small fraction of α-synuclein may be associated with vesicular membranes but most α-synuclein is cytosolic.

Genetic and histopathological evidence supports the idea that α-synuclein is the major component of several proteinaceous inclusions characteristic of specific neurodegenerative diseases. Pathological synuclein aggregations are restricted to the α-synuclein isoforms, as β and γ synucleins have not been detected in these inclusions. The presence of α-synuclein positive aggregates is disease specific. Lewy bodies, neuronal fibrous cytoplasmic inclusions that are histopathological hallmarks of Parkinson's Disease (PD) and Diffuse Lewy Body disease (DLBD) are strongly labeled with antibodies to α-synuclein. Dystrophic ubiquitin-positive neurites associated with PD pathology, termed Lewy neurites (LN) and CA2/CA3 ubiquitin neurites are also α-synuclein positive. Furthermore, pale bodies, putative precursors of LBs, thread-like structures in the perikarya of slightly swollen neurons and glial silver positive inclusions in the midbrains of patients with LB diseases are also immunoreactive for α-synuclein. α-synuclein is likely the major component of glial cell inclusions (GCIs) and neuronal cytoplasmic inclusions in MSA and Hallervorden-Spatz disease (brain iron accumulation type 1). α-synuclein immunoreactivity is present in some dystrophic neurites in senile plaques in Alzheimer's Disease, but is not detected in Pick bodies neurofibrillary tangles (NFTs), neurophil threads, or in neuronal or glial inclusion characteristic of Progressive Supranuclear Palsy, Corticolbasal Degeneration, motor neuron disease and trinucleotide-repeat diseases.

Further evidence supports the notion that α-synuclein is the actual building block of the fibrillary components of LBs, LNs and GCIs. Immunoelectron microscopic studies have demonstrated that these fibrils are intensely labeled with α-synuclein antibodies in situ. Sarcosyl-insoluble α-synuclein filaments with straight and twisted morphologies can also be observed in extracts of DLBD and MSA brains. Moreover, α-synuclein can assemble in vitro into elongated homopolymers with similar widths as sarcosyl-insoluble fibrils or filaments visualized in situ. Polymerization is associated with a concomitant change in secondary structure from random coil to anti-parallel β-sheet structure consistent with the Thioflavine-S reactivity of these filaments. Furthermore, the PD-association with α-synuclein mutation, A53T, may accelerate this process, as recombinant A53T α-synuclein has a greater propensity to polymerize than wild-type α-synuclein. This mutation also affects the ultrastructure of the polymers; the filaments are slightly wider and are more twisted in appearance, as if assembled from two protofilaments. The A30P mutation may also modestly increase the propensity of α-synuclein to polymerize, but the pathological effects of this mutation also may be related to its reduced binding to vesicles. Interestingly, carboxyl-terminally truncated α-synuclein may be more prone to form filaments than the full-length protein.

According to the invention, the proteosomal degradation of α-synuclein is a mediated by parkin and neuronal ubiquitin C-terminal hydrolase (UCH-L1). Parkin is an E3 ligase that ubiquitinylates α-synuclein and thereby tags it for degradation. UCH-L1 acts in normal neuronal tissues to cleave the ubiquitinylated proteins that are products of the proteosomal degradation of the polyubiquitinylated proteins.

The invention provides methods for treating synucleinopathic disorders using inhibitors of farnesyl transferase. It has been now discovered that UCH-L1 is farnesylated in vivo. UCH-L1 is associated with the membrane and this membrane association is mediated by farnesylation. Farnesylated UCH-L1 also stabilizes the accumulation of α-synuclein. The invention relates to the prevention or inhibition of UCH-L1 farnesylation which would result in UCH-L1 membrane disassociation and acceleration of the degradation of α-synuclein. Since α-synuclein accumulation is pathogenic in PD, DLBD, and MSA, an increased degradation of α-synuclein and/or inhibition of α-synuclein accumulation ameliorates the toxicity associated with a pathogenic accumulation of α-synuclein.

The modification of a protein by a farnesyl group can have an important effect on function for a number of proteins. Farnesylated proteins typically undergo further C-terminal modification events that include a proteolytic removal of three C-terminal amino acids and carboxymethylation of C-terminal cystines. These C-terminal modifications facilitate protein-membrane association as well as protein-protein interactions. Farnesylation is catalyzed by a protein farnesyltransferase (FTase), a heterodimeric enzyme that recognizes the CAAX motif present at the C-terminus of the substrate protein. FTase transfers a farnesyl group from farnesyl pyrophosphate and forms a thioether linkage between the farnesyl and the cystine residues in the CAAX motif. A number of inhibitors of FDase have been developed and are known in the art. However, the invention provides novel methods for using certain farnesyl transferase inhibitors to treat subjects having symptoms associated with α-synuclein accumulation.

In methods of the invention, the term “synucleionopathy” refers to neurological disorders that are characterized by a pathological accumulation of α-synuclein. This group of disorders includes PD, DLBD and MSA.

Parkinson's Disease (PD) is a neurological disorder characterized by bradykinesia, shuffling gait, postural instability, tremor, and a loss of automatic movement. It is due to the loss of dopamine-containing substantia nigra cells. It appears that about 50% of the cells need to be lost before symptoms appear. Associated symptoms often include rigidity, difficulty initiating movement (akinesia), small handwriting (micrographia), seborrhea, orthostatic hypertension, urinary difficulties, constipation, lymph pain, depression, dementia (up to a third of the patients), smelling disturbances (occurs early). Orthostatic hypertension might occur associated with the disease or as a complication of medication. Patients with Parkinsonism have greater mortality, about two times compared to general population without PD. This is attributed to greater frailty or reduced mobility.

The term “synucleinopathic subject” encompasses a subject that is affected by, or is at risk of developing PD. These subjects can be readily identified by persons of ordinary skill in the art by symptomatic diagnosis or by genetic screening, brain scans, SPEC, PET imaging etc.

Diagnosis of PD is mainly clinical and is based on the clinical findings listed above. There are many conditions which may be mistaken for Parkinsonism. Among the most common are side effects of drugs, mainly the major tranquilizers, such as Haldol, strokes involving the basal ganglea, degenerative disorders, such as progressive supranuclear palsy (PSP), olivopontocerebellar degeneration (OPCD), MSA, and Huntington's Disease. The pathological hallmark of PD are Lewy bodies, which are intracytoplasmatic inclusion bodies in effected neurons of the substantion nigra. Recently, α-synuclein has been identified as the main component of Lewy bodies in sporadic Parkinsonism.

Although Parkinson's can be clearly traced to genetic factors, viruses, stroke, or toxins in few individuals for the most part the cause of Parkinson's in any particular case is unknown (this is referred to as sporadic PD). Environmental influences include drinking well water, farming and industrial exposure to heavy metals (iron, zinc, copper, mercury, magnesium and manganese), alkylated phosphates and orthonal chlorines. Paraquat (a herbicide) has been associated with increased prevalence of Parkinsonism, cigarette smoking is associated with the decrease incidence. The current consensus is that Parkinsonism may either be caused by an uncommon toxin combined with high genetic susceptibility or a common toxin combined with relatively low genetic susceptibility.

Subjects that are at risk of developing PD can be identified for example by genetic analysis. There is good evidence for genetic factors associated with PD. Large pedigrees of autosomal dominantly inherited PDs have been reported. A mutation in α-synuclein is responsible for one pedigree.

Methods of the invention can be used in combination with one or more alternative medications, including medications that are currently used to treat synucleinopathies or symptoms arising as side-effects of the disease or of the aforementioned medications.

For example, methods of the invention can be used in combination with medications for treating PD. Levodopa mainly in the form of combination product containing carbodopa and levodopa (Synemat and Synemat CR) is the mainstay of treatment and is the most effective agent for the treatment of PD. Levodopa is a dopamine precursor, a substance that is converted into dopamine by an enzyme in the brain. Carbodopa is a peripheral dicarboxylase inhibitor which prevents side effects and lower the overall dosage requirement. The starting dose of Synemat is 125/100 tablet prior to each meal. User maintenance dose is lower. Dyskinesias may result from overdose and also are commonly seen after prolonged (e.g., years) use. Direct acting dopamine agonists may have less of this side effect. Orthostatic hypertension may respond to increased carbodopa. About 15% of patients do not respond to levodopa. Dopamine is metabolized to potentially toxic-free radicals and some feel that a direct-acting dopamine agonist should be used early to supplement a dopamine agonist. Stalevo (carbodopa, levodopa, and entacapone) is a new combination tablet for patients who experience signs and symptoms of “wearing-off”. The tablet combines carbodopa, levodopa, (the most widely agents for PD) with entacapone, while carbodopa reduces the side effects of levodopa, entacapone extends the time levodopa is active in the brain, up to 10% longer.

Amantidine (Symmetrel) is a mild agent thought to work by blocking the re-uptake of dopamine into presynaptic neurons. It also activates the release of dopamine from storage sites and has a glutamate receptor blocking activity. It is widely used as early monotherapy and the dosing is 200 to 300 mg daily. Amantidine is particularly helpful in patients with predominant tremor. Side effects include ankle swelling and red blotches. Unfortunately, it's effect in more advanced PD is often short-lived with patients reporting a “fallout effect”.

Anticholinergics (trihexyphenidyl, benztropine mesylate, procyclidine, artane, cogentin) do not act directly on the dopaminergic system. Direct-acting dopamine agonists include bromocriptidine (Parlodel), pergolide (Permax), ropinirol (Requip), and pramipexole (Mirapex). These agents cost substantially more than the levodopa (Synemat) with controversial additional benefits. Depending on which dopamine receptor is being stimulated, D1 and D2 agonist can exert anti-Parkinson effects by stimulating the D1 and D2 receptors, such as Ergolide. Mirapex and Requip are the newer agents. Both are somewhat selected for dopamine receptors with highest affinity for the D2 receptor and also activity at the D3 receptor. Direct dopamine agonists, in general, are more likely to produce adverse neuro psychiatric side effects than levodopa, such as confusion. Unlike levodopa, direct dopamine agonists do not undergo conversion to dopamine and thus do not produce potentially toxic metabolites. It is also possible that the early use of direct dopamine agonist might protect against the development of late complications of dopamine, such as the “on-off” effect.

Monoaminoxidase-B inhibitors (MAO) such as selegiline (Diprenyl, or Eldepryl), taken in a low dose, can initially reduce the progression of Parkinsonism. These compounds can be used as an adjunctive medication. A study has documented that selegiline delays the need for levodopa by roughly three months.

Catechol-O-methyltransferase inhibitors (COMT) can also be used in combination treatments of the invention. Catechol-O-methyltransferase is an enzyme that degrades levodopa and inhibitors can be used to reduce the rate of degradation. Entocapone is a peripherally acting COMT inhibitor, which can be used in certain methods and compositions of the invention. Tasmar or Tolcapone, approved by the FDA in 1997, can also be used in certain methods and compositions of the invention. Psychiatric adverse effects that are induced by PD medication include psychosis, confusion, agitation, hallucinations, and delusions. These can be treated by decreasing dopamine medication, reducing or discontinuing anticholinergics, amantidine or selegiline or by using clozipine, for example at doses of 6.25 to 50 mg/day.

Methods of the invention can also be used in combination with surgical therapies for the treatment of PD. Surgical treatment is presently recommended for those who have failed medical management of PD. Unilateral Thallamotomy—can be used to reduce tremor. It is considered for patients with unilateral tremor not responding to medication. The improvement fades with time. Bilateral procedures are not advised. Unilateral pallidotomy is an effective technique for reducing contralateral levodopamine dyskinesias. Unilateral deep brain stimulation of the thalamus for tremor may also be a benefit for tremor. Neurotransplantation is no longer felt to be an effective treatment. Gamma knife surgery—thalamotomy or pallidotomy—can be performed to focus radiation. In addition to surgery and medication, physical therapy in Parkinsonism maintains muscle tone, flexibility, and improves posture and gait.

According to the invention, the term “synucleinopathic subject” also encompasses a subject that is affected by, or is at risk of developing DLBD. These subjects can be readily identified by persons of ordinary skill in the art by symptomatic diagnosis or by genetic screening, brain scans, SPEC, PET imaging etc.

DLBD is the second commonest cause of neurodegenerative dementia in older people, it effects 7% of the general population older than 65 years and 30% of those aged over 80 years. It is part of a range of clinical presentations that share a neurotic pathology base of normal aggregation of the synaptic protein α-synuclein. DLBD has many of the clinical and pathological characteristics of the dementia that occurs during the course of Parkinson's Disease. An “one year rule” can been used to separate DLBD from PD. According to this rule, onset of dementia within 12 months of Parkinsonism qualifies as DLBD, whereas more than 12 months of Parkinsonism before onset of dementia qualifies as PD. The central features of DLBD include progressive cognitive decline of sufficient magnitude to interfere with normal social and occupational function. Prominent or persistent memory impairment does not necessarily occur in the early stages, but it is evident with progression in most cases. Deficits on tests of attention and of frontal cortical skills and visual spatial ability can be especially prominent.

Core diagnostic features, two of which are essential for diagnosis of probable and one for possible DLBD are fluctuating cognition with pronounced variations in attention and alertness, recurrent visual hallucinations that are typically well-formed and detailed, and spontaneous features of Parkinsonism. In addition, there can be some supportive features, such as repeated falls, syncope, transient loss of consciousness, neuroleptic sensitivity, systematized delusions, hallucinations and other modalities, REM sleep behavior disorder, and depression. Patients with DLBD do better than those with Alzheimer's Disease in tests of verbal memory, but worse on visual performance tests. This profile can be maintained across the range of severity of the disease, but can be harder to recognize in the later stages owing to global difficulties. DLBD typically presents with recurring episodes of confusion on a background of progressive deterioration. Patients with DLBD show a combination of cortical and subcortical neuropsychological impairments with substantial attention deficits and prominent frontal subcortical and visual special dysfunction. These help differentiate this disorder from Alzheimer's Disease.

Rapid eye movement (REM), sleep behavior and disorder is a parasomnia manifested by vivid and frightening dreams associated with simple or complex motor behavior during REM sleep. This disorder is frequently associated with the synucleinopathies, DLBD, PD and MSA, but it rarely occurs in amyloidopathies and taupathies. The neuropsychological pattern of impairment in REM sleep behavior disorder/dementia is similar to that reported in DLBD and qualitatively different from that reported in Alzheimer's Disease. Neuropathological studies of REM sleep behavior disorder associated with neurodegenerative disorder have shown Lewy body disease or multiple system atrophy. REM sleep wakefulness disassociations (REM sleep behavior disorder, daytime hypersomnolence, hallucinations, cataplexy) characteristic of narcolepsy can explain several feature of DLBD, as well as PD. Sleep disorders could not contribute to the fluctuations typical of DLBD and their treatment can improve fluctuations and quality of life. Subjects at risk of developing DLBD can be identified. Repeated falls, syncope, transient loss of consciousness, and depression are common in older people with cognitive impairment and can serve as (a red flag) to a possible diagnosis of DLBD. By contrast, narcoleptic sensitivity in REM sleep behavior disorder can be highly predictive of DLBD. Their detection depends on the clinicians having a high index of suspicion and asking appropriate screening questions.

Clinical diagnosis of synucleinopathic subjects that are affected by or at risk of developing LBD can be supported by neuroimaging investigations. Changes associated with DLBD include preservation of hippocampal, and medialtemperalobe volume on MRI and sipital hyperprofusion on SPECT. Other features, such as generalized atrophy, white medichanges and rates of progression of whole brain atrophy are not helpful in differential diagnosis. Dopamine transported a loss in the caudate and putamen, a marker of nigrostriatal degeneration can be detected by dopomenergic SPECT and can prove helpful in clinical differential diagnosis. A sensitivity of 83% and specificity of 100% has been reported for an abnormal scan with an autopsy diagnosis of DLBD.

Consensus criteria for diagnosing DLBD include ubiquitin immunohistochemistry for Lewy body identification and staging into three categories; brain stem predominant, limbic, or neocortical, depending on the numbers and distribution of Lewy bodies. The recently-developed α-synuclein immunohistochemistry is a better marker that visualizes more Lewy bodies and also better source previously under recognized neurotic pathology, termed Lewy neurites. Use of antibodies to α-synuclein moves the diagnostic rating for many DLBD cases from brain stem and limbic groups into the neocortical group.

In most patients with DLBD, there are no genetic mutations in the α-synuclein or other Parkinson's Disease genes. Pathological up-regulation of normal, wild-type α-synuclein due to increased mRNA expression is a possible mechanism, or Lewy bodies may form because α-synuclein becomes insoluble or more able to aggregate for some reason. Another possibility is that α-synuclein is abnormally processed, for example, by dysfunctional proteosome system and that toxic “proto fibrils” are therefore produced. Sequestering of these toxic fibrils into Lewy bodies could reflect an effort by the neurons to combat biological stress inside the cell, rather than their simply being neurodegenerative debris.

Target symptoms for the accurate of DLBD can include extrapyramidal motor features, cognitive impairment, neuropsychiatric features (including hallucinations, depression, sleep disorder, and associated behavioral disturbances) or autonomic dysfunction.

Methods of the invention can be used in combination with one or more alternative medications for treating DLBD. For example, lowest acceptable doses of levodopa can be used for treating DLBD. D2-receptor antagonists, particularly traditional neuroleptic agents can provoke severe sensitivity reactions in DLBD subjects with an increase in mortality of two to three times. Cholinsterase inhibitors dicussed above are also used in the treatment of DLBD.

According to the invention, the term “synucleinopathic subject” also encompasses a subject that is affected by, or is at risk of developing MSA. These subjects can be readily identified by persons of ordinary skill in the art by symptomatic diagnosis or by genetic screening, brain scans, SPEC, PET imaging etc.

MSA is a neurodegenerative disease marked by a combination of symptoms; affecting movement, blood pressure, and other body functions, hence the label “multiple system atrophy”. The cause of MSA is unknown. Symptoms of MSA vary in distribution of onset and severity from person to person. Because of this, three different diseases were initially described to accomplish this range of symptoms; Shy-Drager syndrome, striatonigral degeneration (SD), and olivopontocerebellar atrophy (OPCA).

In Shy-Drager syndrome, the most prominent symptoms are those involving the autonomic system; blood pressure, urinary function, and other functions not involving conscious control. Striatonigral degeneration causes Parkinsonism symptoms, such as slowed movements and rigidity, while OPCA principally effects balance, coordination and speech. The symptoms for MSA can also include orthostatic hypertension, male impotence, urinary difficulties, constipation, speech and swallowing difficulties, and blurred vision.

The initial diagnosis of MSA is usually made by carefully interviewing the patient and performing a physical examination. Several types of brain imaging, including computer histomography, scans, magnetic resonance imaging (MRI), and positron emission tomography (PET), are used. Pharmacological challenge tests (administering certain drugs in the presence of various types of movement of the patient) may also be of help in those patients with typical Parkinsonism signs. An incomplete and relatively poor response to dopamine replacement therapy, such as Sinemet, may be a clue that MSA is present. A characteristic involvement of multiple brain systems is a defining feature of MSA and one that an autopsy confirms the diagnosis. Patients with MSA can have the presence of glial cytoplasmic inclusions in certain types of brain cells, as well. Lewy bodies are not present in MSA. In comparison to Parkinson's, in addition to the poor response to Sinemet, there are a few other observations that are suggested for MSA, such as low blood pressure on standing, difficulty with urination, use of a wheelchair, loud snoring or loud breathing, and frequent nighttime urination.

Methods of the invention can be used in combination with one or more alternative medications for treating MSA. Typically, the drugs that can be used to treat various symptoms of MSA become less effective as the disease progresses. Levodopa and dopamine agonists used to treat PD are sometimes effective for the slowness and rigidity of MSA. Orthostatic hypertension can be improved with cortisone, midodrine, or other drugs that raise blood pressure. Male impotence may be treated with penile implants or drugs. Incontinence may be treated with medication or catheterization. Constipation may improve with increased dietary fiber or laxatives.

According to the invention, the term “treatment” includes prophylaxis and therapy, and includes managing a synucleinopathic subject's symptoms and halting the progression of the synucleinopathy. Treatment includes preventing, slowing, stopping, or reversing (e.g. curing) the development of a synucleinopathy, and/or the onset of certain symptoms associated with a synucleinopathy in a subject with, or at risk of developing, a synucleinopathy or a related disorder. Therapy includes preventing, slowing, stopping or reversing (e.g. curing) the accumulation of α-synuclein in a subject with a synucleinopathy. Therapy also includes decreasing the amount of accumulated α-synuclein in a subject with a synucleinopathy.

The phrase “therapeutically-effective amount” as used herein means that amount of a compound, material, or composition comprising a compound of the present invention which is effective for producing some desired therapeutic effect in a subject at a reasonable benefit/risk ratio applicable to any medical treatment. Accordingly, a therapeutically effective amount prevents, minimizes, or reverses disease progression associated with a synucleinopathy. Disease progression can be monitored by clinical observations, laboratory and neuroimaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The “pharmaceutically acceptable acid or base addition salts” mentioned herein are meant to comprise the therapeutically active non-toxic acid and non-toxic base addition salt forms that the compounds are able to form. The compounds that have basic properties can be converted into their pharmaceutically acceptable acid addition salts by treating the base form with an appropriate acid. Appropriate acids include, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The compounds that have acidic properties can be converted into their pharmaceutically acceptable base addition salts by treating the acid form with a suitable organic or inorganic base. Appropriate base salt forms include, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds are able to form. Examples of such forms are e.g. hydrates, alcoholates and the like.

The term stereochemically isomeric forms of compounds, as used herein, include all possible compounds made up of the same atoms bonded by the same sequence of bonds but having different three-dimensional structures which are not interchangeable, which the compounds may possess. Unless otherwise mentioned or indicated, the chemical designation of a compound encompasses the mixture of all possible stereochemically isomeric forms that the compound can take. The mixture can contain all diastereomers and/or enantiomers of the basic molecular structure of the compound. All stereochemically isomeric forms of the compounds both in pure form or in admixture with each other are intended to be embraced within the scope of the present invention.

Some of the compounds may also exist in their tautomeric forms. Such forms although not explicitly indicated in the above formula are intended to be included within the scope of the present invention.

The methods and structures described herein relating to compounds and compositions of the invention also apply to the pharmaceutically acceptable acid or base addition salts and all stereoisomeric forms of these compounds and compositions.

In the compounds and compositions of the invention, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 12 or fewer carbon atoms in its backbone (e.g., C₁-C₁₂ for straight chain, C₃-C₁₂ for branched chain), and more preferably 6 or fewer, and even more preferably 4 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure, and even more preferably from one to four carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

As used herein, the term “halogen” designates —F, —Cl, —Br or —I; the term “sulfhydryl” means —SH; and the term “hydroxyl” means —OH.

The term “methyl” refers to the monovalent radical —CH₃, and the term “methoxyl” refers to the monovalent radical —CH₂OH.

The term “aralkyl” or “arylalkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms “ortho”, “meta” and “para” apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” or “heteroaryl” refer to 3- to 10-membered ring structures, more preferably 3- to 7-membered rings, whose ring structures include one to four heteroatoms. Heterocycles can also be polycycles. Heterocyclyl groups include, for example, thiophene, benzothiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, the present invention relates to a compound represented by any of the structures outlined herein, wherein the compound is a single stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as anti-synucleinopathy farnesyl transferase inhibitor compounds), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants, which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As set out herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routs of administration include sublingual, intramuscular, and transdermal administrations.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In some embodiments, a compound or pharmaceutical composition of the invention is provided to a synucleinopathic subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the synucleinopathic subject. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition) as described above.

The compounds according to the invention may be formulated for administration in any convenient way for use in human or veterinary medicine, by analogy with other pharmaceuticals.

According to the invention, compounds for treating neurological conditions or diseases can be formulated or administered using methods that help the compounds cross the blood-brain barrier (BBB). The vertebrate brain [and CNS] has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood.

The unique morphologic characteristics of the brain capillaries that make up the BBB are: (a) epithelial-like high resistance tight junctions which literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

In one aspect of the invention, farnesyl transferase inhibitor compounds that cross the BBB are particularly useful for treating synucleinopathies. In one embodiment, it is expected that farnesyl transferase inhibitors that are non-charged (e.g., not positively charged) and/or non-lipophilic may cross the BBB with higher efficiency than charged (e.g., positively charged) and/or lipophilic compounds. Therefore it will be appreciated by a person of ordinary skill in the art that some of the compounds of the invention might readily cross the BBB. Alternatively, the compounds of the invention can be modified, for example, by the addition of various substitutuents that would make them less hydrophilic and allow them to more readily cross the BBB.

Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference).

Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl and primary amine groups on the drug to make it more lipid-soluble and therefore more easily able to cross the blood-brain barrier.

Another approach to increasing the permeability of the BBB to drugs involves the intra-arterial infusion of hypertonic substances which transiently open the blood-brain barrier to allow passage of hydrophilic drugs. However, hypertonic substances are potentially toxic and may damage the blood-brain barrier.

Peptide compositions of the invention may be administered using chimeric peptides wherein the hydrophilic peptide drug is conjugated to a transportable peptide, capable of crossing the blood-brain barrier by transcytosis at a much higher rate than the hydrophilic peptides alone. Suitable transportable peptides include, but are not limited to, histone, insulin, transferrin, insulin-like growth factor I (IGF-I), insulin-like growth factor II (IGF-II), basic albumin and prolactin.

Antibodies are another method for delivery of compositions of the invention. For example, an antibody that is reactive with a transferrin receptor present on a brain capillary endothelial cell, can be conjugated to a neuropharmaceutical agent to produce an antibody-neuropharmaceutical agent conjugate (U.S. Pat. No. 5,004,697 incorporated herein in its entirety by reference). The method is conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point of between about 8.0 to 11.0 (U.S. Pat. No. 5,527,527 incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion.

The permeability of the blood brain barrier can be increased by administering a blood brain barrier agonist, for example bradykinin (U.S. Pat. No. 5,112,596 incorporated herein in its entirety by reference), or polypeptides called receptor mediated permeabilizers (RMP) (U.S. Pat. No. 5,268,164 incorporated herein in its entirety by reference). Exogenous molecules can be administered to the host's bloodstream parenterally by subcutaneous, intravenous or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g. between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g. bradykinin) can be administered before or at the same time as an intravenous injection of a drug. Thus, the term “co administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system.

In other embodiments, compounds of the invention can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and the farnesyl transferase inhibitor (and optionally another drug). The carrier preferably is a normal component of the central nervous system and is inactive and harmless. The compound and/or drug, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. Pat. Nos. 4,939,174; 4,933,324; 5,994,932; 6,107,499; 6,258,836 and 6,407,137, the disclosures of which are incorporated herein by reference in their entirety.

The administration of the agents of the present invention may be for either prophylactic or therapeutic purpose. When provided prophylactically, the agent is provided in advance of disease symptoms such as any Alzheimer's disease symptoms. The prophylactic administration of the agent serves to prevent or reduce the rate of onset of symptoms. When provided therapeutically, the agent is provided at (or shortly after) the onset of the appearance of symptoms of actual disease. In some embodiments, the therapeutic administration of the agent serves to reduce the severity and duration of Alzheimer's disease.

The function and advantage of these and other embodiments of the present invention will be more fully understood from the examples described below. The following examples are intended to illustrate the benefits of the present invention, but do not exemplify the full scope of the invention.

### EXAMPLES

### Experimental Procedures

Tissue culture: All cell lines were obtained by ATCC. SH-SY5Y and Cos-7 were grown in 10% FBS DMEM (Sigma). Cells were split the day before experiments including transfection, metabolic labeling and drug treatment.

Proteins and antibodies: UCH-L1 variants were purified according to the published procedure. Synuclein antibody (SYN-1) was purchased from Signal Transduction Lab. Actin antibody and FLAG antibody (M2) were from Sigma. UCH-L1 antibody (anti-PGP 9.5) was from Chemicon.

Chemicals: FTI-277 and lactacystin was purchased from Calbiochem. Crosslinking reagent DE was from Pierce. DMEM and MEM were purchased from Gibco. All the other material was purchased from Sigma.

Plasmids: C220S cDNA was generated by PCR site-specific mutagenesis. For the PCR, the 5′ primer is uchforw SEQ ID NO: 1 (CTAAAGCTTATGCAGCTCAAGCCGATGGAG), and 3′ primer is uchc220s SEQ ID NO:2 (CTAAGA CTCGAGTTAGGCTGCCTTGCTGAGAGC). Wt UCH-L1 served as the template. The PCR fragment was inserted into pcDNA vector. For S18YC220S mutant, S18Y UCH-L1 served as the template in PCR. For the FLAG tagged UCH-L1, the 5′ primer is FLAGuchforw SEQ ID NO: 3 (CTAAAGCTTATGGACTACAAGGATGACGACGACAAAGATGCAGCTCAAGC CGATGGAG), and the 3′ primer is uchrev SEQ ID NO: 4 (ATCCTCGAGTTAGGCTGCCTTGACGAGAGC). Wt UCH-L1 or C220S served as the template. PCR fragment was purified and inserted into pcDNA vector. For the FLAG tagged UCH-L3, the 5′ primer is L3HindIII SEQ ID NO: 5 (CTAAAGCTTATGGACTAC AAGGATGACGACGACAAAGATGGAGGGTCAACGCTGGCTG), the 3′ primer is L3XhoISAA SEQ ID NO: 6 (ATCCTCGAGCTATGCTGCAGAAAGAGCAATCGCA). For the UCH-L3 CKAA variant, the 5′ primer is L3 HindIII and the 3′ primer is L3XhoICKAA SEQ ID NO: 7 (ATCCTCGAGCTATGCTGCCTTAGAAAGAGCAATCGCATTAAATC). α-synuclein degradation assay: Liphitamine 2000 was used to transfect COS-7 cells according to the Invitrogen protocol. Transfected cells were cultured at 37° C. for 48 hours before being treated with 35 μM lactacystin or DMSO. After 24 hours of incubation, the cells were lysed with Tris buffer (50 mM Tris, 2% SDS, 0.1% NP-40), and subjected to SDS-PAGE, followed by quantitative Western blotting.

Salt and detergent treatment of SV fraction: SV fraction was prepared as describe elsewhere. SV was incubated with various salts at designed concentration for 30 minutes on ice, or 1% Triton X-100 or control without salts and detergent. Treated SV was pelleted at 100,000 g for 30 minutes. Supernatants and pellets were subjected to SDS-PAGE and Western blotting.

Membrane fractionation: Cells were harvested by scraping and washed with PBS. Cell pellet was suspended in lysis buffer (50 mM Tris-HCl, 1 mM EDTA) supplemented with protease inhibitor cocktail (Sigma) and homogenized by passing through 26G needles 10 times. Suspension was clarified by spinning at 600 g for 5 minutes. Clarified suspension was ultracentrifuged at 100,000 g for 2 hours and separated into membrane and cytosol. Membrane fraction was washed with washing buffer (50 mM Tris-HCl, 1 mM EDTA 1M NaCl), and pelleted each time with bench-top centrifuge.

2D electrophoresis: For the isolation of total cellular protein, cultured SH-SY5Y cells maintained as described above were rinsed with ice-cold PBS. Cells were lysed in 1 ml dSDS buffer (50 mM Tris-HCl, pH 8.0 0.1% SDS) supplemented with protease inhibitor cocktail. Lysates were boiled for 3 min, and were treated with Dnase and Rnase as described. Lysates were precipitated with ice-cold acetone for at least 2 hours, and pellets were resuspended in 2D sample buffer (8M urea, 0.5% CHAPS, 0.2% DTT, 0.5% IPG buffer, 0.002% bromophenol blue). 2D electrophoresis was carried out according to manufacture's protocol (Amersham Life Science). 7 cm pH 4-7 strips were used. For SH-SY5Y membrane fraction, culture SH-SY5Y cells were rinsed with cold PBS and harvested with lysis buffer (50 mM Tris-HCl, pH 8.0, 1 mM ZnAc2, 250 mM sucrose). Lysate was passed through 25G needles for several times and spun at 1000 g for 5 min. Supernatant was centrifuged at 200,000 g for 2 hours. Pellet was extensively washed with lysis buffer and extracted with cold acetone. Pellet was resuspended in 2D sample buffer.

Viral Infection: Viral infection and MTT assay in SH-SY5Y cells: The viruses were amplified and purified according to the published procedure. SH-SY5Y cells were grown on 100 mm petri-dishes and induced with 100 nM RA for 3-5 days before the virus infection with M.I.O at 75. Viruses were diluted with DPBS to desired M.I.O. After four hours of incubation, 10 ml growth medium was added. On the second day, cells were splitted into 96-well plates and treated with compounds for next 48 hours. The growth medium in each well was replaced with growth medium with 5 ug/ml MTT. Medium was removed after three hours incubation, and 200 ul isopropyl (0.04N HCl) was added into each well. The signal was read at 570 nm.

Viable cell counting: At stated time poins, SH-SY5Y cells were trypsinized with 100 ul trypsin-EDTA for 1 minute and neutralized with 400 ul growth medium. Cell suspension was made up by mixing 0.2 ml of cells in growth medium, 0.3 ml of HBSS and 0.5 ml of 0.4% Trypan Blue solution. Viable cell numbers were counted by standard cell counting chamber.

Western Blotting: Following transfer of SDS gels onto NC membrane, all membranes were blocked with 5% non-fat milk in TBST (50 mM Tris-HCl pH7.4, 150 mM NaCl, 0.1% Tween 20), and incubated with primary antibody overnight with 1% BSA in TBST, washed three times with TBST, and incubated with horseradish peroxidase-conjugated secondary antibody for 1 hour (Promega). Bound antibodies were detected using enhanced chemiluminascence (NEM).

### Example 1

### UCH-L1 is Farnesylated In Vivo and in Cell Culture

The UCH-L1 sequence contains the sequence CXXX, a consensus farnesylation site, at its C-terminus. This sequence is not present in UCH-L3. The possibility that this sequence was modified in vivo was investigated. First, the chemical nature of the previously reported association of UCH-L1 and synaptic vesicles from rat brain was probed.

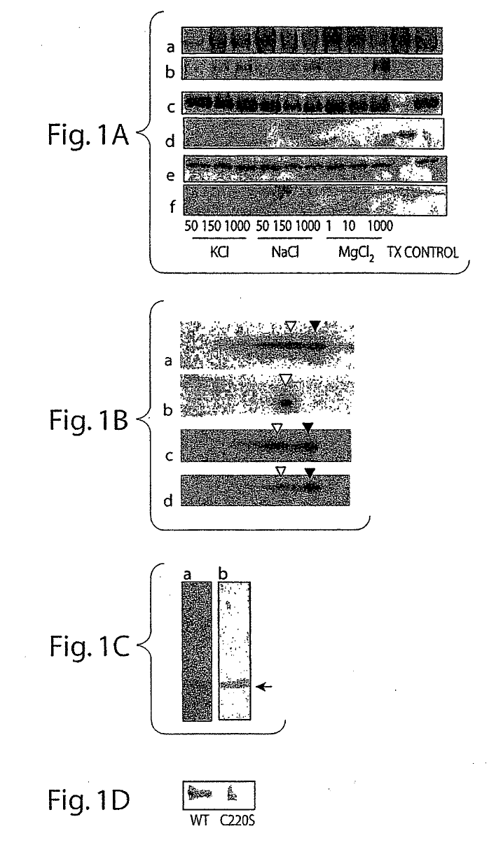

The results are shown in FIG. 1, panel (A): Effects of various amount of salt and non-ionic detergent on the dissociations of synapsin I, synaphysin and UCH-L1 from SV was analyzed by treating aliquots of SV fraction with either KCl, NaCl, MgCl₂, or 1% Triton X-100. Membrane fraction and soluble fraction was separated by centrifugation and each fraction was subjected to SDS-PAGE followed by Western blots. a (synapsin I), c (synaphysin) and e (UCH-L1) are from pellet, and b (synapsin I), d (synaphysin) and f (UCH-L1) are supernatant fractions. Unlike synapsin (FIG. 1, panel A, rows a and b), which is not an integral membrane protein, and like synaptophysin (rows c and d), UCH-L1 (rows e and f) could not be separated from the vesicular fraction by increasing salt concentration. Only treatment with detergent was sufficient to solubilize UCH-L1, consistent with its farnesylation.

Analysis of various fractions from SH-SY5Y neuroblastoma cells (similar results from rat brain, not shown) by two-dimensional SDS-PAGE gel electrophoresis showed two major and two minor species in the total homogenate and one species in the membrane-associated fraction (FIG. 1 panel (B): More than 2 forms of UCH-L1 were present in SH-SY5Y cell (gel a) detected using 2D electrophoretic analysis followed by Western blotting. Only one of them (open arrow) is associated with membrane (gel b). Treatment of SH-SY5Y cells with FTI-277 (gel d) results in a significant decrease in the amount of membrane bound UCH-L1 (open arrow) without affecting the amount of cytosolic UCH-L1 (close arrow) when compared to cells treated with DMSO (gel c). This species was presumably the fully processed species: farnesylated, truncated and C-terminally methylated.

Consistent with this premise, treatment of the cells with the farnesyl transferase inhibitor FTI-277 decreased the amount of the membrane-associated species. In addition, a UCH-L1-containing species was immunoprecipitated from whole cell lysate by an anti-farnesyl antibody (Calbiochem). Finally, treatment of the cells with 14C-mevalonic acid or with 3H-farnesol resulted in incorporation of radiolabel into UCH-L1 (FIG. 1, panel (C)). UCH-L1 was modified with [¹⁴C] mevalonate (gel a) and [³H] farnesol (gel b) in vivo. (b). Transfection of the C220S mutant into COS-7 cells prevented radioincorporation and eliminated the membrane-associated species (not shown). FIG. 1, panel (D), shows that WT UCH-L1 but not the C220S variant was detected in the membrane fraction of COS-7 cells transfected with either of the UCH-L1 variants).

### Example 2

### Removal of the Farnesyltation Site has No Effect on the In Vitro Enzymatic Activity or Aggregation Properties of UCH-L1

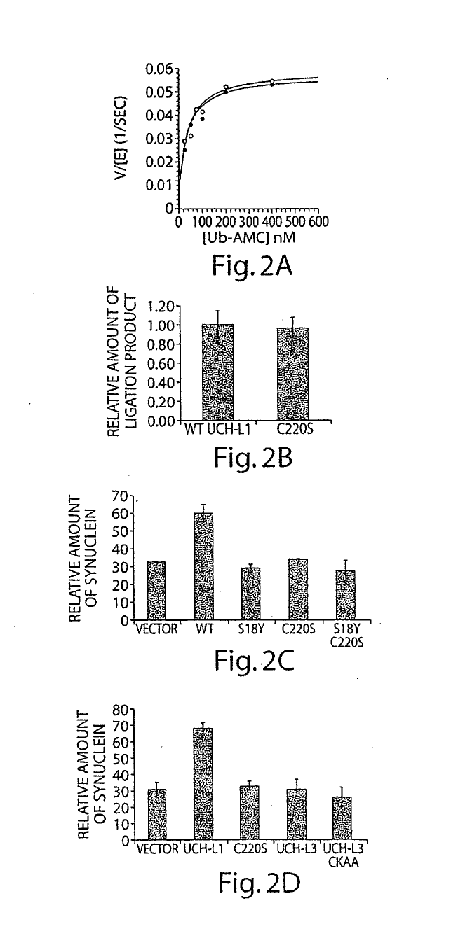

The C220S mutant as expressed in E. coli and purified using a published method. As expected from examination of structural models of UCH-L1, the point mutation had no effect on the in vitro hydrolase (FIG. 2, panel A) or ligase (panel B) activities. (A) Michaelis-Menten plot of various amount Ub-AMC titrated against either UCH-L1 WT (close circle) or C220S (open circle) showed comparable hydrolytic activities. (B) The mutation does not affect UCH-L1 in vitro ligase activity. In addition, the C220S mutation did not eliminate the propensity of S18 to oligomerize. This finding cleared the way to examine the effects of C220S in cell culture.

### Example 3

### Farnesylation and Membrane Association of UCH-L1 is Required to Promote Accumulation of α-Synuclein in COS-7 Cells

The C220S mutation eliminated the ability of S18 to promote α-synuclein accumulation in COS-7 cells but had no effect on the S18Y polymorph (FIG. 2, panel (C): the relative amount of 16 kDa α-synuclein was quantified and normalized against the amount of actin in transfected COS-7 cells with the presence of UCH-L1 variants. 100% accumulation of α-synuclein was achieved in cells treated with proteasome inhibitor lactacysteine). This finding suggested that farnesylation and membrane attachment of UCH-L1 are both required. In order to isolate the latter possibility, a mutant form of UCH-L3 was constructed in which the UCH-L1 farnesylation sequence was added to the UCH-L3 C-terminus. This protein did not cause accumulation of α-synuclein (panel (D) The relative amount of α-synuclein was compared among COS-7 cells transfected with UCH-L1 and UCH-L3 variants), although it was farnesylated and incorporated into the membrane (not shown). Thus, membrane attachment of an active hydrolase was insufficient to cause accumulation of α-synuclein.

### Example 4

### Inhibition of Farnesylation Rescues Cell Death Caused by α-Synuclein Overexpression in SH-SY5Y Cells

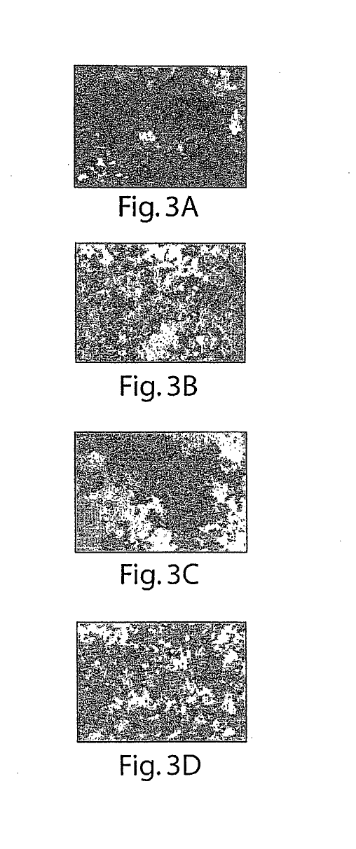

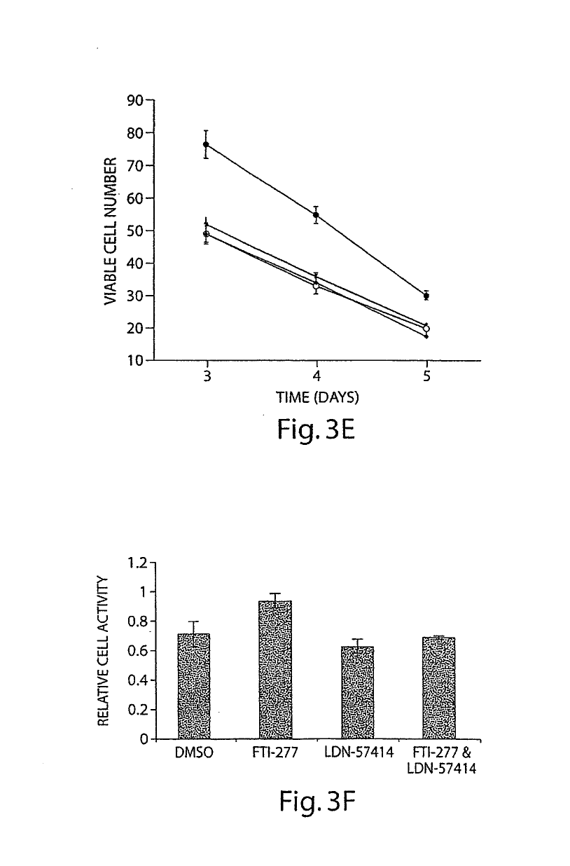

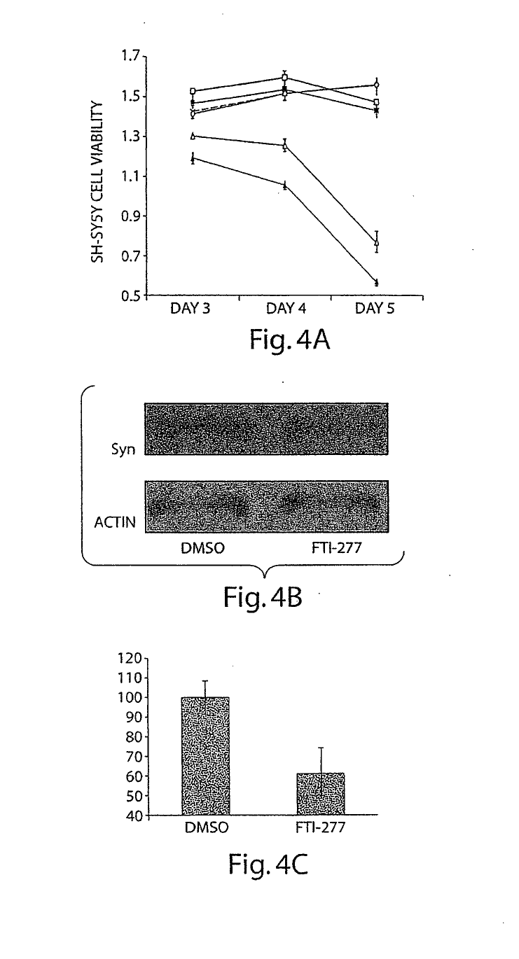

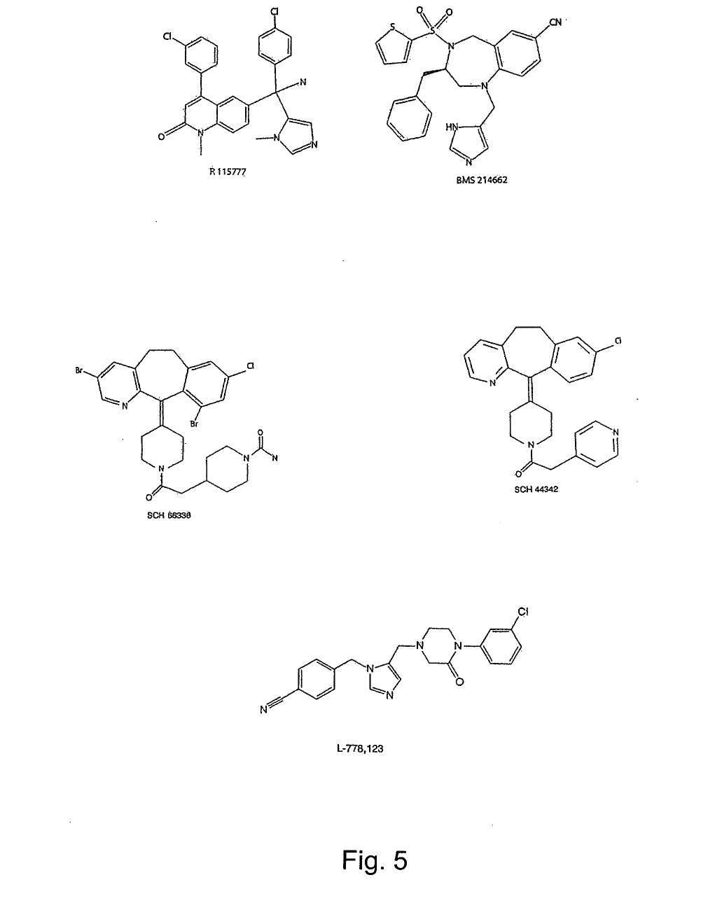

Since α-synuclein neurotoxicity is dose-dependent, it follows that accumulation of α-synuclein, caused by UCH-L1 farnesylation, should promote its toxicity. We demonstrated this to be true in mamallian neuroblastoma SH-SY5Y cells. This dopaminergic cell line has been used to demonstrate the rescue of α-synuclein toxicity by parkin, an effect that has also been demonstrated in primary dopaminergic cultures. These cells express high endogenous levels of UCH-L1. The α-synuclein gene was overexpressed (as compared to endogenous levels) via infection with an adenoviral vector and toxicity was demonstrated by the Trypan blue (FIG. 3) and MTT assays (FIG. 4). FIG. 3 shows SH-SY5Y cells infected by α-synuclein-expressing adenovirus treated with DMSO (A), FTI-277 (B), LDN57414 (C), FTI-277 and LDN57414 (D). (E) Viable cell numbers were quantified by counting the cells treated with either DMSO (lower dark circles), FTI-277 (upper dark circles), LDN57414 (light triangles) or LDN57414 and FTI-277 (dark triangles) that did not stain with trypan blue. The unit of y-axis is 10⁵/ml. (F) Cell viability was assessed by the amount of metabolic activity using MTT assay. FIG. 4 shows: (A) the viability of SH-SY5Y cells infected by α-synuclein-expressing adenovirus after treatment of DMSO (closed triangles) or FTI-277 (open triangles), and of cells infected with lacZ-expressing adenovirus after treatment of DMSO (closed circles) or FTI-277 (open circles), and of cells infected with empty adenovirus after treatment of DMSO (closed squares) or FTI-277 (open squares) were assessed using MTT assay. The effect of FTI-277 on the α-synuclein accumulation in the SH-SY5Y infected with α-synuclein-expressing adenovirus were analyzed by Western blotting (B) and the amount of α-synuclein (C) was quantified using NIH Image program and normalized against the amount of actin.

The commercially-available small molecule farnesyl transferase inhibitor FTI-277, which had previously been shown to reduce the amount of membrane-associated, farnesylated species (FIG. 1, panel B, row d), resulted in a significantly decreased loss of cells (compare FIG. 3, panel B to panel A). This neuroprotective effect was eliminated by co-administration of the small-molecule UCH-L1 inhibitor (not shown), suggesting that the FTI effect was primarily due to its effect on UCH-L1. Treatment with FTI-277 reduced the total amount of UCH-L1 in SH-SY5Y cells and increased its rate of turnover (pulse-chase experiment not shown), in addition to reducing the amount of membrane-associated protein. This treatment also reduced the amount of α-synuclein in these cells (FIG. 4, panels B and C).

The following publications describe useful farnesyl transferase inhibitor compounds, their structural and functional analogs and compositions and related synthetic methods: U.S. Pat. No. 6,545,020, U.S. Pat. No. 6,458,800, U.S. Pat. No. 6,451,812, U.S. Pat. No. 6,420,387, U.S. Pat. No. 6,187,786, U.S. Pat. No. 6,177,432, U.S. Pat. No. 6,169,096, U.S. Pat. No. 6,037,350 and U.S. Pat. No. 5,968,952 and W0 2002085364, W0 2002064142, W0 2002043733, W0 2001064252, US 2003212008, W0 2001064246, US 2003022918, W0 2001064226, US 2003027808, W0 2001064218, US 2003125326, W0 2001064217, US 2003078281, W0 2001064199, US 2003181473, W0 2001064198, US 2003050323, W0 2001064197, US 2003125268, W0 2001064196, US 2003060480, W0 2001064195, US 2003186925, W0 2001064194, US 2003100553, W0 2001062234, US 2003060450, W0 2001056552, US 2003027839, W0 2000001411, U.S. Pat. No. 6,545,020, W0 2000001386, U.S. Pat. No. 6,451,812, W0 9855124, U.S. Pat. No. 6,365,600, US 2002091138, W0 9721701, U.S. Pat. No. 6,169,096, U.S. Pat. No. 6,420,387, W0 2002024687, US 2003199547, W0 2002024686, US 2003207887, W0 2002024683, W0 2002072574, U.S. Pat. No. 6,358,961, WO 03/080058, WO 2003092671, WO 200307660, WO 2002028409, US 2002077301, WO 2001076693, WO 2001060815, US 2002052380, WO 2001060368, US 2002010184, WO 2001032149, WO 2001007437, WO 2001005430, US 2002136744, WO 2000070083, WO 2000059930, US 2003220241, WO 2000025789, WO 2000025788, U.S. Pat. No. 6,329,376, WO 2000016778, WO 2000016626, WO 2000001702, U.S. Pat. No. 6,562,823, WO 2000001691, WO 2000001678, U.S. Pat. No. 6,160,118, WO 9909985, U.S. Pat. No. 6,387,903, WO 9910525, WO 9910524, WO 9910523, U.S. Pat. No. 6,103,487, U.S. Pat. No. 5,859,012, WO 9900654, U.S. Pat. No. 6,060,038, U.S. Pat. No. 5,856,326, WO 9630343, WO 9854966, WO 9844797, WO 9745412, WO 9738664, WO 9736889, WO 9736888, U.S. Pat. No. 5,919,785, WO 9736587, WO 9630343, WO 2003041658, WO 2002085819, WO 2001072721, WO 2000042849, WO 2003076660, WO 2002080895, WO 2002072085, WO 2002056884, WO 9730992, WO 9901434, US 2003162965, US 2002169313, US 2002002162, U.S. Pat. No. 6,537,988, US 2003134846, US 2003073677, US 2003092705, U.S. Pat. No. 6,645,966, U.S. Pat. No. 6,011,029, U.S. Pat. No. 6,387,926, U.S. Pat. No. 6,602,883, U.S. Pat. No. 6,455,523; U.S. Pat. No. 5,925,757, WO 9804549, WO 2003072549, WO 2003047586, U.S. Pat. No. 6,358,968, US 20022119981, WO 9857970, WO 9857962, WO 9857948, U.S. Pat. No. 5,719,148, WO 9630363, U.S. Pat. No. 6,576,639, U.S. Pat. No. 5,874,442, U.S. Pat. No. 6,143,758, U.S. Pat. No. 6,214,828, WO 9857959, WO 9723478, US 20040006087, US 20030229099, U.S. Pat. No. 6,358,968, U.S. Pat. No. 5,939,416, US 20020119981, U.S. Pat. No. 6,576,639, U.S. Pat. No. 6,214,828, U.S. Pat. No. 5,874,442, U.S. Pat. No. 6,143,758, U.S. Pat. No. 5,696,121, U.S. Pat. No. 5,719,148, U.S. Pat. No. 5,714,609, U.S. Pat. No. 5,807,853, U.S. Pat. No. 6,365,588, US 20030055065, U.S. Pat. No. 6,242,458, and US 20020068742. The disclosures of these and all patents, patent publications, and scientific publications are incorporated by reference herein in their entirety.

Having now described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method of treating a synucleinopathic subject, the method comprising administering to a synucleinopathic subject a farnesyl transferase inhibitor, or a pharmaceutically acceptable salt form thereof, in a therapeutically effective amount.

2. The method of claim 1, wherein the synucleinopathic subject has a synucleinopathy selected from the group consisting of Parkinson's disease, diffuse Lewy body disease, and multiple system atrophy disorder.

3. The method of claim 1, wherein the synucleinopathic subject has Parkinson's disease.

4. The method of claim 1, 2, or 3, wherein the subject is a human.

5. The method of claim 4, wherein the effective amount of the farnesyl transferase inhibitor or a pharmaceutically acceptable salt form thereof comprises about 10 ng/kg of body weight to about 1000 mg/kg of body weight at a frequency of administration from once a day to once a month.

6. The method of claim 1, 2, or 3 further comprising administering to the subject an amount of one or more non-farnesyl transferase inhibitor compounds effective to treat a neurological disorder.

7. The method of claim 6, wherein each non-farnesyl transferase inhibitor compound is selected from the group consisting of dopamine agonist, DOPA decarboxylase inhibitor, dopamine precursor, monoamine oxidase blocker, cathechol O-methyl transferase inhibitor, anticholinergic, and NMDA antagonist.

8. The method of claim 6, wherein each non-farnesyl trasferase inhibitor compound is selected from the group consisting of Memantine, Aricept, and other acetylcholinesterase inhibitors.

9. An article of manufacture comprising packaging material and a farnesyl transferase inhibitor, or a pharmaceutically acceptable salt form thereof, wherein the article of manufacture further comprises a label or package insert indicating that the farnesyl transferase inhibitor can be administered to a subject for treating a synucleinopathy.

10. The article of manufacture of claim 9, wherein the synucleinopathy is selected from the group consisting of: Parkinson's disease, diffuse Lewy body disease, and multiple system atrophy disorder.

11. The article of manufacture of claim 9, wherein the synucleinopathy is Parkinson's disease.

12. The article of manufacture of claim 9, 10, or 11, further comprising one or more non-farnesyl transferase inhibitor compounds effective to treat a neurological disorder.

13. The article of manufacture of claim 12, wherein each non-farnesyl transferase inhibitor compound is selected from the group consisting of dopamine agonist, DOPA decarboxylase inhibitor, dopamine precursor, monoamine oxidase blocker, cathechol O-methyl transferase inhibitor, anticholinergic, and NMDA antagonist.

14. The article of manufacture of claim 12, wherein each non-farnesyl trasferase inhibitor compound is selected from the group consisting of Memantine, Aricept, and other acetylcholinesterase inhibitors.
